SEQUENCE LISTING (35 sequences)

SEQ ID NO: 1 - DNA, 295596 bp, Homo sapiens
  source 1..295596: mol_type = genomic DNA
aggagctggcggagggcgttcgtcctgggactgcacttgctcccgtcgggtcgcccggcttcaccggacccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggtacccgcgcccgcgccgcccgtcggggtggccgccgcgcccggcaggagggagggagggagggagggagaagggagagcctagggagctgcgggagccgcgggacgcgcgacccgagggtgcgcgcagggagcccggggcgcgcggcccagcccgggggttctgcgtgcagcccgcgctgcgttcagagtcaagttctctcgccgggcagctgaaaaaaacgtactctccacccacttaccgtccgtgcgagaggcagacccgaaagcccgggcttcctaacaaaacacacgttggaaaaccagacaaagcagcagttatttgtgggggaaaacacctccaggcaaataaacacggggcgctttgagtcacttgggaaggtctcgctcttggcatttaaagttgggggtgtttggagttagcagagctcagcagagttttatttatccttttaatgtttttgtttaatgtgctccccaaatttcctttcatctagactatttgattggaaatatgtcagctatgatgatgactttctgggaagcgattcctgtcacccgctttcccctcctccccaccccacgtcctggggctttagagagcgattgggagttgaatgggtctgatttcggagttagctggctgagtccgcgctggagcggattgctggcatgtgacttctgacagccggaaatttgtaggtgtcccgcgagtttaaaacaagccatatggaagcacaagtgcttaaaaataatctcctgccagcccagtgacaagcctgtcccacccggggagaatgccccggagtggcgtgcgggtcagccagggtctgcgcctcgcagccactgtggaaggagcgcggccggtccaggacacaggagaccactttgtgacttcaatggcgaaggttgtgtgtcctcattttaatttttttccctacaagaattgttctttctccctctcctctccctcccattttctcttgcccagtttctccttttgttttttgttttttgttttcctgatgggcctgcagagggattaggtgggcgcttctggtgaacaccttcctaggtggccacaggacaggtgtaccccggactgggtttggaagcttcagggcgccacatggctgggtcctgaattaggcatttcccaactgtacactggtatccggactggtgtccctatatctttctgccttgtaagccgtggaccagtttttgttcagtattctgtttccagggatatttatagcagaaggaaggggactaaagtgcagtttggccccagaggatactgaagggcagattctgggggtattcagtgtgcatcttcagccgccttggagaaatttagagcatcccacagccacgcagatccaagctgtctttactcaaaagacaaacaatgaacaaaacttttaaaggttggcatatttcaaattaattttacttgttttaatttagggttaaaacagagaaaaaggatttcttctgcccacctttttttttttaaatggaagaacaaagtacagcgattaagtctaattccacacaacatttaaaactgcttgatgtgaaggaaggcactggtatgatgtgaattccataaccttatgatggactccagaaaccattttcttccctatttaattttcagttcttttattgcaaattaatgctgctgaatttcaatgggcactaatgagactgctccttggtagattatttactgccttgctaataattacaaagtgaacctggtcaaatacagaggggatcgcatcttattcaaaattgttcatcatcccagtgataagtggtatcagtgtaatatgccctatcttacactttctgcattacatgatattcaaacactcttagaataataaaaaaagagacaaggaacttaaaaattaaaaaaaaaacttgcacaaatgggactctgtgtggaaattcagttttagaatgatttttcctgtgttttatttcccggattatctttcctcttttgttagaattctgcctgttattatccagcaaggaaaagaagcatctatgcaagttcttcatatggacagatattatttagtatttttcccctctcagtttttctgcttaaatgactctgggtataaaggaaaggattgattgggctcttttaggaaactttaagtttcttaagtagttctcaaaagttttggggctgaaagcagtgttttcaaactgcttgtcatgacccagagggtcatgaactcagtttagtgagtctagaatattttttaaaaggactaaaatggaaaggaatataatagaaaatatcagagtgcatggtatttcgtaaggataagttttgtttcctgaaaatctgttttaattatatgtgcttctgtgtgctgattgtgatgtaaaatgtatttcttactgtggattgaattcaaagaaaaaattagaaagctaatggcctaaaatattatatgttcagtagaaaacaaaaaattcaggcaagtggctggttgtttttacctatacaaatcaaaaggctattttgattgtcttcattttccccttataaattaggttggtgtctttagtcatttaggctaagttttactatctgattcttaacttttctattgtagaatggtgctgtcatgtggactgtcctcccgagtgtcccactggatgttcagagaatttatgtgaaggtcacgtcatttagcattgagatgctgtggttaccttcttccatttcttccataatatgcagccacatctatgtgtgaagaaatgtaatagataaaatttctctggacgcataataatgtgagaaagattgtcacatgtcccagcaaattgttattaatataaatttgttacttggcaagctgagattttgcaagatgttactcaaaatttcacaatgaaggaaacagggagtcatcttatcctgggttccttttttagatttcaaacaacttaggaactttgaataaaactaaagatgaagcttaactatatcaactatcctttttaaagttctaattaggaatttaatgctgcatgcttatttcagttttattactcagtattcttaaaagttagacgtctctcacttctccaaaaaacttggcaaatgtataaatcttttgcatcaaaatcaatgccctgctaatttgtatcctggccatctgcatattttggacaactaatttttccactggtgatcatttgaaactctttctcaactttgaatagagactgatttccaaagtgagatttaagtgactaagtttcaagtttccgatacatttttccttttacttagataacatttcagcccccttcctttctgatcttacttttttattaatttaaattgttactgattacgtgacactttgtgctggtctaagaatagtccagagtcacatattccctggtgaatgagcatattttcggatgaaaacggaatcacatcttcaatccccatttcattttcacctcctccatgtggcttgtacctgtttggaagaaagctcctgaaggataattgccacttattctaatctttctcacactcatttaatttggatccctggctaaagttgttatttacttttgtgattatacttagtctatgacattcataatttgggaaaattctcaggtttgagaattttggcggcttgggatttcttttagtttcttatagttttaaggatatgtaagacaggtgtaagaaactgccaaggggaggaaccatagatatcaggaaaaactagaaaagatgccagacttaccattaatgaatgatgagacaatagtaactttgttaagtgagattgtatatgtgaaagtggtatagaaactaaacaaacattaggtgtttttattattttactcacatgttaatatttgttttggtgctttcataggctaaaaagctgggaaataacagatttaagtggtcaggaattttgttataaatatagaatgatgattatatgaaatcttttcctgtgaaagtcaaatttaagtaaaatctttatcaccatctgcaacatttgtctgcagcctggcttaccaggttatcataaagaacatttattttacagatacattaaagaaagtcaaaaccctgattatgtgtaaacaattttacataaggaaatatatgaattttaattatatttttctaaaatccgtactcagcatgaaattaatacatcttaacccctccctgtgacttcattattatttttaatgtaactttagaagaacccagtagagagagcagcgtgctaagtgtgtttctttcttttccagacaactttgaatggagaggagcaaattagtcttttggtttaattctgtctcagtttgcttatctaaagaaaggaaaacagagtggctacacttgtttagaaccatatgcatactccagagaaagatgctctattaatccaaaaaatacagccacttgaaaccagccaaagcgaaagtgtaagggacttcatggaaaggaggcagttcaccaaagttattgaggggttttatattttaaactccgccagtgaattgacgtgtaatgtcacttacaaaaaaaaaaaaagtatgtctgagctgttcgctacttcgtctctaaaatatactcatactgatctctgaaatcccagaatttaagtgggctggaggttacgggaagcacctttataatatccttaatctcatgagggaagaaaccataattgctgaattctctgccttggataatatcaggagggactctgaagaaagttttgcagtaatcaacaatgttttaaattatgtgtatatttttagatcacctcaaaaaatataggaagcacagaatgacaactattctggtctcaactgacacaattttatgtagtttaataaagtaataatttcaagaaacgtgggcaaataagaaagagtatgactttcttacaacccgcttgtaagtgatgtggtggtggtaatgatccatgattttgatgatgacgatgatgatgaaaatgaagtttttgtctcagtttgggtaggtggtatttctggatgcctcctatggaccctggagatgttcatcctatacagaaatccaatcctttaaatctacttggctcattgttttagaattctaattccatagtctgaaaattttaataatgatattaccaataatattagaaacttattaagtacctataattgctatacaaaaaatttaaaagaacccaaaattccaagcaagactgaaaattttttgtctctcctctgaactatttagagggacaaattagtttgttcttataatatctactttaaataaatgtgccatctttaataagatagtagacttctttgtttggtaatgttctattttttggagatcctatgagttacacttgggaaaattataaaagttcacttaaagttaataaaatccattaagtaatgttcagaactagacatttccaaatgagcccttgaaaagctcaggtgggttctttttgagagttccccaaatgttgtcaaccccaggaggaatggaagacctctgcagttttgttattcagattctcatctccttctcagaagccgtagaactggccgggccctaaggtccacgctccttggttccagttctgtcttccatccttcggtcccgggctcattctgcctgttcctaaacggtggcaagttaggggccccagcagccaacttgtgcttacctggcactacttcctgggcagttttcttggctccttgacttgttgggcggcttgggatttcttttatggccctgaaagcaaaagacaatgttctcttttagtttcctgcaattaaatgatgttagaaatagtcatcttcacattggcgtacttcctctttcttctgtaggtcttttagaattttgagtccattctcatattttcttgtttcatttgctttattttctaatacatagaagtttaaactccctttaaagagtttttggcctcttttaccctattaagctttcttttttcttttctgttttagttgttccatctgtgtattctcagatatttttctttcaccttttctggtttatttctttattgacctgtctcatctgttattttaatgaaatttggaacagggctaaacagagttcctacctcagccagtataagaatataccgtaataactcagagtggtattaactagattaaaagtttcaaaaagtgatgtttttcttgtctctgaggatagaaacttcaacaaaataaagaagaaattttcaattagtagaatttctttgaaagtttgttcattcattcatttggctaccttattccaaattgagtcattcattgagggcttagactatataaagtgtggttttgttttcccagcagttcatgcaacagcattgcacctagcagctgggaagtcttatagcatgaataggtgagattctaataccagaatctcctgcatgtgtaaactaacagtgtagtcttgactgttgtctcccagtaaacttggtttcaggagttttagatccatgtgaacgtgtacaaggcatttttgctaactgtaacttcccacttaatcaacaaaaacaaaaacactcatttctgaacattcagtgcattcatgattaatcttaattacaccacaaaggtatttttcaatggtgattttgcgggagtggggtaacagtttcgaaagcaacattgtcagaaacatagttgattttaaaggttctttctggtgactttgacttctgcttttttagaagaccttacacagagttgtatttatttctcctggaatatttcaagcaattcagagtgaaagggtatacattccaatttgcgtatgagataaaatttagttacattgagaagctattttctttagttacagggaaaaaattgtagggcttttggaagcctctttgatttctaataggaggaatccctgagcactggtccaaacagaaatcatctcttcttcattgctgtatttccctcaagctcttagcaaagtgcatggcacgtgaaagcccggagaagctgttggttgaaagaatggatggtggtgggcaggaagcatcagggacatggtttgcttcagtctattggctgggagaaaggccatttaggaagggatccttagatgccactggaagaatgtgggaagtttgtgaatctctctttctcaggaacaaaagtagaaaaaggactccacacagcattccaagtacagtcggccctcattattcatggattctgtatttgcaaattcgctgacttactgacgtttatttgtaaccttcgagtcaacactcacggtgctttctcagtcctttgcagacgtgtggaatggcaaaaaaatttgagttatatgacgtatatgttcccagctgaggctgagcaaggctcacttctccttgcagccctcagactataaacaagtgtccctcttgctatctacttcgtgttatgatttttgcattttcataatccctgttgatgattttgctgtttaaaatggcccctaagcatggtcctgaagtactgtctagggattctaagacaaggctctgacgtgtcttaagagaaaatacgtgtttgataagctttattcaggcatgagttacaatgctgttggccatgagttcaatgatggtgaatcaacaggatatattaaatacagtgtttttgaacagaaaaacatataaaacaaggttatgtattaatgagttggcaaaaatgctgtgaccaaaggctcccaggaacctaccctattttcccctcaatgcaatggttcagtatttgctaattcagtgtttgaggtgactttatagaacatgagtaccatgaataatgagaatcgattctgtataatagagtgatgaaagcacaggtctgggagccagcagctatatttctattctggcgtgactcctgtgtagttgtcatcactggcaaattgcttaactgtgtgcctcagtttcctaatctgtaaaagctacatcgtttggatgatgtgaggattaaacaaattcatagatgtctagggcttataacattcctggcacataacaagtcattattttttattactacttcggaagggaattgagtactataccctgaagaaggtgagtatgggaattctctacgggtctggaatgtccctatatttgtttattttgccttcaagtgactaactttaataccctattgtgattagaagttaaacttctgcaaccaaaaggaagcaggaagctagtatttcttgaagtgcttattacatgccaggtactgtgctacaaaaacaaaacaaaacaactgtaaaaaaaacttcaaatttggctgcgtgcagctgctcatgcctgtcatcccagcactttgaggaactgaagggaggattgcttgagtccaggagttccagaccagcctgggcaacacagtgagaccctgtctctacaaaaaaacaaaaacaaaaacaaaggcactccaaatcagtaaaaattaatcaatcaataaaaagagtgaggggcattaagtattgtggactgaagcaatcccagagagggaattaattgaagctgaggtaagcagcttatggagaagctatgatgtacagagggcaaggaaggaatttttctgtaatttggaaaaatgggaactgtgagaaagaaggagttggaagctcatacttagggagcatctacaaggacgtctttttcacgttggttggaatatccaaatcaaggattatttcagaatcacccagatgattaaaaaactactgagatccaggttgtatttcagcagttctgacaattgctctgggtcgaagcttgaatcagtagttaagaaaaacaacaaacaaaacaaattttggggcttttctcactgatttacagttaaagctcatttactcttcctatgactttagatggaggatatttccaagtcttcaggatggagacatggagggaagtgagactagtgatgtgcctcaaggttttgctgttgttctaaccatgaggagcactattcaaacccaggtctgctagatttccaagtcttcatttccttgggcctcttggatttcagaagcagagggtaaaaggagtgctggggagaaagatcacagtagctttcaattctactcctcagctttccaaaataagtttcaagactggccgttgcatttgatatggaataaatacaaagaaggtagattgaagggtatgaagatgcagatttttgataccagatatgaagataacattaggaagcaatctaaaacatggacacaaacacacacctgtgccagttagcctgtataattcgatttttgttaagtgtttagataactgaaggtaatttaagccctcatatcttcccttcatagggttctttttccctctggttcatcagagagttgccaccaattcaggctgttagtggtacacataacctctagcattgttgatacagctataaaatcccaaatatcagtacaattgttgattgcataaaatttccagttgcatggttggaaagtcctgtaagtttgaatccttaaaccagtcttaaatgtggaggaggactcaattaaagctctcctcgtgtcctccctctgacgtatttgcaaaatcctttccacaaatagaatactgtttttaatgcttccccagtccaattttgcgttgtagaagacgaatttatggatgagggaagtggcattcaggcactccagcttggtatagaagcccatggtgtctggtcctcagtcctcaagcccgctcatttcctcatgtgaactcagaataagcagctgaaagcaagtcttcaaaatctcagagatatgtataaatgcaagtgtttgggtgagaagtgaacatgggtctcctctagtgcccacactacttgactaacaggttttgggctccacacaatgagggattatcaacccctgtcccagggctctctgggtcttggttctttgtttttgatgctcagcaattgtgatcagtgaaaccaatgttgcttttctatcaagagtccaacccttttctaagaagggttgtgtttgatattagggaatagctagcaaagttatcaagtaacttgtagaaacattcttttgcaagagttcttatactgaatgactgtagttgacagcagtgcagtactggtcattttctaggacatcttaaaaacactgatgagaagtttcctctcagatgtctgtcatgtcattcttgcctttctctacacagggtcagttttctcttattgctgttaggagttcctcattggtttttcagcttttgggctttcaaactctaattaatcataagctactagagtgtactacctaaagtgtgtatatacacatatatacacacacacacacacatatatatacctggtatacatatatatatataatatacatatatcatatatactccccaacctgatctggttcttcctctgcataaaagacctcaggccagtcagagaaaacatgtatgttccatggtgtggcaatcaagcccttgtatttggttccaatcagtctcctaactattactccaagaagctcttgttgaaagagccatgtttaaatggcatgttcctactttcttcttcatagtgatcttcatctgtaccatgtacccttctttcttcttgttccatctctgttaggctgattctacccagaagtcaaggttcagctcaaatgctatccctatcaggtgaattttccacctggcatttgttcggtgtgcattgtgtgcatacagcaccttttcccggtacctttactgtaatcaccagataattcctttcattttagttgtaaatagagttgtcttccccctctatggaatagattttattaatgtatagagcagcagtccccagcctctggaccatggactcgtactggtttggggcctgttaggaactgggccgcacagcaggaggtgagcagtgggcatgcaagtgatgcttcatctgtatttacagctgctccccatcgcttgcattatgcctgagctccgcctcctgtcagatcagcggtagcattagattttcataggaatgcaaaccctactgtgaactgtgtatgtgagggatctgggttcttcttatgagaatctaattcctgatgatctgtcattgtgtcccatcacccccagatgggactgtctagttgcaggaaaacaagttcagggctctcactgaatctacattatggtgagttgcataattatttcattatatgttacagtataatactaatagaaataaagtgcgcaataaatgtgatgcactggaatcatcccaaaaccatccccagttccatctgtggaaaaattgtcttccatgaaaccggtcatggaactggtgccaaaaatgttggggaccactcttataaggcatattagagtaatttcatagatttcctaattcatttatcatattcattcactcagcaagcattactggatgttgatcatgtactggctttggtggtaggtgcagagattgggaacattgtcatcaaggagtttatggttgagtgagggagatgacaagtggatagacaatgaaaaaacagtagaataagaactgtgatagaaaagagacagccaggagcattgaggagaggcacttaaccagatggaggatcttggtccattgatatggaggtcaaaatggtttaatagagcaagtgaccctttcaactgaattttttaagaatgaggatttagccagacaaagaagggcaggtgaggttgtgaagaggaactgagtggtactcttcagagctccagcccagttccttggacagaataaatgcttactaacttatagagctgaatattgaattaataaaataagggtaaactgttaagaatcagagaaataacttaaagaacactgatagctagtgttttttgaacaccatgtacccaggtgccttgccgaaaaccttaatgatcatcttgtttaaaccttacatttctcataagaggctggtactattgttattctcattttatgggacgtagaaactaagacttggagaggggaagtgacttgcccaaggtcatacaaccagtactggagaattagggattctagatctagaatttggactctggagcttaaggttttaacccacgacattatgcagagaaattgacaggatttttctgttgctgatcaatttacttggcagttagtttgttacttccttgtctttattttagttgtgacaatgctttcatcttagactgtgtcccgaggctgctgcttttatttttatgggaaatggctatttttatgatccttgctaaaagcatgtttaaacaattttccattaagtagggggatgtttttccttctaatatcagaagccaataaatgaaattctacaaagacttgctggtagcaaccttaggaatttctttgcatgtgaaacccatctgagaacttaaaatctgggtaaaattgtagtgtaatttggtgcaatcgtctctttgcacaaataacatcataaaatcatagtattgtcatctaggaggggccttagacatgatggaatcctaccttttatattttccaggtgaagaaatcaaagtctagaaaggtgaaggaacttcccccaaagtttcccagctggtagagacagaaccagggctaggtcctctattctgactcctgaccactacctcacacctaatagatggaggcatgcccagttcctgttcaccgagggcatcagaccatgccatactcattgctactgttccagcatttatagtagaagctcaagcaagcaggatgacagaatacctaattctggtcactacaacattataatgatggctaaagtgaatgccccagccatgcttgtctagacaggccatctgtttaattggtatatggttcacgtgagaatttttaacctctgtttgtcgagtcggtgttagttctctagtgatgaattatttcctatacttccatttagattatttactcttaatttaataaccatacattgtttactttggtattgaagattcccttgtttttcttctttttttctgtttccagggcttaaaggttaggagtgaccttgccagacttccctggagacttacactgtctcctttcagatttctgaagcagttgggtgctatttttagtccactatcaccaatgtgaaaatggaacttgcattatttcattatagatatttcactttagtattgacagaattaaaaaaataatttgatctgtgcttgatctagcagccaggttacaatagacatttttagttacctggtccacatgttgaaaaacatgtgtcttctctgagactaatgactaagcccgatgttggttatatactgtttactattaaattttccccttgtagtttaatattgttccaggaaatgaaatgaaagtttaataagaatggcaattgatggacccatatgtcggaagtataactaatgtccccgttacatgtgttaaagaaaggcatggctggtgggttgtaactgtactacaccaagatgatttgacacaacttattctacagagatatatatttatcaggatagaatttataactaaacaaaactatagcattttttcactttgattttttttaaatgagtcaaagaactgctagaattgtcagttaaaaaattttaaaaggagatatgaaaaaatcttacaattcacaatgctgtaaagagataatgtagggattaatatgttcttgatatcaatattttatgacttttatacatgtagaagcaaaacaatttgaggtaggtgaagttagtatggacttcttgagattgtccttcacatttcttttcctttcggtgaaaaattgaaggccaaaatgtattttcttctggttttgaaaatactgtcaagatccttgcaacaaaatgagttcctctaaggagctgaaaacaaagctcactcccctcgtgatactctgagaggctttgctcagcatcctgcattctggtgattccttggagacagatgatgctaaacacaggaagattaggtcaatggtaactttttctaagtcaatatttcttctccttgggagatgatcattttaaatcttcccgaagtccaggctaaacctttctaattgaatctccatgaaggagagctccagcaggtggagaggaagtgagaaagagaaatgaaagctgcacgcctcatgacgctgtgccagggagttcttaaaggtgagggagtttctttttggtaacctaagctatgtgaatcagaaggttcattagcttgtttctttttcttttttgtaaactcctacataattttagtaaacaggaacagtaacctaatgtgatatcccactggcccaagacttagtgcatcttcaaagttgcttaattatgtccgaaacagacttttgtctcttgatgagaaaagcatggttaaacgtgtgatgatttcctattgtcctgagctcagatctgtaattgtggccagattcatgcatctctgctgccttctcttagaagaatcatatgtaggcttgtcagataaaacaggatgcccaggtaaactggaatttcagttaaataacaaataacattttagcatgtcccatgcaatattatactaaaatattatttgttgtttatctgaaattcaaatttaattgaatgtcctgtatttttgttggttacatctggcagccctagccatgctgcctttctgcttaatgggcttaattttttgaaggctggaggttttctgttatggtgcccgtttccacctgcttttctaccaggaaaggaggcatgctgatgtagaatttgcatccttatttttgtcattattattgattataacagatgacataggtttagattaaacctacaatgacattgctgtcattcagataattgtaattattgctaattgtaaagaaggataattttttttgaaatgactattatttgttttttgtttttgtttttgtttttctttttttctaattatactttaaattctagggtacatgtgcacaatgtgcaggtttgttacatatgtatacatgtgccatgttggtgtgctgcacctattaactcatcctttacattaggtatatctcctaatgctatccctcccccctacccccaccccacgacaggtcccggagtgtgatgttccccaccctgtgtccaactgttctcattgttcaattcccacctatgagtgagaacatgcggtgtttggttttttgtccttgggatagtttgctgagaatgatggtttccagcttcatccatgtccctacaaagaacatgaactcatccttttttatggctgcatagtattccatggtgtatatgtgccacattttcttaatccagtctatcattgatggatgtttgggttggttccaagtctttgttattgtgtatagtgccacaataaacatacatgtgcatgtgtctttatagcagcatgatttataatcctttgggtatatacccagtaatgggatggctgggtcaaatggtatttctagttctagatccctgaggaatcgccacactgacttccacaatggttgaactagtttacagtcccaccaacagtgtaaaagtgttcctgtttctccacatcctctccagcacctgttgtttcctgactttttaatgattgccattctaactggtgtgagatgatatctcattgtggttttgatttgcatttctctgatggccagtgatgatgagcattttttcatgtgtctgttggctgcataaatgtcttcttttcagaagtgtctgttcatatccttcgcccacttgttgatggggttgttgttttttttcttgtaaatttgtttgagttctttgtagattctggatattagccctttatcagatgagtagattgcaaaaattttctcccattttgtaggttgcctgttcactctgacggtagtttcttttgctgtgcagaagctctttcgtttaattagatcccatttgtcaattttggcttttgttgccattgcttttggtgctttggacatgaagtccttgcccatacctatgtcctgaatggtattgcctgggttttcttctagggtttttatggttttaggtctaacatttaagaagaaggatacttaaagtataagggaaaatgttacaatgtatgaagggaacatgaagaaatagaatctggtaaaaaagagttcttgcttttgggaggccaaggcctcctggctaacatgatgaaacctcatctctactaaaaatacaaaaaattagccgggcgtggtggcacacgcctgcagtcccagctgcttgggaggctgaggcaggagaaccacttgaacccaggaggtgtaggttgcagtgagccaagcttgcaccactgcactccaggctgggcaacagagcgagactccatctcaaaaaaaaaaagaaaaaaaagagttcttgctttcaaaactatggattaggtaacttttgtgaatgagtaagatcatgagtattataaaaatagcacctttcttttttgtcttggggaaattatcttattttttaattggatttcagaaaagagtatttcagagaaataaatctctgaaatgctttttgaagtgtgaaagatttagaagacaaaagcaaacctcctgtctagataaacattaaagagatctgccctcccctcctctacctattcaggttgcaacactttgggggtggctgccttggtagagcttgatcgtgactctggtggcttgggagatggcatgctgcacaagggattcatggttacagcgggcttgtgggactggggctctccaatacgtggttgggtttgtaaagaaatcagagctatggtgtgaacaaaaggatatgcatgggagacagtgagacaaggaaatgctccagaaattattggaatataggtcagataactaactgtacttgtgccattttctgggggaaaattctctgaaggctttttgggaaaagaatggaagtgagaattctcaggtcctcaaaatatttccttttactcagtcctaacctgaggccgttaaagaattcccagagtcacgatggaaggcatgtttgggagtaagagccagagtgagggttagaaatgtgttgttggccaggtatggtggatcatgcctgtaatcccagcactttgggaggccaaggcaggtggaccacctgaggtcaggagtttgagaccagcctggccaaaatggagaaacctcgtctctaccaaaaatacaaaaattagccaagtgtggtgacacgtgcctgtaatcgagctcttcgggaggctgagacaggagaatcacttggacccaggaggtggaggttgcagtgagccaagatcatgccactgcactccagcctgggtggcagagcaagactccatctcaaaaaaaaaaaaaaaaaaaaagaaagaaatgtgttttccagggttctgggtacttaggaatttggttgcttttgcaggtggaagtggaggtgactaggtaacagctgagtgattttgccccagttggacatgagccaggttgagcagaaagccctgggatgcggggaggggggtggcggggaaggaattgaaagttggttgtgtggtttggctttggcttcatggcatgctcacaccttgcttcgcatagcatgcttagactacagcaggagcatcaggaagtggatttctgagctcaatacaaaaagttataaataccacctataagggcaataaagatatatagttgattttcttctttgcaaggccaaatcttataggaacataagagcgaatgagttacagcctgggaatttgagccttatattcagagattttaggttgcttctgattccgctgtctagacaaaaccatgagaggatagtgtctagaaatgagaggaagctcttccaatgcagaggctagaatgtgtcagcctgtgctgcgaggcctgggatagatgtttctgaaaagtaaaagggcagctttcctactggatacttgatcctcaggctctagaaaactctgctttattaactttgttgacttcctaggcaccacatgggatccttgttcttcctccttgtaagcagtaattgaaatcagtttggcagcctggtttacagtgaccatggtggcttgtctcccgtgctcttacctcactctgttgatgttgtaaaacctccagctaacttcatggggtggctgacccacgttgctcatttattcattcaacacatattcattgaccatctactctatgccaggtattgttatcagcactgggaatagatcagtgaactattgatctatttgtctaatgggacaaattgacaaattgggaaagattccattacacaggtgacatttaagcaaagtcttgaataagggagggaatagtaccatgagatatcctggtgaaaagcaatttaggctgagggcacagcagggaagaggccctgatgtgggaacatccctggtgtcttgaggtacagaggccagcatggctggcacggagtaagaagttggaggtgccgggcatggtgactcacacctgtaatcccagcactttgggtggctgaggcagatgggtcacctgagcccaggagcttgagaccagcctgggcaacatggtgagaccccatctctacaaaaaaatacaaagaaaattagccagatgtggtagcatgcatctgtagtcccaattgcttgggaggctgagatgggaggatcaaattacttgggaggctgagatgggaggatcacttgagtccaggaggtggaggttgcagtgagctgagatcatgtcagggtgacagagcgagaccctgtctcaaaaaaaaaaaaaaagaaaaagaaaaaagaaaaaaaaagaagttggaggtgagtaaggagaggaacgtgggggacagagtcctcaggactctggcttttactctgagtgagtcgaaaatccaattaaaggtttgaaagagaggaatgacctgatctgacattttattgtgaacgttttcaaatctttacagaagtggaagagcataacgatccttcatgtacacatcgcccagcttcaactatgatgtttcatttgtaaatatttccgtctacacttccaaaggatgatgactatttttaaaagtccaactataataccattatattttaaaagttaaaacactatgtctttaaatatcaagagtttgtattgattcgcactttgaaggtcgagctgatgaaatttcctgaggggttggatgtgacatgagagaggagtcaagtattgcatggtaattaaaaacctttgcagcatagtccatttaccgaaagactatatgtatgcacttcaaagcaggttttaaagattaacatcaagcatctggcttcatgagttttaacttcttttcataaatgttatacaatgtcatcatctctccagctagagaaaatgctattattcttattttcaaatgaggaaaatgacgcagaattatttacatattatgtaacttggtcccaagtcccttagatactggtttagaaaatcctagtaaactggaagtgacttatccaaaattaaaatttattttgctctattgtcttttgttgcctatgggaactttgtgcaggtaactaggcacatgtcaggactgatttactgacctctcaaggtatctttaattattttgggggatatcacggaatgagttctacacaattcatttgaatcgaattgaacttaagaaaattcaaatgatgcattggctgcctcctatttattacatgctgctcataggcataacagcatagtctaacaagtataaaacctgtgtaactgtagctttcagtgcagtgtgatgagggctgagaagatagtggtacaaagaagagaggtagcagagtgaagctgagtcaatatgatgaagatttctctagacttgaaagggctagaaaaggttattcttggcaggaaaaaaacatgagccaaggcataaggataagcacaggcatggcagatttgggaatgtcatgtaatttgttgctgggctgcaaagtacatggaaggggagtgaaggaacagaaggagatgaatctggagggagaggttaaagtgttccagagagcaatatgtaggtgttactctaagtcaaagaggtcgtaatagcatgtccagactccaaaactctaaacaagtcatagaattgctgccttggtagggcatatcacacacatcaacccaatcctctgtcaccatgacatccatataactgcaactctatacatttcccagcctatgttcccagagtctccagatgacattgtctgcaaactgcactgcagaaggctctgctatgtcttcttaaaagtaagcaagactgttttcctttgttacatgagcagcaaaaggatagggtgctctttgacctcacttactgtagggtggataggaaagtcaaggaagagtaacccagaagatttagttttaactttcgcatcaaagaggtcccttagcatctgctcagagatgtcacaatttctggtgtgtgattatgtttaagaattcggccttgccactgttgaagttgttctgtggaaaaagaacctctcttaattttacatgatgcccaacttctcttttattccagaatcactcatatgctgttggactctttccagccatgtgtgctaacctaggcaatgtcataatagatgaattatgtttactttgtctttgatatctcagctcttttatcttctattcaagttcccacctccatcattactgatagtgttcgttgaacaaagaatatgtcagatatacagaagtgtttctccccttttctctgtctctcttttccttcctttcctttagtttcctttctctgtctgttttctgatgcctcattttagaaaagtgattttttttgtgggaaaatcattttagcattagaaacgcaatggctatcactgacagcttcctctgatgaaacggccatttgtcatcattacacggtcatgggagtgctaagaagacttaaatgcagggctaccaccccttcccaattcatcttttatccattttatttctctaaggaaagggtttgaaaaatgggctttgccctcttggatgcagtgaagaaattctagctggctacagatgttattgttggtcggaggcaagggataaaatcatggtcacaccattgtagcgccagatggggaatgtagcaaacatagttgtaatttctcattttacagatgaagaaactgaggtgcagaggggttcggtgacttgtttaaggcatgtaattgttattggcagctttctgttcagaacttaagagtatgagtcagtctagatcttttcatcacaatactctgctcctcttactttttcctgaaatttgtcacattgacagcaatgtgatccctaatgacacacagattcccaaataatttttgtagtaaaaatttccatttgcaattctggacatgtgtgtgtgtggaattttatgtgatgacatattggtcctatcttttgaataggatcataaatgaaatgacttatggatcacattcaaaagcaggccaggggccaatgtgtaagcaggtgggttttcatatttggagttctgtacttttgtgttagtcagtgggtctaggactcttgtagtgtatttcccaagggccaaagtcttctgccttgaggtgtcagctttccaaggcagaggctggatgctttctcttccttctgggctcctttctcttaggcttcccccttctcttctcctccatttgtatctgtcctttttctcggtactttccctggctggtctcagctagatgctcactcaatgctgttgaataaatgaatgaatttcgtagtaattctgcaggtaaatcaagttattgtctcccaatacggtgctatgctttctgaggaaattagactggaagtcaggctttttaaaaaagaagatgtggtgtcaaattgcagctctctctctctctcgacttaccttttttctatcatccatatgccttctttcttgtatctttgggttcccagacctcaccattcattagcacttggaatggattggtaagaataaagaaaggagaggtggtgaaactcagcttgggtcatctggttacacattagtaactgacaaaagataaaaagatacagactaaatgggtttttagggaactttttccagtctattcttgtttcccattagtgtgaaaaatcaacacttgcttgtattttggggtgaagacatttttcttaagtgagtgggaaagcctcttgacattttaccgagagccttaaattttgaatggtgaatgctaatgttctttgtgcataaagaatttcagaacttgtatatatgagcattaatgatgcatcattttctatttgtgagttaaactaggtattatctgtaatcatatttttaggaaacattcaaactttcatcaagtcattctcttatatgactctcagctccattaactctgttttcatggaactcaacagagttcttaacgtttgcattataaattaaattagcatttcccctcaaagaagtattgctgtccttacaataaataattgtagacaatttcttttcttttcttttttttttttttgagacaggttctctctctgtcacccatgctggagtgcagtggcacagtcacagctcactgcagccttgacctcctgggctcaagcaatcttcccacctcaacctcctgagtagctagaattataggtgcacaccagacctggctaatgtttaaattttttgtagagttggggtcttgctatgttgcccaggctggtctctaactcttgggctgaagcattcctcccaccgcagccttccagagcagtgagattacaggtgtgagctaccatgcccagctaattgcaggtgatttctaatgggatttagtatttctgggtttaaggatgagatctgaggtaatgactttgtttccagatgtgaaataatttgctcttgggttgtgagccctttgggtgggctcccaaggatcctgctctcttccaggagcccaggctctggggtcagactgcctgggtccttgactccctgttttctgattgtacaactttggtgagtggcctaattcctctgtgccttggctaccttggttactatttctaaaacaactggtgttgtagtagtactgcttagagtactttcaagggttaaatgaattaatccatgtaaaacgcttaaaatagtgcctgccacaaccatcaatttagtgtgaaaatctgctcacctgcttggccagcccctttcactttattaaaccaagggtcgtgctgggttttccagaagtctaagttgcggtctaatctttgtgcagaagctgaaatagcagccataacgttctccctagatgatttcgtggagcttctttgaactgtatctatctccagtcatttttgtggaagaaattttcttctgtactttttagggatgagaattacctgccttggtttattaactaaaagacaccatgattacaaataaaattaaataaatattgtatcactaaatagataatatgagatagatgtattaagttttcagataaacagtataaaagagctagagtaatttgtaaaaagttgggaggacctattttgtcatgcaggaaacaatttttaacttgcctaccccagaacatagctaccacatggttagggtttgcccaaacctggcccaggagtcatttaccttgagctttcctaaaaaggaggatcaggattttcctctccagactctatcattttaggtagagtccttcttgtcaattctttttaagaacatacatttacttttgtggaaaataaatagatacaaaataaatacatacaaaattgcatagcaattagaaatacccaggaggtatgttatggtcacagacacaaactgcctccaacttctgtccatccatagtgatatttaaagcagagagaggtacacaggtaaccacatttagatggactgggatgttgccacacatacaagcattgataactggcttctcattacctgaatacattcttctgtcagagcaacagactcagctatgcttctggcaaaattgttcttaattctctattgattaatttattcggtaagtatttattgggtattttctgtctgaaaagtgcgattccaggtgctttatgtgtctctgtgtgtgggtgttatataaatacttataatactgtatccatactcttgaaaagcttagttgggaaggcaaggcatgcaataaggaacacagaattttagtcattccacaaccatctgttgaatggctgctattgttagtatcgtggtggaaactgagaagcaaagatgactataataggatctcttttctggagatgcacagtggacacgtagttatatgatgatgataaggactccagaatagttctatacatgatgctctggggccacatgcagattctgatgagaaacaattaactctttttggctgctacctgagaaggggtaattgtcactcaggaggtttttgccttttgaccaacatagaaaggagtgtgagtgaaggctagaggtgtactaacttggtcagggcagggtgacacataaaattaaccatcacagggaagggtagggctggagaggcagactgtggccaggttacaatgcgctgaggctaaggagactgtgtttatcctgtaggccagtgggtcttactctgaagtcttttgggtgggacattcatggacttcaagagacctgtgaatgccctaagattataagtaaaatctgtgagtctgtaactaaagctaaagctatttttctggggcccaccatctaaagaagattctgaagccttagggtagccgtggaggagacatgaaggtccattttgcatggtagaaccctgcctggctcttgctgcagtgtgggaggacaggtttgcaatgtggaggtgtggcaggcatggatttgggaggattggcagaggactcaccatgtccatacactcactgagatggcaaatatttattaatcatccaactgtgtatcagacactaagaataagctgggaggccatggcaagtgaggtcaccacagtccctgccacagtggaggttatggtatacaggtaaggcagggaagagcactgcaaagggtttgcccattgcatcagtcatttatttatgcacatgttgattcaacaattatttctatgccaagctgtcttcaaggtgctggaggaaatgaagcgtacatttcactggggaagacagacaataagtaaacacattaaaatctggcttggcttgatgttggggaggggtgagtgccatagagaaaacaaaccatttatgcagccaacaaacatatgaaaaaaatctcatcatcactggccattagagaaatgcaaatcaaaaccacaatgatataccatctcacgccagttagaatggtgatcattaaaaagtcaggaaacaacagatgctggagaggatgtggagaaataggaacacttttacactgttggtgggagtgtaaattagttcagccattgtggaagacagtgtgatgatccctcaaggatctagaaccagaaataccatttggcccagcaatcccattactggctatatacctaaaggattataaatcattctactagaaagacacatgcacacgtatgtttattgcagcattgttcacaatagcaaagacttggaaccaacccaaatgcccatcaatgatagactggataaagaaaatgtggcacatatacaccatggaatactatgcagacataaaaaaggatgaagtaatgtcctttgcagggacatgggtgaagctggaaaccatcattctcagcaaactaacacaggaacagaaaaccacacactgcatgttctcactggtaagtggaaattgaacaatgagaacacatggacacagggacgggaacattacacacctggggtctatcagggggttgggggctaagggagtgatagcattaggagaaataccaaatgtagatgacgggctgatgggtgcagcaaaccaccatggcacgtgtatacctatgtaacaaacttgcacattctgcacatgtatcccagaacttaaagtataattaaaaaaaaaagaaaagaaaacaaaccagtgtaagaggatggaaagtaataggctcgtttagaatggtgtgagaaagccaggcagggagaaggcgctgagacagggaggtcctggatgtgtttgtggaagagctgtggcagcacctggaacttggggagcaagggaaggagtgtgggcaggcaagggtgagggtgcagggggtcatgctgggccttccaggtcacggaaggacttgagctttactcttgttgtggtgagaagctgctgagggcttggagttaggggagtgaaaagatctctactataatagggagagttcgggatctgtaacttaaccccaggagccagcaaagctccctggaggaaatgcagtttaagctgagaatgggaggataaacaggtgtttttcagagaagaggaagggtgctctaggcacagagaacaacatgctggaatgcttctactagatcataggggcaaaatgggagtgcaggagtaggagagggctttctgggaaagatacttattttaattttgcatgcattgagtttttgaggtttctttggtttgttcatgtggaggtgcagagtgggtatttagcacataggtctgaagtccaggggaggggtgtgggacagcagttggatgtggcagagattccacaaagagcaaatatcatctgagaatggcagagggctgagggcagagccctgaggaacactggtgtttaggagcctgctggagaaagaaaatactgcaaagggaacggaagtggagtggttgccagacatagaagctagtgtctaactagatgtcatgagatgtggggaaggtgttacgtatctaagaatgcaaagttgaacccctgtgaactgtaatacttaagataagtcgtataaattgtctggaactagagcttgattttccaggagagatgaaatgtgtgtaggtgacaggaaacaatgaatatgtgggcgagtgtagtgtgagcaatttctcagaggtgaatttgacagcattttgcttaggaagctacaaagagaccaatgctagttggtgcaaggaattcaagaatttggacttaagtctatataatgatgattttttttttttaacttgagtttcccggtttatcactcccagaatataggcagaagtttgagatttttatgtgtattttctggaaaagatagtttcagtgttttttacattctcaaacaggtttatgatccaaagaaaaggcagtggtcacagatacatgaaacgacaaggtattcaaaggagaacgttgtactttatgacagttctttgggcagtggcttgcaggatgagtttgaggaatgattggaggcaggagagtaattctagtaattcaaatgtggagtattgttgatctctcagacacaaatggaaaaacaaggaattcaaagaaagataggcagagtgttttgaagaaataattgatgaaatttggtaatgagttagatgtaggagatatatttagcaaatatttattaaggactgtattaatctgttatcatgctgctaataaagacataccaagactgggtaaattataaagaaaaagagatttaatggactcacagtgccacgtggttggggaggcctcacaatcatggcataaagcaaaggaggaacaaagtcacgtcttacatggcaatagagtgtgtgcaagggaactgccatttataaaaccatcagatttcatgagaaatattcactatcatgagaacagcacagacaaaagcctgccaccatgatttaattacctcccactgagttcccccaggacacatggaattatggaagctacaattcaagataagatttaggtggggatacagccaaaccatatcaaggacctactgtatatggttaaaattgggagcaaatgagacatgattcttgccttcttggagtttactgtttactaggggaacatacacttgtcaataatcacccaaatataggattggaaattgtggtaagtgccatgaaaaacaagtatagggaattttgagtgtacatagctttggggacttgatttgatgagggagccttatgaagttattgcactagaactgaattaaaccacatttctaggaagtggacatctatttgttggttctttaaatttagctttacagaaatatttcctttaaaaaccaaggcttcttaaatttttaaaactgcttggctaatcaggggaataatgcttttggatagctggtatcgttatttatggttggaaaaacaacagtatttgattacattgagctttaaacttttcctttgattaatgaaaattttattggcccatagtttttattatgctctgtttttacttggtccaagagattctattctctggacccaatatgaataccttcagacatccctctttttttttttttttttcacccaggctggagtgcactggcacgatctaggctcactgcaacctctgcctcctgtgttcaagcaattctctgcctcagcctcccgagtagctgggattacaggcacctgccaccacacctggcttatttttgtatttttaccagagatggggtttcaccatctcggccaggctggtcttgaactcctgacctcatgattcacccaccttggtctcctaaagtgctgggattacaggcatgagccaccacacccagcccagacatccctcttaattatgttgaatatgtaatatcggtgatttcatttgaaaatatttagtagtcgaactagatcaaggcagttaagcttcctatttccatagatgcagtggtattgtgtcttttttatatgatctctcatgcttctggacatccttttttctgctattcttcattccttagctacacttggtgcttcgtggttgtaatgcattgtcatagatgcgttcatttctcattcgatcttcagctctatttctttccagagaatctctacaggcatctgttaggttgaaggacatctaatgtcttaatgtgtagcttggtaaaccagtcaactttctatctgagtcttaagagaaagtgtccaagatgagaaacggtacaggtttggtgacaactcagtgagaaaaagaagaattttacaaggaaggaggtatcttagtaattttgctaaagaagtaggtaaaccttcacttataataaagggatagggctcggttagggtttgtgaagtctccccttaggaaagcaaaccctgaaatattttgaatcttttaaagaaggaaaataagagtcttttaaataaatttttaaaatttattttatatattttttatagacaggctctcactctgtctcccaggctggaatgcagtggtgcaatcatagctcactgcagccttgaatgcctgggctcaagcggtccttctgtcccagcctcctgagtagctgggactgcaggcatgagccaatgtgcccagcaagagacattcattttggtactgtgatggtacagaaaaacaaagggcctttgaggccgaaggagcagaagaaggatggacttagacatggtataggcactttctactaaagagctgtgaagctaaaaatgccaggtctatgacaggtgcagtgggccaaggccaggtagagagcagcaggaagagaggaggtggggacctgtacctaggcccatctgctgggactgatctagccataggtactcagagaagcccagattggtgcctgacccacccttatggcccagacatggacacctcccagtctgttccttcctgctgcccatggatgggctgtgttagtctgtattctgaggacacagctctctgtctagaggaagttatgttatcttgatctgatggatactcaacgtgaacattatttcaacgtgccacagggtcttggagcccagaggaagaccgctcttgccttttagtttatattctttgtttttttttaaataacattttgacagtctttatggagtaagtctgggccaaaatgataattgacaatgttatttacatggatttctaagttggctaaaaaagttcctttatggttagtgaatatagcccatgtagtttccccgtcttctttagatgccttctatttctatgcccaaagtctgcagttgattttcagtaagctgggggtcatcttagagataaaatgtagatgaatggcattttgctgacagcatacatctttgctatttctgaggaaaatgggctctcgctattaaatcttttgtcaatatttataaaaatagtatttacatattctatctatattgtggaaactatacatttattgattcagtcatttgatatcaatgttgttgagtccctattccaagtgaggcactatgctctaagcacatggcattttaaagatgaataagacaccaagaactttgcagatagtaatggaaatgagaattaatcaattgaagattaatatagtaagtagcagaagagaaataaaaaaatcttctagagagttcagaacagggatgttgattcaagtttatggggattaggagtggctggtaagggaggcattcaggcaaaagacataaaaatgcagtattcccctcgcactcattaggatggctactatattagaaaaagaagagagtaagtgttggagaggatatagagcaaatagaaaccttgtgccttgttcatgagaatgtaaaatggtgcagccactgtggaaaacactggtgattcctcaaaaaatcaaaatagaattatcatatgatccagtaattctacttctgggtatatatctaaaagaattaaaaatctgggtcttgaagaaatatttgtatactcatagttatagcaacattattcataatagccaaaaagtagaagcaatccagatgtctatagatggatgaatgggtaaacaaagtctgtgtagtatatacagacaatggcatattagtcacatcatggaccttcaggacattatcctaagtgaaatatgctagacacaaaaagcaaaagtagggtttcacttaatgaggtatctagaattgccacattcacagagaacaaaagtagattggtggctgctaggggataggggaaggagaaaatggggaattattgttgaatgggtatggagtttcagttttgtgaaatgaaaatgttctgaagactggttgcacgatgatgtgagtatatctaacatgattgaattgatgaacacttaagcgtggttacgatggtaaattttgtgttatatatatcttaccacaatttaaaaaatatagcattttattatgtaggcgtgggtgggaagatacttgacacattggaacttctggccatgcgtatactgttcactcacttattccttcattcattcaacaaacatgtattgaatgcttgctatgtgctgggcactgagctagatataacaattaataaggcttataagacattgaatctatcaatttcatgcttgctaaatatctactcccacctccaaaggcactaagcttctacagttagatattcatagctgcttcctactgacttgaatcatgcataggatattagtaaacaagcaataaaaagatttgaggttgatgggggtgggttcaacagcatggtggtgaaatggaaagagatgggtaacagaatatgaactagaattgaaaactgtgagccagtgctctctaatgaacattaaaaaataaagaattcctatttgaggctgccaacctcagaactaagttatttagaatggacgaaattggcaaagtcagacgtactcaacccaaggagccaatattttgtgaatattatggcaaatgtagtttgagaaccactaccacaaaattgtgaaccataataatgactgagaaggcagggagaggttatacaatttgggctaaaaggaaagacagggcttgtgaaggggagcgccagtgaaagtcagtgtggttcgggtatttgggtggggactggaagcaggaagcttgagcttcctttgccaagagaccctgctggaagggctatcatcaattgactttagctcatcttaggattttcattttttaaaaaatgttcacaggaaccttcactccatctatactttcaatgtctgcctacctttctttcttatacaactttgaacactctctccattcatttaaatatattatggagtgccaactacatgccaggtactgtgctgggctcttattccacctttatttgattgcacatgcctgccaagtcctgggccaatataacatctactcctatgtctggtctggcgagagatgcaaactcatcttcctctactttccttacctccttccttccagtcttcttcaagttgtcttcattgaggcaatttcttttacctgtgtttttaatcccaactcctctagtttccttcttggctttattcttttatcttcctctttgtgctttcaaacattccctttctcctggcccatgcccttcagtctacacgaggccttctcaagtctcttcattctaaaaaattcattttcttgggtcttatattcttcagctgccaccctatctgtatcttttcctattctcctccaagttctcaaaggaatgccttccctcattttcatctccttacattccatctgctgaattttggcttgtgcctgtacctgtctaaggaaactccttgctaagagtctgctttgtcaggtctgaattcacttaaccagtctttgctttgttggacttctctgccccatttgccattcttgatcatcctctccataaacctttctacttaaagcattttacttccttattttcttggttttcctagaatctccttactgttcattttcagcttcctttctgtgttcctcttctcttcctacatttttttttagctttctactttcttaaagcattttacttccttattttcttggttttcctagaattttcttactgttcattttcagtttcctttctgtgttcctctgattgtctctctttctacattttttttttctgtgttcctctgattttcacgcagtctggagttgtcatgatcaatcatagcctactgcagcctcgacatcctaggctcaagtgattctcccacctcagccttacaagtagctaggactacagtcacacatcaccattctcagctaatttttttaagaagcatttttatagagatggagtcttgctatattgtgcaggctgggctcaaactacagggcttaaacaattctcctgctttggcctcccaaagtgctgggattccaggcatgaaccaccatgctcagtctctacatgttcctaaagaggagttttgaatattgaagaacagtattttcaaattacattattcaagttataaaaactgatatccagggttatgtggcaatgacgtaaaaatttgaattgttatttttttgacacatgttctgtgttgtccatcagttcatctgagttccaaatgtcccagctgttttatgctttgtctctgtttcccagagaccctgagtgtggtctagagttgggatgagcattggtctctaatggttctgaaataattgtatattcctgcaaaaacattaagtctattagaaaccagctaatttcattttgtcatttttataggtaacatattctggtgcaggtagtatgtttttaaaacaagtttgcaataaacaatttcccctcaaggttaatataataggcaacaccttttgctgcaacagacggcaagaggtaatgaaagattagcttacattatgattcattatttcaaaatgtcaggataaagtggatctgctgcatctcccagagagtgcatgttttgcttttctaatgttaatggatttactgtttttttccccccaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggtaatagtgtgttgaaaacgacttctatttttgatcctatgagcagatcctaagagccaaagcgactgaggaaggaagacatagaatcagccatttgtacaaaacatgaatccctagtaggtccactagtatctttggtagaaacatggagaagagacaggatctcaggagaaggagttgacacatggcagggcagctgaggctgagtaattccgcttccttcctttggcaagactcaatcagtcttgagcaactctacagaagaattccactagctggatctctgaggaaaaaagaaatgttgtctgtgccctgactggggaatgccagatggacattcatgtttggtaggcaactttgcctatatgatctggtatatgctgttaattgtccatgcataattatctctctactcaggccttgtccaggcaaatattctgttttgttctagtttagcttgttctcccctttctctcttccatctctttcttgtctcaatggatgacaggatattttgctatgagctgactcagtggttggtgtcttgtaatggggagatatcatctttatcaaacagttattaagtatctacctgtagcatttcattttcccgcctgcctccattgttttcttgtctatagtttgccaattatagctaatatacggagagctatactttatttctactccagaaatgtctctattattgcattataataggataccctggggaaacactaatcatttttactacctaaaatacctatgctgaatatcctttatctgataggaacagagatctgacagcagcttaggctaaccaaattcattttttatcttaagtgtggggcatttttctctcttcttattctttaccttttcagcttaagtgaaggttagtataaacactaagaatatttctgatggagttttcatgtgattccttctacaaaaacccagatttaagtaacttgttgaaaaccagagtccgctaagttaataaacactgattgaagaagtgattctcatggactttctgtgatagctctttcctgccctgatatgagatgaaagctgggggatggtatatagtatttatttttccttccgttgccagtgggactttttttttttttttaaaagctgttcatatcttaatcgagtagcatgtgaggtcaacatggtctattttaaaagcattttcttcgacacattgcttttaacatcttttagaactctgctgtgagacacatggacttttttgttggtatttttatacaattaatgatattctcaatagtaatctttgtgtgtgtatatatatagaaataaattctaaatgtaagttaatatatttattatttttctaaacatatataaatatatatatgcacacaggctatttaattttattagatgatgctattttaattcagaaaaaaatgacatttatattttgatttaggttagtataagcccttagaggtgttttgacaactctcttaatttgtggttttactgtttatttgattttatataatctaaaataccattgtttttaccaagcatttaatttggcagtgaaagagcgtctgacagaggtatggttagtagataggtctaactgcacaactggatggattgagctgagactgtttcctcatcagtaaaaatgatttgaagcagtggttggcaaagtttttctgtaaagggccagataataatattttaggctttacaagggccatgcagtctctgttgcagctaccgaactggattatagcctgtaaggtgacctgtaaacacatggaagtgattatgtgctaataaaactttatttatcagaataggtaacagatcagccctggcccgtggccgatccctgatttaatgtttatttatctgatctaaatacctttatttatggaagggaataggggatttttaaatctaaagttttgattattcacattttactgagaacttactctatacctgattagatgttccgagagaaataaaaaaaaagtgtaagacataatccataataccacaaaatttaaaatgtatttaggaaatttatttgaggaagtaaatgtacttgttctcatgatacaatcagaaagtaagtcagtattgataaagtgttacctgtatgagaaagataaggaaaacaatagagagatgtaagaaatgaaaataccagttataaattaaaattattaagattgaaagtggaaatgatcttcctccgagaaacaatggcaatattctcacaaattttttacatcatttttgttcagcatttaagataaaattatataaattcccataacatttagtattgtctctaagcattaagaacagaaaaaacagaaggaaaatatatttctaaaaatcaacgaatacagtgtgagatgtttcattggtatggcattatctcaagttcaaacattttgaaaaatgtctgcttactctttgatagttaaaaacaagtatctcagctggcgtggtggctcaggcctgtaaccccagcagtttgggaggctgaggcgagtggatcacaaggtcaggagatcgagaccatcctggccaacatggtgaaaccccatctctactaaaaatatgaaaattagctgagcgtggtggtgcacacctgtagtcccagctacttgggaggctgaggcaggataattgcttgaacctgggaggcagaggttgcagtgagctgagatcatgccactgccgtccagcctggtgacagagtgagactccatctcaaaaaacaaacaaaacaacaccaccaccactaacaaaaacctcttatcgccgtcttgtatacgcagaccagctagtagaattttactgaaacagtagcctataaaaatgcaattccacttggtttcagaaacttcttgtgtatcatagtgtgaagtcacttatcttaggcttttaaaatgggataaatattgagtccaaagttctggaagaagcctagaaagaaggcagagttattaacttttagatatagggaggaaccttaaaattattcagttcttcattcattcacttattcattgactagctttactaacaaagccctatgcaagaccctggaaatgcaatgatagaaaaacctggtccctaccctcacagaacttgtgaggtaaagggggatacagactgataaaccagcaattagatgatggtgtcaagatagaggtgaaggcagtgtcttataggatccaaactccactcagtcctggtggtggttgagtctggctatcagaggtttcctgattaaatctggagggtgagtcaagggagcatggtgaagaaggagggaatgcatgtttagccatgtgaatgagtccatgagtgaagaccaggaggaaaggcagagcgcggggaattctatgcgtaatatttaacaaaattaatgtactgttaaacaaagacatttctgggccatggatttaatcctagactgtgtaaaaaccaagtaattgatttcctttatactttaaaagcatttccatgtatttgatttgtttgtgtgtataaaagggaaataccacaacaagtttaagggtttctagttctgctttctcatcatagtcttgataacttggaactaaaaagtttttgctgaaattgtctgtgactctttataaatcacactgcccctcaaacacatttaaggatggtgaagggtctgacacgtaggtgggaagttctgaagatgccgcagctctccctgttttccttgttacttaagaagagagctagaaatgagtgtacatcagattattctcatgttctaagtgttttggttgaagaggtaaagtgtttggcttgaaagcatacaaattttcatccactacttagtgtacaaacttgattacttaagagattgagtaatggcctccagtgaaacgcattctttttaaaaagcaaagtgaaggatgctatttaagtcagaaggggcaaaattggatattttatgagtttattaatcattgcaggcatagaagtagtgttccttaagatgtgttttagacagagtccctgggatgagttatataagcagatctggttgtagcttcagcagccagatactacctttgagtattacttcaaggaaaaaggactccactgagctcactgcttctctttcattattatttcagaaggttgtgtggcgtagagggggctcaggcctacctatacaccactagctatgttgccattttatattatttctataaggtgccaacagaagctgctcatcagatcagacagacatagcccaggcaagtattgatttacagatgatctttggccaggaagacatggtatcagggtagagtctggttatgggtcaatgcagtggggaccttaggtcctacaggtataactgagagcctgatccaccaggccttagaaagcttcagggtgagacagtccagcaccctggatagctcctttaacagctgtggccggtaagcaggcacttatttgctaaagaactcaagcccatttagctggcttcatctgctttgtagagctctgttaaaaagagttcctatttctccacatcctctccagcacctgttgtttcctgactttttaatgattgccattctaactggtgtgagatgatatctcatagtggttttgatttgcatttctctgatggccagtgatgatgagcatttcttcatgtgttttttggctgcataaatgtcttcttttgagaagtgtctgttcatgtccttcgcccactttttgatggggttgtttgtttttttcttgtaaatttgtttgagttcattgtagattctggatattagccctttgtcagatgagtaggttgcgaaaattttctcccatgttgtaggttgcctgttcactctgatggtagtttcttttgctgtgcagaagctctttagtttaattagatcccatttgtcaattttggcttttgttgccattgcttttggtgttttggacatgaagtccttgcccacgcctatgtcctgaatggtaattcctaggttttcttctagggtttttatggttttagttggtgggactgtaaactagttcaaccattgtggaagtcagtgtggcgattcctcagggatctagaactagaaataccatttgacccagctatcccattactgggtatatacccaaaggactataaatcatgctgctataaagacacatgcacatgtatgtttattgcggcactattcacaatagcaaagacttggaaccaacccaaatgtccaacaatgatagactggattaagaaaatgtggcacatatacaccatggaatactatgcagccataaaaaatgatgagttcatgtcctttgtagggacatggatgaaattggaaaccatcattctcagtaaactatcgcaagaacaaaaaaccaaacaccgcatattctcactcataggtgggaattgaacaatgagatcacttggacacaggaaggggaatatcacactctggggactgtggtggggtcgggggaggggggagggatagcattgggagatatacctaatgctagatgacgagttagtgggtgcagcgcaccagcatggcacatgtatacatatgtaactaacctgcacaatgtgcacatgtacactaaaacttagagtataataaaaaaaaaaaatttaaaaaaaaaaagagatcttagttcttttgggcttggggactcactcttgttcacttaaagtggattggttcttacatttattttcatagttgtgtctggtcaccttctggcttgatgtatgcccctatgtctggaaaaggttagagaatggggtagaagtggaggctgcccgcccatcatgtaactgttttatcttttcagagatacaattgggatcctaactctttggtctccgtatttccacacttgctcttatacgattcagtttccatgcaacagcaggagagatattttagatatattaatcaggtcctatggtcttgagtttacagctctcagtgtaaatcttactctcaagagaaaaattttccttctgcagctgaaaacattcgacatattctctctcctattccttctcccccattcactgtactccaggcacaccagcttttatttttatttattttttgctattccccccaacacgccaacatgttccttcctcaggatctcagcacatgtggttccctttttccctttgcttgaaatacccagatctttacttggctggtttcttgtcattcagattctgcccaaagtcactttctcagaagtagtgttacccttccacgagtaatactactcttacttctctttacagctctcaaattcttatgaagttttcctatttattcatttgtttgattattgtctgtctgcatcaggtaccgtgcaaacttggatgtaaactcagtgatagctgatttctgttcagcattgtttgttgctatagctccagtgcttttagtcatgttgggcacgtccctactaaatagtagaatatggcgaagggtcagagtcatgatagatagccggatgtggtggctcatgcctataatcccagcactttggtaggctgagatgggcagatcacgaggtcaggagttcaagaccagcctgaccaacatggtgaaaccctgtctctactaaaaatacaaaaattagccagttgtgatggcatgtgcctgtaatcccagctactcaggagactgaggcaggataattgcttgaactcgggaggtggaggttgcagtgagctgagatcgcgacactgcactccagcctgggcgacagagtgagacttcatctcaaaaaaaaaaaaataataatgatagataaaaactgctagaggctctctgagaaggaagaaatgtcactgggttaggttggtaagagaagtgttaatgcaagtggtacatttgatataagcatgcaaataaatggtagtttggaggaaaattccagaaatgtgaaacagtattgagaaattgagtaagccagctttgagagaacacacggcttattgtcctctccttctgttttgctcctcagctctctaaagtgggcattctctagggtcctgtcttcaatctcagcttctttttcttcttcatccttctctttccacctagtttcctagggaattatatatatatatatatatatatatataattttgtatatatatacaaaattatatatatatataattttatatatatataattttatatatatataaaaaattatatatatataattttatatatatatacaaaattatatatatatgtctggtacaggctgaattgtgtctcccagaaagatatgttgaagtcctaatgctcagtgcctcagaatgtgaccttatttggaaataatgtcattccaggtgtaattagttaaggtgaagttatactgtggtgtaacccacagtatacttccagtatatacttcagtatatgggcccttaatctaatgtgactgatatctttagaataagaagaaaatttggacacaaacacagagaagagaatgtcatatgataacagagagagagagagagaagagagagagggaggagagagagggaggagagagagggaggagagagagagagagaaggagaaagaaagagaggagacagaagagaaacatctacaaagcaaggaacaccaaagattgctggtgcctaggaaactaatataataacataatatttggggcctaaagtgttacagctgcaccaatatctcccaaatcttgctcctagctcctgccccagcctgattttctcagtgactcccctgtgtttccctctgccaccagcccctccttctgcctgctgtgtgtgacagttcccttgccactccctccggcatccgtacttcaaactggggagttagttatttccattgtttcttattgcagcctctggctctgacgtttccagagctgttgcatagctctattgagtctatctccttaaatgcattttcatccacttaccattgctgagttagctgcattacctttcaccaggatgcttgcaataatttattgtttccatttgccttctccttcatctaagccgtttgcttatgtcttttttcttgttgcgagacattctgcacattgccactctattagtggtcataaagcaaatcactgatcatgtcaatacgcagtacaagatcctttaatgacctccatagcccatggaatggtccttaaacaagagttcaaggactgccacaatcttgttctagcctatctttctagtcattttaaagtcaccatttttacttgtgaaataggcactccagacacagtgaattccttgttcttctacaaatatgatgattccatttctttgctgggaatttctttccagatttacctgtaaaaattcttcgaaccctaatcaaaagtgactgttgttaagcccctgaagataatgcagaaattctctctctcctgggacctcgatattagtttattccattgtattgcatatatttgattgcccatgttctgtcccatactgactgtaaagtccttaaaggtgagggcccaatattctcagagtcactcaataaataaataaaagaataaatggaaattaggatcagtttgtgggctttagcaacacaaaaacattatactttttcaacatgggagaggtatgatgaaggagtttttttttttttttgagacagagtctcactctgtcactgaggctgcagtgcagtggcattgtgtcagctcacaacaacctccgtctcctgggttcaagcaattctcctgcctcagcctcccgagtagctaggattacaggcgtccaccaccatgcctggctagtttttatatttttagtagagacggggtttcaccatgttggccaggctggtcttgaactcctgacctcaggtgatctacccgccttggcctcccaaagtgctgggattacaggcgtgagccgccgcacccggctgatgaaagagtctttaatgcagattaatctggcagaggtatataggagggaccagagaagggaaagaatcaaagcgtgaagaccaatttggctgatattcaactagcttagatgtactaaaaatctgtactttttggtatttgtgaaatggaaagaaggggaatagaataaaggatattataatgaaaggatatacattgcttgaaggtaattaaatatgggttatccaggagataaaagagttaaagaggttcagacatagactgaatgaactgagaaatgaatgacttggtcaccaagaggaaggccagtcatcagggggtaggataagttcaattctagacatgctgcatttgagatgatagctggatgtccagatggaattatccagcagccacagaaacagaatcagctctctgcggatattccaggggtggggatttgaattaatttcctcaattaattttaaagaaacttgatgaaaagaatggtctgaatacttcttgaaggttgcacattattaataatggagaaataactctaaaaccttcctcttgattttcataataatataagcattccctgaatcttaccaaaccttgtaagaaacactcttattataaaaagtgtatgtgcaaagcccttctaaacaggaaaatgataaattagtcctacagggccaaatgcagctctctgggagcttacaattcagaaagaacatcctgctaccagcacattaagctgtacaaatagtaaactgcagaaacaaatataagcatttttatgatgtccaaacaagaaccaagcaggtgtttttttttttttttttgcagattatttatactgtggcagttcatagcctccttttcggacccagagcttgcataatccttcccttatttctacttacgtgttttactctccatcatgtgttaacatacatactgtgcaacagaaatgactatggaggctgagggcagcagagttttagtgtgtacacatatgagctgtcatgtaattttcaagtgaaagcctttgcaatgaaactttttaaaagaaagtcatggccgggtgcggtggctcatgcctataatccagcactttgggaggctgaggcaggcagatcatgaggtcaagagattgagaccatcctggccaacatgatgaaaccccgtctctactaaaaatacaaaaattagctaggcatggtggtgtgcacctgtagtcccagctactcaggaggtggaggcaggagaatggcttgaactcgagaggtggaggttgtagtgagccgagattgcaccactgcactccagcctggcgacagagtgagactcgtctcaaaaaaaaaaaaaaaaaaaaaaaaaaaaagagaaagaaagaaagaaggaaaaagaaagaaagagaaagaaaggcgggcattaacttcaggtattggtaaatttgctaggtgtttggctactgtttctcatcagagaaatagaaagacacaccatgaaagtcaaggcctgaaaacctcattccatgtaagaatgacatccccagtgttaagtgcttgttagtggttaatgcgatcctgtgaaacttagatgtgtttgtgcacacatgcacgcatatatttgaagaactcagaagagttaaatcacagcctttcaacctgtgaaatgacagtagttcttcttttttcctctccctttggctaagtcatctttatcttggagataattaagacaaaaatgcctctgacaaataaaatcagtatagaacccctatttcttggcagcttttgtggacacagctgaagctttcagaggtcttgaaaaaccatggcaacaaatgcctttgaagggtaagcaaaggttccaaatgtttttaatcgctggtgttttttctgctaccacttcaagcatttttcttcattttttgttcatctgatcaaaattaaatttccaatttccctactaagtggttctgtcctggtcatgccattgactatttccattaaagtagtagagtttgtgcccacatatggttgttaagctcatcaacaattccattagaaagctttgtttatcagtggcaataatttcccataaaaattatagataggttttaatgggcacattttcaaaaggcatcaactcgtcctcaaaattatgtgctgacactgttcttacaaccatggttcgtggcctaattccaccaaatttctctctttttcatagagaactgttgtcagtagctttatatccttttaaagagaatggtctgtatctctgatactcattcagagaaatgagtattttagacgtaggttgctaattttaagctatatactacactatgtcagcactatataggttatcttgtaacctgcttgcctggctatttggctgaaaaataacaacatgtaaggaaaatctatttcataagtctaacatttactttgtaaaacttgttctggctactctgttaattttccacttacgtgtgggttagagagcagatttgatttttttttaagcgaaagatatggcttacctgagaaaagaacatagtggggaaagcactcctattattttcctcatatttccattttcctttagcggaaataaaaagacatttcagtttttcagttgctaagaaatgaaggaaccaaagacaaaacaacttaattattaaattacaatttatttctgtaataagcactcgttctctctgttttcctggggaaagagtatgtggactttcaattttatccaaataagcatcatctttctctgattagtgtggcagtttcaaaatcatgtattaggaagtacagagtgaatgagtagagaatttctaaattagcacccaaggttgggtggctagattatgtttataaatatgaacttttgtattaagtgcaatgatttaaaagaatgcctgcatcactttagggcatttcattaagtgctgtgcacaatatttttccttatacatcataaaagataaattatagttcataaaatagtataaattcctaattattttgtgcttttgacacctcagagttactaataagggatttcgttttaaaatgatatttatttatttatttagagacgcagtctttctctgttgcccaggttggagtgcagtggtgcgatcttggctcattgcaacctctgcctcccaggttcaagcgattctcctgcctcagcctccagagtagctgggaccacaggcatgggccaccacacccaactaatttttgtatttttggtggagacggggtttcactgtgttgggcacactagtctctaactcctgacctcaagtggtcctcctgccttggcctcccaaagtgctgggattacaggcgtgagccactgtgcctggctcgttttaaaataatttaaaagtatattttgccaccactattaaccagttaagccatgatggtatattataatcaccatggagatggtttttctctttattttattttgtttgttttctgattgctagcatgctgattactcttcctattctacaagtgcctgcaggccagccccatttttgctctcttcacttcatttttcatttctccctgtttcactcttcatagcacatttgtactctgtccacacctagagacctccctgttgaagttcttcacattctcttccctagagagggttaacttgttgagctcagagacttaaacttaaaataaaatgtaacagatatgtattgaatgactattttattttagctctaggagaaatgcaaagatatatcatccatagaccctgaaatccagctaagggttctgcttatacacaaggtgaaaggttgtgataatgcacattaaacaatagaagagttaaattcagcactataagtgatgcctcaaagcagcataatgggaaaaaggggtatttcagaaaaaaatggtatgagttcagaggcaagagaaaacagaatgctctagattaaactcaaagactttatggaggaggtggtgtttcaatagatacctttccacagaaccatgaagagagttctacttaattgtaagtggcctgtgatttgtcgttagtcattgcatgctcaattctgcatgtgtgataactgtgtatttttatggcaaaatcgcaattacttttgcaccaacctaatagaatagtgtgggtttaaaaaaactatttgtaatatattcgaacatatttcttatatatatgtttcaccatcattagcagtaaatctattttgcatccagagattgaataatattttgtttctaggatgtgagtgatgtactattttaacttacttttgaagtttgacatgggagtgatattaataaaatctaaagatgctcctaaagaggagcaatagataatgtaggtatagataatgtagctattatggaggtaagtgtatttgatactttttattatgatatgaagggaaacaactagtcccaagctacaatttattaaggtaggcttagcttatacgccataatttttatgtttgcaactttcttgattttcttcctctggctttacttctatgggatatttatgaaaggatgtttttaaaaatggctagagacttttgcttctaagagttgacataaacttttccacaacatgaacaattagtagatgatacacttagatacactcttgaatttttaaaattgtggcagtaaaactgatctcagagaaggaagaaaatgctttggccagcttttatcaattatatacaacgtgtttggaagattcagttcctctcgccttctttctccctatataatactttttcttaaggttggtactattgatgcacttggtgtaaacacatgagataatagcaaaatatgaatctaggtcccagaaatgggcttctttcactctgccacctgttctgactgcatttctcctgactttctgtaaatcccggggaggagctggaagagcaaggtgtgtgctagcataactttggggatgaagtacccttctttttttctgattttgttcccacatctccttactggaccaagtaagggcatcagtgtcaaacttcctgattgaagatacatttttgccctaccatgaagggctctgggtaacatttattgcttataaaatgcttttttggtggctactttgtttctgaatataaggcaagataaaaagtttgattcaagagtttcttgtaaacactttgtgtgtgcgtgtgtattcatatatggctgcacacgtacataagtccatacataagtacagtccaccctccatgtttgtgggttcaacatccatggattcaaccgactgcaaatcaaaactatttggaaaaaaatggatggtagtgtctgtgctgaacacatacagagattttccttgtcattattccctaagcaatataacaactgtttacagagaatgtacattgtattaggtattataaataatctagagatgttttaagctatatgggatgatgtgcgtaggttatatgcaaatattatgtcattttatataagggacttaaacatctatagattttggtgtctgaggagtcttcgaacaaatcccccacagatcctgaggggccactgtatatatatatcttcataaacacacccacacacacaaacacacccacccacccacacacacacacacaccaatgtgtatatacttttttttcttttttttaattcgagaccaagtctcgctctgtcgcccaagctggagtgcagtggcgcaaacttgcctcattgcaacctctgcctcccgggttcaagtgattctcctgcttcagcctccctagtagctgggattacaggcgcctgccaccatgcctggctaatttttgtagttttagtagaggcagggtttcaccatgttggccatgctagtctcgaactcctgacctcaagtgatttgcctgcctcggtctcccaaagtgctgggattacagatgtgagctaccacacccagttgcaatgggtatagacttttgaaatgtgtactacaaaatattaacagtgactatctctgaatgataggattatggataaattttgttctttttgcatatctgtatttttatttttccacagtaaacatcttttacttctgtaataaaaacttcattaaaaaatccatgctgcaattgattaattatatactaacttttttttactcttccctcagtacagaaagattattctgtatttaccaaatctttacactttatttgcaaatcattttgactgtctgtttgcaagaggtcaaatttgcactcaagttcagctttcgttcattatccatgagcccaagagaaattgtgtcaggtggacatcaggtagctgaggccaatacaatattattcctttgggttgttgagaaatcatagcagaggaaagcctgtgttgataatagagtaccaccaggaatctaggtctttacagctgctggcctgaccagcaagggtaaaatatttcagtcacgcagccagtgattattaaccctagctctatattccagagaggaagagctagcctttttgggttgtgatctgtcactgattgcagttgcacatatactagccatgaggtctacacacatgtgaagagcatgctacacacatagactagatacactgactcatcagcctggaaacagtgcctgaccctcactttgattgtttgaatgtgtcctgacccattaccctgcctttggatccgttcctgtttttttttttttttttaaagcattttatctgtgaattaaaatagtactttccattgcttcatttttttttccctgaggagtttagctttgtttaattagaaacaagacaacaatcattactggtctggtgcaatcttgtttaggactcctcttttgcttagaactatacctggattagggatcactgtgatttagagtgtgcagtatgaggttatttaaatacttaatagtatatttgaaatgaaaagagtagaaattaaagactgaatcatttaccaagaaataacagaggagaccaatttacttctgtaagatttagtagaggctgttcccttctttataattggctttgtttcagttaccagttaagtggggtgaactgttattaaaaggctatgggtaaggttacagtagcattcttctaagaaaaactacaggagtagtcactgtgcagctatctgctttgtacctgccaatatggcagtagatatggaagggtcagaaaaacaggacttcatgttcaataacttttcctttttcctttctttcccttatttttttcatgtcttgtatgcaagatagcatgcctattctttaccttcaatgtatttttgaatttttaatccagatcatattttatcacaatttgtaacatgtagctaataaaaatggttattacatgagaacttgctttacaaattatagatcaaatttttatacaaattattatgtcaccatagaaaaatagttacgatttatctgtttagtcttagaaaaattatttcacatattagttaaggcttttagttttgagtgagcaaaatccaattcaaactagctttgggtagaggtgggggtaaaagagaggaatggatttcaatggaaggtttacttgcgtgtcttggctaactgcaaacatggcagggtgcaaccagaccttagtgatcacagacccagggactcagaccctgtgaggaccttagccttaccgccactcttctccatgtgtcagcttcgcgttctctgtctgcaagtatgcattttccctaaggtgggaagcagagttggcaacagctgccagggttgacttcctgtggcattcttgcagttagcaaatattagagaaggactctgcttggcccagctttgggacacacctctggaccaactagctgttctcaggaggcagtaacatgtacagaggtgatccctggggctcgccctgtgaaatcattgtgagccaatcactacctccctccctgatgctgatgtctactatgtttgatgtctctgaacttccatttcctcaattgtgaatggcaccctggttcctaccttgctggatagatgtagtgattagagttcatgcttaacctacaggtgctctgtaaagaatcattggcatcattacctcttgttggctctatcccattccctatagatatctttctgctctgagactaagcagagaaaataatttaacatttaattattgtgcttctaaatccacttttacctcttctttctttacttttctctttattatttttaattatttcaaaaataaaattgtatatatttaaggtgtacaagatgttttgatatacatatacataatgaaatgattactgcagtcaagctaattaacatcaatctcctcacctagttatcatttttggggggcatggagtgagagcacctgaaatctctcttagcaaatttccagtgtataatacagtattgttaaacatagtcatcatgttgtacattagatttctagacttgttcatgctacataactgcaactttgtaccctttgaccaatacctctctattttctccactattccatgcctgaaattgtgggatataggtatatagtgtgtgtgatatatatagagggagtgatatatgtatatataattgtgtgatatatatgtgtgtatatacaaacacacagacatacacacatgtacatatgtatatacatgcacacatacacaatgggatattattcagctttcaaaaaggacatcctgccatttgcaacaacatggatgaacctggatgacattatgttaagtgaaacaagctggacacaaaaagaaacatgcaccttacttcttagacacaaatttctttctcatagccagcacctgaaaatagggccaagtattttgcatgatttacaattgactgtataaatgtagcatagccagttatatgatgtgttaccatttctgctataaaatcaaaagaagagatcagaacatttaaaaacttccctgtggatttctacctcttacctaagctttgttcttgtttttttaaacctcatgaacacgaagctttagatagcggaggtcatgtatccttgggctcaaatatacagtttcttagcaatctttgattgctacattgcccttttttgtgtttttaactggccttccctgacttaaaatatactttaaaaaataacaaaggtggtatttagtttgcacagtatggaattaaaggtaaacaactttaaattaaatttggttggatgtataaattattaaaatatcctttctctcatggctgggtaagtctgcattttccattcagcctgctatgtctctttccatgagaacactcccgaagaatggggccctgcttccaaacaagccaagcattttgtagtaaattttaaaaagctaaatttcataataacctttataattgctacctgaaaatggagtttggaatggcaccaaattattaatcagtagaaggagaatagattgcaaaaaggtaattatatcaaattgaatgcttcttgaaatatattctgttaaataaaatacagttcctcttccctgttctcatttgtaagcaatgaggacaattcaggtagcatgaatcaggaatcagagtacttttctgttccgcagattggcttaatcattttgggctcacttcaaactgtagatgagcattgtcctttaacctgccgggcagcagaggtgaccacgttcactgcaaagcccagaagtgagttgttcatctgggttagggtctcaggaagaggcatgttatgacttccccacattcctggacagtttagactgtaattcttggttgtttaaaaaggtgtttgaatgtattttgttcagaggcttgagtaaatggctcttattcttacacttttttttttgcctgctttttccagcattagaatagtttcagccctgacaattagccttatgctgactggagtggattttttgcgtaggaccaagtgttttgcatgatttacaatggactgcataaatgtagcctaacaagttatatgatgtgaagaaaattacttaagggtaaaaatggattgtttactgagaaaatgatgttattataaactggtttgagggtattgtggtttagatgctgtccttacaaatttgagctctgttggtttggagatttaatatctaataataatagttaatgtgtaatgaatacttgccctgctccaggctctatgttacgtgtcctgaatgcatcatctcatttaatcctccataaaatttgaagcagtcagcattgtaattaatctcacttttcagatgaggaaaccaaggtatgtaagaaacaaaaccagtgttgaatctgactagtacgactgcaaagcctgactcttaaccattgtgttctgttgccttctccaagggaaaggagtgcttccagaaaggccattggaagtgaggagtgtcacctgataatagctggctcctcatcccactaggttaacaggcaacagatgggggccacagagttggctggagttagtcctggttgccaagggtaaatggggctgttaccatgcaatggatagaaactaggatgctcctgtgtgtctccttatatcctaccaggtgtagcatgaaggttaaggacaattccgtcctcttatttggagaggagaccaaggccctgtaggactaaaggtttgggtgagctgaaagccaagggcaattggcccagcttggggaggagagttacaaatacatttgtgactatcagaccagaaacagactgcggtgttcacctgcggtttgggtacctgctgcttgttctttgctggggtatctgaagactgaaaacacagctcacacttagcctttttcctattctgtgtgaaagaactgtagtagttttctcttgcttaacaaggttctagtggtgaagtttattgcttagtctgtgagttgtagggaaataaccacaggatgggatggtagcagaacgagaaaagaaaagaagattgtagatgccaggctggtgtcggcttgaaatttttaaagtatacagttcttctttatggattattaaaaaaaaacagcaaatgaatacacaagattaaatgttaagcagtacaacattgtacatgatgaaaggttaaagtctttctctggtctcattcaaaaatctacagtctcttgtagtatagtttgaagtcaggtagcgtggtgcctccagctttgttcttttggcttaggattgacttggcaatgcgggctcttttttggttccatatgaactttaaaattctgtgatgaaagtcattggtagcttgatggggatggcattgaatctataaattaccttgggcagtatggccattttcacgatattgattcttcctatccatgagcgtggaatgttcttccatttgtctgtgtcctcttttatttcattgagcagtggttttgtggttctccttgaagaggtccttcacatcccttgtaaggtggattcctaggtattttattctcattgaagcaattgtgaatgggacttcactcatgatttggctctctgtttgtctgttattggtgtatagaatgcttatgatttttgcacattgattttgtatcctgagactttgctgaagttgcttatcagcttaaggagattttgggctgagacgatggagttttctagatatacaatcatgtcatctgcaaacagggacaatttgacttcctcttttgctaattgaatactctttatttctttctcctgcctaattgccctgtccagaacttccaacactatgttgaataggatggtactggtaccaaaacagagatatagaccaatggaacagaacagagccctcagaaataataccacacatctacaaccatctgatctttgacaaacgtgacaaaaacaagaaatggggaaaggattccctatttaataaatagtgctgggaaaactggctagccatatgtagaaagctgaaactggatcacttccttacaccttatacaaaaattaattcaagatggattaaagacttaaatgttagaactaaaaccataaaaaccctagaagaaaacctaggcaataccattcaggacataggcatgggcaaggacttcatgtctaaaacaccaaaagcaatggcaacaaaagccaaaattgacaaatgggatctaattaaactaaagagcttctgttctttgctggggtatctgaagactgaaaacacagcaaaagaaactaccatcagactgaacaggcaacctacagaatgggagaaaatttttgcaatctactcatctgacaaagggctaatatccagaatctacaaagaactcaaacaaatttacaagaaaaaaggaaccccatcaacaagtgggtgaaggatatgaacagacacttctcaaaagaagacatttatgcagccaacagacacatgaaaaaatgctcatcatcattggccatcagagaaatgcaaatcaaaaccacaatgagataccatctcacaccagttagaatggtgatcattagaaagtcaggaaacgacaggtgctggagaggatgtggagaaataggaacacttttacactgttggtgggactgtaaactggttcaaccattgtggaagacagtgtggcgattcctcagggatctacaactagaaataccatttgacccagccatcccattactgggtatatacccaaaggattataaatcatgctgctataaagacacatgcacacgtatgtttattgcggcactattcacaatagcaaagacttggaaccaacctaaatatccaacaacaataggctagattaagaaaatgtggcacatatacaccatggaatactatgcagccataaaaaaggatgagttcatatactttgtagggacatggatgaagctggaaaccatcattctcagcaaactattgcaaggacaaaaaaccaaacactgcatgttctcactcataggtgggaattgaacaataagaacacttggacacagggtggggaacattacacactggggcctgttgtggggtggggggaggggggagggatagcattaggagatataactaatgtaaatgatgagttaatgggtgcagcacaccaacatggcacatgtatacatatgtaacaaacctgcacattgtgcacatgtaccctagaacttaaagtataataaaaaatatttaaaaaatctacagtccctttctcaataggtaaacactattaacaatttcttctgaaaattacatatgtacacacacacacaccccacacatagatattttgttaacatacattaccttatgctctacacattattctgtgcctagcttttctcatcgaataatgtgtattggagatctttccatataagtacatattggtctcactcattcatttttaatggctgtatattagtccatagtatggagtaataataccttttattggcacaaagttttgcagttgacaaagggtttgcatatattgtttcattgggttgtatagcagtcatcaacggacatatcaagaatcccagaagggtaacctgggcttcacccaggatcgcatggagcttgggactcaaacagctaacattccctaaacaggcccatattcctgcaatttagtgcagctgctacaacatttaggtaaactctttgaaagcagtaaaaagatcaccaatctgggacatcaagaagccagacgacaaggagacatagacctcaggtgagtgagaaaagagaataattgaagtttagaactgaatgggcactaacgaacaacagatacatctctttcctttcaccgatgaggacctgggtgtggcctgtggatgttaagtagcatgtttaggccacacagctggattgttttctttcctgaggctagctctggagaggggaggaaacggagatgctagatttttccgggctctcttttctcatcagctagaatgggatgtggatggaatgcagcttgagaagccaaacacccttgagaaatgagaactctgctttctgatgtggggcgcatcctatctgagataactctcctgccagcgcagtatgaccattgcgacttgagctgtgtgggtgcccaggtggtgtagaccttaaaaccactcctgcttttgtttactgaatgtctaatattcacacagttctttgtctgagtgcagggccaaagcagcaaataacgaagaagctgtgcccaaccataagaactatgaaatgctccagatcatctacgctagtttgagttaaatttaccatgcaaataaatatagaacatttcttcttatgcatagattgtatttgttctattaaacaaccatcctgtaaaggttttattcattcatttatttttatttgttaggagaaattgttatttaagctaagaaagtagactagtagttatttctttttttttgcttattatactttacgtttggggatacatgtgcaggttagttacgtaggtatacacgtgccatggtggtttgctgcacccatcaacctgtcatctaaattaggtatttcttctaatgctatcccttccctagccccccaccccctgacaggccccagtgtgtgatgttcccctccctgtgtccatgtattctcattgttcagctcccacttatgagcgagaacatgcagtgtttggttttctgttcctgtgttagtttgctgagaatgatggtttccagcttcatccatgtccctataaaggacatgattgcatccttttttatggctgcatagtattccatggtgtatatgtgccacattttctttatccagtctatcattgatgggcatttgggttggtttcaagtctttgctattgtgaatagtgctgcagtaaacatacgtgtgcgtgtgtttttagaatagaatgatttataatcctttgggtatatacccagtaatgggatggctgggtcaaatactagaaggctacagtaaccaaaacagcatggtactggtaccaaaacagatatctagaccactggaacagatcagaggcctcagaaataatgctacacatgtacaaccctctgatctttgataaacctgacaaaacaagcaatggggaaaggattccctatttaataaatggtgttgggaaaactgactagccatatgcagaaaactgaaactggaccccttccttacaccttataagaaaattaactcaagatggattaaagacttaaatgtaagacctaaaaccataaaaaccctagaagaaaacctaggcaatacaattcaggacataggcatgggcaaagagttcatgactaaaataccaaaagcaatggcaacaaaagctataattgacaaatgggatctaattaaactaaagaacaactgcacagcaaaagaaactatcatcagagtgaacaggcaacctacagaatgggagaaaatttttgcaatctatccatctaacaaagggctaatatccagaatgtagaaggaacttcaacaaatttacacacacacacacacacacacacacacaaacaaccccatcaaaaagtgggtgaaggatatgaacagacagttctcaaaagaagacatttacactgccaacaacataaatatgctgccaacaagcatatgaaaaaaagctcatcatcactggtcaagagaaatgcaaatcaaaaccacaatgacatactatctcacaccagttagaatggcaatcattaagaagtcaggaaacaacagatgctagagaggatgtggggaaataggaacgtttttacactgttggtgggagtgtaaattagttcaaccattgtggaagacagtgtggcgattcctcaaggacctacaattagtagttatttctaggtctgacgggctgtcttttagtttgttttcttattctgaggtggacttaaaccaattttaaaaacagggatgactgaattcgtgtgttaatcttgtactacaggaaccctttgacattgattcagaagcacccaagtgtttgttcattaatccttttttatttttgcatattatttatttttcaacttttattttaaaaatcaggggtacatatgcaggtttgttacaaaggtatattgagtgatgctgaggtctggcatatggatgaatctgtcactcaggtaatgagcatagtacccaatgatagttttttgtacccaatgatagttttttgttttctttgtttttttttgtttgtttgttttttgagatggagtctcgctctgtcacccaggctagagtgcaatggcttgatctcggctcaatgcaacctccgcctcccagattccagcaattctcctgcctcagcctcccaagtagctgggactacaggcgtacaccaccatgcccggctaatttttgtatttttagtagagatggggttttaccatattggccaggctggttttgaactcctgacctcaggtgatctgcccacctcggcctcccaaagtggtgggattacaggcgtcagccactgtgcctggccccaatagatagtttttcagtccttgcccctgttcctccttcccacttctagttatcaccagtgtctattgtttctgtctttatgttcatgtgtaccaaatatttagctcccacttataagtgagaacacatggtatttggttttctgtttctatgttagtttgcttagatagacctccagctgcatccacgttgctgcaaatgacacgatttcattcttttttatggctgtgtagtattccgtggtgtagatataccatatattctttatctaggataactgatgggcaattgggttgattccatgtctttgctattttgagtagtgttctgatgaccatatgggtgcatgtgtcttttcaatagaacaatttattttcattactcttatcaggatacctgggaggaacttctcctacgaagttaatttggggggactcctgaagatgagtgaaaccccttattaagcacttagagggtcgaaagtgtacaagggaacgttcaggtgtgacagcttgagagacatgcatattccacccccacaccagagcttagtgaaccgtcttttctattttctcccaaagcaccaaaatggcccagaaattggaagatggaaaatggacttatttatgagtctgggaaggcagggcagtaagcacagtcctgttcagggttctgagttttaccctcttgctctgattgcgagatcattttcctcttccttgcttcttcagcttaggacaagaacagtttggaaattgtttctactactttggacaccatagtttagatttcaaatgagtacaagtgggaggaaagcttggataattctctggaaataatcgaacagagtgaaggagaggggtctacaggtgagctgaatgctgcatggcattgaagtaatcacaccagtcgtcacaattctctcctctttgatggttatctgttgtcccagaaggaacaatttcagaaagtccttttctggactgtagaatagcacttgcttatttgatgagccctgagaagcattactgaaagcggttcattgtccctgaggtattacaatgagatggtggtcactgatttcattatgttttcctttattgcagctgttggtttgatcctttgccaggtgcttaaaacaattgtggttttgcagatggtaagttagaggttggacaaaaaaagggatcatgtcactgccctggccaaaatttcaacagactggggtctagtgagggcaaatagatagaggctttcctcttcactttgtgttatttagaaaaagaaactttccaggacaaatttctttcctagaattccttttttaaaaattttttttctttgaaaatttacttagatgcaaataatatatttttcttccttttaaataataaaagtaagatgtctcttggaggtggtggttgtcactgacaagattaactagaactgactagctgtaaaaatataatttgggatgcattattaaggcatgccatttttatttgcatgccattgtgtacagatgtggttgtgaaatagttcaaatcatggcacattgaatgtcctcactggatttttaggaatgtgttcactgagacagccaaatcctatttcattttctttggctcattgcattggctgtaaattggagatattcactttaatatgtgagtcaaaatttatttccaaacataatactgcagttgttctgtcacagaatataaatttcttatttatttccttaatacgttgctttctactttttctttttttctttattttattctggagtatgtggaaaggttttccagaaagatttgcatatgccataatctactgatgaatactttttttgggttactctttcatattttgggagatataactatggaagtgttaggaatcatgggttctggaaatagttttattactgcttctgaaatgccctcccaatgataccatatagtaattccatcagggaataatatttttattatagtttaaaatataacttaatatttaggtgctcttgttcagtcatcgtcaagttctttttatttcaccaaccctaccatggcactcctgaaagacttgtgaatgcgacagacctggatttaatcatggctttgccatctgctagccaagagaacttgaacaagtgagtcaacttcttggagtctcattttctgcttctgtaacatgggaactagggtaatctaactcattgcttgtgatgattagatgaggcaaaatgctgagttcacctagcccagcacctggtccatgggaagcatgtgggttctgctgctacccagtccttggcccagtgcatggtgcacagaagggaatctgaacaggccaactttattcctattcttgacccaccccatgtagatgcttcctacatcttcagcttcttcttcttcttctttttttttttaaggcagggtctcactctgtcccccaggctgaagtgcagtggcacaaccacagctcagggcaacctcgacctcctaggatcaagtgatcctcccacctcagcctcctgagtaactgggatgacaggaccacactaccacacttggctaatttaaaaacttttgtagagctggggtcttgctatgttgcccaggctggtctcaaactcctggattcaagtgatgccctcacctcagcctcccaaagtgcctggaaaacaggcctccacacccagccttcaacttcattttaaaaaattgtggtaaactatacaattcatccatgaaagcagaaaccacttgtaccccaaaagctattgaaatttaaaaatatatatattaaaaataaaaataaaattgtgataagatatacataatatgaaatttactactttaatcatttttaagtgtacggttcagtggcattgagtacattcacatttttgtgcaaccggaacttttcatcctcccaaagtgaagctctgtacttattttttattttattttattttttgagatggagtttcactctagtcgcccaggctggagtgcagtggtgcaatctcggctcactgcaacctctgcttcctgggttcaagagattctcctgcctcagcctcccaagtagctgggattacaggtgcccaccaccacacccagctaagtttttgtatttttagtagagacggggttttgctatgttgggcaggctggtctctaactcctgatctcaggtgatctgcccgcctcagcctcccaaaatgctgggattacaggcatgagccactgtgcccggccagctctgtgctcattaaacaatgactccaagttccccttccccacatctcctgctgacctctcttctactttctgtctctgtgagtttaactattctaggtacgtcatgtaagtgcatctatgtgatatttgtccttttgtgtctggcttatttcacttagcataatgtcttcatgattcattcatgttgtagcatgtgtcagagtttccttcctttttaaggctgaataatactccactgtatggatagaccacactttatttatccatttgtctgttgatggacatttggatgatttccatcttgtgggtatactgagtaattttgctatgaacatgggtataaaaatatctatttgagtttctgctttcaattattttgggtctacacctcaaagcggaattgttggaagtggaattgctttccacttgttggaagtggaacatggtctcccacttcattttgacacaacttccaagcttcagaactgtatttacaacagcgtgctgggaggtcgctttgagtttatgacggaaatctcatatcaacaggttttaaattgttccatacgacttcttgccacccctgtcccaattcagatcttttttttgattaatggtatgaacaattttcaaaatatcctggcacaatctattagaatatttgactttctcctcctcctccctttagccctaactatttggcaaagctgttgaaaacttcttcacatcttctctctgcctcatctactttgctactcaaagatgattagtagatgttttggtatgattttttttccttggcataactgtgaatacttttaatccttcatgttttatgacaatcatacattcccacttttgtgtaaaaacattttgaattcttctttctataacaaattctttgccagtcttccttttctttttgcttttagtgagttccaatgatatcattgtttgaacctgttttctaattctatgctgctattcccatactccctctttgctttggttacagtagataatgtgggtggtcctaccaagacaaagcatcactcaggagtcaagggctggggtagatacagacactaggtactgcagaaagtcaatatctttctcagagtgtaaggcaggacaagacttctgttcccttgtaccgttgtggctggggagttggactgtgcatttcatccttgtcatataagtcaaagtattgcccaaataacttaattttggtgtggtcttagtaagtatttgtctcgtagatatgttaaatagaagacagtaacattgggttggctgtgttaattcctcactttttctttcctatacatgagcttcctaagaagcggaacacttggtggtcagacagttgcagagattccttgcatgattgcaatctggaaagtagataccattcttgaatgaagagcttgccctttggagaagctgggctttccatatatggaggttgttagaatgcataagagttcagcttctggaatgaaaggagaccttggttcaaaacccatctgccacttgcttgctgtgtggcttaaatcaggcttttaaacttcaattataattttgcatctgtataataggattaatataattccttcctcataaagttcctagggggttaaatgaaacaatcaatgtataacacacttcctggcatgtgatgttcagtacctagaagactcggtttccttggtagagagaatatttggctagacaagcttatggaattacccccagataggaagtgagcacaagtgtgaaatgaacaagccagagcaggaacggctcttggaagcgtctactcaggcctgggcagttgctggtttatataacagtgctttgaaaattcacgtgcagattcttactattttcccaaatgttacagctcaaaactatgttgtctgtaccttaacacctaaaggataatatagtctttcactgataaaactaaaatgtcataggttttcctttggccaaatatgtatagaaacttgtgatttcacatcagatttaaagctgtatttaacactctatgaaaacatactgatgcttagaagtagaaaggaagtcagattttgacatcttacttgtcaacttaaattatttatagttcctggatgcttcaaaatgtgataaaccatagttaattttatgtaaatattcgatgagtgcctttaataaggagactgtaaaggtagccaagctttatatatgttagctacatttatgggtcaatcgggtataaaaaataggacttcgaaaataaaatattattttgtcggactcctccaatgaggctttttcgcaggattagctaaaattggctcttatttgatgtgtgagtgcttaaacattggagaattcatttttcttttagaattcatttttatttctgagccttaaaatatgaacagttagctaaatgtttgtatatgttgataaggaatgctaagtgtttattccttaatgggacgacaccttttcccggtttacataacttgccttttaatctaaccttatgaaagtctccttgactttaattttttttcagagtactgtatatctctttagggaatgcatttatttaaaaaattataaagcaagaatagatgtgatatattttgaagttttctagtcacaaattaaatccctagatgtgttgtagtttgtggagcactttgaaatgtgccaattcaagatggaaatagcaggaaagaaccattcaagtacgatttctgactccataaagttaggaagttatgataaaggaaaaataactacaccacatacttatggcacagaattgcattattgggacaaattgatcttcaaatttgtaggctatgatggaagcaaatatttgtagtatcttaatattcaactgttaagccaggagacgagtactctgaacttcagcttctcataaaatcagtcagttacatgacagtttatgtagtttatatgtaagaaaccctttgatcaagatatgccttttcttcagccttgttaatacttcatttataaggatttttatttctaggaaaataataccatagacctattttatttaaagctaaagtgtttcctggtgatggtggtgaatggggagatgattcaaggaaactgctaatcttgtagagtttagtaaaatcttggaatagaaattttaaaaagttaaaacacactatgaaaacaaatcattattagtaaaatgaaccatattaaaatgtctccataaccaacgtattatagcaggggaaaatggcattttaattcagaaaaacatttctatataaaacaagctttggaataatttgaatatgttgatttttctttggggcaatcatgaaatacagtcatattaggaaagaggcaaggcctcaaaacggaaagagtagtgaggataattcatgagcaatgcgtggcttcatggatcccttcctggccctcttcatctatgaacatctgctttatgttcatgtctggcctcacccagtgctgaagagcagactgcccctgcttagaaccaagccttgttcttgtggatttgagttttggggtcctgaggtagaatggccatcatattgttcagggtcctcaccttcccactcattacttcctttatagaaccccagtcatccccctcaggaggcctgcgctccacaaatgaagttgggggtgaggggagctttagcatctcagtattgttcagatattggatacttgttgagctccactatgtgtgtgagaatgcgctgggctcaggatcaatagccataaatgagacagatatggcccatcccttgccatgcctaaactgatctgggcattgagacaagcagttaaaacccaacatagtaagtcctatgatgagacacaaactaagcatggctggaagggcagataagatttcacagaggaactgacatcttgactggcacacttagaagatgagtaggagtcagttgggccaagaggagaaaaagtgtgtttccatgaagaagaagagcatgtgccaagtcctgggggtgagaagcatggcatgtgtcaacagaaagacatgatcagagcttggatgggagttagagcggggagagaaggcaaggaaaaacataagagctttgcaaactgtgaaggcattgcatttagatgtcatttttagagcttggaggagcctcaggacattgaggcaggatagtggcctgatggcgtatgattttaggaaaaatcactgtggccaccctgtggggaaggattggcgggggaaggctggaggcaaagggtcctgcggtgggctgtggtaatgatctgggctggagaaggtatggtggtgcccagcgcaaaattggtttcgctttgaaaaataattagaaaatatttgttttgatctgtgtgcctgtggtgggaggtggggagggagggtcaagtgaggaaggagattgaatctagaataatgcccatgtttcaggcttcacgtgcagacatttcttggttcagctgggaagtgaataggaacagactggcgaggaaagaagacagtcgtcagttctgtctggggcacatccatcttgacgagtgtgagagagtcaagtcttgatggacaggggcaggtggacattcaggaaactcaggaaagagatttgatctgaagtgaccaccagaaaatactgacggaaaagaggtgacaaaaggagagacctgcaatattatattggctttctctcaggcaactgcttggcctgtctttatattccttttgaatctctgcatatgtacgggccattatttattttcacaactaacaaatgcagactttctgtgtaatgacaacaaagccaaaccagctgctaccaaaggagggaaaatcagaagagaaggaaaaagaacaagggaagcttggggaaaagctgaatgtgggtccttctgttgctgcaggggctggggtgggcccggtgattcctactgagaggcgttttctctccccgcttcctgtctttctggttccatctcattcacctcctgtcccctccacttcctgccagtcaaaccttagattcctccagaggctttttatttttatcttttgatgggcaagaaaatagtgcggattatttttccaaaccttcacctgaacatcacatcgtggctttggccctatgggcttggttcatccgggcctgcacagaaggacttttcgggccagtctggtcacatacatcgagtcctgtcttttcagttaaaaaaaaacacacacacacacactgctatgtttcactaagacaactggtgtgagttgttttttagaaaatcaactctacttcagtaagattttctcaagcattatcttgagaagaccagataataaaatttaaaaagaatttcttcttttttttttattatactttaagttttagggtacatgtgcacattgtgcaggttagttacatatgtatacatgtgccatgctggtgagctgcacccactaactcgtcatctagcattaggtatatctcccgatgctatcttaatgcaacttaaatcaattgctttaataacacatattgaccaagttacactcattaaggaaaaaaaactactttgttgtttttcttcttctgacgtgagctgaagacttagaaatagttgttaatagtgtttggttaataagaatttgttttaattgcatatatcaaatatgtatttattaaacttttctttttgtcagtttatcaattctagctttgtcacaaaaggtttgccgtatgaacatgattctgttgtacatctatttccatttttgttaagagacgaattcaattgtaaaaatctagtaccttttattcattaaacatgttagttcaggaatttcacttggttctacaaagatacatatctacagtggatggccagtgcaaacatgagactcagccaactggtctctgacccaattcagttctcctgtcttcttctggcttacaaagtaactggctctggggagaaagtgagtcaaagtaatatttggtttgaatggttattgactattttcttctgaaacttaatgtatactaattaattttttattttattcttttttttttagagatagggtctcgctctgttgtccatgctggagtgcagtggtgctatcacagctcactgcagccttgacctcctgggctcaagcgattctcctgtctcagcctcccgagtagctaggattacaggcatgtgccatcatgccaggctaatatttaattttttttttttgtagagacaagatattgttatgttgcccaggctggtctaaaactcctggtctcaaactatcctcccacctcagcttcctgaagtactggaattataggcatgagctgccacacctggccatatactcattttttgttaaaagctgaaatatatcagcatatactgcataaataccacaggagactaaacactgaaagtttctttagggtatcagaagaatacactttttgcttgcagttagcatctgcacagataagttttgtttctggttctattacttcttcagtttgaccctattaataaggacaattctaaaaataataactgtgtctggatatctgaatccgtgtgtggtcttttactgaagttacaggtttataactctgctgactagtttgctggtttctgttatgcaatagaagagtgcaaatgttaatttgatctgaagcccgtgtatacagtgacttttataatgtatatttaaagatggaaagccaagttttatgaggccagcatttcttgtcaagtcctcactccaccctcttctaattgggcctgacccttaagttgaataaagaacaaagagctctgtagttaaaacatttcactgcatgttgcatcttgcccttagtaaatggaaaaaaatagagacttaagcagagaatctgaactagggtgtgaaatatatattcagttttggggtgggagaatgagaaccatgttttacaatagtatacataacttccttagtctaaattcaggacatttccccaagatatgtaagaatttagacttatgcagacagactttataaaaacatgccaatatttattagtttgtgaattttaatattctgctccctataaaccagatttattttgagggataaagggatggaggtgacttctaaatcttagagcagaaacttcctgtgggcagctggacatatgtaccaggagctcagagaagagggttgtgttgggagcgtacattctgagtgatctgcatttggtgatgatggaagccatggacatgcactagattgtcttgtgggagaatatggagtagtaagagaagaagaagacctaggattgagccctgagcacctctggcttaatgttggatggaggaaattgaatctgtaaacaataccgggaggctgcagcctgagaggcagaaggaactggggtgtttgggattatggaagccaagggaaaaagcctgtctcacagcgggaagggaggtatcaacattgtaagctgcttcagataggttatgtaggatgtggactgaaaaatacctgtaaaatttggcaacgcgattcattggtaatcctagggaagttgcttttttggggtaactaaggtggaaaacagattggtgtgggttgagcaatgcaagagaggtgaagaaaaggagatttcatgtgcagacgtttctgagttcagctgggaacaagggataagatagaagatggaagttagagagcatgtggggcaagggagactcttttatgggacagtctcgcatgtgatcaaagccaatgagtaaggagcatttgagggagagagagtcaatcaacaagagagaaaagaaagagggttcatcataaaataacggtaacaacaacgaacatcttttgagtgttttctatatccggggaactatggtaaactcctaacctgcattctcacattcaattcttagaatcgttggatgtggtgggttccatgatttcctctagattagcgaggaggaaagagagatctagaaatgtcaggtagcttgctcagagttctccaggtagtcagtcatggactaatttgtgaactgaaggactgaacttcgtcaccacccagcccaccaagccggcttgactttaggtattctgtgctgcatgtgagtaccgacttaaattatattttaagaagggctactttgaaactctctctctgaaaactctatttctaaaagctctaccctcacaacaattttggcaagcagtcttggtaaaaccaaaccaaaccaaaccaaaaccaaaaaaccttatctgctgagaaaatataaccacataaaatatggtgctacaaaatatagactgtgtgaactgaaggtgacttgcccaaaggactcctgaagcaattggctgctgtagaaattaagtccacgggaggttttttgttctgtttttttttttttttttttgagatggattctcactctgttgcctaggctggagtgcagtggcgcaatctcggctcactgcaacctccgcctcccgggttcaagcgattctcctgcctcagcttcctgagtagctgggattacaggtgtacaccaccatacccaggttttttttgtatttttagtagagacggggtttcaccatgttggtcaggctggtcttgaactcctgacgtcgtgatccacctgcctcggcctcccaaagttctgggattacaggcatgagccaccgtgcccggcccatgagaggttttgtttgcacttcaagaaggacagaaaaaggcaggcaggctggggagcaacatagtaaggctgaggaagtgataggaaaacagcctccaaaaggtttccctgtagattctgactggctaagtttcctgaaataatattaattctgtcctcttgcttttaataggacataacgactatatgtgtccagccaccaaccagtgcaccattgataaaaacaggaggaagagctgccaggcctgccggctccgcaaatgctacgaagtgggaatgatgaaaggtggtaggtacatctctcccaggggcccttggggatggccctggccaccgcccagtgctggctctacccattggaataacaccatgggaattttgtgtttttttcttttaattgttttttttctattcttatttttctttgcaacaaaagtattttcataatccattttattttaaaaaggtggaagtgtctggaactggaaattctaacatggcattttgtgttttggattttcaatgtaaataattatattttaaatcaaaggtgtgtgggaggcggtgatggaaggaaacgaagagtgcttagtaaattattctagaaatatttttcagttactgtttatgttgcaaatgctagaaaatgatatctgaggataaactttccctaaattgagacttgtaaatgtgaaagctgagtagctaatttatagccttccagtctgttatcatcccttaaggaatgtgaatttcaatcaaaaggcagttttctcctttagaacctgagtgaaccagccactttcttaacctcagtgtctagcatggtgctggcatagttgattactgagcactacactaacaagtgtcagagcatgcatggtttgtgactgtgggtttgtgtttttgtggtctttgtggctgtgtgtgtgtgtggttttctgtcttctcatgtatccgatcttccagttttgtcatacaggaatctggaaactgaatcccagttctgggaatattaggagccccataaatgtggttgcctgattgacctcattgtattcttgggagtctcatcttgaggaacattgcttctagtcttggatagccttcagtgtagtggaagagaacaagtagaaaatgtgtaattaaaataaagtcatgaggctgaggtgggcagatcatctgaggtcaggagttcgagaccagcttggccaacaaggtgaaaccctgtctctattgaaaatacaaaaattagctgggtatggtggcgagtgccagtaggcccagctacttgggaggtggaggcaggagaatcacttgaacctggcaggtggagggggcagtgaactgagatggcgccactgcactccagcctgggtgatagagtgagactctgtctcaataaataaataaataaataaataaataaataaataaataaataaataaagtaacggattcatttagtgtttcagaaggatactgaagggagggaagggctgatgatggtgctacctgctgattgtaatagggaaaagcccgtttcttttctgaaggaggtgaagcttggggtgatttaaggagaacaaaatttcaatgaaaaggaatagtgattttgcaaatagtgggcagctaacctttaaatcattcttactgggacgcaggaggaaggagaggaaagaccaagaatggagattatgatgaagacaagcttgattgttaacaagcttacagtgacaggtttcatagtcccggggtcataaatacgccagacaggttgaggtttggaatgcagcatttggaataaattctcctggtgaagagatgcaatgttgaatttacgcattctgtgagctgtgactatgactatacatcttgaacatctgaaaaagcagctttaattcaaagggtaattaattccaagaatttaacagctacattcagaaaatgacacttgagtcatatggattaaatatttaaggaattcatctttctttgtactgttggagaattagtcaggcttaattaaacttacaaaatgcgtcttaaggtaacttggtaagtgacaggaatataacagctgcataagaaaagctttatttgaaacagtggagtcgaggtttaatattcttctttggcgattatatgtaggttaagcacaggctcttccatacattctccttgaaagctgaaataatcaagtcatgaatatgtccaaaacaatttttaaaatgtgaggggtacccatgcattgaccccatttcataaaaccgattctgtttttttttttgtaattaatatccagatacggtctggctgcatgaatataaattacgcattctcatttttaatctaacaaaaattcatatatgcaaagacataataaaagtctccccactgttttctcctaggaatctagataatttgactgtacacaacagactgtggatggctccatacacacttgcacatgtatattgatgactgtaaaatatatatctgtattagttttctagggctgccataatgaaataccacaggctgggtggcttaaacaacggaaattaattttctcccaattctggaagctggacatgcataatcaaagtgctggaatatttggtttctagtgagccctcccttttttttttttgagagatggagtcttgctctgttggccaggatggagtgcagtggcactatctcagctcactgaaaccttcacctcccaggttcaggcgattctcctgcttcaacttcccaagtagctgggactacaggtgtgtgccaccatacccagctaatttttgtgttttttttttttgtagagacggagtttcactctatgttggccaggctggtcttgaactcttgacctcaggtgatccgcccacctcggcttcctaaagtgctgggattataggcgtgagccaccatgcccggcctgtgaggcctctcttctaggcttgtggctagccgtcttctgcctgtgtcctcacatggcctttcctctgcgtccgagcactcttggtatctctttctcttctcacaagccctgttggattaggagtccacccttatgacctcatttaaccttagttaccgccaaaaaggccctatttccaaatatagtcataccaggggctagggcttcaacatacaagttttgtggggacacagttcataacaggctgccctcccaaaataccatagactgggtggcttaagtaacggaagtcaattgcctcatagctctggaggctgaagtctgagactaaggtgttggcaggtttgatttttcccgaggcctctctccttggcttgaagatggctgccttctctctgtgtccccacatggccttttctcggggcatccacagcttcttcttcttcttacgaagacaccagtcatatcggattagggccccacacacaggacctcattgaatcttaatcacctcacttaaggtacaatttccaaatacagtcacattctgaggcccagggggtaagatttcaacatatgagttttaagggtacacaattcagtatgtggcaatatcccataggggttactcagtgattgttcagtatttctttacatgtaaggaaaacatgtatctttctggcagtttttatatgtacataagttttaaatgaagctttaaattcataaaattatagcccgtgagaacatcttattttaatgatatgtataaagtacttgccataaatcaatataatttattgtattttctacaacatggaataccacacatggccacaggataccctctataacaactccttgaaaaatcccattgaataaagccttaatttacaactggaagtacaatgataactaactataaaatgtgcagtcacatggcaaaggccagctgtgactgaataaatctttacaacatgttttcaaccattagggttcacaacaatccactttcttaaccaggctggttaccaactaggatattgaaactctacctatgaaataaaaagtttagtttcagttaaaagagaaccacagacctactccctattttcatagcaaggctgaaaaaattcactgaagatctattgtaaagatccaaaagagtgcatgatttgtctccatgaactaacaattttaatctgttataatacttactgcctctgaaaatcccagcatcattattagatttggttctgaatcatggtagttctataagatgcaggatttgaattgcactttcgggaatgtgcttttctatcctagtacatgtgctggtttctctgcattgctttttcctcttcatttgtctccttcctcttgaactttaggttgtagattacatgtctcttcttcaggaaggcctacacagttacctgacagttcctgaggctcgaagttcaaaatcaaggtgctggaaaattcatgttctagctttaatggcaaacagctcttctcttagctcttatcaccattattattagtcattcaatgtctgtctttacatatggtccataaactccatgatggcgcgggggtggggggtctctgtgtcttgtttaccgatcttgcatgaaacaagtcacaaggtaggtgttcaagaagtatttttttgcactaattaatgaattgattcaattcatttaaatagttatttgtgtagggcttcttatatgcaaacccttgggaataaagcaaatatgaataagattgtagtcccttccctcaatgtcctcacagtataaagagaacatttgcatgggaagcggcaaataaaaatctcaacggcaagtggagactgggttgcaagatggctcaaatattgaatgatcagaatgggagtctagagctttaaacaaccactcccaggtttagtccaactcagagtattactcagacacctgacatccaagacatatattattactcaagaatgtggcctttttgctgagtttggtagaaagaggaactcttccaaagataaatgtgttcaaatgctggctcctcctcaaacttgctgaacaaccttgttaaagtcacttttatgagcttcactaatttcaaatatgaaatggagataaataaattttattgtccaagcacggtggcttacgcctgtaatcccagcactttgggaggctgaggtgggcggatcacctgaggtcaggagttcaagaccagcctggccaacaaggtgaaacctcgtctctacaaaattagccaggtgtgatggcagaagcctgtaatcccagctactcaggaggaggaggcaggagaatcacttgaacccagtaggcacaggttgcagtgagccgagattgcaccactgcattccagcctgggcaacagagtgagactctgtctcaaaaataaatatataaataaacaaataaataaattttacttaatgagtttgtcatgaagatgacaaatcgtaacatgggaaaatataaatctaaagtgccacacaagtgttagctattacatatcttttttgtcttaaaaataattactatcttagttaaaaatttcatcatatacgagcactgctgggttttttttttcagctgaaagaaagagaatggcaatttcatcacttctacaagatataaatttctaatttctttgtggagaataaaaacttggcttaaacacctaagcttttttttcttttctgatctcatatgctgaatatagctgaatgacaagtgagatgtgttatttatcattttatgatgccagagatttacacgaatttgtgaaaggttttttcttaccatagattcagaggcgtgtttgaggcaaggttttcattgtgcagtgaggaaattggcacatagaaatcaattgatgtgcaggactcttggactgactattctggttatgtttccttataacacattaagaagaaatttgagataaagtagtaattgtcaactagtctttatttattttaataacaaatgttgcagtataatttttagaaatattccaaaataacaaataaatccaaaaattaacgggataattttaaagtgagaaaataacatactactaatatagacagggaaatatacatgtgtccagaatgattcagaagcccaatgaaacactggaaaataaaatattttcctgttgcttgggtttaatatttatcactatgagttatcgtaatttgctttattgatggataaactcccaacctggggactgcgattggaatcctctttaatcctacattggctatcctttatcaaaagtaaacaaccggctggccatggtggctcacacctgtaatcccagcattttgggaggccgaggcgggtggatcacctgagatcaggagtttgagaccagcctgaccaacatggtataacctcgtctctactaaggatacaaaaattagctgggtgtagttgtgcccacctgtaatcccagctatttgggagcctgaggcaggagaattgcttgaactcaggagacggaggttgcagtaggccaaaatcacaccactgcactccagcctgggtgacggaatgagactccatctcaaaaaaaaaaaaaaaagtatacaaccaattattaatgttcatgatggtagaatcaaattaataaattaaaaatgtagatgtgaattactttttttggattgccgaataattgtggaagatttgtagctgcatgtgaatccaagtttcctcaaaaggtaggttggggtgctcctatttaggattgaatttaagaaaaatgttaaagaaaagctagctgctctttaccaaagagagactggttgttttaagcagtaatctaatctatatatttatgatattgaaactattattttattaattcatataatagatatttattaaaaactccccatattataattttttatttgcaaacaaatatactgaatcaagtaagtgaatcacatatggaccttaaagactaatgaatccaatgtttgacagtattttatatcaatcaagctctggtttccacaagcagaagcagatcgtcatctatgaaacagataaaagttgatttggcataccctctctttattcattatgatgttcatgagaaaccttcttagaaccctagggctccaaggaacacagtttgcaagccactgatctaatctaactccttcatcctctaggtatgaaaatcaaggccctccattacctattagggatgaaggcaggtcagcctctgtgactgtcagttcagctggaggaagcatgagtgtaggcagcacaacaatccctataaggaccatgttcttaagctggttttttttttcctagtagtttttacagtttcaggtctaacatttaagtctttaatccaggttgagttgatttttgtatacggcatgagataagtgtccaatttcattgttcggtgtgtagatatccagtttctctagaaacatttatcaaagaaactgtcctttctccattgtgtgcttttagcagctttgtcaaaaattaattggctctaaatgaatgaatttatttctgggttctctattctgttccattggtcaatacatgctgttttaattactatagctttatagtatattttgagtcaggtagtgttatgcctctagctttgttcttctagctccagattgttttggctattcagggtttttttgtggttccatacaaatttttggattgtttttccacttttgtaaaaaatgtcattgcaattttgataggcattgcattgaatctgtagattgatttgtgtcatatggatatttttaacaatattaattcttccagtctatgtatggtatatctttccatttatttgtgtcttcttcagtttcttttattgatgtctatagcttttagcatacaattctttcacttccttggttaaatttattcccaaatattttgttttatttttatagctattataaatgaaatttcttcttgatttctttttttggatagtttgctgttagtgtatagaaacactactgatttctgtatgttgattttctcttctgaaactttactgaatttgtttattagttctaatagttttttgtgtggagtctatacttacatgatctatgtgtaagatcatgtcatcagcaaacacgggcaatttaacttcttctttttcaatttgcatgcctttattttttttcttgcataattgctctggcaggaactctcagtactatgttgaatagaagtggtgagagtgggcatccttgtcttgttattgatcttaggggaagacttttcaaattttcaccattgagtatgatgttagctgtaggcttgtcatatatggcctttattgtgttgaggtacactctttctatatttaatttgttgagagtttttgatatgaaaggattttgaattttgtcaaatgcattttttcctctattgagatgattgtatggtttttgtccttcattctgttaatgtgtgtaccatagttatagatttgcatatgttgaaccatcgttgcatccctgggcaaatcccactcgatcatggtgaatgattctttttcatgtcatgacaatcaattttgctagtattttgttgtggatttttgcatctatattcatcagggatattggcatgacatttaattttcttgtaatacccttgtctggctttagtatcacagtaatgctggccttgtaaaatgagtttggaagtattccctcctcttcaatttttgggaagagtttgaggattggtattagttctttaaatatttggtagagttcagcaataaagcccattaggtcctgggcttttcttccatagaggactttttattgctgattcagtcttctttctagctattggtctggtcagactttctatttcttcatgattcagacttgctaggtttatgtgtctaagactttatccatttattccaagttatccaagttgttggtgtgtaattcatcatagtactcttataattttttgcatttctgtgctatcagttgtaatgtctcctcttttatttctgattcagtttatttgtgtcttctcactttttttcttagtctagttaaaggttaatcaattttgtttatctttttaaaaacaaattcgtagtttcactgatcttttgttttgtgtttttagtctcaattttatttatttttcctccgatctttattattttcttccttctactaacttggggcttagtttatttttatttttctagttatttgtggtataacattagattgtttgagatcttcttctttgatgaagacatttattgctataaacttccctcttgaaaatactcttgctgcatcccacaaattttggtatgttgtgtgtccattttcatttgtttcaagatatttttcaacttttctttttatttcttctttgacccactggttggttctgagtatgttgttttattttcaagtatttgtgaattttccaaaattattcttgttgatttatagtttcatattatttatggttggaaaaaatacttgatatggtgtcaatgttcttaaatttattaagacttattttgtgtcctaacatatgaactatactgttttttttctttttttttttttgagacagagtctcactgtgttgcccaggctggagtgcactggctcaatcttggttgactgcaacctccgcctcctgggttcaagctattctcatacctcagcctcccgagcagctgggaatacaggagcacaccgccatgcctggctaactttttgtattttttagtagagatggggtttcaccatttgggtcaggctgctctcaaactcctgacttcaaatgatctgcccaccttagcctcccaaagtgctgggattacaggagtgagccaccgtgccaggccatgatctatactgttttatctaacattcagtggataatgttccatgtgtatttgagaagaatgtgtgttctgttgctgttggatgaaatattctgtatgtatctgttaagtccatctgtgctaaagtatagtttaagtttgaactttcattattgattttctgtccgaataatctgtccattgctaaaagtggggtactgagttccccaatattattgtattacagtctatcttccctttcatatcaattaatatttgtttcattcatttaggtactccaatgttgggtgcatatgtatgttcacctgttatatcctcttaatgaattgaccctcttgtcattatttaatggccttctttgtcttattttatagtttgtgacttaaagcctattttatttgatataaatatagctgtctctgctttcttttggcttctattttcatagaatgccttttttggtcccttcgccttcattctctgtgttttcttaaccatgcattgaatctcttctagacatcatataacagggtcttgtttttttaaatccatttactcactgtatctctttcttcctatcttgctgtcttcctttgtgattaggcgattttctatagtggtatcctttgaattcttactttttgccttttgtatatttgctacaggattttgctttgtgattaccataaggcttacataaaagatcttatagttataccaggctatgttcaactgtttataacataactttgattaattttttaatactcaatttatttgattgcttatttagagataattttgattcctatcagaagaaaactgaggtatctttatgtaccttttcaggaatgaccacgtctcaccttaccttgaatgttacaagaaatgctggcagtgactcaaggccaggcaaaaatttcagaaagggtttatcttgtttgtacaattttcttttgcaaaaataaatgattcttgatatgatgtaaggactttttccgtagatagaacctataaaatgcagaactttggggcagtaattatatgaatgaattgctactttaaaattctgtaagtccccaggaatttccatatgcttttttttgtttgtttcagtggttgtgatcaggctaatgggtagtttttagagctgaaaagaacttaaagatcatctagtgcacctttttgtttttcagttggattgttcttgtccaaatcactaagctaagttaactgctcagacaggaaaccaagtctcctgacccatggtttaatgctcctttcattgttatgcgtctgcctggctgacctgttagtaacataaagtgtcatgagagatagacatatgcatgagtaagatattaatatcccataaaccaaaactgtgacttaggaaatgttacggtgggcacaggatattactcctcttcaacaaaaacatcaatcatatgttggcttagttgcacagcagaagtaccaaaataaatttatatagacggtgtcacagatactttaaaaagacctacttaagatttaattacctaatattatttaatatttaatataatttaaaaggggcatttttatctttcagccaaggttttaattgtttttagataattctatttttgaaagtctgattttgttttaaaatgcccattctggtatataaaaacatcattttgtcaaataggagtcccagctaagggccaagcgtgcaattaaacttgactctttacctactacctaggaaccttgggcagatcacatactttttcttcactcagtttttacttctgaaaaatgccagctgaagtatgtttcaaatcttaaattttatgttttatgatctaagttcagattaacaatggaatggttagaatccaaagctaaaggtagtatatacaagtgtatattgggggttgggagcaactgccgcagctatcatatttacatttcaagctcaatttagaacaactgcgggcaactgtaaaaaccggtaggaatgggcagagttattgtcttcactagtcattgtggaaagtgaatctcttctgaatggagagtcacaaaaatgagtttgatgctttcatgatagtgaaatgagaagatggccatatattgctataaatcaccagcaacataaaagaaaaaattcaaaaatcaagtttgttgaggcaggactctgaaaagataactaagcttttttttttttttttttctgttagagatgtaataatcatgactaagactggaatcccaagagctggtgagactttctagtttctgccttgtggaaagagaaaggaagaggccaaaaaagataatgaattttggaaagttcctgattaaatatcgcataggctagtctggttatgtctgcttgaaaacttatccagtaggtataaaccaacacacttgaggccagtctttttggaaattacagaaatatatgtcagccttaagagacatcctatgatgaagagcagagggagttgtttataaagaaatgttctcattggctgggcatgatggctcacgcctgtaatcccagcactttgggaggccgagctgggtggatcacttgagatcagaagttccagagcagcctggctaacatggtgaaaccccgtctctactaaaaatataaaaaaattagccgggcatggtggcgggcgcctgtagtcgcagctactcgggatactgaggcaggagaatggcgtgaacctgggaggcggagcttgcagtgagccaagatcgtgccactgcactccagactgggcgacagagcaagactctgtctcaaaaaaaaattagccaggcatggcggggagcgcctgtaatcccagctactcaggaggctgaggcagggaaattgcttgaacccaggaggtggaggttacagtgagttaagatggcaccattgcactccagcctgggtaacagagctagactccatctcaaaaaaagaaaagaaatgttctggtcatgcctggagataatacatggtttagtttatgcttgattcagcaaataaaaattggtgaggaacatacagaattatatttaggtgattatattttttatattttatatataaaatttatattttttatattttaggtgattatatttttaaacgcttgctttacaagactaatcattattacttttttaagttttgtgatatattttatttaactaatataccccaaatagtatttcaatacagaatcaacatttaaaaattattgatacattatgtatgtttttttccttcactaagacttccaaatcctccaagtgtttcacacttacatcttattttggaccagccagattttagtgctcatttgaccatgtgatctgtgcttaaggaggctgctggagctgaggctgggctgcagtaccaacatagaagtctttatctgtcaaaccaggagtttgagatccctgagggatatgggaaccataaaggatcttaagcagagaagtgactttcttaattttgaacctatcctggtgttctcatagattagaggagagagatttaggagtgagggatcaagcaagaaggtggctccacattgttttccatagtagttgtactagtttacattcccaccagcagtatagaaatgttcgcttttcactacatccacaccaacgtctattattttttgattttttgaaaatggccattcttgtgggagtaaggtggtattgcattgtggttttgagttgcatttccctgatctttggtgatgttgagtattttttcatatgtttgttggccactttgtatcttcttttgagaactgtctattcatgtccttagcccactttttgatgggattgtttgttttttttcttgctaatttgtttgagttccttgtagattctggatcttaatcctttgttagatgtatagattatgtctccttctctgtgggttgtttgtttactctgctgctgctccttttgtgcaaaagctctttagtttaactaagtcccacttatttatctttgtttttgttgcatttgcttttgggtttttggtcataaaatccttgcctaagcccatgtctagaagggtttttctgacgttaacttctagaattttatggttccaggtcttagatttaagtccttgatccatcttgagttgatttttgtgtaaagtgagagatgaagatccagttgcattctcctacatgtggtttggcaattatcccagtaccatttgttgaatagggtgtcctttccccactttatgtttttgttggctttggcaaagagcagttggctgtaagtatttgggtttatttctcggttctctactctgttcctttggtctacatgcctatttttataccagtaccatgctgtttcggtgactatggccttatagtatagtttgaaatcaggtaatgtgatgcctccagatttgttctttttgcttagtcttactttggctatgtgggctcttttttggttccatattaattttaggattttttttctagttctgtgaagaatgatggtggtattttgatgggaattgcattgaagttgtagattgcttttggcagtatgatcattttcacaatattgattctactcatccatgagcatgagatgtgtttccatttgtttgtgtcatctatgatttatttctttgagaggtgttttgtagttttctttgtagaggtccttcaccttcttggttagtttttgttttttttggtttttttttttttttgcaactattgtgaaaaggagtgagttcttgatttgattctcaccttggttgctgctgttggtgtatagcagagctactgatttgtgtacattaattttgtatcctgaaactctgctgaattcattaatcagttctaggagctttttgggggagtctttagggttttctagttataaaatcatatcatcagcaaacagcgacagtttgaattcctctttactaatttggatgtcctttatttctttctcttgtctgctccggctaggacttctggtactgtgttgaatagtgagagtgggcatctttgttttgttccagttctcagaggaaatgctttcaacttttccccattcagtattatgttggctgtgggtttatcatagatgccttttattacagtgaggtaccttgtataccgattttgctgaaggttttaatcataaagggatgctggattttgtcaaatgctttttctgtatctattgcgattattgcgatggtcatgcgatttttgtttttaattttgttcaggtggtgtgtcacatttattgacttgcatatgttaaaccatccctgcatccctggtatgaaacccacttgatcattgtggattatctttttgatatgctgtttgatttggttagctagtatttcgttaaggatatttttttgagatggagttttgctcttgttgcccaggctggagtgtagtggcactgcaacttccactttctggtttccagcgattctcctgcctcagctccctgagtcgctaggattacaggcacctgccaccacacccagctaatttttgtatttttagtagagatagggtttcgctatgttggccaggcgggtcttgaactcctgacctcgtgatccacctgccttggcctctcaaaggactctcaaagtgttgggattacaggcgtgagccacagcgcccggccagatttttgcatctatgttcattaggaatattggcctgtaggtttctttttttgttttgttctttcttggttttggtacaagggtgatactggcttcatcgaatgatttagggaggattccctctttcttcatcttgtggaatagtgtcaataggattggtaccaattcttctttgagtgtctggtagaattcagctgtgaattcatctggtcctggactttttgtgttgttggtaatttttaaattaccatttcaatcttgctgcttgttattggcctgttcaggatttctaattcttcctgacttaagctaggaggtttgtatctttccaggaatttacctatctcttctaggttttctagtttatgtgtgtaaaggtgttcatagtagccttgaatgatcttttgtatttctgtggggttggttgtaatatctcctgtttgtttctaattgagcttatttggaccttctctcttcttttcttggttaatcttgctaatggtctatcaatttcatttatcttttcaaataaccagcttctttttgtttcatttatcttttgtatttttttttcaatttcctttagctctgctctgatcttggtattttctttcttctgctggctttgggtttcgtttgtttttatttctgtagttccttgatttgtgaccttagattgtctatttgtgctctttcagagtttttgagtaggcatttaaggctgtgaactttcctcttcgcattgcttttgctgtatcccagaggtttggatggttgtgtcactattattgttcaattcgaagaattttaaaattttcatcttgatttcattgttgacccaatgatcattcaggagcaggttatttcacttccatgtatttgcatgttttggaaggttccttttggagtcgatttccagttttattccactgtggactgagagagtagttgacatagttttaattttcttagtttttttgacacttgtttgtggcatatcatatggtctatcctagagaaagttgcatacgctgatgaatagaatgtatattctgtggttgttgggtagaatgttatgtaaagatctgttaagtccatttgttccagggtacaatttaaatccattgtttctttgttgactttctgtcttgatgacctgtctagtattgtcagtggagtattgaagtcccccactattattgtattgctgtctgtctcattacttaggtgtagtagtaattgttctatacatttgggagttccagtgttaggtgcatatatatttaggattatggtattttccttttggacaaggccttttatcattatataatgctcttctttgtcttttttaactgctgttgttttaaagtttgttttgtctgatataagaatagctactcctacttgcttttggtgtccatttgcatggaatgtctttttccacacccttaccttaagtttatgtgagtccttatgtgttaggtgagtttcttgaaggcagcagatacttggttggtgattgcttatccattctgcaattctgtatcttttaagtggagcatttaggctatttaaattcaatgttagtattgagatgtgaggtactattctgttcatcatgccatttgttgcctgtataccttggattttttttagtagtatttttgttttatacctccaatgagatttacacattaaggaggttctgttttgatgtgtttccagcatttgtttcaagatttggagctccttttagcagttcgtgtagtcatgtagtgctggcttggtagtggcgaattctcttagcgttgtttttatctgaaaaagactgtatctgtccttcatttaagaagcttagttttgctggatacaaaattcttggctgctaattgttttctttaaagaagctgaagatagggccccaatcccttctagtttatagggtttctgctgagaaatctgttaacttgatgggttttcctttataggttacctggtgcttttgcctcacagctcttaagattatttttcttatcttaactttagataaccttatgacaatgtgcctaggcaatgatctttttgtgatgaatttttcaggtgttatttgagcttcttgtatttggatgtctaggtctctaataaggctaaggaagttttcctcaattatccccccagatatgttttccaaacttttagatttctcttctttctcaggaacaccaattattcttaggtttggttgtttaattctgtcccaaacttcttggaggttttgttcatttttttttttttttcttttcttttttttttttaattgatcattcttgggtgtttctcgcagagggggatttggcaggatcacaggacaatagtggagggaaggtcagcagataaacaagtgcacaaaggtctctggttttcctaggcagaggaccctgcggccttctgcagtttttgtgtccctgggtacttgagattagggagtggtgatgactcttaacgagcatgctgccttcaagcatctgtttaacaaagcacatcttgcaccgcccttaatccattcaaccctgagtggatacaccacatgtttcagagagcacagggttgggggtaaggtcacagatcaacaggatcccaaggcagaagaatttttcttagtacagaacaaaatgaaaagtctcccacgtctacctctttctacacagacacggcaaccatccgatttctcaatcttttccccacctttcccccctttctattccacaaaactgccattgtcatcatggcccgttctcaatgagctgttgggcacacctcccagacggggtggtggccgggcagagcggctcctcacttcccagtaggggcggccgggcagaggcgcccctcacctcccggacggggcggctggccgggcggggggctgaacccccacctccctcccggacggggcggctggccgggtggggggctgacccccccacctccctcccggacggggcggctggccgggcggggggctgacccccccgcctccctcccggacggggcggctggccgggcagaggggctcctcacttcccagtaggggcagctgggcagaggcgcccctcacctcccggactgggcagctggccaggcggggggctgaacccccacctccctcccggacggggcggctggccgggcggggggctgacccccccacctccctcccggacggggcggctggccaggcggggggctgacccccccacctccttcccggacggggcggctggccgggcagaggggctcctcacttcccagtaggggcggctgggcagaggcgcccctcacctcccggactgggcagctggccaggcggggggctgacccccccacctccctcccggacggggcggctggccgggcggggggctgacccccccacctccctcccggacggggcggctggccgggcggggagctgacccccccacctccctcccggacggggtggctgccgggcggagacgctcctcacttcccagacggggtggctgccgggctgaggggctcctcacttctcagacagggcggttgccaggcagagggtctcctcacttctcagacggggcggcccggcagagacgctcctcacatcccagacggggcggcagggcagaggcgctccccacatctcagacgatgggcggcagggcagagacgctcctcacttcctagatgggatggcggccgggaagaggcgctcctcacttcctagatgggatggcggctgggcagagacactcctcactttccagactgggcagccaggcagaggggctcctcacatcccagacgatggcggccaggcagagacgctcctcacttcccagacggggtggccccgggcagaggctgcaatctcggcactttgggaggccaaggcaggctgctgggaggtggaggttgtagcgagctgagatcacgccactgcactccagcctgggcaccattgagcactgagtgcggttttgttcattttttaaattcttttttctttgtctttgttggattgagttaatttgaaaaccttgtctttgagctctgaagttctttcttatgcttgttttattctattgctgagactttcaagaacattttgcatttctctaagtgtgtccttcatttcctgaagttgtgattgttttttatttatactaactatttcactgaagatttctcccctcatttcttgtatcatttttttgacttccttaaattggacttcacctttctctggtgcctccttaattagcttaacaatcgaccttctgaattctttttcaggtgactcagggatttcttcttggcttggatccattgctggtgagctagtgtgattttttgaggggtattaaagaaccttgttttgtcgtattactgggattgtttttctggttccttctcatttggtaggctatgtctgagggaagtactagggctcaaggctgctgttcagattcttttgtcccacaggttgttttcttgatgtagtactctcccccttttcttagagatgtggcttcctgggaaccgacctgtagtgactgttatttctcttctggatctagccattcagcagggctaccaggctccaggctagtactgggggctgtctgctcagagtcctgtgctatgggctgttttcaggtctcacagcgttggattccagcacctgctctgatagaggtggcaggggagtgaaatggactctgcaggggtccttagcttttgttgtttaatgcactatttttgtgctggttggcctcctgccaggaggtggcactttcaagacagcgtcagctgtggtagtatagggaggatcaggcagtggccagggccttagaactcccaagagtatatgacctttgccttcagctaccaggatggatagggaatgaccatcaggtggggcaaggctaggactgtctgagctcagactctcctcgggtgagtcttgctgaggctgtgctgtggggcaggggggtgaggttcccaggtcaatggagttatgttcccagaggattatggctgcctctgctgcgtcatgcaggctgtcaggaatgtgggggaaagccggcagttacaggcctcacccagctcccttgcaacccccaaaaccggtctcactcctgtgccctaccaacagcatcaagtttgtttgcaggcagcggatgagcaaggctaagaacttgccgcaggctaccagcctcccagcaaagaatgtaagtagggctttcatgcctcccttcctgttgaatctgtacaccaaattcactccctcccccaagttctggccaggtgacttcatgtttggttggaattgttacaaagttcagctggaggtttccttctcgctgtggtcttttcccagttcctctggccaccctccccaaggacccctgtgagacaaggtagaaatggcttattaggggacccagagagcccacagggcttttccccttgctttctctaccctttatttcactcagctgtctaattaaatcagctccaggtaagttcatatccttctcccatgatctggagctgcagattccccagtgagggtgtgtgttcgggggtgggctatccccctttcctactttcacagcttgggcactcacagtatttggggtgtctcctgggtcctgtaggagcaaactgcttccttcacagggtctgtggattctcttggctttcctggtatattcctgtagtagttctggagcagaagtttatggtgtgagtttccacacactgctctgttcatccaagtgagagctgcaatctagtcctgcctcctttccaccattttttggccttgacgtattacatttattttatgtttgaagttttggcagttttattttgaaaattttacataacaaatgccaagccaaatccaaacttcttggcctaaatcatttacagataacctttactgagcatttactacatgaaggtgttgttgacacaggggatttaagatgtgatcactgaccacaacatccaggcaacctaaaagtgaaaagaggtcattcacaaaaaataggaccaaacttcctccattcctcaagggacctaggactggagttggcctaaaaattcctgctgtatcacctcacccaccagttttgatcttgctctcagggatcaaggaagctcaaggtttctggcccttttggctcatgttcttggaggtatattctattgtcaagttaaagtcgtacccatcctagatctgtttctctctctttgtggttccagtctcatagaaaacataactatgtgaaaacaccaactaccccataaagctattctgagactcaaagacatgaactctattttgataatcacaaattgatgtatagatgtcagatatcattcatgtattatgtagcattgtggtttaagtcttagctgttggaatcagactacttgcattcaaattttagctctgttgataggtgtgtaaccttgaacaagatacgtaacctcttcagacctttgtctctttgtttgtcaaataacgacaataataatactaacttagtaagtatgtataaggaggcatgtaaagcttctattgtatttggcttatggtgagcccttcatataaattggttacaatgggtaatgaaaagtacgttcaatttttcccacaaggatttaatctaatctctaaataaaatgccagttttagtatactgtcatcattataaggaaactataattcttaagtaattaattactttggttccctttggataattagtgatacataaaatgcagtaccttgccctgaggaacctacaagttagcctccctctaatcctggcattcaaagtcctccaccttccgaattacacctgttttttcaggcattgtctcccactttcctagcaagcctttggctccaggaaactgttctgttcattgacgacagaatgtattttgtcccgtatgcctcccttcaatctgaagttcatttttagtaattttttttattagtagtgactttccccatttactaatgcctttctctttagctcatttagaattccctggaagtcatcatgttttccttctgaattgctatcatattctgtctctgtgtctaatcagaaagttaggtgagctgctgtctcagctcctgtgttaatccaggtactgagctctttgtaccctgccagcttgagggcaacttggatgcgctggtgtgtagtgatttgcaactgtgagagagccgtttccatcaacaagtgacagcttttggttctaaacactgcttgacctcattctggtttgaacatatactttggcctctgccctctacctcacctccagttctgatcttgggcatgccacagaaccccatctacactaggctttcattttggacttctaagtcctttacttgaccacttattttctttaattgctagacttaatcatccatgcatatgacagccactcagctctgaccccaatacttcattgaggctcatgccagtctgacctctcacagggaagagcacccctgccggtgagcccctggcccttggattatgctgtcccaccagtgcccagctggctgcttggcactgcatgtgatgtatctttgtttattgatcgcttaattgaatgggatataggattatattccatgttccagatcaagttgagccaagctgacaacttagtattaaaaaaatatatttatctcctatctgtccaattaaaattttaatttctagaggtagagacagtcacattgctatttaattcctagcacagcagtatttttgtcaatatttgttattagtggtgagtttaaattataaaggaaaaaagagaagtggtgatttatgtcagcgcttggctaaactattcccttgatataagtctattattcaggtcacataagcatgactgaagtaatatcacaaccataatatcataattatctctcttgctgtctctgtcactttctcacttttgtttgtgcatagtgcatagacctgtagaaaatttattagtaatctgaagcataaaaccaaatattacaactatcataacaaataaataaaactgtgcactcctttagggagtagtaaagccatttctaaaattaagtcaaagtaagattcccttattattgagatattttagtggtatacactatcataagtaagatgaataattttgttagcagctgacactgctatatagatatttatcttgggaagttctcagtgtaacatctttactgtgtttttctctttagattatacttttatgtttattattttgtttcgtgggggattcaaaaatatgcattttatgccatacttggggattccctataaattattagttgtaatatgacagttccatcatgaattttccaggtattcttttatgcaagcagataatagctcattttggtttttaacacattcatgcatattctttctctctcaataaatatggttttaatttattaaatgaaagatttaaaaatgtgctaagcattttaataataatcgattttggattaacttgttatgtttactctagggctgtagttcaccatttattcagttaagtccttccccaaattcaatattgaacatgtggaattgattcgtttcacgtggatgtatcatttactcccaagatgttggtttttggcatttagtactggtaatgggccaggaaagtgctcatctatatttgttgttattcactgattgcatcttgcccttgcacccactgagacgatgggaaagtagcaacaatactaggtgatttctttgatttaaacccaattaaaagaattagagagttgtctgatacaaggccaaagaactaagaacagaaacaaaagcaaaacaacaaacagcagcacaaactccagtgagataaatttttaaaacattgggaatatttaaaaaataaaaacactccaatgaaccacccaggttttattaaagagtagagaactcaaacagcagaaggcagagctggtggaggaaactcagcaactgctcagaaatgaaacaacctaggaaggaggtgcgagtgacctgaaactccttttaaaaacagaaaggacaaaaagaggtatgggctgacaaaaggaaagtggtagattactgatgtattgcattatgcttagaatgtctcagaatgcgagaggcagtaaacacaccagaagaggatacaatatccggaccatgtgcaactgcaaataaatgtttgggttaaaacttggttgattctaaattacatgaagagctgatgataattgaggcagaactgatagacctaacaatagaaaaaaatcgatttaaattcagagaagaggtcatttaagaaagtataatgaagccacttaataaaaggaaatattcctctaactaaagtttgagatttagggcataatcctcaaagacaggctaatataatctattttttgattaaaaaaaggaacttttggtttaaagtaagatgttcagctgctcttagagtttatttctccatatttgggcatataagaatttaaggaaaaatatagaaatataagaaagatttcatgagaatcacaagcatagtttataggcaagatagccttttctgtttgaaagcgaaaaatactatttcaacatgtaaaaacctaagtcagttttcactggcatgtcccacaatcatgcctgaaataatggttgaagacagatgtgagacatttcaagggcaatagattaatacatgaaacccacttatgcctagcgttccattattggaacgctaagcatgtgggagttatttatatcctattgctcaaggtcatctccaaggtctgagttttcactcatgcaaaaattcaaaaaattgcaacctcggcgtaaatgggttaacaaaaagttaatgctggacagtaaaataaactactaaattagacacaccatattttttaaattataagagattaagaactatgagatatttaaaaagccacccacagaagtagtaggacaggtagagaaggataaattctaacaatcaactgtatctccaccccaccttgtaaatgacaaattagtttacttagtcaattcaggaaattaaacgtatacattttgtgttaaaacagaagactttttttaaaaaagtgtgttagttaaggttaattttgacctagttaagaaaaagcagtttagagtcttgaaatggaaatgagaaatacataattacccttaaaaataagttgatagtgaattttatctaggattcttaagacatttttaatatttaatgaaaatgaactacacaattattaaaaaatagttgcctgggtttagaaaaatgatcccttaataagacatatacaacagcctgaatttttatttccaaatactatagcaacaaaataccggcaagaaaaatgcacagaaacattggatagtctcttcttactaatcttttacagatatttatatattctgaattctaacttttaatttattttatgtattataaatgtcatctagtatgcagcttgactttgctttatggtatcatctgtttcacagaaaattttaagttgtggaatttatcgtattttttctttatagtttgtgctttaagacctgtctaataaatctttccttccctggacgctataaagttatttgtctatattctgttaaaaggaataaaactttgctttttttcacattttgttacttatttcacctgaaagagaattttgtttattatgtgaggtaggaatataacttaatttttttccattgaataaccaaattgtcccagagcaattaagcaatacattctttccctgttgatctgtgaagctacctccattaggcatgaagttgtcctgttgaagagaatctgattctgagctctcttcactatttctttatttgtctatgtctacactaaaagcaatttaatttctgtgtctttataattactttgactttataatccttgaaagggactttgttcttcttcaaaattgttttggctatttatggctacttttgctttcaaatgagtttttaaatcaaactttaatgagattgcattaaacttataaattaatctgaggaggattgacatcttcatgatatttagtcttcctacccattaacattatataactctccatttattttaggtctttttgaatgacttctaataaagtttttcaattttctccaaaatatcttacatattaaaatttactcatagctgtctcatcttactgatattttaaagacatatctttttaaaaattatattattaaattgtttcaggtatgtctttttttttgaagttttacaaatttgggattatactgcctcttcattcaacattttcccatgctgttggggatccttcaagtcaaaattattagaggagctgcattaatattccagcatggacatactataatttatttaattatttcccgaattgtatgtttttgtttctaacttcacagttttcaacatgactgggaacagcttttcttgctaaatccttgcacgccacagtaattatctcactgggccaaaagataggtactttttcatgactttcgccatatattgtcaaaatggaaccaatggtgatttgttcagaaatcatggtacatcatataaaaagattactctgcaagcatcacaaactgtgatatatagaaatgtgtttattgacatgaaatagatcatgaagaaagtgattacaaatggtatttaagcattttaagttatataggcttatactgagcaataacaaagagggtgaataaatgaatgaatatgtgattctggaagggtatgtaacaaaaggttaacaatggttagccctgggtaaggggattatgtatgacttttatttccttctttttttcttcctttccccttatgtttttaacaatgtacatgaatcgtttatgaataaaataaaaaactttgatattcttatataggtttagtaggataacatttctccttcatttctttcatttttagaaatctttcttcacggaaattcttttttttaaatgtttatttctgtctttgaggagtgagattgactggctatcttttaatacctgtgtgtattatcattattgtatcttgctgataaatttcacaacagttaattattttattccccatttcatacttagctctattttaggcaagttcacaaatatcattgaatgagttcattcaatgtcatcatgatgatattgaaatgtcaatagcagatgacttgtttctggggtgttaatatgcctgttggttacatttgtgttttttggtgatggaaggcagccactgaggcaaacagcaagaatacagctaagcctctaactgcatactcttctcactcgttgcttaaaactgaagcgtggataattggtgtgcattccctaaaaagttatgtcatgatgcagtagagaaataagttaactttcctatccctgtatcaaagtttcttttgaaggtaactgagacagcgccaaataaggagcagagtttctgtgaatcatgaaatctttctaaacaattgagaaaaaaaaaactgttctgaaacaaaaacttgtgccctatccttttatttgaatgtgttttcttaaggccacaactggcagagatttgatgattttatgtttaaagcaattttttttttttttcatagcaaggagtcccactgccatcaggttttgtttttggagagattctgtaactgacatagggtaacttactctgatggcttgctccacattcactacaagtacatactgttctgaaaaatctcaaagttgaaatatttttatttgggcaacttctgcaatcaaggtaattcactctatctttggagaataaaatggaaaagggcaacccaatctctgtgaccttaaacttccactgaaacttagagacggttagcttagctggtgagagcgtgctgctaataacaccaaggtcgtggtttctatcccattacagggcagtcaagctcaagggaaaaaccctgttctttggcacagcttccatggcctgctagccgtttacagcgaggcaaaagagtagagatgttttagtgaactaggccatgacttggggagtctccaccttgaatctggttgtgggaccaaggaagagaatctgctgaggctgggtagagagactctagagcaccttaggccacaggacagcagaggatgagaataacagaactggcttcagaaagtaggtgtcagtccaaggcactgtattcaatacatgtagcaaagaatgagcttctgacaccaggaaagtcttcagatggcagtgactgcataaaccggtgtcaggatgtctgactataaaggatgggacaccttccatcaggtgagagctctggaggtggagtctgttcctggaagaagcatgggtaaatgtcatcaaaggttccctcagccaccactgggttcagggctgaggtgcacctaggaaggcttgggcagaagcatgacccctggaacttggccaactgagccatcagagagtggtgcaggtgaggagtggtagatgtgtagtgaaccactcagctgtctgcccaggccttgggtgtggaagtagagtcttctgtgtctttgggcagactgaattataagcttcaccagatcgccaagctaggaatgttgacatacctggcttggggtcagggagggactgaactgtaggtggggggccccaacaaatctaatcataagaatctatgagcgtggagacagaaggaccaaagaagggatgctgtaaccaattatcattattgaaaaactaggcagagattacattcaattggcagtaccaccctttttatctctgacatagttccagaaggatatactatgtatctagtgtgacactgtctgatagaattgtctgaaatgatggaagtgttctgtggctacccattttgatagccactagccacaggtggctattgagcacttgatatgttgttggtgtgactgaggaactaaatttttaattcaatttaaattaaatttaatttaaatttaagtagcaacatgtggctggtggttactggattgaaccgaacagatctagatactagagtaagtggtagcagcaaatcctatattggaacaatgtaaattaacaattataaatatatgagcaggacaaagcccaaatgggcctgtactcttagttttgctatatattcaaccataactagcactcattttttaaacttataaaatgcaaactcatttggttgtgatgatgaatgctcaggtaattgttgagtttcaacacagtgacagccaggaactcattttggagacttggattgtgccacagtttgtccctctataaaatgatgatgaaaattttatcagtatctgtctcaagctgtagttaatgggccaatttctaaatattcctgtcaaattgtatagccattgttttcaaagtagttttgaaagtaggccattggtaaagcaatctattaaagtgcaggaagaaagtaatagaatctcttactttttaaagtctcattatgaaaattatatttcagtttgtatgccttcgatgtacataatatattaatacacaaatatattaatgcagcggtccccaaactttttctcaccagggactggtttcctggacgacagtatttccatggattgcagcggggatggtttcaggatgaaactgacccacctcagatcatcaggcattattaggttctcataaggagcacacagccttgagcaggtgcagttcacatatagggtttgcgcacctatgagaatctaatgctgccgctgatctgacaggaaatggggctcaggcagtaatgctcgctcactgctcacctcctgctgtgtggcccagttcctaacaggccatgaactggtaccagtctgtggcctgggggctggggagtcctgcattaatgtaattaaatatgtacatttcttgagagtgtttgttcaaatattttttcctaatagagacgtataacttagaaagtatggaaccctctggtcagtagcatattttatcaaaaaggctatccgtcgtgctaaccaatgtgattaaatgttcattttagtctcagatattttgataacataataatagggaagttatggttattgcatggcttttctttactgtattttcaataactatattaatgggttcagttgttactagttttataattctgttttattctcaatgagtcttcctgtttctccccacgtctttcctatggttcatttccctgcatctttacagctccctggaacattctctcaaatcaatcagaggcttcaaaactgggcagtctcattttgtaacctgctctgactcgcctctgggaggcctccctccccactcacagctgccttcctgcatgatgtaaaatgaatacagtgccagaaagactgatccttggaacctcttccttacttagtttctttatagttctgattttagaaatgaccattttacttccagtgcccttccccacaccagttagctgagactctatgcaggaagttcccagtgatcagaactgttcccatagctcccaggtgattctgacatgcacccggcattgagcaccatggacatggctgctgtttgttatcctcgagctctgcaaagcagtgtcggatattgcttgattgttgcacattaatctactgcacggtccggtaggtagccactagccacatgtggccatttagatgtaaatgaatttaaattaaatagtattataagttcaacacctcagttgcactctctgtattcaagtgctcagtagctacaagtggttggtggctaccattttgggcagtgctactatacaacatttctatcaccacagagagtgctgatgaataatagtactaacaactggaaacattgaaatggagaggtgaaggagagaaggcagaatataagagaaacttcaaagcattcagcgtgtctgataggcgctgtatcttacattatgtttggagcatagtttttgaatttcttaattttcaaattcggttgaaacagattttggtcaattgaagtgaaggactatgcctgctttaatttgcgtagcttacctttgtttcacgtgtctcccttattacagtgtttgtagcagcatctaactcaatccctaacttctttttggtgcagggaccatttaaaaacaatttgatcatagaggttatctcctttgcaatccatattgttttatggaacaggtgaacagctggtgacaatagcctcaaggtttctaatcccaccaaaggaaaatatggtgtcagtaattaccttgtactatgatatttgtgaaatccaagagagggctagatctcaggctctaataatacatccgtataaacatctcagtagggcaccacggaagagtcttaggatatcaggaacaataagagcgggagaatatcgaccttaatattctttacacttattaaaaagtgaaaagacagccataaagcagaatgagttcctgtcctttgcagggacatggatgaagccagaagccatcattctcagcaaactaacacaggaacagaaaaccaaacacctcatgttcgcactcataagtgggagttgaacaatgagaacacatggacacagggagtggagcatcacacaacggggcctgtcgggagttgggggactaggggagggagagcattaggacaaatacctaatgcatgtggggcttaaaacctagatgacgggttgataggtgtagcaaaccaccatggcacatgtatacctatgtaacaaacctgcacgttctgcacatatatcccagaactaaaagtaaaataaatgaaaaagaaataaaaaataataaaataaaataaaataaatgaaaagtagccattttaccagttgagtatttactgacttttggtgaaaatcactttctaagattactagaaaccttatgaccctcggcctctactcccaccacgtgcggattttcacctccacctactttgtcacgtcacatgcccttctgctctttaggacacttgtattgggttcctcccccacacgtaaacagaacacacaggtctgtgaatggacctcattatgctcatttgtctttgccaagaactattgaaaatgatcacaaatgactatgtaattataattaagattttgtacatgcatacactttatttttaacaagtttgtcctgttaaatgtcaccccattttgaatagtaacattcactggtgcaaggaagaattattaaaatgacaatttaactgatatccttgacaggtaagccagttattgtatttcataattgtgattattacaatacctatgaagattaattaggacaatttattctataggacttggttatcaaaatgatcttttttcccattttagtccatacatcttcagttttaatttccttcttccaaagaactctgagtcttgcccagtgtgatacttcagattttgtaaaaatataatgaagaagaataacatgtatcagatcttttttttaatgtactgggctaaaaattataagcaccatcaaattactagaaaattcttttaacactctgcttcttagttaaaaaatgattcctttttgtgataatcttataattttcaaagtatcattttagaaattttaaaaaattattcttgttttcattcagtccatttcatcagaacttagattttttcttccctctacagtatgtggtcttgatgtgctttctccttgcaaaataaaatctttagtttcaggatctctgtgttctattccttcaatttaaaatcataggaaaaataactatcatttccttatgtctgtctccaattcccccatcattttctgtgagccaattttcttctttttcttctgtctactgtaaggaactttttactctgctctattctctgcaattcagggctcacaatttctttgacttctcagattatcctttgtacttgtgtctactttttcattaaaaaaaaaaaggcttcggtcttattttgactttatcatctttatattgccatatttaaaacccatctgcttattgattgaaccaaacttttcatcttacttcatcatactgaatgtataacttctgaatgacttaaaaatcatcacataatgtgagagaattttctatcatattcaggtgactgcattgtaagcaatgaagttgaagaaaaatagcttataatggatataataccaagaaataataagaatgagaatctgcgtatatcatatactgggttaacaagctcacaaggcaaatcaccattttattagatatttaacaaatcatactagtactggaggaagtatagcagaaaaaaggagtatggacattgggattagctgccttgctaatcttttgtcagtccctagtgacatgcttttgggttgagtcattgttacctccctgagctgtttgtttataaaatgggggtaaatatctcagatgtttcagtgttgtgacatataaagcgtcctccatagtacataataccgtaagcactccataaggtaattcttgtccttttaaaaagtatacacttttgagttcaggacacgtacactgtaacctagttgtaaccattttaataaaagaaggttttgatcatttttgataaatgtactacatgaatagttaacttgcctttcacttcctataccaacggaaattttggtaacaaatttaaagaaagcattaggttgaatatcccatctgattaaatgtcaactaaatattgtttctggcttttaaaatatttctaattcagttataaaacaggtctattactcaggaggctgaggcaggaaaattgcttgaaccagggagtcggaggttgcagtgagccgagatcatgccattgcactccagcctggcgacagagtgagattccgtctcaaaaaaaaaaaaggtgaaatgaaaaacaaaaaagaggggtgaaatttctctgcattctccctttctgctgttgggaaagccctttcattaccaagaatgcatggaattctgcttattaatttctccatctatgtgtctctgtgaaaaatactaattatttacaattttgtaagctatgaataaggtctatgttttctctgaaattatagcaacaggagcatttcccccttatttgggccgcaatcttttcattctgattcttaccataacttctgacttctgactttatcatgtcttctgatttctactaaacatgaacagaatgaatgacttctagaaatggatacaggtgtaatacatataaggccagggaaccacttaattatctttacaaagtactaggacttcagaaaaatcaaatgatgaaatggagctagctttagtatgttctacttatcattggactattaatcatgtctgtctgatgagttacctaggacttcaactgtttttatagatttaaagaaaatattataaatgtatcttacctggatttaagactggacagctgattttggatttttttctgctaccctgttgttgatatcactgacatcagcctttgcattttgactggaggctcctttctctctttctctctctctctctctctctctataggaaaccagtagttatgtcaggttgctaagtattttcccactgaaggacaaatattctggagcagtatatatttagcatttgacatttgataaacaagcttgtttgagatattttcaatacaatttcagctttggcaattgtaagcaaaggatccagttggtaattggtgaaatgaacagatgtggacacgctgcctgcatcgagatgcattaatttttctgaaaagtggataagatcttgtgagaatgaataatggaattcaaatcagatggggtgagatatttgatattcaggaacaaatcacagtataatagatctgctactttgtttggttttaatggggagttgctaatgctgaaatatagtcagagaaaaggaacgtggaatttctatagaggagagcctggctctggggtaatactactgtccttggtggaatgttgtcttttctgaagacattggttcaattcaatacatgaaacattcatcgattattatttagaaaagctaatctactgacaagacacagagttaagtagtgaaaatacagagacctagacacagttcttattaagaaattttttaatttaggtggcatatttcagagagaaattctctgcttggggcaataggtacctcctttggaataagcatggtgagaaagaattgcttctggcagattagtgagaagagtgagatattttctttcgatgacctctctattaaaatttgagtggtaaaactttgtgatgattcaggctcttcataattcttaattttatcatcctctaataccaggacttgcccagatcaatattttaggaaacagtcatgcttgctaaacccagggatttctattaaccactagataagacataagtttgtcatgcaacaagtatttattgagtccataatttgcccagcactgggctttggtactctgggccttgatactctaggttgtgtcagagatgtctaatatatttaaggtgacagagttcacaaatgagactttatgaaaatgaatatccctttttaagtctcgagaaatatgatgtgcacccgcttgaggctttattaaatctccagcgagcatgaacttgtttgtgacccaattgtagaattgttgtatgatgactatcccactggcaggcacttcttgtccaagagaatgtaattggatgttggtgactcaagcaatcctgggaagactcctccatgaccaaattaaagacaacaggggcttggtttgtactcagctctacaccaatcagcatgagacaaagaagagatccatggcaaagtggggagactactgtttattttacaagctagagaaggaaaactgcagttcctgagttgcaaaactgctaagaaatgggagaacacagaactttacaggctgaaccttggtgtcttagttattcctcttggctacagaatgccaaccagtaggagattcatcactgagatattacagggaaatgaagccaggcaagaaaaatatgtattccctttcttctcagcccataaattttgttattaataaatcagactcttatagaacagcttgccacggctgtctctagaaatgtttttattagcagaatttttattaaaataaaatacacagtagttttaagagtgaacattatagttttaggtcataaggggtgtcatggagaacacatgtggatgctacttgccagttacttgatctaggccttgactcttggttttctgttgctcggataagttagcatgatttgacatgcagttaaaggtgtggtaacctgtgattgatttcccattcttgatgctccctgcctggacttctcggaaaatttcaaaacaattcattcttctatgagggctgcctccttggttgcttcggctaaacagtgttgagcaacaagttgggaaaggaacgttgcagtaacttttaaaaattaagttagaaacaaatgtcatcaaagtaaatgataatttgccaatatgattgagcaaaaagatgaagatgcacgctccctacgtatttgctttgcagaagaattaatttgaaaaataataacatattttaaaatgaattgtaaatataaacacatgctaattgcagaggggaatccctgtgattattcagcagtttgtgtttgtcaagtgcttttcagtcagtccattccagctcagcagggcagaggcttgggctgttgtaaacttgtgggcagatacccagtgatggggcagacatgagcaggtagggctgacagcatgtgaattgagttttcttcagtcatgctgttgcagctgctcccttccctcattgctgagtttgccacagcaggtaggaacctaactctggagcctgggatgaaggagaacccacattgggcttgaggaacaagatctgccaccctggtaggccctggttaaatctcatgcaagtgtagcaatgagagagggtatgattgagttctaatttaggaggaaaggggataatgtgccctttgcacccccaggagaaatcattgctcatctgtgcctaagtagacttatcaagggcagttggttcacaatggtgtatcaccccaaaggactatagtgttatgagaacttgccagtgtatttgaatttgggtgctggcagatgacatcatgaggtattatggttacccataaatatgctagtttattgagaaggtggtagacatgctagtggatgagagggaaggacagaagcagttagtaaacagcatctgcaacaattcagttaactggtggttgtcacagtagcatggtggaaaagttggcaattaataacttctaagaaaactgaactaatgaaccaatcctgcgtgtgctatgtgtataacctccttctcactattaacagatttgttccaaacttatataagacaatgaaaataaagcttggcaatatagggaagggatggagggataaagctgtaaatcacgtcacaggcaaattaagatataccactggatcaagggatttaatgcagaaagactgatcctaacttatttcttttatttagcaataagatttgttacttacattgattatttaaaatgagttgcattattagaaaggactattttgaagacaactataataaaatgtcagtaactgatagtagccaagatattttaaatatatcaaagttgtgtcattaatattaatgtgtcccttaatatgaagtcctgcccaggcttatttatgtattcaacagacacatacctgttgaagtgtaacagatattctggacacaagcaatggtaaacaagacagatgcagtccctgctttcatgcaatttgcaattgaatggtctttgacattttattgtgattgttttagttatttaattggagaagtttttaatttaaatttgtgttatattcagtgttaaggaacaaaaatgtaatgtgcatttctgagactcagtaacacttctggtttttcctttttcatttaaagaaaaatttagtgcccaagataagctagaatttttggaatcaagtaattgatgacctgggagccaattttattacaatagtgtttttagtggtcttagaacttttcagaggtggtagctctgaaaataacactgtaataaattcacacatacatctatcatccaataaatgttaattgaggcccactacagcattgtgttagattctgaggttacaaattgttgaccccatgtcgaacatgttgaccccatcaatgtaatcagtttgacattacacagtcatttaaacattaaagtgtcagcggatattttagttgtaattttgataagtgctctgaaggagaaaacagtgggtgttgtgaaaagcagtatttggttgattgattattatagaggatctgagaaaacttacttgaggaaggaacatttggctgaactcaaagagatgagtaggagttaagtaagcaaggaagaaaagaagacacatgaaggaggaagaatgttctagaaacacacacacacaggtatatgtatatgtacatgtatatgtatatgcatatgtcttccttagaaacatataaacagctgcaacatgattgaacttatttacccaattaccaaagcttattttgcaccatgaaggtggagataaaggctttttaagcagcaaggatagaatctttgtagtttttatttataggctggttcttctgacaactttaatttttcatctttaccaacttcatggtcttcagtacatacaatgcaatcattattataaaattatattttgactcaaactctaaggtaggatgattcagctgtgcgccatcaacatagcagcatgaatggtagagactagtcattccaaacagtgaaggggcaacgtaaaactaattttaatattatatgaaagtacttcttgcccttgactgcttttttttttttttgaagaaagcaaactttaaaaatttattttagatttacagaattattgcaaggatagtaagagagttctcatatatgcctcacccagtttcctctattatcgacatcttacattatatggtacatctatcataactaatgaaccaatattgtttcattattagtaactaaatctatactttattcagattttctaagttttcctctaatgttctttttctgtcccaggaccccatcaggatatggtatgtatagttgtcatgtcttcccaggctcgccatggttgtgacagtttctgagactttcattgtttttgataccccaggtagtttaggcattttgtagaatgcctcccagtctgaatttgtctgatgttttcctcatggtttgactggctttaatgtgttttggggaggaagaccacagaggttaagtgtgattgtcatcacatcgtatcaagggtacatgccatcaatatgacttatcactgttgatattaaccttgatcatctggcttgagatagtatttgtcaggtttctgtattatacagttactcttctccctgtccatacagtactttttggaagaagccattttgtgcagctcattttttttttaattttaattttaagttctggggtacatgtgcagcatgtgcatgtttgttacatagttaaacgtgtgccatggtggtttgctgcacccgtcagctcatcacctaggcattaaagccaacatgcattagttgtttttcctaatgctctccctcccccaaccccaatctgacaggtcccagtgtgtgttgttcccctccctgtgtccatgtgttctcattgttcagctcccacttctaagtgagaacatgtggtgtttgggtctctgttcctgcattagtttgctgaggataatggcttccagctccatccatgtctctgcaaaggatttgatatccttcctttttatggctgcatagtattccatggggtttatgtaccacattttctttatccagtctatcactgataggcatttggtttgattccatgcctttactattgtgaatagtgctgcagtgaacatatgcatacatgtatctttgtaatagagtgatttatatttcactgggtatatacccagtaatgggattgccaggttgaatggtatttctagttctagatctttgaggaattgctacaccatcttcctcaatggttgaactaattaatttacattccgaccaacagtgtaaaagtgttcctatttctttgcaacctcgccagcatctgttgtttcttggctttttaatgatcaccattctgactggtgtgagatggtatctcattgtggttttgatttgcatttctctaatgatcagtgatgttgagcttttttcatatgttctttggcctcatgaatgtcttcttttgaaaagtgtctgttcatgtcctcttccaactttttaatagggttgtttgtcttttcttgcaaatttgtctaagttccttgtagattctggatattaaacctttgtcagatgtatagattgcaaaaaatttcccccagtctgtaggttgcctgttttctctgatgattgtttcttttgctgtacagaagatctttagtttaattagatcccatttgtcaattttggcttttttttgcaattgcttttggcagttttgtcatgaaatctttgcctgtgcctatatatttattgcatagattttcttctaggggacttcaaactatgctacaaaacttcaataaccaaaacagcatggtactggtacaaatacagacacatagaccaatggaacagaatagagaactcagaaataagaccacacatctacaacgatttgaccttcgagaaacatgaccaaaacaagcaatggggaaaggattgcctatttaataaatggtgctgggagaactggctagccacatacagaaaattgaaactggaccccttccttacaccttatacaaaaattaattcaagatggattaaagacttaagtggagctcatatttaaggagtgagaagatatgctctacctctttaagggtggagtagctctataaattatttggaagtgtctattctcctccattaatttatttagtcaacaattagtatcagccatctagaacccatgaatatttatgctttgggtacagtccaatactattttattttgtagctcatcttgttccagctttggccatttggagatttttcagttggctcctgtatctctttggcttcttacatatcattgtagggttttttaaaagccttttcttactttctgtcactacaagatagttcagacttatcttctgtattttttgccccagttctatgatcagccacttctccaaggagcaataatttccttcaatgaaaaccaagatatgggctgttggtgtacttgttgttattgtgtgttgttacttctagatcctctaagctgatagtgcaaagagatatatgtgtgtgtaccaacctatatatctacacacatataaaaatatttctatttgtaaccatctgtatctatcttaggctaaacctgagtacctactgatgtctccaattctaacctgcaacagcatggaacattctagccttctcctcttacttatctgtcacttcctataccaatagtgagaaacctggctcctaccatctgctatttatttacttaattatttaattccactatacttctatggaagtttcagaattgttaatctgtactcatgtacgaaacaactttatcaactagagtatagtgtttatatacagttcctttgcctttattctaacagattccacttactcattttccgagtcacttaggttagcgccttattttcctaagtccattagtgagtttgcttcatgtatttgtcatacatttaaattcttttgtaatattgtgcattccatcccagtttcccctgacatcctaaattaactttttaagtttggatacattgtggtctattctttgttctgtaaagctttatggattttgacaagtatttaatgtattgtatcaccattatagtaatatagttcctatggaatagaatagtttctatcgctgtagcatagaatagtttctctactctataaaatatcctgtgtttctctaattcaacccctccttcccaccttgaactcctgacaacccctggtctgtttaatatctttcttttgtctcttctagaatatcatataattgaaatcatacaatatgtagctttttcagactggctactttcacttagcaatattcatgtaagtttcatctatatattttcatggtctgatagctcatttctttttaatcactgaataatacttttatttatccactcactggtgaagaatctcttgattgcttctaaattcatggcaattatgaatgaaactgctctaaacatttttgtgcaggtttttgtgtgcatgtgtattttcaaattagttgggtaaatatctaggaattcaatttctgcatcattgtggtaataatgtgtttagcttcataagaaattgccaacctatcttccaaaatagctgtaccattttgcattcccaccagcaatgaatgagagttcttgatgctcgacatccttgtcagcatttgatttttgtcagtgttttggattttaactattgtaatagatgtgtagtagtgtttgattgttttaatttgcaatttctttttcttttttgagatggagtctcgctctgtcgcccaggctggagtgcagtggcacgatcttcgctcattgcaacctctgcctcctgggttcaagcaattctctgcctcaagctcccaagtacctgggattacaggcgcctgccaccatacccggctaattgttgtatttttagtagagatggggtttcaccatattggccaggctggtcttgaactcctgaccttgtgatccacccgccctggcctcccaaagtgctgggattacagacataagccacggcgtccggcctgcaatttcttaatgacaaaaaatattgaggatattttcacatactttttttgccaactgtattttttttaattaatttttatttttattttattttgtaacttttattttagatttggggtacatatgtacatttgttaatacaggcaaatttgtgtcacaggggtttggtgtacagatcatctcgtcacccaggtactaagcatagttcttgatagttctttttcctgatcctctccccactcccactctgttccctcacgtaggccccagtgtctcttgttcccctctttatgcccattggttctcattatttatctctcacttaaaagtgagaacatgcagtatttggttttccactcctgcattagtttgctaaggataatgtcctccagctccatccttgttcctgtacaggacatgctctcgtgttttttttctttctttttattttaatggctgaatagtattccacggtgtctatgtactacattgtttttttttaaaccctgcataccattgatgggcatttaggttgattccatgtttttgctattgtgaatggtgttgcaatgaacctacatgtgcatgtgtctttatggtagaacaatttatattccactgggcatatacccaggaatgggattgctgggttgaatggtaattctccttttaggtctttgagggatttccacactgctttccacaatgggtgaactaatttacactcccaccagcagtgtataagtcttcccttttctccataacctccccagcatctggttttttttgtttgtttgtttgtttttttagtatttaataatagccattctgactggtgtgagatgatatctcatcatggctttaatttacatttctctaatgattagtgatatgtagcattttttcatttgttgccaactgtatgtatttttcaatgaggtgtacatcagatcttttgcccattttaaaagtggggttttgggctgggcgcagtggctcacgcctgtaatcccagcactttgggaagctgaggcaggcagatcacctgaggtcaggagttcgagaccattctggccaacatggtgaaaccctgtctctactaaaaatacaaaaattagtcggacatggtgtcgggcacttgtaatcccagctacttgggaggctgaggcaggagaatcactggaacccaggaggtggaggttgcagtcagctgagactgaaccattgcactccagtctgggcaacaagaatgaaactccatctcaaaatacatacatacatacatacgtacatacataaaattgggtttttgttttctttttgttgagtttgaggagtttttttgtatattttgattacaagtcttttatcagccatgtgtttcacaaataatttctcccagtttgtggcttatcttttcactctcttaattgtttttttcaaagtagaaatttaaattttaatgaagcccaatttattaatttttttcttccatattgtgctattggtgttgtatataaaaacttactaccaaattcaatatcatatagaattttttctgttttcttcaagaagtagttttataattttgcattatatgtttagatcaatgattcaccttaagttttgtttaaggtgtaaggtttgtgtataagtttttctttttccacatcaatgtccagttgcttcagcaacattttctttttatatatatcttaagggtaatcagcgcaatattttctgaaaagatgacctttttctcatttaattggctcttctttgtcaaagatcagttgaccttatttgtgtggatctatttctggacttcttactctgtttcactagtccatctgtttatactttaaccagtaccatactgcctttattactgtagcctttatggtaagtcttgaaatgaaatagtgcaagtgctccaccttcttcagaattttgttcttcttcagtattaaaggctattctaggtcttttgcccttctttaaacatgttggaatcaattgtcaatatctacaaaatagattattgggatttggatttagattactctgaatctgttaattaagttgggaagaattgacattttatcaatattgaataatatgaacatgtaataatattgaactttcaatctctattatctcttcatcattttaagatcttctttcatttttcattgttttatagatttttacatataggccttgtacatactttgttaatttatatcttagtattgaatgtactattataaatggtattttctaaactttgaattcctgttattcatgatgatatgtaggaaagaaattgacttttgtatattgacattagatcctttaaccgtggcatcattacttattagttccaggggagattttgttgttgttgttgattcattggaattttctgcatagataatcatgccatctgtgaataaagatgttttatttcttccttcccaatctatatatcttttatttctttttttgccttattgcacttgctggtatttctagcataatgtataataggaggaatgagataagatatcttagaattatcctcatcttcaggggaaagtggttagttttttgtcattaagaataatgttagctattgtttttttaaatttcctatatgaaattgaggaaatttctgtctattctgaatttgctgagtttttaatcataaatagctgttgaattttgtcaaatagttttcctgtgtcaattaatatgatcatatgacttttcccgttttcactgttaatgtggcagattatattgatttattttcaaatgttgaatttgccatcagacatggaataaatcccatttgttcatgatgtataatttattttatgcatcgtttgttctgtcttgctaacattttgttgagattttgtgccagtgctcaggagagatattggtctctagttttactttcttataatatctttatctgatttgggtattaggataattctagactcagaatgagttaggatgtgttttctctgcctgtttactaacacagattgtagagaattggcacaatttctttcttgaagatttgttagaagtaatcttgccaccacctgagccagatgatttctttagaaggtaattagttattgaatcaatatatttaatatatatagagatatttaggctatttatttctccatgtgtgagttttggtagtttgtgtatttcaaggaattggtccatttcatccaaattatcaaattcgtgagcatagagttgttcataatattcctttattatccttttaatctccaagagaccagtcgtggtgacttctctttcatttatgatattggtaatttatgttttctgtctctcttttttttttgccagagtctaactctgccacccaggctggagtgcaatggtgtgatctctgctcactgcaacctctgcctcttgggttcaagtgattctcatgtgtcagcctcccgagtagctggtattacaggcatgctccaatacacttggctaatttttttttttgtatttttagtagagatgaagttttaccatgctggccaggttggtcttgaactcctggcctcaagtgctctgcctgcctcggcctcccaaagtgctaggattacaggcgtgagccaccgtgcccggcttctttttcttaattagcctgaatagaagtttatcaattttattgctcttttaaaataaccagtttttgtttcactgagtttctttatcatgtttctgttttcaattttattggcatctgctctaatttcagatgctccttgacttatgatggggttgtgtcccagtacatccactgtaatttgaaaatatcataagtcttttgacttatgtaatgcatctaacctaccaaacattatcgcttagcctaacctcccttaaatgtgctcagaacacatacattagcctacagttgagcaaaatgatctggcaacaaaacacactatagagtattgatggtttaccccgatgatcacatagctgactgagagctgcggcttgctgctgctgcccagcattaagtgagagtattgttccatatattgctagcacagaagatctaaattgaaaattcaaaatacagtttctactgaatgcatgcatattacttttgcaccattgtgaagtcaaaaaaaataataaatcaaaccatcttaagttgggaactgtctatattattctttctcttctgcttgctttaagcttatctagtttttttcttctctagtttccttaggtggtggcttaggttggttattgatattatatatttttcttatctaatatatttacttaatgctatgaatttttctctaagcactgcttttcctgcatcccacaaattttgatgttctatttttatcttcatttagctcaaaatagtttaccatttattttgaggctttttctttgactcatacgttctttaaaagtgtgttgttcaatctctaaatatttcgatattttccagctatctttctgttgatttctaatttatttccaatttggtgtgagagcctactttgtacactttctgttcttttaaatttgttaagggtgttttgtgacccagaatgtggtctatcttggtgcgtattccatcagaacttgagaagaatgtgtattaagttgtggtttgatggagtattctataaatattaattagaccatgttgattcatcataccgtttaggtcaactatatctgtattaattttctgcctgcttgcactagcaattactgacagcggaatggtgaggtttctaagtataataatggtttgggcttgtctatttccccttttagtttcaatcattttgcttcatgtgttttgattcacttttgttaggtatacacatatatacacatatttatgattgttgtatcatcttgaacaactgacccctttatcatcatctttatccttggtacttttccttctttggtagtctgctttgcatgaaattaatatagccactccagctttatttttgtgaatgttagcatggtgtatctttctccattcctttacttttaacatatcagagttattacatttaaagtgggcattattactaggataaataccaaataaagtattttgcatgctgggcttaaaacctagatgacaggttaataggtgcagcaaaccaccatggcacatgtatacttatggaacaaaactgcacattctgcacatgtatcccagaatttaaagtaaaataaaaaataaataaataaaaattaaaaagtaaaaataaattagctgggtgtggaggcagccagctactcagggggctgaggcaggagaatcaaaataaataaatcaataagtggggctttttgtagacaacatatagtttggtctttcttaaaattcaatctgacaatttccatcttttaactggtatatttaaacaatttatatttaaagcaagtgttgatatatttgaataaaaataaacctgtgtttattaactatttcatatttgttgccaaacgcttctttttggtctttcgtagttttatttgagcaatgtatgtcataccattttatcttttctcttagagtattaactatacttcttttttttttttttgagacagagttttcctcttgttgcccaggcgggagtgcaatggtgcgatcttggcttaccgcaacctctgcctcctgggttcaagcgattctcctgcctcagtctcccgaatagctggaattacaggcatgtgccaccatgcccggctaattttgtatttttagtagagacggagtttctccatgttggtcaggctggggtcttaaactcctgacctcaggtgatccaccggcctctgcctcccaaagtgctgggtttacaggagtgagccaccgcacccggcttaactatacttctttaaagaatttttgtagtggtgccgctaaagttcacagtatacatttttaagtattctaaatacaccttcaaataacactattcctttttacatgaaatatagggatattataacatagtattctcaattcctccttactgtcccttgtgacatagctgtcatttatttcatttcacttacctatatactataatcacctatacattgctgctattatgattttaaacaggcagttattgtttacgtcaattaagaatttagaaagaatttagaatcctgtgtaaaataaatttccttttattttaccttaattcatgcattctctgattatcttccatgctttatgtagatccaagtttctgacttatatcaccttcctcttgcttgaagaacatcttttaacatattctgcagggcaagtcagctggtgatgaattctctgaatttttgtctgatttttttttaatatttccttcacttttgaaggataatttccctgcatctagaattctaaattggtcacttttttcaacattttatatattttacttcactttctttattaatgtacggtttctgaagagaaatctgctgtatttcatcctgttctctatggttaggtgcttccctgccctggctctggctttttcaagattttctccctgtctttggtttttctacagtttgaatacaatatgcctaggtgttgtttgttttgtttttttgtaaggagtggcatgtatttatcttcttgatactccctgagcttcctggatctgtggtttggtgtctgtcattaattttgaaaagttctcagccattactacctcaaatatttcttcttcgcctttctttttttttctttctggtattccaattatgcatatgcttgttataccttttgctttttcattcttttgattctttacatttcagttggggacgtttctgttgacttatctttcagctcactgattatttccttggccgtgttgaatcaattgattagtcctcaaagacatttttcatttctgttacaccatttttacatttctagcatttgcttttgattctttcttaaaatttctatctttctgcttatattacccatcttttattgtatgttgtcttctttttccattagagccctcaaactctttttttttattatactttaagttttagggtacatgtgcacattgtgcaggttagttacatatgtatacatgtgccatgcttgtgcgctgcacccactaactcgtcatctagcattaggtatatctcccaatgctatccctcccccctccccccaccccacaacactccccagagtgtgatattccccttcctgtgtccatgtgatctcattgttcagttcccacctatgagtgagaatatgcggtgtttggttttttgttcttgcgatagtttactgagaatgatgatttccaatttcatccatgtccctacaaaggacatgaactcatcattttttatggctgcatagtattccgtggtgtatatgtgccacattttcttaatccagtctatcattgttggacatttgggttggttccaagtctttgctattgtgaataatgccgcaataaacatacgtgtgcatgtgtctttatagcagcatgatttatagtcatttgggcatatacccagtaatgggatagctgggtcaaatggtatttctagttctagatccctgaggaatcgccacactgacttccacaatggttgaactagtttacagtcccaccaacagtgtaaaagtcttcctatttctccacatcctctccagcacctgttgtttcctgactttttaatgattgccattctaactggtgtgagatggtatctcattgtggttttgatttgcatttctctgatggctagtgatgatgagcattttttcatgtgtctgttggctgcataaatgtcttcttttgagaagtgtctgttcatgtcctttgcccactttttgatggggttgtttgtttttttcttgtaaatttgtttgagttcattgtagattctggatattagccctttgtcagatgagtaggttgcgaaaattttctcccattttgtaggttgcctgttcactctgatggtagtttcttttgctgtgcagaagctctttagtttaattagatcccatttgtcaattttggcttttgttgccattgcttttggtgttttggacatgaagtccttgcccatgcctatgtcctgaatggtaatgcctaggttttcttctagggtttttatggttttaggtctaacgtttaaatctttaatccatcttgaattgatttttgtataaggtgtaaggaagggatccagtttcagctttctacatatggctagccagttttcccagcaccatttattaaatagggaatcctttccctgttgcttgtttttctcaggtttgtcaaagatcagatagttgtaggtatgcggcgttatttctgagggctctgttctgttccattgatctatatctctgttttggtaccagtaccatgctgttttggttactgtagccttgtagtatagtttgaagtcaggtagtgtgatgcctccagctttgttcttttggcttaggattgacttggcgatgtgggctcttttttggttccatatgaactttaaagtagttttttccaattctgtgaagaaagatattggtagcttgatggggatggcattgaatctgtaaattaccttgggcagtatggccattttcacgatattgattcttcctacccatgagcatggaatgttcttccgtttgtttgtatcctcttttatttcattgagcagtggtttgtagttctccttgaagaggtccttcacatcccttgtaagttgtattcctaggtattttattctctttgaagcaattgtgaatgggagttcactcatgatttggctctctgtttgtctgttgttggtgtataagaatgcttgtgatttttgtacattgattttgtatcctgagactttgctgaagttgcttatcagctgaaggagattttgggctgagacaatggggttttctagatatacaatcatgtcgtctgcaaacagggacaatttgacttcctcttttcctaattgaataccctttatttccttctcctgcctaattgccctggccagaacttccaacactatgttgaataggagcggtgagagagggcatccctgtcttgtgccagttttcaaagggaatgcttccagtttttgcccattcagtatgatattggctgtgggtttgtcatagatagctcttattattttgacatacgtcccatcaatacctaatttattgggagtttttagcatgaagagttgttgaattttgtcaaaggctttttctgcatctattgagataatcatgtggtttttgtctttgtagagccctcaaattcctaatcacaattgtttatctctttcctgacattccacatccaatccatcagcatgtcttattggctttactttcaaaataaattaaactcagccacttctcagcatttttaaaatcaccctaatacaaaccccctgctacctcatcaactgcaatggcttcctaacttattttgtaacttttgatctttgagttcttccaagagccaagagttcttccaaagctataaaccctatcattggcactcctctgctctatggaaagcagtggcttctcatctctttcagagtataatgcaaagctctcaccttagctggcatggccctgtgggattggcctcccttgtcttactttttccttgttcaggctgctgtgacctctctggtctttggctcttactagaaacctttgaacccgtttccttcccagtgtcagtatttgtgtgttgttcgttcacccttctcacttcattctggtttctacagcacagagaagtagtagtccctgatctaaacaccctctccacctgctttccactttgttttttcccctagcacttaccattatcagatatcatatatttatttgtttattgtctaacttcctcacaaaaatatgacgttgtgaggatagggatttggctttttgccccagagcagtgcctgactctcaataactttgttgtattaagtgaatgaataaaataaaattaaaattgattcaaagtgtatgagcatgtgtacattttacataagtgatacatgacattcttccattccttgggggctcttttaaccattctcaaccagttgtagtacctttaaaaaatcatgatgaagatgattttgagagctaattttggtaggagacagcagttttagcctgtccgctccatgtgcagaataatagcctattttattacatctgatattcaagcactagaatctatcaataggtaaataatttccaaaaataaaagcatcagtgggaaaacagggaaattatttttaaaaaagaatttctagaccaggatgtgtgcaatttgtaatctctaataagaaacgttatagctgataattccagcaattacagaatcatgatcatatccataatggatcagtagaggctctggcttatataatagcttgccctctcaggattctagagccccatatgtaaacacagaacacatttatattgattgacagtgcatgtaggtttctacagattgtgccagtcagtgttttctaaggcagttatgccatggtaattaaaattatgggctctgaaatcagcctgcctgggttcaaaccccagcttcatgtgtcagcttccttggctgtaataaggaatactaatagcacctgccttgtggctttgaaaattacatgatgtaactttgtcaagagcttagatgagcacttgggatatggcgagtgctcaataaatgttagtttaccatcatcatcatcattattattattactgattgcaagcaagacaaactggttctcacttcagcagaaaaagaaattactgaagtaattttggttagatcacagatttaacgtgaaggatgaataaccagacttggaaaacaggtggaaaccaagaggcagtcagcatggcatagcagccagcacttcaccagtgtggtgccttgggggcagcctggcaggagccactgccttcactcctggactgcagatctaccacaggacagcagactgaattgtcctatgtccagacttcacagtcacagggagcaggctgcatgcaggtggggcccaggtcacctaccttcaccctagagtctggagccacagaaaacagtaattgtccttgtagcttttgtgatggaaagcaagtcctgacacccaccaactcacatactagggaattcaccaaatgtaggacggcagctaagatgctgggaaaccaagaattaacaaatgagtattacaccaattagttaattcattacagatttcaatattttccaaaaaacatcctacaaagaacagctcactttaatatactccaacaaatgatgaaatctctccttggtcatcagcttttctaaatttctgtaactttaggtgattctgaatttcttagtgattctgaacttctagaagattctgaaacagaacagtcttactttgggatgtattcaaatcctagagatcagtcacatcaatctgtgccctttttcattatgtaacatgaaacaggaggacctttccaacttgccttgggaacctgctcctacaacagggtatattatcacatttaataaagaaagagtaatatttgcattgtgacccttcatctgaggtcatcgggactgtgctgtcctcccaagctccactgtaaagggatagacaacaaccttgtctcccagtcattgtaatctgtatcagagagcatcattagatgaatgaagatggaaatgttctcacggtcagcaggtggggaaggcaagacttgctgattggaagcaatgaaccatagttcattctaaggcttggggagagcaactttgaggaaatgaggcccctaaacttgccatgtaggagaagttggggggtggtaaattgggcaccctttatctactaaacgtaatctttacttccccaccctctaatttttctcagttgggcttgaaattgtttttgcttgttacactttatgggaaaagaaagggaaatcctaataaggtccatgccagcaaaactctagaagaagaaactaagaatttcaaattgacagtttgttaatgagaaaagacaaggttaaaggattttgttaaacagatgaccaaaataattattggaaacttctgcttctggccaaggtggaataataagggctacatttaccttcccaccagaaaatataataacaaacaaaaactggacaaaacactggacatgacactgttgtctttaaaccttggacatcaatcagtgcaggatgttattttgtaagaaagggaaaaaaattggggtgagccttctgattgtttccagactggagagaagtttcaggccacagcaagtggggaggaacctaggtgtaagtatatgtataaggtagcacatcatagtggccaaatgaatggcacaggaaagaaaaactacaagaacagaggaggtagggccacaaaactagagtggggtccaagattcactatggataaaatggggtgttaatttggttttcaaggccaatacaagaggaggcatagaaaaagaacaaggtggtgttgggaagtagtataaaaaggggcacaggcttatccttgttttataaaaaacaaaggtttttggaagggatttgtagagataagtttgaaaagtagtttgagactatgtgtggaaattcatgaatcttagagtttgattttattcaggagatcagtagcttttaaaatgtaaaataaaaccagtaaactctatctaaaataaattcttcccctaatcccaatataaaaaatacatataaaagcgtagctgctctagacaaaatggcgggtatgagatgaccagaaacctcaacagcactacctctggtagccccatagaactgccaagagttttcagagtatagttaattcctgctgacggctaaggcagtcattgagtgattggaaagaagacggaagatagacaagatcagttaattgggttggagggaagactgaagataggcaaggtgacaaattaggaactgttgcaatgatctaggcaagaaggaacctatgaactaggagggaggcttaaagacagcaaggagggaactataggcagggacatgtgaggaaatgcttagtagttaaggtgagtttgtaggtatgaaaggagagaaggaaagaacattaagtagcattaagtgaaccagataatttatagctccttgacctgaaacatttagaggctgtgataacactaataataataatctataattacataaattacgtagtgtaatgatatatcatgatataatcatcttaccaatgctttacagtcaatgacattgtggtacagagactttggaggcagatagacctcgattgtaacctctgtgctgcccgtggtatgaccttgagcatggtagttaatctgaacttctttcttttctgttaaattggggacaatgacagtacttaaccttatagtacttgaagggagagtgttagaattgatactggggagtctctccacagaaagttgtattagttattgtctttatttcattcttaatgaattagcactcccgttttctttaacatgttgaatttaaaacctcttagtgtatttctttgtcttgccttttaataccccatgaattattgagaaacaaaagaaagtgattatgcaaatgtttgaaatatctaataatacatgaacgtagtcatgggaaactggagaaataactttactgatattatactagttttttttctggaagcatagcatattaagaaaactcatttcaatgaaagaaaatttaaaaattagagtgcattagaagcataatccaatgaattctattcctaatgaatactcaggcagtatgttaactttttctgagatacaatagccaagccaaagaatttaaagaatgaaaaaaacagatgattaaacaaactgtgaagtaattagaggtagtctttgaaaatgcctcattaggcatttgactcattaggacagatccttttattttagggccaggacataattatttaagcagttgatgtgtctttagctcctttcaccttgccacaagttgtcgcctgtactgcctttccacctagcttccaagtccaggccgactttgaagagattccttagggctcacctcccctggagagtgccctctgtaccatctctctccctttcctcctcattcctattgcctgagtttattgttttaataaattcccataagttaacacctcctagggtggattagaatcattcaaatccacattgattgttactaagtagaaattttatactgagcctttctaaatccttacaacaacgtgacgaggatattatagttgctttaacccaaggagatgaaattcaaactggtggcattgcacacttacagtgggcttgcaggatgaaagagagcttggtatctcgatcccttatacaaaacaggtgggtcttgttagaaaaattcttcaataattgttaaggttaaaattttgaaaagtggttcaaagaaatttgctttgatgcaaatattgtctagtcagttacataacttgagctataatgacagtgtacactagctatccagggcacgacatctctattgtgctgttgaatatactgctcaaccattctggaaaatggcatgttcatgtatgaatgacaatgtatcttgtgtgtatggcattccaagccaacatgttggtccctgcaagtaaacttcttgactgtcaagaggctgcctgtttaatatttgcatgatctaaactaattgtttcctttgtttttttcctctgctttatgagctggtagtttgaccttttggcttttcccctgagagttaacaaaagttagacagttggggggtgaacttaagtaaaatccatatcactctatgttgctgctctactaacaattcaagaaaatgtcttggtagaaagcaaggaatgagttttaaattttctctttgaatttcaattatccatgccactcctgtggtccaccattctatataattaagaataagctgtatgttccatagtgacacaggctgttatttctgatttgcagttttcacttaacctagggtaaaattgaaatagaagtcgcatccttttttttttaccttcttccactcaattctagttagatatttttgaacttctataaaatgtctatgcaatattatcaccaataatgtgcaaaattactactcccatgtagaagaggatccattcttcattgaaccatacccttgggatcctatcacatgcagttggccatataatattttatattattgtttttttgttttgtttgttaagtttgtacctacataatggattaaattcaaaatctctgtttaactgaaacaaaatctgtttaactgaagcaaaaaaacaaaatctctgttttttgtttgtacaaaacaaaaaacaataatatttttgttttgtttgtatacttcatagggactttggaatttggaaaatatcgttcatttagggtatatatattagtccattttcatactgctataaagaaatacttgagactgggtaatttataaagaaaaataggcttaatgaactcacagttccacatggctggggaggcctcacagtcatggcagaaggtgaaggggaagcaaaggcatgtcttatatggcagcaggcaagagagtatatgcaagggaactgccctgtatgaaaccatcagatctcatgagacttatttactatcatgagaacagcacaggaaaaacctgcccccatgattcagttacctcccactgagtccctcccacatgtggggattatgggaactacaattcaagatgagatttggatggggacacagccaaaccatatcagtacattaaagaaaatatttgttagcgctaagtgaatctactttcacctacttgattatttgatgattttataaatagtcatctctttcctcatctttccagtcgtattttgttgttccaaagccaaaacctgtgtgtcttgtattcaacaacctgaatcatacagttgtgagacgtaaatgggtgtggtggttccacttacttctttacatagtactacaactaagccatctaagtagtctgctcctaaattcctccaagaagggaaatatcatgatttgatttaaatgtgacatcctttcttagagaagttttctttgtctccttcttccccagtcacttcctcccatccactgtgctttcggcagttcattcgttcattaattcattcaacgaatattgaataacttttgatcaatgtcttcctccctccaagctgaaaacttgatggagggtaacatgatctgtctttgtttaccactgcatcttccgtgaatatacattgtaagtattgaacggatttttactaaatgaacaaatactttgataaaatatttttgattactgccaatatcagtttctgaattgattctaaaattcttctgctggaaagtagattagcgttagtatgactgctgaagccttttaaagtgggtggtaatactacgtttcgttttgtttcattaacctaagtgctgttctttggaatctttttaaagtaagataaattacattcatgaaagaagcatattatttttaaagtactttattttggaaaggtaaaatgcttgtgtagttataatttggttactcttgatttcaccttaggaaaaacaatatcaccttctaaccatttcttttttagtcaaatctcttgcttctatttctctctgtagatccgctattaaagactgtaatcactgctgcatctttcctgtaaggcttgatcgcattgttaatttctttctaaacttgtagagtaggtgtataaatcgtatttgggtaatacactgactaatactgaagaaccaggcattttcttaccccagtcctcacacgaagtagggaaatacaagtcagaacttatcttctcaaaactctgacctcagaatttcctgagaaatctgaacctaaaaagattctttgcttttgacattttttctctggtgtccaccgcaaggctcttgctcttacattttttttttttttctaatgtttcaaatagaagatggtagagtcatatagtacaagctcagcatcggaggggctactggaggtcaactagtgcaaatgctttctgaataatggaatcctttgggaatacccccttgaaagtcttcatctagcctcttcttggaaaccctcagtgatacccctcatcgtctcctatgatggcagcttctatctttgtcgtcagctctgactattacaaactgcttccttacattgagctgtaactaactaaaaagtttccacaccctgcttctattttagcctttgggctcataaagaacaagttgaacccgtcttatgcagaacaacccatctacgggagatgctcaggtttcctccatgttttcctttctctaggctaacccttcgttgttcctcacttactcactatataacctgatttcatgtctccttacccttcttgttactctaacttgagtaggctagtttcaggatccagcaataagtgtgatacatcaggtatgctctgatgagttgagagtagagtggacatagcatctcccttaattcagatattgtgatctaattggcaaatctggcaataaaagttgaaatgcctgatccaggacaatggctggtcaggtgccattgttctcatcttttacttttaggtgtccctcaatttgttaagttagtacctacgtaatgtcctgaaacttgttaagtttgtacctacataatggattaaattcaaaatctctgtttaactgaaaacaaaagcaaacttcttttcagagccagaatctggaatcatacgtaacagagaatgatattgtacaagttgcttcatctctttaagtaacggtttctcaaacttacaagattattgtgagaactaaattagttctaaagtgcttccatgtaaagtgtatgtaaatgcttaaaatatatagtaagtgctgaatgcatattagaaataataataatctttattataatttttactatttcatgagaagtacttattttaatactgagcaaataggaagagttcatggcttctctatgtttttgaagtgtcatttaatatattatatatataaaacatatatatatttcagtcacacatttgtccaaataccttgacaaattaaaacaaaataagacaaaattctcacgctagttatttgttataaagtaatagaacaagtgatatgttataaagagcatttatttctcatgtctttgatattaaaaatagttgtattaactttttatcaaaacgattgcttccttcatataaatctaagaattatgctgtctgataaatattggagagattaacttctttgaaaatatagaagctttttgtctttttaaaatagttgtttttttgcagatgttaatacatttcagcagtacagtatggcctttttcaggttaaggtgctgagcccaaacctcaaagaatcactgcaaaaagattggatcccccctcttcaccccatttcgtaatttagttagtgagaaccacaactggctaaacctttgtggggggccgggcactgtggctcatgcctataatcccagcactttgggaggccgaggcaggcagatcacaaggtcaggaaatcgagaccatcctggctaacacggcgaaaccccgtctctactaaaaatacaaaaaattagccgggcatggtggcaggtgcctgtagtcccagctactcaggaggctgaggcaggagaatggcgtgaacctgggaggcggggcttgcagtgggccgagatcccgccactgcactccagcctgggtgacacagcgagactccatcttaaaaaaaaaacaaaaaaaaacaaaaacaaaaaaaaaaccacctttggggggaaattatcaaataaaacaaactctttttagaattttacaacttttatgttaggaaaaaacaaatacatttgtgaaaagcttaaaatccagtaaatgacttgagggacttggggcaatcctagggtgatgaggagcaggttagtaacagtgaaggacttagcaccccagggggccagaggctgtaatataccttatgagcaagtcattcttatttagtcttgcccattaagaagtctacttggactaaatgcttttaaaaatgcccttttaatttactattaaaagaatattcctagcagaagtagtcttggatgctaaattctattttaagaataactaaaattagaattctgttctttttataacacctgttacacacacacccctacctagtgtgtcggaatcagtttgtatgggctcaccaaagcctactgttcaattttcaggagttttgtaagccatttgatgtcagacaagtggcctgaagtttgttatggtggtggtatttacaccatgaaaattggcatgttatggtggtagtatttacaccatgaaaactgctacaaatagaaatctttttcttcttctcttggagagccacttgttgaacacttaccagctcacctgtgcttgaaagtatttcttcaaataaaatgaaagctggttagctttgaaaattttttgtataaaagtttacacgggaaaaaaataaactaatttttttttccacctgtgttttcagggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggtaagaatgcgaagcgcagcttttaagagtcaatagcttttcaagaacttgttgtgatgtcatgggagaaatagtgggggaaaaagaagcaataacatgttatgtaattggtttcaaggttacaggagatgtgttcattttcagtatcaatacactgtaattttccaggagattaggaaataatatttttaaatcagaatctagaagactgaaattcttaaattgacataatttatttttaacccatctcatttaccaaaaagatttagggtggacactacatggtaaaactatttaatagtgtatgttcacagtagcagaaacttttaacactaaatgaactacaaaagtttgtaatattaatgacctttgttgaaaacatctcaattattaatcaaacgattttatcttaaaaagatttttaagattcggtgtggtggctcgtgcctgtaatcctagcactttttggggctgaggtgggaggattgcttgagcccaggagcttgaggccatccggggcaacgtggcgaaaccctgtctctacaacaaatttttaaaaattagctggatgcagtggcacacacctgtggtcccagttatgggggaggccgaggtgagaggatggcttgagtccaggaggtcaaagctacagtgaaccatgtttgtgtggagtgccactgcactccagcccaggtgacagagcaagaccgtgtcataaaaaataaaccacacaaaaaaagagaaagatctttatggattaaaaagataataataagtgtatttactgaatgccaattatttatccaacctggtgtatgcttagtgttttaggagaaagagaaaggcaatggaaaaataaattaaggtatcatccctgaaagaaacttttagaaagacacagtggctgaagtgataccttgttccttcagttgattctctcagaactggtgttctggtaaaattggactgttactcctgttattcagggagaagaactcaagtttgtatggcaacaagactagaaaatgactttctccctgccccagtgtattcgttcaggagctaatgtagataaaccgaggcaagaagaagctaaattttttttctgggcttataggttaaatgagtgatagatttagttggaggttttctcatttggtttcttttaatagatgaaattaattgtttcctatgaagcatgaaatgttttatatgaaactaaaaaaatgtggagtttgtacttgcatttcaagggtcactgctctgttataggccaagtgaacttatgtctggccttagagaatcttacatgtatttgcatctatcagtatataaacatgtgggccgtagaataaggagccagcagtaccagaacccagccttgttagaggccaccattttggtggttgagtggttattagtttacatggaagcatggagaataataggcaaatgtaggttttcagtgtcagtcgaactggcaaacaaaaatttctggtcatcttcagaatgaaaagttttcttgagtacctacatattttagcattttcatatgaagcagatacattataagttaattgttggaatctaattgtaatatggctgtaagttttttctttatatttgttattgccttgttcttatattatagggaaagagaaacaaacaaacaagcaaagaaactaatggtcatatatttgagagccaactcttggttgctgttcagttttattttcctgtaaacatatattttcccttatgaaatcttgggaatattagctctggagcactgctgaactcaagtacagacattttcatgtgtatcagtagattcccatcatgacatttttataataatgttgaagagcattttaaacaactggaattaagctcaaatacattaccagtggttgaagaattcacatcaataatcttctgaatttaggaataaaatggagaagtcaaggaaaggaaaatattatacacaggctagcaatagttaaaatacaattattaaagccagagctagacaaaattatggcaatgagatgtgtaacaaaaccactctgtaacttcatcatgttcgtttaaaagacctgaatgattccaaaatccttagaccaataaactatgtcttcttacttgataattaaaaaaaagaattataaaggcaaaaatgaaattatatgtatgtgtgtttgtgtgtgttccatgggaatacagctgttgtaaatcaaaggcctacttgcctgaccaagcagaataaaaatagtcattgattttaaagagactaaaagtgagggaagaaaaaagtcttctgcaaaagctctacagatggttgtcaaacttttcagaaaaatagacaaagcaacattttgaaaaggatttatatcttttatatattcaaataccatctgttatttaaaaaacatagcccacactgaatatctgatatagacaatataacgtttatgagataatagtttgagaatgacaataataatagtaagttttaaaagaggaaataagccgggcacagtggctcatgcctgtaatcccagcactttgggaggccgaggcgggtggatcacctgaggtcaggagttcaagaccagcctggccaacatgacaaaaccctgtctctacaaaacagtacaaaaattagccaggcttggtggcacacatccatagtcccaggtacttgggatgttgaggtgggagaattgcttgaacctgggaggcagagactgcagtgagccgagaccccacaactgcactccggcctgggcaacaatgagactctgtctctaaataaataaataaagaaggaaatagtaaatgtcattagtaagaagaatagcaaaatttcagttctagaaatacccagaattagctatatgatacaaaaaccctcatgaaacatttggataatcctcagaatacctatcaatataacaaaaactatgaattgacttcattttgaatgatgaatttttaacaaaaatagtcaatactttgggcttagtagctctagaagtgcttctttcttctttcttttaattaacagaatattgtcagaattttcaaatgttgatgattataagttgaattttccttttcattaacccctggggcttattttttgacttgatatgtttctctactcagaattaagatgtgaagagctttgggatagaatgcatcatacaagtgtgagagtcaagttccaatccataaagtacttctggtagacctggataaagatgaccttaagaaatgattttttttctctttgcagtttaaaaacaacagtagcaacaagaatgataaaacctatgaagccagctctgacaaaagatgttttttttataacacatactatgtatctactttttgatatttatctatcgagaagacttttctccttattgtttgctctgaaatttgtttatatttaataggaattttatagccttatccctgggtagaaattcagtttttttaaaagtagataattaaatcttaatttactattattttacacatggtgaaactagtcaataaatgtaatatcattgagctgaaggttaaaaaggaaaaaatatgactgcaagagtggttgtatttttgatttcagcatcacactcaattgcatcattgagtggttcattcttagtttcagtgttaatgaaatacatctgagtttttttccccagaagcttgaattgtgtcaccaaagtgtcattttgatttattgaaagttaagccctttccaaaattcaccataattttacatgtctcgaaagcaattttatacttcaagtctgtgctatagttctatatattttatgaagatttggatagatatctagccctgagttttttattgcctgttaaatacttataacccaaagtgtagcagcctcatgaactgctgctgggcacagatctgggagagtgcactcagagtgttggcccaggaagaggaaaagaggaaaaggggcagttcagtggcagcttggcttcctgtgaaacatgtcaccttaaccacttcctcattcttttagtggcaaactagacgaccttctccttcccctttcctgccgagtccccctctaatcaacactctagaaggcccctcttcctcctcagtcctgactccctgtcatttacttcaggttgcctccttgctcagttttggccccaggggtaggcaatctcctctttctggacatgtctcccaccagccccaagttcagatttcttggaagtttctgtgttgtctcgaggatgcatggttgtgtgagttattccagggctcaaggcctcttctgtgtctgtattccccaggaatccaggaattcacttctctgcctctcatcctcatcctcatcattaaaatgagcagatttgagctcaggaagtgcttgaataaatgaataaatgaacaaatgaaaggtgatttttgaaagtcttaattttaagaagtctctgagccctgttacagctcaattttcccataaactagaactgctctctaaggctgtgacatttctcctttttcctctcctcaaaatctacctgctgttctgatatctccctgacagccaccatagtgagattctatttccatttccaatctctcttttgtaagggacagagaaacagctagagaaatatggagccatgcgctctgaggactttagcaggcttcaactctatctgcaagtgagtttcacttagcgaatgaaattggaaacttaaagtgggtagggatgagggttcccggagaaggtgtaatacctcagtctgggaattgggagcatctacaaggaacacactcaattctgggaggttcctgtagatttcagagatttagcagggcttcccagcactctgcttcccaacctgtcattggaccagtaaaccctgctctcaaactgttgtggtgtccagcatgctttggcaaggtaatgaaagataacatgacatggacatatgggtgacatcctggagagatgacagaagcctctgtttaaggacaagatttgccatttagattttgcacccactgtataataagagccttaggattgggctggaatcgccctagcaggcatgatggcagcccctctggactggcaatatgcagcttttttgcaagtgttccatgtccaagtcaccccacccagctttctactgctcccgtggaagctctggtgaaacgcaaggaaagcagctgctgttgactggatttttctttcacatgaaactttgaagcctcatagatgcttattggcctggatgctattaaacctttaaaaatccctttctcatcttgagagtatttgagaaacatgtctggggcattttgcccaccctcctccaggttctgtgtcagtgagtgatatggtttgcctgtgtccccacccaaatttcatcttgaattcccatctgttgtaggagagacccatggcaggtagttgaatcatgggagcaggtctttcccatgttcctgtgatagtgagtaagtctcaagagatctgatggtgttaaaaaggggagtttttctgcacaagctcttcttctcttgtctgccaccatgtgagatgtgccttccaccttctgccatgattgtgaggcctccccagccacaatggaactgtaagtccattaagcctctttcttttgtaaattgcctactctgggatgtgtctttatgagcagtgtgaaaacagactaatacagtgagtctgggtcagtgtgtgtttatgttgaacgtagtggacttgtggtgtccccagggcaccctgtggggaatgttggcctgtggctttgctacttccagggggatttggcatggagaatgtgtgttttaagtaatagatagattatgattgaagtgtgttatgggctgagttgtctctcctcaaaaagacatgttaaagtcctaacccccagtatctcagaatctgaccttctttggaaatagtgcctttatataggtaatcaacttcaagtgaagtcattagcacaagccctaatccaataaggactggcattctaatgaaaggggaaatttagacatagaaacagacatgcacagaagaagatgatatgaagagacacaggaagaggacggtcatgtgactggagtgctgggtctgcaagctgagaaatgccaaggtttactggctaacatcggaaactagaaggggcaagagtggactctccccctcagagagagtatggccttgatttcagacttctagcctccagaactgtgaggcgtacatttctgttgtttttgaagccacctagtttttgatactttgtatgggagccctaggaaattaatacaaagtgtattaaaaatagaattttcttctattgtatttttgaggcaaaagaaataaggagctattgatttaaggaggaaggtttggctcatctagtatagacttggctaatcatttactcttgtatatttctcttctgcctgcccaagtagttcactgaacaggcttagaagttgatttataattgtaagagtttacacatcagaagttaatttataattgttaacagtttcatgcatctacctcataaataccttgtaaaagattttacccttcagaataatgttttcctctggtactttagtagtttatcctttttttgcatctttgatgcatttgaattcatcctggtatgctgtgtgagctggggctccaatgtcgttttcctacagatggctccacagttgtttcggtttcattcgaacagcctcctacagaagtatttttcagacttcttttatacttgacttccaagtttactactggttaagagcttttaaagtagggggaaagatatgacccattggcctactcgtttgattattttttccaattctgaaatcaaaaattaaaaaatttattcaaagttatagtttcacttaatttatattaactgcatagatagtgtgattcatctgtcctgcttggacatatttagtaacttttaattaatcttagagataaaagatagaaatctatgagacttgagacattcaaataagaccagtactgataagaggtaatcaggtgaagtgccatttggttgtaataggacatcaaaattgttccccaagagtgaaggatgttccattctcacttttcccatctgtcctcccaaattctttcactcttcctgatgctttgaaccaacttgaaaagttagtccactcagaacctcagctgttaaacttttagcctctgcaaacaaactagccgttttcctctttgtccctttaccatcaagctcagttgcttctcctactcctctcccttctgccaggggctccacccttgttcctcattcaacagtggaatcatctgtgtgcctgtctcctctttactgtgtaccattgggtcctcttcttcaccatctctaaatttatgcttcttccctgttccactgtcaccagcttggtctacatcctactttgatcatttggatttaggtcccaccctaatcgagtatgacttcatcttaacttgattgcatctgcaaagacctgatttccaaatacgaccacaagtaaaggttcaggtggacatgaatattaggagagacagcacttcaattaacagtttggcattaggactttattatctagcttagtaattgttttaacagataaataatagtatcccctaaagtttaaaatgcagaaccagattatgcctcagggtgcctaacctgagaggaagagtctttcttcaacagactctgggaaagacctttgtaaaaaggctggtgaccattctatttgatttctactttccaatattaactgcttaagtcagacaatctccttggcccacgctttcccctgccattctccccttcacataatgtcttcattttatttttcccttgttttattccctgaactcccttctctctattcagattccctgcccattttttctcctaatgagacgcctgacttcctacctcctttttgaaacttcaaactgcttagcaaatacaattttgaattgagtttgctgtcacagaacaaaaactagtccttggtccagagtatcttgcaggactgaatcaactgtggtctaagacacaataataactcttaattattttcttgtttccttcatggtatatttgttgctggatgatgagtttatattatggttgaatttcctttttgaaatgaaaagatgaatagtctttattggctgggtatctatttttatccagaagactttgcaaatgagaaatatttctacaatctgagtttaatttgcttaagcaatgaacttgttctttaaaagtcgtatatgtgcaaaataaatttaaattaaataaatcatattttaatgtagtcagggtagctttcatttaaataaaacctatgacaaatatccacgtgaagttatcactcctttaacaaaacaagtgttctccttttttattgaaatactagttttcacaaattgagattccttaatgtgatgaccgcttggactgactatagtatagctgcgttcttagaatggccctgaaaccacaaggcctctccaagatgcatgtggctgactttctgaggtgactgcattgaatctttctggcttagcttattctgcatccagatccctggaagctatttatccccaaacagtggtatatttagacccattgtgtgctaacctctctgtgccctaagtaccaacatagccaagccaaagttcgaactttgctgccttacaatggaggttacatcgtagtgatgaaagaggttaattttctttaggagttgaagaccagagaactgcaaaaaggggagaaaatcagtgtggcctctaatgagagctaagtgagtgaggcaatagaaaacagtgaggaccggccagtgcggtggctcatgcctgtaatcccaatactttggaaggctgaggtgggcggatcacgaggtcaagagatcaagatcatcctggccaacatggtgaaaccctgtctctactaaaaatacaaaaattagctgggtgtggtggtgggtacctgtagtcccagctactcgggaggctgagcggggagaatcgcttgaacccgggaggcagaggttgcagtgagtcgagattgcgccactgcactccagccggagtgagactccgtctaaaaaaaaaaaagaatacagtgaggaccttggagggaatcttacagtggagcagaattaaaagagaagaattcacctatttcctatataactccctctgtaaccgataactttctagagaaagctcactgaatatatttgaaatttgtttcataccacttttatgttattgccttctttcacaggaaccagagagctctaacctgtagatttgctttgttgtctttactttagactacagcctaaactgactgttgatttttggcacaattattacatctctcctttcaccccgcctctagccctttcttaattacccccattaggcatttttcttgctagcgtgaactaaatcccctgtctcaagcacgcgcctgtacattgaaatagaatgttaattcattgacgaaattaactgttcttgattgggaacccctagttccagggaatcttaaggtttcaattctgttgccttggttactaaactgttccatgcagtttgctcaactttgaatggatacttctcttaggaacattctcccttgtaagtaaattgcttgacgtgttaaaatctaagttctgttttacaattcttgagacaaattcaccatgatttagttttaaaaacaagtggccttgtccttttagtctaggatttatcacttagctgtatctagcaaagcccctcacacaatagtaggaaaaagcagaagtgtgattgctaaatgaaggggaaatgtaataaaatgatggaagtgcccttttttagggggaattaaagtttctctttgtagtgttacatgtattcattatgtgactttcataaatacaaaccccaataaaacggtgggcagttccgtctctggagatgttaagtgtctcttcatagaataacatggcacatcataacatttgattccttttacctggtcaagatgtttgcttatggcatttgaaatgcatattttcaaactaggtagatgaatggtttaaatataaaaattcattcaaggccctgagctatagaagaatatcttccaaaacatgcattcaagttctatctgtcagtgtttttctttttgcatcaataaggcagcaatggaatacaatcagatttattttttctttattgctgtctacaattcagagatataacctgaaaaatatgttgtctcttcccaagtagatcttgtaattctctgaactgtgatccagaccaatgccgcctttacattgggtctgtggaagtgggagggtccactgaacctggttatgagaggtttcaaattgaaaagatttacaagtatgtaaataaaatcaaatagtcaattagtatctaacatatacatgtcaaaacacatagccccaatctcataaaatcaaagagatctacctaaatagatttgtttgcctaacaaatagcaagaccactttgacagaatctttagcatgacagtgattgtcaaaatgggtaaatggttattattgcccagttaagtaatttctgatttgctttgccttgattactcttaataaactaaacttaacataccatagaagcaatattttttaaaaagagggtatattctagagctagatagggtgttgtgcaacaatgtgaatgtacttactaccattaaactgcatacataaaaagggttgaaatggtaaattttatgttatatacattttaccaaaataaagaaaagttataatactttccgtagcatatataaaaaagaaacagacatttcatagcaaaaataggataaaaatgatattaaattcataggggatttctcttatctaaaactttagatttgattcttagataagaatataaacacattgcaagaagataaatcaataagccaatgaagtaaggtggaataaataaatagaaataggtgatacttgcggctctgacagcatctgataataatagtacaaacagtttcatttcatttaacagttatttgatgaactcctgtgtgctaggaactgttatttaaaaaaagtaacttttattttaggttcaggggtacatgtgcaagtttgttatataggtaaactcatggctcggggcttggtacacagattattttgtcacccaggtactaagcatagtacccaacagtatttttttttccgaacctctcctcctctcaccctccctcacgtaggcccagtgtctgttgctcccctctttttgtccatgtgttctcattatttagctcctacttataagtgagaatatgcattatcaggttttctgcttctgtgtcagtttgctaaggataatggtctccaattccatccatgttcctgaaaaggacatgatctctttctttgtttttatggctgcagtgtattccatggtgtatatgtaccacattttcttcatccagtctaccattggtggacatttaggttgattccatgtctttactattgtgaatagtgctgtaatgaacatacgcgttcatgtgtctttatggtagaacgatatataatcctttgagtatatacccagtagtgggattccttggttgaatggtagttctgtttttagttcttttagttctgtgtgtgaaaacacaccagagaacaaaattgaccagatctctgccttcacagaacgttcattctactgtaccagttatttcatgaggaatatgaatactgttattactctctgcccatttcacacatgaaggagcagaaatggagcagagtttactaatttatctgttgcagatagtagctaattgtgtgccaggtgctgttccaagcattttacccctgttaactcagctgagtgcttttattttccccattgtatgcccaacttatggatgacagcactgaggtagagaaaggttaataacatagaatagtttagtattagggaaacaatttgaattctaggacacttttactgaaacttagcataggctaaagaagtgatcgtgcatgtttaaagagttgtatcattttatcattatttgactttcttttccaaaaaaaacaacccttctcctttctctctcttccatgtgacaaatacatgtctgtatacatacatatatatgtaaatataaatatatataaaaatatatataaatataaataaatatatatatatatatataaatatatatatatatataaaatttttttttttgagatggagtctcactctgttgcccaggctggagtgcagtggcatgatctcggctcactgtaagctccgcctcctgggttcatgccattcttctgcctcagcctcccgagtagctgggactacaggtgcccgccaccacgcccggctaattttttgtattttttagtagagatgggctttcactgtgttagccaggatggtctcgatctcctgacctcgtgatccgcccacctcggcctcccaaagtgctgggattacaggtgtgaaccaccacgtgcgacccacatgtctgtatattacagccgttgaaggacacaacttttgccattgggcgacccagtcccctagtaagtgttcaaaaatattcgctattatcattgttggtgatttcgaggatttggatataattaatttgagcctaattttttaagactattacataactatacaaaaaaatcatgagagagcctggaattttgcaaaactaggatagaaaccagtttctaaaaagcattttaaacattcagcttgtgtatttcttttcacttgtattttgtgatctgcctccatgctttcatgcctttctcttcaaacaatcttattaccactaaccaaaaacaattaaaatttgattgttttgtttttacatgttgttacagttaagaaaaaaaaattctaggtattgacccctgccagttttcggagttaatactaactagtgatttggggctacatttaagtgctttaagctatcctacaagcttaaagtagttatacatgcaaatacttaaatgaattaggtacaagaatacagaggtaagaaatagaatctattgtagaataaatcctatttcagatgtgactgttttaggatttgaatcaaccacatattacagaaaaccccaagataaagccttggcataaccaagacaaagtttatttttttctctcacataaaattagtccagacttattttaccagtggtatggcaactccacagtcaaagggacccaagctcatctgaccttcgattcccttatcctgggacaggcttacattctcaaggtcgactcctggcctaatggggctactggagcttcagccatcacactctgtgcttcctgttatatctactggtcagaacttcagatacatctgtctaaaaggaacgctgaaaatacagtctttaagctgtgcaattgtacctggaataaatgagttcttttaataaaaagagttgaatggatatttggtgggcaaatagcagtccatgctacaataataaacttcacaattttagaattagatatcttgtaatgaatattctgacgtggtgtcaacttataccatagatagattataactttgaaaaggaattagaacacaataataataaattcacactaaggctcttttggggaaaaaaaatctctaccatttattgagtgtttattatcttcacaacatccctatgagttagggattatttttgtggacattttacagataaagaaacagaggcattgagatgataactagcttgactaaggagtggcggagctctgggcagtgtggttcctgtgtgcccataaggccattgcacaatgctgcttcatcatgtaattcagaaagtattgcaggatgtggcagttggtcatcatgaatgttcattgttatcttgtggctttgtgaaaaattctggtcattcaaacgatttgactgttgagattctgtgcacatgagattgtactgtgtacatgtttctaaaaatgtgtgttatagatcaaaatgcacacactcatttacctctaagaacagttcttatattgaagggaaattatctgtacgtgagagaacaacgtggttttgagtacaagtggtgaacagacattgaatagttgttttcagaaagcatcactcccctgtacctcagaacccagcctcagtgctggttgcgagccaacagctttgatgtcctgaagtttttgcatcacttctcttttgcccctctttgggattcaaagatgataagtttgaagtggagttctaccgcttccattggaagagaaaaagtttccgtgtgtgtgtgtgtgtgtgtgcgtgtgtgtgtttacggagaggagttctatgcatctgcagagggtgctgccatcaacaaggagaacagagtgggatgaagggtttgggaagccaggatgggcatctctgacgagctccagaactctccacctaagtgtgcaagtgtaaacactccctgatgtgtgaactggccatctcaagactaagtatttagcaggaaatgccccctattcaacccttgccttctcaggtgtagaggttgggttgtctcttccatctttgtttgaattatgtcattaatcaaccttagtgaaagatcacttagtcatttgtgacagcataagttcttaattgttaggaatcactggtgcggcacacatctttttctatccatgaacaacgtcaaatgcttattttctcatgagcttttattttttccttttaaaaaaagtttcttaggataaacacagcctttttccttgtctctcccttgccctctcatctttttctcaatctttatattcctatatgtcactgaagagtccccgtgccaacgctgtgcagtgggaggctcctacctccaccagcttttgaggaggttgtagtcctgcaaccttagaggttccacagccaagctgggggtctttctggagcatgggtggtgaatctgagatctatgcacccaggaagcctgacacattattgtggtgtctcaattcttttttttttaattagaaaaattgtatcaaattgcatttggtgagagcaaaaataaactgaagttggttgagctttggaagactacaagccactgtaatatttaagatttcttgacctccagaactaacatttgtcctgtcagagaaaataattactcctgttgagaatacatgcattaaagtaagatgttcactactctatatgatcaccaaacattaaataatatgttttacacatgtatgtactatgtgttcaagtgtttataaatccatggagccaatagaatgtaagttctatgagggcagaaattttaatccattttgttcctagaacagttcctgacacatactggtgttaggggtaggtcttcagattttatggatcaaatggaatctccacacttaaaaaaacttagagaaaaactgccttaatgtgcccatagtctggttggaaatggcagctccaggttcattgattgctttgttcattcggcacttttcactggacatgctatgagcccagcagtgttctgggtttttgggatacaccaagccctgcctccccggtgcttaacattctagtgggggagacagaaagaaagcaaacatggtggacaacttaattatctatttttgaaagttgacaggaactgtggacaaaagaaaacaagagctggagacagcagtgccagggcagggtgagggtcaagttagagtaagcctcattgagagatcttttgagcaacacctgaaggaggaggtgagaaagttagccatgtggagggagcagcattccaggcaagtggcccaccaagtgcagagtcctgacagcaagagcagttctggaatattctagaaacaggaagaagaccaatgtgactagagcagagtgaggcaggagtgagagaaaatgtgatcaggaaagtaaggtcctgtggccactaagattttggcttttattctgaaggaaatgaggactcattgcaggactttgagagcatgatctgacttgtcacaagtgttctctttgtctactgggttgagaagacatccaaagggggccaaagattgaggcagggagagcagctaggacaggctctagcaatctaggtgataaatgaggaccgatggtggctctgacaagggtggtaatggagacatggtgaggagtgaccacatttcaaatatatcttgaggtagagctgacaggattttcccgatgggctgggcgtagtatgtgagagaaagaattcagttatggatgattattttgtcctgaacaatgataaaagttgagtcaacaactgatatggggaagtctacaggtggaacaagtttttgaggaggaaatcagttcagtgttggctatgttgaatttgagatgtttttagaccatctgagtggagaaatctggagtttaggagagaggctggacttgatatgatgttatgggtcctcagcatagagatgatgaatgagattagcaaagtagtgagtgtaaaaaggaaagggaaggagaccaaagattgagccttgggacagtcagaaagaagagagggaacccgcaaaggagattgaaatggaatccatctcactgtggcatcgtgaaagacaaatgaagatggtataagatgaaaaagtgatcagctgtatcaaatgctgctattgggtcaagtagggtgagaattgataattttccatcttgtcaagagcagccgtgatagagggagggggtggagatatacttggaatgagttcatgaaaaaatgggagggaagggattagaagcagcaagtataagcagctctttcaaaggagggagatggggaatggctactgggatgccatcacatagataggaaggtggaaccctgtgcactggaggagggagtgtcctcacagaggaggacaggcaggaaggtagagtgggctgctttgggcacatgtggttctgtggatgttctcttttgacagcttcagttcttcagggaattgggagcaagttcatcagctgagagtgaacatggggcagacagtgtgagaggtcaaagcggcaagagtagttgtgccacagtcttttaggagagagggaaagtaaatggaatggggaaggaaagtaaatagcatggctgctgagctgcgttacaagcccacccggtgttgggttgtgaggtgtagttgtgggctcttcttcagtcggattgtcagcatgggcggctggcagagagttgaatctgacagggcagcagttctggaaaatgagtatgatgagtcaagaaagggaccaggaagtggaaactgtgtgtgaggcagtgatgctgatgaatgactgtggaaaacaatggaggtgaggagggagtggatgttgagtggctaggggacagtgaaaagggagtaggatccactagcttgctgcttgtctcttataattaggatcaagagcttctgccgtcttctaggcattctgctgcttcaagggagtctctgagattgggttctgcatggatgtattgaaacatgcacctaggtgtgtttggagtcctgccagagacctgctttctctttattcttatcctgaggccctttaggaggctcagatggaataatttttatggtttcttaaaacccaagtgggttacctaagccatcatagttctgccccctgttccctgagaggaccacgggagcaccctgaaatgggttttccactctgccatcccccacttgcctgcttgggtgttgacctcagccatctacacatatcccggatgtagtgttgggcccctggttgatgtcattccttttcaaatgggatcttttactacagcaatccagcaacaaactaaatagcgatagagttttctttcccttacaaaccacaaccacaaccaagggaaaaaccaaaccaaaccaaccaaacaaacaaaacaaaagtatgtggcttcttaccacagccccctatctcgcccaagccttgtggctctttgatctctggtgttctgaaattgcgtaatcatgtgcttgctctttttcattcattgtgctgggcacacacttaatggaccctttcaagttggttttcttttatggggtaagggtaggcaggactattttttaagatttcttccactttgtcttccaattgttctaatgagttttttaaaattgtgcctgtcctgtttttaaattccaagagatattctttgtcctctaaatgtttcttataaatatatataaaatgaataatataagcatatagttttgcatagatttgagttcgctgttacagtgtccttttatttctctgaggatattcattataggttttttaaaaaacactttttctggtccccaaattatctcttctctctagacttcctccattcatgccttccttcccccctccctcccttcctttgctcttgctcttgattttggaggcttgtcataaatatttgaccatccattcattttttaaccataacgaactaaaaatgttaattggagccctgtgtgttccttgagcagaatgtttggattaatgagcttccgtgcaatgttttctctggaatggattaatttctccagatctataatttttcaatctctttccagggggtaaaagcctggttgtctgtttcatgaaagtaaaaatgtgggtaagagtgatcccagcattcagaatgcagacttagtagctgcttgttttcagtcctgggacttcacccaccctcaccacagttggtatcccaaagtgtgaaactccttagattatctttctcaagaaaatgaaactcagatattcgtgtcctttggccatttttgattagcttatttgttttcttccttttgaattgtttgaattccttatgtattttgaatattaaccccttattagatttatggtttgcaaatattttcccccactctgtgggttatcttttcactttgtcaattgtttactttgctgtgcggaaagctttttagtttcatgctatcaaactaattttgcttttgttgcctgtgcattcaaggttgtatccaaagaactcattgcccagatcaatgtcatggagcatttctcctatgttttcttctggtatttatatagtttcagtgcttttctttaaattttaatccattttgaatggatttttaatagggcataagtgtcacttccatttttttatatgtggatatccagttttcccaacaccatttattaaagaggctgtcctttccctattgtgtattcttggcacttttgttgtaaataagttgatcttacttgtgcgggtttatttctgggccttctaatctgttccattaattcatgtgtctgtttttatgccagtatcacactgctttgattacaatagctttataatatatcttgaaatcagagagtttgatgcctctagctttgttctttttgctcaagattgttttggttaattggggtcttttgtggttccatacagatttaaagattattttttctatttctgtgaaaaatggcattggaaatttgataggaagtgcattgaatctgcagatcactttggatagcatagattttttttaacaataataatttttccactccatgaagaggtatacttttttcatttatttttgttttcttctatttcttttatctgtgttctgtagtttttagtattcaggtgtttcacctccttggttgaatttaccccaagtattttgctgttgttgctattgtaaatggaattgttttcttaatttcctttttggataattctttattattatatagaaaagctatggatttttgcatgtttattttgtatgcttcaactttactgcatttgtttatcaggttttaacagttttttgataaaagttttggtgttttctgtatatatgatgatgtcatcagcaaacagagacaatttcatttccctattttctttctttttttttttttgagatggagtctcactctgtcacccaggttggagtgcagtggtgcgatctccactcactgcaagctctgcctcctgggttcacaccattctcctacctcagcctcccgtgtagctggggctacaggtgcccgccaccacgcccagctaatttttttgtatttttagtacagacggagtttcactgtgttagccaggatggtctcgatctcctgacctcgtgatccacccgcctcagccttccaaagtgctgggattacaggcatgagccaccgcgcccggcccctattttcttaatcacgcgatatatacaggcttgttgaaaaacatgaaaatgataaagaagtttatgtattaagaggcaaaagctatgatgttcaactcctctaaaaccacttcttagtaggtgaacattgttaaacagttttgtgaatctgcttccagattttttctaggcatgttcatacacatatacacacataatttatttttacccagttgcgatcatgtatcatctgatccaaaactcgctatttttcagttaatatgcagtgactgtctttccaaaccatagtatatatatatatctctaagaatttttcaaatctgctatagtttttaaacatatttattgttacatacatatgtgtataagttacttatttgcttatttctttgaatatctttgtctagctttggaatcacataaataatgagtacaaatggtaaagaaatttcatgtggaaggatatatagaaaaagtacaagtctctcttctcatccactctatctcattacctaaaaggttatccttagagctttatataatacatccctgaagtttagatcattttattgcctcccaattgttgagagtaaggaatttagtacatatacctttcctttcactttcctaccctctacctacttaatgagttcgattatagtttttcagttcacccactgttaaataatatacctatatttataatatacctatataatatacctataataaacctatatttttgacttatcaactttggacatatctattggtgacttgtgatgagagatgaaataatttgtacatttacccttcttttccattcttttcccctacccattctcccctttttggctaaatgattgcttttaggttgataaggtttataatagctactgttctgtggttataaaatttgtttcatattttatgtgaaatttgattatacaactagcattcaaaacattattatgtaaatattagttactgtaatcacacagtgacgctcaaggaggtatttactaagcatacagatcattccttctggatccaactcagcagttgctttgctgtgccttacctcatctttcgtggattccttttcttttgattgcaatatatccttgagtaatttttcagaatcgatatgtactgtctgaactctcttatacccaaaaaatttgctttcacatttgctaatagtttgacagagtatcaaattctgctttcaaattctttccccttcatggctttgaagatactgccccactatatattctaaaatccattgctgctaatgagaaattctggtgtcactctgactcttatttctttcatatcacttggcattttcttccgagaaaatattacttatcttcagaatgatgaaccttctctatcatgtctctaggtgttagggcttctttcatttaatcctgtaggtcctcaataggctttttaattaaagtctcattttattcgggctctgtgaaattttttgttatgttttggcttctttttctcttttgttgtctctcttttctccttgaatgtctttctgatagatatagcattcccattatctatcatgtctttcagattatcctacatttttctctttgttttctttttctaccatgtttagaattctcttacacgtaagacatcacttactagatcttcagtcatatcctttttattattcagtctgtgcatttttaattttcaattttggcaaccacatttttaatttctaagaatctattttgctaatagtacttttgtcctgaaactttttatatcctgcaatatttagaaagagcacacccttatccacagctgattaggagtacactctgaatgcagggcacgatgggcgaacattttgttactggcctcctcaagcctcaatgcccagaagttattgcctccatcagaaacccttcttttgttctatccctcacttccaaatgaagttccagcttaatttttgctgtcactgtagtgggacaagcaaatccctatttcacgtgatcattgctcaaacctctattgacaatcctgctttgcattcccttatttagagtgggatggaaaataaaggctatgaagggggaagaatctgatcccaccattctcctttttcattgcagttttctccttttttatggatgcaataagctgttgcaatctctcaggatggaaaagaacattttaaaactactctactgccagaatgatctcttttttttaattatactgtaagttctgggatacatgtgcagaacatgcaggtttgttacataggtatatacgtgccatggtggtttgctgcacccaccaacccatcatttacattaggtatttctcctaaatgctatccttcgcctagcccctcaccccttgacaggtcccagtgtgtgatgtttccctccctgtgtccatgtgttctcattattcaactcccccttatgagtgagaacatgtggtgtttggttttctcttcctgagttagtttgctgagaatgatggtttccaacttcatccatgtccctgcaaaggacatgagctcatccttttttatagctgcatagtattccattgtgtatatgtgccacattttctttatccagtctgtcattgatgggcatttgggttggttccaagtctttgctattgtgaatagtgccacaataaacatacgtgttcatgtgtctttatagtagaatgatttataatcctttgggtatatacccagtaatgggattgctgggtcaaatggtatttctggttctagatccttgaagaatcaccacgctgtcttccacaatggttgactaatttacactcccaccaacagtgtaaaggcattcctatttctccacatcctctccagcatctgttgtttcctgactttttaatgattgccattctaactggcataagatggtatgtcgttctggttttgatttgcatttctctaatgatcagggatgatcagctttttttcatatgtttgttggcctcacaaatgtcttcttttgagaagtgtctgttcatatccttcgcccactttttgatggggttgtttttttttcttgtaaatttgtttaagtaccaaaaacagatacatagactgatggaacagaacagaggcctcagaaataacaccacacacctacaaccatctgatctttgacaaaccggacaaaaacaagaaatggggaaaggattccctatttaataaatggtgttgggaaaactgactagccatatgcagaaaactgaaactggaccccttccttacaccttacacaaaaattaactcaagatggattaaagacttaaatgtaagacctaaaaccataaaaaccctagaagaaaacctaggcagtacaattcaggacacaggcatgggcaaatacttcatgactaaaataccaaaaagcaatggcaacaaaagtcaaaattgacaaatgggatctaattaaactaaagagcttttgcacagcaaaagaaaccatcatcagggtgaacaggcaacctacagaatgggagaaaatttttgcaatctatccgtctgacaaatggctaatatccagaatgatctctattttattttttatgacatattaatcatgtttgttctcttgtgtcctctctttctttctctcgattttccagtggtccatggtggtctatttgtagggttcatatttacaaatgggagtcaaggtgactccactacagttatgtgaaaaagtttccctgccatgcctctccctacactgggcgagctgatctggttccgggtcagtggatggagcatttcctctcgcaggcacaccttcaggctggtgggagcaacttgggccatggactggaaccatgtcaatggagaagacttcctctgtgccaggtcctggtcagagctgccgtcttgatttatttgtatccctctttccgtctgtggtaaagatctggaggttcttaagctctctctggcctcaaaacccacagcaggaaatttcctcactcagatcacccacatttttagcaagcagagcttcagttttgagtggagggcaggagtattgggcagtagggaaggacacagttctctacgccgttctgaatgcagtgcattaataaactctcttcacagttgctccagggtgctttgcattggtgaacttctggcttccaggaagaacctggttacctctgatgtgttgggttgttgttccatctactctaaattctgtgccaatccatcccatctgtttcctaccattggtttatttttaaaaaatattttatatttatgtagatttatgaacttgattattataccatcattagagtttgggtccagagtcaaacttctataaacctgtaatcaaaaattggcctttattttcatgtagaaggaagtagcagaccaaaatggagttctatattccatttttctaaatgagccattgagatgtagagcaagcaagtgcccttttgagggaatgtgtgtgtgtgtgtgcacacatgcaagggcattttcccatgtctcccatcctttaattattccagtagacagtgggccagcagcaggtggtggcataacttctccatttgcagtgctgtggctgtggtgggatctgaacaggaggcaaccttggcaacagtaggaatagaatgtggagtcagcaggaagcgcttttccctcccacttacctgtcttcttgtagcagacagcagtgcgggcagcttcggctgacttgcttagcctgtttccattgtgagcataaacaccttagacgttttttcatttcaggacactggagcttgatgaaattaaactacactcattgtctcaggcaaatctcctaatcctattctgtgcttttgttgttgttgttgttgttgttagtggtgtgtttctctctccacttcgctttccttttaaaacacaacaaatttttttttttaattttaaatgaacttattcattgcctccctatgtgtcccctcctttctagtgatattgccgctgttcattcccagctgttcttgattcttgtacaagactctttgctgggcttctgccttctagacgcttcaccttcccgttatttcttccctctgcccccggaaaggcccttaccatggctctgatgatgatgacagctgacccttttccaaggtggaatccacgtttgattctctgatcttgtactggcttagcacagtgctcggcattcagtaaataaggttaaggaaggccagtgatatagagtagtagaaaacagcagttaatgaaggcttgctttctgtcacaggttcctctcctccaggactgcagcatctctgtctttctgtcgtagcatacaagacagcatctctgtctttctattgcaacttccaggtgagaagccctcccacaagcatatacaaattttatggaaacagacaaaaatttgtagggaagcaaatcccttcaaggttgagtctttctggtcttctagaaggtaagcttggtgatggggcagtcacagccctagtgcttatggaacttggccctttggggacagatctcagttaactaaagaaatgctatggcacagagtaccttgaatctcctctaatttatgctgtacagggaattattcacactttattgttaataacacattacatgggggaacaccagacagctgaccaggactcctgtgcttgaaagcttctgctcttcagcagggcagccagcctcttctcggtttagcacagagcttttattctcctcgctctctctaaactcctgttcaagacagtttccaattctgttgaattcactcatttacctcatctgtgcctttccccacctcattctctccccttcagagtgcccatttctgtgtttcaaatcccacaaggccaagcttagatgctattatgtctatgacattctccacctctatcccaataaaaacctttgatttcatcttgctgtttgtatctttactctgttatatttcacccataccagagttgtttgtggacatgcctgatttccttcaggctgtaggcatatttgattcatttttgtactccatgagtgcctaacatagcatcttatacaccatagataattaatagatgcctcttggctgcatttgtattaaatttttaccatgtacttgtcctagctagcagagacttggggggaaataatgatgactgatttttacttatttatttattttttattcccacaggtttttgaggaacaggtgatgtttggttacatgaataagttctttagtggtgatttctgagattttggtgcatccatcacacgagcggtgtacactgtatctagtttgtaatttttttatccctcactcctttccctccctttctcccgagtccccaaagtccattgtatcattcttatgcctttacatacccatagcttagctcccacttatgagtgagaatatatgatgtatggtttttcattcccgagttacttcactttgaatagtggtctccaattccatccagaaccctcaaaaccatgcaaaaccgtggaaacaaaatagcctgctcctgaatgatcattaagtcaacaatgaaatcaagagagaaatttaaaaattctttgagctgaatgataatagcgatacaacttaccaaaacctctgggatacagcaaaagcagtgtcaagaggaaagttcatagcattaaatacctacatcaaaaagtctgaaagagcacaaataggcaatctaaggtcacacctcagagaactagggaaacaagaacaaatcaaacccaaacccagcagaaggaaagaaataacgaagatcagagcagaactaaatgaaattgaaacaaacaacaacaataaaaaagataaatgaaacaaaaggctgtttttttgaaaagataaataaaatggatataacactagtgagattaaccaggaagagagaggatccaaataagctcaattagaaacgaaacaggagctattataatcaataccacagaaatacaaaagatattggaagctactatgaacatctttacacacataaactagaaaacttaaaggagatggataaattcctggaaatatacaaccctcctaggttaagccagaaagaattagaaactctaaacagaccaatagcaagcagcgagattgaaatgataataaaaaaaattgccaacaaaaaaagtccaggaccacacagattcacagctaaattgtatcagacattcaaagaaaaattgataccaatcctactgaaactattccacaagacagagaaagagggaatcctccctaaatcattttatgaagccagtatcaccctaataccaaaaccaggaaaggacataacaaaaaagaaaactacagcccaatatccctgataaatatagatgcaaaaatccttaacaaaatactagctaagtgaatccaacagcatatcagaaagataatcaactatgatcaggtgggtttcataccagggatgcagggatggtttaacattcacaagtcaataaatgtgatacaccacatgaatagaattaaaaacaaaaatcacatgagcatctcaatagatgcagaaaaagcatttgacaaaacctagcatccaattatgacaatttttaaatatggggaatggtcttcatggaaaagtagatgttaatggcacctgtattagggttctctagagggacagaactaataggatagatgtatatataaaggggagtttattaagaagtattgactcacacgatcacaaggttgggttccgcaataggccgtctgcaagctgaagagcaaggaagccagtccaagtcccaaaacctcaaaagcagggaagccaaaagtgcagccttcagtctgtggttgaaggtataagagtcccaaagctgaagaacttcacgtcagaaattcgagggcaggaagcatccagcatgggagaaagatgtaggccagaagactaaaccagtctagtctttccatgttcttctgcctgcttttattctggccatgctggcagctgattagatggtgcccacccagattgagggcgggtctgcctttcccagctcactgactcaaatgttaatctcctttggcaacatcctcacagatacacccaggagcaatactttgcatccttcaatccaatcaagttgacattacatattaatcatcacagcaaccaaaagattatctcatttaatccttacaatagctctgtgtagtgggtatatattttccttttgctgaagaggaagtaggcttagaggggttgagtaatttgcccagaatacctaggtagtagaagatagtagagccattatattctgtctgcattcaagagtgtgctgctttatttgttcctgaattgcacacaagctaaagaatacaactggatgttcagtctactcctattttgataacttggctaactttactgcagagtctgcagaggtgaccaattttactctgggagataccagggaatccgccatgtaattcctcaggctgagtgttttggaaacacaaagctgtcactgctattaagtgatgttttttcctgagagtctactatgctcccagtaccagcctccgcaggccctctgtctaccccttttctgtcctgatgtgttctagcaggcactgagaaagtcattgtagaagttaccatgactttccaaatgcttccagaagcagaacacaatttcacaggagggccaataagtatgaatgccaccttgatagaaagtgaagttttgcgtcctgttgaacactcagtaaatgcttcctgaatagatgcaggattggcaaaacatagctccccagctttcataattcagactcagaggcccttcgtgctcctgttattcttgttacttcatgtcaggtacctaaaagggttgtttttcagttttcgtcattgatttaaagcagaggttagtaaactttttctgtaaaggccaggtagttagtattttagcctttgtgagtatttggtctcacaaaagcagccatagatgatatgtaaatgaatgtgcatggctgtgttctgataaaactttatttaaaaatgaggggtcatctgggctgtggtttttcaacccctgatttaaaacaaaaacaaaatgtgctatgcctgttagatgctactacatttgattctgtgttttccatatttttctaaaatgtactgaacaggaaatgaacaaactgatgacacaatgatctaatttaatcattgagataagagtggcgccaactcttattgagtatttgttatgtgtcaggcacttggctaagcactttacatgttttctatgatttataaagcaaatattgttattatcttcattttttgctgagggaaggaataaagtcacagccagcttacagaggctgccggaggtcacacatcaccagtacactgaaacacgtggtctgattacaggatccatgatctcctgtacagatcatagtgatattgacctgagaggaaagagcctaggacttacaatcagaaatctgggttcacatttcaactccgttccatgccaaattacattaaaactgggcctcagctttcccacctgtgaaagaggaataatactaggacttgccctcccaaccttagagattgctgtgaggataaaagcctatatgaatgtgctttataaactataaacaccatacaaatgtttgttataagtctgccatttgaatacatcttagtctttttttcctatgtgaaataaaacattttaagacaagcaatgaaattcttgaaactataacaggtttttaagggtgttgatccccaaagccgtcttcactattttgctccctctctgactcgctctgtctctgcctagcagcgctagtcttttctacgtgtttgatctgttgttcctgtgtttaggcccatccctactacacagcggaagtcagctattttttctctttcctttgctctgctgagcaagattaaactcttttcacctggacttttcctagattactcaggctgggtggaaatggggtagctgtgaggctgggatgttactgtacaattcaaatttatgatctgaacttaactagatcttgctgaagattgacctggctagcacagttttgaggcacctatctttcagtcccttctgtcctcttggcctggccactggccacatgttagtagcttacttctaaggatggaatgccatgcatctcctacaaggttgtctctatgcttcacacaaagtaaaagaatggaggcaactttgaatgctgctcattttcactcaggcaatccctttacaatattgtgcttagtaaactccccccactctttttttcccttagcttgttttttttttctaataaaagtctatatcaatttgatgtattagttagggttctctggagaaacagaaccagtaggatcaattgattggtcaatcaatcaatcaagacaaaaagagattcatctggaaggagttgattcatgccattgcggtcattggctagtctgaaatctgtaaggcaggctggctgactggaagctcaggcgggatttctctgctgcagtctcaaggcagaattccgtcctcttggggaaacctcagtctttgctcttaaagctttcaattgattggatgaggcctacacacattatggagaataatctgttttacttaaagtctactgattgtaaacattaattatatttaaaaagtactttcacagcaacatttagactagtgtttgaccaacaactgggcaccacggtcaagccaagttggcatatagaattagccagcaaacttggtattcacaatctgtcattatagtcttatctataaacctaaggtttagagaaacgctggtcattctgaacaattccctgtgaatgcagagatcaaggagcctccccttaactgggtaggcagtgtgaaatacctgcagtgtacttgaccttactgttcacccactggcagctgttcctacagcttggcttcagatatataatattacattggctaacttcaaagagcattgttttccaaggatagagatggtgatggtgtatattacttttaccagccccagacctgtaagaggttctcatctcttttgtttaagattttgttgtttttttttaatgctcaataaaagaagctttattctttcattaaaaaacaaatctcagaagaacagggaagaagagggtagcctatgaaagtgtgattgttatttgataccatcgtgacttagcttgttttcacctaagactctgagattctattctgtttatgtgagatttgggctgagttcaactactatccatattaactgaatcataaaacaattaatccatcatctggttacattttggtggggagggagcatttaacacaacactaagccacatatttcactattttttttttttgcagcaaattgacttaactgcttgctttcacaccatgcctataaaacctttttggttttagaggttctatgtgtctggaaagtacagtgaagtatctcatcagaatagtattaatatttcttatgacatttcatatgtcctgatatgggttaatggagaaatatcataagagtagtaattctcttgctgttattatttgtcctatttagacaaacagaacatagaattaataagaattcaaaaactttaatcactgatagagattaaatgatgtttatattagtcatcatcacctaatatcttaaatatttaaccttttatgcagtgtttgcctctctacttgggtatgcccttaacagtaaaaagtgttgataatgctctcccttttggcactcacagaattgctatacatatatatgtataaaatatatgtaaatataaaaatacatagttaaaaaaataaatctaccactaaatatagtaagggagttttaaactgaggtttttgaagtctttaagaatttatttagagacttcttttgttggaagtatggtggctagatgccatgaaaaacccactgaaaacaagtgtaagtgctaggtaatatgttgaaacatatcttttgaaatgtattactgagctggcaagaaagtatgccattgtagagagtgaaaataagtgaaggcagaatcctgggaggtaagccagcctgaagatattgatgacactggatggccttaagtttccattttgactggcatgtaatccagccaaagattcccacagaaatccgaggttccaaatggtaaatccgtggtgacattggggtgaccgagaaatgaactctgtgcagaaaggaatggtaacaacacttgtctcgtacaactttggctctgggtaggggaaaggaaaacagcttcctagagaaatgttaaccacaagctagccctcatgagagatttcagctaggatggatgctatctgtgtagtgcaaaaaaaaaaaaaaaaaatccatagccagattttgtgctttaatgtggacctaggtcaatagtgactgcaaagccagaagcagtggctcacccctgtaatcccagcactttgggaggccgaaggaggtggatcgcttgaggtcaggagttcgagaccagcctggccaacatggtgaaaccctatatctactaaaaatacaaaaatgagcctagagtggtggtgggtgcctgtattcccagctacttgggaggctgaggcacaagaattgcttgaacccaggaggcggaggttgcagtgagccaaaatcgcaccactgcattctagcctgggcaacagagtgagacaccatctcaaaaataataataataaaaaaaagtgactgcaggggactgggaagaggaaacacaaatctttactggggaatggaatttcaagtgtatgcaccaacccttagaaatcacaaaacatctgacccttctgaaaataaccaccatgagtgagacagcaggaaaaaaaaaaagaaaaaaaaaaaaaaaacaaggaaagagagggacaatagaatcagactcatacctacaaaacttaaaaattaaataaaatgaatcagactcagaggcttcagagtttgatttaaatacattatacaaaagttctgtgtgaaatatgtttaagatgtagaagggtgcttgaaatgcacaaagtcaacagctgaaaaattgaccaagcagatatgaaaataaaatccaatagagggcatctggaaataaaaaagtataataattatagtgaaaaaagttattagatgggttaattagtagaatggatatagcagaagaaagatttagtcaactgaaaaatatatccaaagaataggtccagaattaagcttagggagataaagagatggtcaatacgaacattaggagacatggagaacagtgtacgaaggtgtaataaattctaactggagttaaagaggagatagtaaagaggaagaaaaaacacaaaactcaaaaaatgtagtggctgagaatcttccagaagtattgaaagacaccaatccacagattcagaaacctacaatttgcaagaaaaagtaaaaagaatttcacaccaagatccatcttcatggactggcagagcataagagatgaagagcttatcttgaaaatgcagccagagagaaaaggtctatcgcagttaaaggaagaggcaagcagatgactgagttctcaatgccaactgaaaatgagaagccagtggcacaccttcaatatgttgggagaatataattttcaacttaaaatagtgtacattgtaaaattacatttcaaaaaaagagggtagtataatgacattatcaaataaaaaccatcccttataacaaatatcactaaagaaactgttggaggatgtagtttaggtttctagaaaggatgtctgatatgcaagaaagaatggtaagtcaaatattgagaaatatgtgaggaaatagaaacaagaataactaatgaatcaatgatactgtctaatttgaaggttaaatgattaggtagaactaaaatatgaaacaataatagtatgtaagtcgggaggaggaggatgaagcttgaagtgctctatgcatcttgcatctctggtgtctttcttgtgtccaaactttttcttctttcaaggacaccagttagactagattagggtccacctaatgacctcattttaacttatttgcctctttaaaggttctatctccaaatacggtcccattttgaggcactaggggttagggtttcaacgtataaattttggggggacacattccagccgatagcaattataaatacaacttttatagtaaaagcccatcttctagaacaggtagagaacaaggttacttattttttttaagaacaaaattggtttaaattatcctaaaatgattttaggagaaaatggacattaaatatacaacatttcctcctttattattttattgatgacttgatatgttttttaaaactcagtaaaataaatgattagtctcataatcataagagagttatgcccttattttaatgccataaaactgtgacttctgaagaaatacaatcatataaaatcatattttaatttacttttgagaatgtacagtaactcaggaaggcagagtgtggaacctgctttaatgaatagataagaatgactagcaattaaaaattaattatctataattcttctgaagttcttgaaaataatctcttctaacctgggtaaatattcctccttttcagaaatgtttgcaatgttgcttttttttaaattaatctctttttcttagataatggaacataaatcttaggcacatttgctttgtaaataccaaattgcacacagattaaaatttaatgagcttttactgctgtaatctcaacaagcagagtctttgctgttggtttttcaataagaaatactttattttgttatacagtggtcattaagaaccttaaagataatcaggacagattttccaaattgcataatgctattgcctagctatttgcatgtattttttttaaatgacaagacatttcgttaaatatttacttgcctgacgttgaaagatttagacaaacaatgtacttctagttgtcactttttcaaaaagattcttagggaaaagttagccgttttgttggttggtaagtgatgggttttcttggaacgtgttgcttgctaattttaccagagaagaaaataaattcaaatggtaagctacattccaatttgtattcttcttctaaagtcctccctgtttgcattctcggattttgacactcctacacaccagttaaagcacttgttagagtgtccaaaaaattgttgcaattaatttcccagtctcttctctctttgatgtgtcactataatgctttgagtaattttcagtgtctggcatctgttcaaggacaatttctggattttaggagggggagaagcaataaaggaaacaacatatgttttttcctaaataggaggcattttttgtttgtttgtttgaacaaaaatcaaaaactttcttttctatataggtgacaaaaagatacacgactcattggttgttaactaccattgcattcttttctctttggagtagcccttaaggaccagattagaccagccatctgcatcatgtagcatgtctgggttacctttcccaaaagaagttcagatagatgaactttgcaaaccagagccatctgaagtttgttttttctttttgagacagagtctcattctgttgcccaggctggagtgctgtggtgcaatctcagctcaatgcaacctccgcctcccgggttcaagcgattctcctgcctcaacctcccaagcagctgggactacaggcacgtgccaccacgtccagctaatttttgtatttttagtagagacagggtttcagcatattggccaggctggtctcaaactcctgaccttgtggtccacccgttttggcctctcaaagtgctgggattataggcataagccaccacgcccagtccatctgaagttttaaaatggctgcaggtatcttagtaggagactaattttattttccaatgattagaaaaaaatgaccagaccaagaactctagggttatgtttcatctcatatcctgtgcaggatgttctgagaagtttctatttgtatctgtggagtggttctagtcttgttcatcttgcattttaaggtttggaggtattttatcttgccttgcttctttaccaccttgctttaatttccccctatattgccatttctaaagtaatgactcattttcaagggtgggtctgaaaggaaagaaataataaaaccagagatctggagaagtttctcgttcatggtcttattcactttttaataagagcatctagaacagtgagtggtgccccagcagatatctggaaatttttgtggaattgaattgaatttgttcttagagtaaatgacaataaagaaaatagtattatggaaactttcgacccaagggaaatatcaaggctaaagttgctatattatttttcttctgatggatgctgagtttttctccttaaaaaacaaatccacatttaaacataaggcattgtttgatggcctatcacattcagtagaatggatttgagaaatacgtgtaggatgcctattaaaaactctgttttgaaattaaacctaagactataagattgtctgtttaaaaaaattattgctgctgtaatttgagcaaatctgaactatattgaactttagaaattcaaatgtatgcaatcttgtaatataacttactccatcggtagacatagttctttgtcccacctggcttccagcagtttgtctgtctgtcattaggctaaactcttcatgttcctaaacatgttttcctcaaaaggggacatatttcttgggtcagatgtaaacatcaagctaaggagttctgctgtgcgtctagacaaataaggttcattttcatcctctacatagtgttaaacttgaatttaagatcacacagattcctttacacgttaccaatgtaatgatatagacaatctcactttattcattcactctgtttaattcattcaataaaaacgtatacattggctgcctactgtgtgccagttggtgaagatataaagatgattaaggcaagggtctgtgctgacatgcctaatgggcaggctgacacacctaaacgccagggaaaaagagggatgagacctgggcctctgaagccctggccacctggctcgaagctcactttaatgtgcttctgggtgtattccatgactttttaaaggcgcttatgtttcctttctggtaaagcatgtttaacatgcaaaatgcttatttcattgagtagcttttaaaaagtgttcatagaatgtctagaacagtgcttgtctaatagaagtataattcaggtcacgtaggtaatattaaattttctagtagccacattaaaaagtaaaaagcaacaggcaaagttaatatttttttttttcctgagatggagtcttgctttgtcacccaggctggaatgcagtggtgtgatctcggctcactgcaagctccgcctcccgggttcaagccagtctcctgcctcagcctcctgagtagctgggactataggcgcccgccaccacacctggctaattttttgtatttttagtagagacggggtttcaccgtgttatccaggatggtcgttaattttaataatatattcattaattttaataatatattcatttagtcctatataaaaaaattagcatttcaacatgtaatcaatagaatatattattagtcaactattttgcattctttgttttgtaccatctttgaatttcaatttgtattttatacttacagtgcatctcaattttaatgctaaatgtcatcggagatattcgatctctgtttagctttaatatatttttgggttgaaaaagtaaattcacataaccaagttcttccaaatatacttgaaagtgtttctgataactgagttatcaacttaaaaatgtaagttaatgacaataaatgaaagaaaaattctgttccttagctgcactgaccacattttagtgttcaatagtcgcaggtggccagtggctatcatattggatagtacagctttagaatgacagttcagtgcaacaagtgctataatagactcatgaactggaagctccgggaagttaagaatgggatcaatgaagtgggtcttgaaaaaccaataaaaattcaccacgtagagaggaggagggcgactactccaattcaatcttttgaaagcatgtgaaggagcaacagtagacttctttaattatttaatagtaatttattgaacatgaattatgggaggtgcatggtgtgggtgctggtgagacatagttcctgagctcaagtagcttggtgtctaaatattcatgggcccatttttcagaaaggatggatatatgtgtgatcctgggtgtgccaaatgctgtggcttcctgaagcttagatttccagcttgtcaccttcaaggttaccttgtgaataggacttttttgagctgtaagtaaatttactttgcctatttatttccaatggaaaaaaagcttttttaaaaaataaatctcatcttattgcctatgattggccaagacaacatggcccatacagaaggtttttgatggcttctgaggtctgttatcatttgcttattggcatttcagctgtcaaccagggttctgtcaaattccattcctgcctttagctcttttacttgaatacctggggcaatggcagaagtggttgctatttgtcacctttccaggatgttagtcgtgtccttgagacaaatagaaaatttaaagtcagatgacttgattcctctgccagttaagacccttagagagtcctcagaatgttgcttatttttaaatttcaagtccctggtaaggatcaagtaaactccccaatttgcagatttctatccagttgactatggattttgcctgttgctttgtttccaccaactctccctgaagatgaggcgcacagacagacaactcacaggcaagaacagcctggtccatcttgaaagattctcaagactattctccacaagataattgtctacttttaaaaatattcagtaaagggaatttttgctgttatccttggttgtgtttttagtatctcatcatccttctagttgtaggaggcttttcctaacatctaacccatatgtctgttgtctcatcaggtgtttctattaaggctacttccccatcaatcttaattttttttttaatcttctgagatgtataggttaagttgaaatcagagactttcataaaatggtaagatggccatttaaacgcaactatgaggaaataatgtaagcaggattccattggagaaccaaacactagcaacaactaacttggtaatcaatgttgggacttgaagttaggctaaaatcaatagtaatggcactcgtatgtacaaatgcagaactttttacacacaatgatttttccctctgttattatacactagctgtgtctgagtagacagtccagctccactacctgcagtccaccctgggctgtgtaatctgagcactgatgggctgttatttgtacgtattacttctatgacactgttttttcatctgtgttagaaatgtctttctttcttgaataagctgcttcaatttcttatagacagattctgcttttatgacccttgtttctacaaagtaatctctactcatgctgaaatctcagacaattttaacaaatatttagagtacttaatttttctttgaattctaaatattatccttctttagttcaccatagttggatattttgagataactgtggaggaaacagcactgatcctggggccatgaaacctgatttcaagatctagtcccttctttaattttgcacaactaatttaatcacattcatccttaatttcatcatcattaaaatagcagagaataattcccgtttcatggagttgatatgcaatgaaatagcatatcaatgtatgtatgaatgtcccccaacatagagtactacacaaatgaaacgtgtcactcagcataacgttatgtgctccttcctgcaactggattgcactccagtgggatttcatgctggtgagatggctgtgctgctggacttcatgggacaccaatctttgaaaataagcctgatctggctgggcgtggtggcttatgcctgtaatcccagcactgtgggaggctgagacgggcggatcacctgaagtcaggagtttgagaccagcctggccaacatggtgaaatcctatctctactaaaaatacaaaaaaattagctgggcatggtggcatgtgcctgtaatcccagctacttgggaggctgaggcaggagaatcacttgaacccaggagatgaagactgtagtgagccgagattgcgccactgcactctagctggggcaacagagtgagactctgtctcaaaaaaaaaaaaaaaaaaaaaaaaaacacaaacaaaaaaaaaaaagaaaaagaaaataaacctgatctatagatgaggccagtggatcttgtgaagagttgaaaggtcaggtatcagtcttataccatgtcggatggggcaatcgtcataactttttaaaaaattatttatttattttgagacagtgtctcacgctgtcccccaggctggagtgcagtggcatgttttcggctcactgcaacctccgcctcctgcgttcaagcgattctcatgcctcggcctccctagtaactggattacaggtgtgtgccaccatccctggctaattttttgtatttttagtatagacagggtttcaccacgttggcccaggctggtctcgaactcctgacctcaggtgatccacctgcctcagcctcccaaagtgctgggattacaggtgtgagccaccgtgcctgccccaattgttacagctttttaagtggattatatgcctgcttcaggcattagacataataagttaatctgttgataagtttcttcatttcttaattttcagttgtgttttgatgtgtgtggtctgggtgagcggctcagtgctgctgctgtggagaaggcgtgatatggaagggatgtgactgtccttcttcatcttcataacggggccaggactggggtgaggtgtgtgagatgaatgaagcactggttttgatacaaaatttaagaaaaatcaaattcaagaggtaatattttaatacgatatttgtaaaaaatcaaaattagtgcaaaaatttgtgatgagcaaaatgttagataaaggcattgttgcttttctttttctgtgtattgtaacacaccatcacttagtggagaaacagctctcactttcctcctccccagatacaaccatttatgttgaattaaacttctagttctcctatacctgtgagcatcccccaggccccttggtatggagaaagcagtttatttgtcttagtaagattataaaagtggcaataaaatgtgaatgtaatgaacaaaatgcacattttgtggaccaacttgttgttttagatctatttttgaaaactcattaggtatgtgcatgaggcaaaataattcaagtgcagttggatattaaatgaaaaagtgtcaggtccccttttctgccatattctcttctgtgtcttcattagtggaaaccattttgaccagttcttataattctaatgatttgctgatggctctaattcttggtttatcaacttcaagcagaatgtcttgacatcttggtatgaaagttaaagaatatgaatcatttgttctctcccttctcctttcctctttctctgcctctcagtttttgttgtatgctcacggagtatgtgtgtgagcatgtgtgtgtgtgtgtgtgcacgcgagcacgtgtgtgtgtgtgcgcgcccatgggtccatttttgcatggcaggctttgttttagaaagattggttacaacagctgccccaccaatctcctctacagggaaggattcctactctgagaccttggtcttttccaggcagatcacttacatttcctcacagattatttctcatcctgcagcctgggtccaaatgggctttgctgtgaacagtgactctcaggcttctggtctctgctgtgcttgagtgtcgttgagatgctccatctcctcctactgcacccacccagcaatcatctctctttcaggaattcaccaaagtcttttttaaaattattcttttgttttgagacagagtcttgctctgttgcccaggctggagtacagtggcgcaatctccgctcgctgcaacctctgcctcctgggttcaagtgattctcctgcctcagcctcctgactggcagggactacaggcatgtgtcaccacacccagctaatttttgtatttttagtagagatggtgtttcaccatattggccaggctggtctcaaactcctgacctcaggtgatctgccggggtccattttacgtggcagactttattttagaaagataggttacgacagttgctccacccatcccccctgcagggaaggagtcctactctgagaccttggtcttttccaggcagatcacttacctttcctcacagattatttctcatcctgcagcctgggtccaaagtgctgggattacaagcgtaagccaccacatccggctcaccaaagtctttgggtggttgatgtcatatgcctccagttgatagcacttttaagaatttttcctttttgtatgctgttattatttttagaaggcttttggattattattgttaaagagtatgccatgtctatcttttaacttttaagcatctattgctttgttacttgtactttatatatatgtatatccttcccccaataagacaaagagtgcaaataatcacctcccttgcctgttgtttgggtttaattcaggagtcagtggaattaaaagcctaaactcagtatagttttaaagcagcagtccccaacccttttggcaccagggactggctttgtggaagacagtttttccatggaccagtgtgggggctggaaggtgattccaggatgattcaggtacattacattaattgtgcactttatttctattattattacattgcaatacataaggaagtaattatacaacttaccataacatagaatcaatgggagccttgagcttcttttcctgcaactagacggtcccatctgggggtgatgggagacagtgacagattatcaggcattagattatcataaggagtgcacaacctagatcccttgtgtgcacagttcacagtaggattcgtgctcctatgagaatctaatgctgccactgatctgacaggaggtggagctcaggcagtaatgcgagcaatgggagtggctgtaaatacagatgaagcttcacttgcacgttcactgctcacctcctgttgggcggcccagttcctaaccagggttggggacccctgctttaaagaatatgtttgtggattcatagagggaaacaacacacactggggcctttcggagggtggagggtgggaggaggcagatgatcagaaaaaatattaataactaatgtgtactaggcttaatatctgggtgttgaaataatctatacaacaaacccccatgacacaagtttacctatgtgagaaacctgcacgtgtactccagagcttaaaataaatgttaaaaaattccctccaaaaaggatatatttgcatcggagttatatttgtatatgagtatatatttgtatatataaatatacttgtatatgaaaaaaatatcttttttctttcattttattttccaaacattaaagtcgggcacattggttaggaatttcaccaatactttttacaaaactgaggacttggatgataaaggcactttttaaaagattacacactggcatggggaataatacctttataaagatgatctatgtgttcctgaatatcccgatgcatctcctagctggatcttccgctccacaaaaattcataagatgagattcttgaattagaatgcatgccttaatgtattaagtctatgtagagagagttatttagtccatgtaaattaagtgggaatatttttatttgattactgttttatctggatcttgcccacttaaggccttcaaaaatgaaatttaaggcctgttaagcagacccaacatcaacagcatattctctctctcttgtaagtattgtctagttgataaaaaatttcaaaaacatgtcttaatcaaaaacaagatgatccagcacaggtttaataaatgttttgtgaatatggcacattcgtgtctcattactacagttttcctatgctgtctttctcaataattccccccaaaattgtagtgtttacattatggcatttatagttacctctgaacctaaaaaattaacttcaaaagtatttaaaaaaatcattatatttaaaaccatttcatgtttaaatggtttgtacagggcaatgaaaggaaatgtagtaataaacacagaatcagatttggtcactaatatttttctgcagttgaaatatatgtagactggcttagggtctaaatagcattgaatcctgttaccttccatctataatcaattaatgtattatgacattgttgtcctcagatcactgggatctcatggtaagtaagtaaagggattattctgtgcattttcccaatatctatattagttttaatatacctttatgttaatgtatgacactgacacttagtaattggattaacttcctatcagaatttgtttttcactcatactttgtatacatgtcttaagggtagggtagatgtacatttttttctgtgttagcctatctgtttctgtgacactacatgctttctgtcctccaatttgtgtttctttccgtgtacaaatatgcatcatctaccatctacatctacaaaacatgtaggcttccaatgttttcatgtaaatacatttcatgagtcctcagtagagtgttagatgagtggttaattagtaatgttaataaatgtaaattacaataaaaacatttataatgttaataaatataagtttattgaacttaaataatttaaacatcttctgaaatgtagtagaaattatgattagccataactagtacagtattttttgcttgtttaatttttttggtgatattccatttgtggaatgagtgtgtgtgtgtgtgtgcagtacagacagaaaggagagaaacatatctgcatgttatgttagaaagagtagtgttagctaccctaccaaatttacatactggctgaacacagtatgtaaaaaattatttttttagtccattgctggtgttccttgttgttgggtgactctattccacatggtgatgcagggaaccaatctccttccttctgtggctgtgtaatcccttgcaaccttgaagtcctctgcatctaactttaggatgaggaaagtgaaggtggaggatatagccctcttgactgccttgacctggacctggaacatctcttttgctcccattccatggggaagaactagtgacatggtggcccatagctgcagaggggaggctgagaaatgcagcctctggataagaagcctactcccagtggttactgtacattgtgggagggaagcttgaattctgatggacagtgagaaccattctccacacaataacgaaatgacgtcttctcaaccttggcaatgtagcatcctgtgacttaagtatgtaaataattatcagtacaggtcagtgaaaaattaggtcacctttcccttccacttttaattctatagtttaatttgcctatctgctacatattatatatatgggaatagataagattatctataaacatgtataaatttgtttcctacactaaaaagaaaaatgacaaagaagaattaagttagttgcccaaatccacacaataccaccaggagaaagtctaggaaccataccttctaaatccaggtctacacttctttagtgcactaaattttttcctaatatagctctgaggagactggtgtttaagaggagaatgttaagccaaaagcccatttcacttggctctgtacagacagtaagttaatttccaggatgtataactcctgattttctgtgatgacagagaaaatactgacctgatttgggtaactctgaggtttggagatcttgaatagcctcatgttcctgaacttctttaccatacatgaattacttcttaggaagaaacatttattctgtagaatttggtttcgcattttatttttattttcttagacactggtagttggaaattcaggaagaatttgactacaaaaaacatttattaaaaagagtttctaatctcacctgctaagtgctacaaggaaaaaaaaaaatacattttgagccctcaaggggctccaaatacggttgaaggaaaattagacccagaatagtatatgattaggaacaaaacgatggcagagaaatgagagtttagagaaaggagagataatcctgagttaccttaggaagagaaaatttcacagagaaaacggcccccacactgatttggggaagatgattcagacttgtctcaatggcatgaggagcaggggccagatgggaaaggacatcgtggtggaaatgacacgatgagcagaagcttagaagtacttctgcttcacgtctgtgattctgcctctccccttgtttctgcagtgctaagttgagtgtagttccccacacaaaccctgagctttcttcccctccaggctgctccatcagtcagacaggagtgccttctcaggggatcctcttcctcatcttatcgggccactcatctcgtcgcagacattcccatactcctggttgacttgctcttccctctttttaactgacatgagcaggcatcacctcttccaggaaacttccctgacaccaggctgagtctctctggtgtcggggaaggcttctgtattactgcatctgccattttgatttaaggttttctgctcattccaactcaacagtgagctctttgagggcagaaattgtgtcttattcatttttcttgttatgccctaaaaatgtattgtgataatatataagtaacgtacaatttaccattttcacctcttgaagtgtatagctcagtggcgttaagtgcattcacactgttgtgcaaccattatcaccattcatctccagaactttctcatcatcccaggcaaactctgtatccattacacaatagctctccatttcctcctgccctcagcctcaggaaacctcccatctactttctctttctacgcgtttaactactcatatagatggaatcatccaacatttttccttttgtgcctggcttatttcacttgacataatgttttcaaggttcttttatgttttcttatgcgtttttatatctctctatctttgatagcacccaatacatagaagacaatgaatgtttttgtgagtgaaaacataagaccagaaagaaataggatgtcatattctagggacactcaagacactttcttgaggacagaggcactgtagcttggaatttggaagatgaggttggcaaggtgggtggggaaaggatggaaaagtgatacgtttcttgaagcatgtggatttgcttccataagcagtggtggaccagtgggcacccttatctattaaaagaatgattttttttgtgactcgtgtagagcactgtcttagttacctaccccagtggtagataagagaagcggattaaaaagagatctgaacctgaggaaatggagactaccaagttttttgtaaatatgtctgttataacatatattatctagtacttgcgatgcctgtgccaaagcatgctttttggtttagttaagcctacttagctcgctaatttcagtaattttggcttgaattgcaaaaagttgatgggggagatgggggagtctctgacatccttcccccactcacaggtttgaaaaataaaattatgaagagcaaaaggatctttttctgtggtaaattgtacttcatgacaataaacgagttgtggttttagtggttaatttataaattaggccatctggttcttctgtaatctaaagatttttatatatatgtaaaatacaaagttgtcctgggcaactgccaatctataaaggagaaaacatcttgaactctgtggagataataaaaccatattcagctgccaaagtgctgctgacatactgtccaatgagacacccaacgcctttgcattacagccagcaaggtgtggagctagatgtattgatctatttaataatattagcttctcttctctgggtcggacttggcccagagcaagtgaacaggacctatctatctccttctcaaaagtcacagctttcttaccaaattgtacaactcaagccacaaagaaaacaagtcaaggaaaggaccagaggagcacgagggcacatgccttgttcccagaccctgggaccagctccagctctggctccttgcagatcccgctcttctcaagttctctggctttgatcctagttttctgccctactattcctgacatgtacagcacttagcaattcataacttgctttattaaccaatatctcaacagaacttttaaacaacctgggaggaagcagagtgggtgttactgtcctcattgcacagataatgaaactgaggttcagaaaggttgagtgtcttgcccaaggccacacggcttcttagtggagaagccaggatctgcaccaatgtcttccattggacacactgccacttaatatggtacttagtttttctttctagatgaagcagtatgaggaattctaccatggatatctctctctatttttttaaactgcaaaataatgataatgataataatgaatacttctagaatacagtctctatgccaggcactatatgctttatatgtactaacttgcttaattctcctaacaaccctagaaggtagttactaatattatcactatttttaaatggggaaactgagttacagagatggtaaataagttgcccaaggtcacagaggcagccagattagtgacaaaagtttgagagtactcagtttgaattttacatgttccgtggatatggtttatgccaaataccttctcctttctagacctcagtttcctcatgtatcaaacaagtggactgcactatacaactctgaggtcccttctagcctgaagattagagtcttttatgcccacatgatatggaaaacattaatgctggctctttaagactggaaccttgtcatttaaaaaaataagctctactaaatttgcagtaaactgtccaaatacgatctctggtctctgatttgtatctctcacagagcatgatacaatgctagacacatatatactcattaaaaacttgctgaattaaattgtaaaacattcctccagatagaccgtaagggtgagctgatgccctaatatgctcctcagctgctgacttaggacccgtgggtgacaaaccctgtccttgttgttccatctgtgacagtgggcagtgtccaaaatgagtctcttcatcaaaagtgagtccaggaaactggatcagagtaagtaaggagtcttccttgcttctttttctctaaaatacaatacagtgcattctagcaaactaccagcatgcctataaatctcattttaaaaatatcaggaaatagatgctcattgataaagagggtaagaatgagcaagggagagagtcaagcctgtacatttgtgcttagggtattttcatttgagaaattatcattggaaagatcaatttccaaggcaggcgttttgtcttgacttaataaactaatctttatcaataataattaaataatgtcattttcccagatacaaaggggaaaccataatgcctttcatatgccttctcatatcttcagtgtttctcaaacccagctgtggggtctttttagggaagatacaaggagggtgagaccatgtggagcacacactcagcagccactgtgaagtcatgggtcccttgctacctttggaagctgacaatctgcatgaaaagcagcccctaggaacatgggtagtggcccgggatccaaaactcctgaatccataaaagggcttttgacttcttgttccaggacaagtgacatctgacatgcttgcctgcttgtttgcctttcttccttctcctatccgtctttccatccatctgtccatcagtctatctgtccatccagtcatccatccatccttccatccattcgtctattcatccatccgtccttccatccatccatccaagatttattgagcacttacatgacaggaagtatgagacacaaaagaaaattctacacaattctcctagggacatgaactaatggttagcaaaaatatatgggaagtcctgcttcaagtaaaattatgacttaagatatacatcagatatttgaaaaagatatttttaagttcaacattgaaattctaactacattgtgtccttaatttctattttctagttagacttttttgaagatacaaatcatttgaattatcattattattgttacttgattttatttattccacatgaatggtaagaattttaaaggaaggtgggtgccctaaaaatacatttatgccaactggattgaatcaccttgaatcacaatgagtacaacaactgctaatgagctaacacttaatgagtaatgaccaggcaccagacactgttctagcactttatacttactgatttctttactcttcatgataaccctatgaggaaagtgcttttttgttaagctccattttcagctgaagaagctgaggcctggagaagttacatagctttctcatggtgtctccactatgaagtagctgagctgagatctcacccaaggccctgctgggttcaggccacactcttaagtactcacagctgcatcacactgaatatcatctccctgcactgccatttcatttgagcagggctttgcctgtcaagaacattcatatcttttgataaacagtgcattttttccctgatagtctgtatgggacagcctccttgtttgcatctgaaggacagggacttcaggacctcctgctgtggtcaccaaggaatcacattcaaacttctcagcaaggtgttatggtccttctcagtttgcctacgatccattgaggcaaaggtttccttctctcaatacaccctctgctttagccagggggacctcccactgtcctctctatgtgttctatttgttcctgactgttagtacccgcttgtgtggtttccatgatccttcttctgcctggaatgaggctcctcttgagtcctgagagcttcatttctgtctgtcttttgggctgtgccaaagcccctctgccttcgtggactccccactgattcattaccaattttcctctcctctgggctcttcttagattgcaaatggtgcctctggattgggtaccaagacttgcagtgccttattttgataccactaatatgagaccatgttatatgcctcttgagatatcattccctttctagcaatagacagtgataagcatgcgtatagagggtaaacgtaggactttgaggttcatccatctcaatagacaaaagtaaaagtatgctaatttcaatccttcacacagtaaattgaatgtaataggtcttcagtaactatttgaggaatgaaattgttattgatatattgatttaactgagcaaatccataagtgccacgcttggagtaaagtacagggaatctcattttttgggtaatttcaaacaagtaggagaccatccactatctattaaggacaaaggagatttgagtgacaaacatgttccagaccatccaatggtgttattctactacttcatcccagctagtaaaatggggcaataagaaccaaaaagcaggtactgtccctatgtaagtagacttcctagtcctggccaaaacaaaaacaaaaaaaaaacaaagcaacacaaaacaaaaacaatgtgtgaagagcatcttaccgttcattgctttggatgatttgtgcaaatctagttaatgggggtggatctgatcagtctttgtaggaagtcagtattggcatatgtctggcttctgaattttctaaaaatataattatttatttttaaatccattcaatcaacactgaatttactaatcaactactaaataagtaaaagatcctatagtagatccttcaagaaatggaaattgagacattaggtcactataaaccccaacccttgtaatctttttgtggcaaccttaggaaacttggaatgctttttacactctgataaaggtttaattgtaatcatagccccagtgttgcaaaatgagtcagaagatacaaagtatctcataggataatattatagctgtaggtacttcaaatggacaagtataacacattatgactgctaatatggatgcattgggaggaaaataagccctaagaagcaatatgcagaaaagctatgattgctggagacagatagatccaagaacctaggtaatttttcagcaaactgtggttccactttcgcctctttcttgacattgactgtgtttttaacagggaaaaagttatctgaaatttttgatagagttcaaactttaaatcgtgttcactttgctaaaactccactggatggagtggcagaagagaaaaaacacagaaataaaagaaaagcagtaatggagaatggaaaatctttaaagatgctaccatcctgaaaagattcgaaagcaagcccaggaaagccacggggagggaaagagcgcttagaggtgtccccaaagctttcagaagtgatgagggaaaccccagctcttcttgttacttcttggcattttgttcctctatacgttagcttttggttaatctttcatgttacttattatctctgttttaaaatttcacattaactcattggtttcatatcccacttaacaaattagatattttccttgggcttattaagaaaattatttgctctcatatgttatggctttagattttctgtgtagctaggactgttcttaaatttttatctttacctttgttatttctctgaagcatccccattggggtggcagtggggggtattaaatggttatgtaaaatttacatgtaattgtgcaatttacacatagcctcaaatataatgaggcctagtctagctgcccataccaagaggatattatagttgccaataaaatcagtattatttcttttttttttctgttcttttttagcttcttgcaaacattcactcagaatcctttttgaatatcagttttgtgaaaagcaccgtgctgggttctttgggaaaagccaagatgagggcaaatgcatcctccttcctagtgagtctagcacacagatgactcaggaagtcatgcactggaggaggagtatcttctagaactggggaaaggttaaggaagggatcagaaacctttttcactcaaccaattacattttcctgaaagcatgttattaatcgtacatgatcttgcagaccccagatagagactatatcactatatcaattgttttcttgaacaactggagttaattctcatatgtttggttacccttcaagtatttatgtttaggatctgttttcttcctcttaacttacattccaaagtttaggtatccttcccctccatttgctgtccaaaacagcatcctttcatgtagctacatgccattggaatgatttggtttttgccatggattcctagtcctaaatttggacttttattggtatctaagatgtcatggacagaacccacagatgttgtacaaaaagggctgagtggacagtccaacagagttacatcatctgttccaaacacccttgtaatggttgtatcagcgtcaatgtctgagttgctgtcctggttctcctggccagtcctcagctggccatgcctgttcaggcaccagagaaagcttcatacctcaggcaaatctttacaagggatttgaaaggcaagaaacatgtattttggaggttttacctgtttatttcatgtatagcttcatattaacagattttgctatcacttaaaagtaaataaaatgattttgatttctcaaaatcatcttataatttattaatctattttaaggcttgcatttacatttaatcttcaaatcttgatgcattttcacagagtttctttagttagttaaggatattaatgacttcctttaatagttgatgaagtgagtcatcatcgacagtgagtcactaaggccaactcttgtacctgctcttaaatagcagttgttttactagatttgtactgaaacctttttaaaaatctgtttgtcttatatataacaagcatttccagtagttaataatactttcttagctcttcaatcattttatgagagcattacaagaaaagcaggagatggggaaagagatatcttgtttgccatgtaggatgcatgaaattcttacttagtctgttgtgttttggattcgagagaaaatatgtaatgaatcaaaatgtttcctacagtcctctgaaaagatgtaatgagacagtcttccagcagcctttgatcatctgggaagtcattgactttgatccttatcctatcaggggctcttaccctggggtccataaatacccaggcatttttaagaagtggatttcagtataacttgtttcattttgtgatttttaaaaatattttgttttatgcacttaaaaccttaatctgagaagtcatctgtagactataccagatttccaaatggaataaaaatatttaagaactctcttatgtgtgtgtttcaacctataaattttatatgtgtagctcatggttaggtggtaagtagagaatttgctatctgtgtaatatttggtactttttataatgaaaaaggaagtaatacaatgataggtaccatttatttgttgaccacccatattttccagggaatgtgatagatactttttatatattctttctagttttcaccataacatgctgcagggatagtgttaccattctcattttgcagatgaagaaattgaaacccagaacaattcgttcattcattcaaaaaatatccatagagcatacactaagagaaggcactctgctaggcaccagaaattgaataattgaccccaaatcacctaactcataagtggcaaagctggacctaacaaagcccattatcccttctatcacacattccatcctcataatatcatattctacatagaaaaacttaacagtcctgttgagacacttggaactctaagaaggtagtcaacagatacttttctctttatataaatatgatatctaattagcttttcctggaaaggagtgattctgcctttttttaattctagcattccctgagcaaattagagcccaggtaatgatttcctgagataagtcaattctcttctaggacaatctctagtcttgtcatcatgagatggaataacaacggtctatgtcatttcagtgacggcacctgcatgtccttgtatctaagaaaagacacctccttctctgccttgctaaatatttgtaaactttgctttaattggtatatgtgccactgttttgtatgtgggaggggtttatggttttgttttgttttttggtcttttttagtaaccccaatccaaactattctcatttgtaacctaaaatacttagaacaagaggctttgcaatcttgaagaccgaatacatttttactactaaatgtaaggcagatttgagccacccattttgtagtgcttgattatggcaacccgaggaaactaatacggttactctttatctttattaggaaatactgctgtagttcctgttgtaatccttagggagccaacattgttgatgagacctagtaccaagccagaggccatgttgaactagcaagatatgggatagtttttactttcaaagtaattagcatctagagtctagagagtttctcttacagggtggcctggtgtcttagtctattttctgttcctataatagaatacctgagactgggtaatgtacaaattataaagatttgtttgactcacaatcctagaggctgggaggttcaagatcagacagccacatctggccatcctttaatgagggcctcatgctgtgtcataacacagcagagaattggaaggagaaatgggcacatgcaaagagaaagaaaggggacgaaaggggctaactcactttgtaactatgagactaatccattccctggagaactaatttattctcatgagaaagacatgaatacatcttaacatcctaatcacttcttaaaggcctcaactctgtacactgctacattgtaacaggcaattaaatttcagcatgagttttggtggggacaaactacatcccaactgtagtacctagctacccactttatttttagagtggctatttaatgacctggcaagatctactgtgcttgtaaaagaatccaccatgggttaaagaaaaatagtttctggcttccagtctccttttggcctctctctagaatggcatatacaagttcagtggttactgacttcaaaggtggggtggaagacactgaaatgaatttttctcctttctttggttggttgctctctctctttgttgctgaaatttgtgtaggataatggaatgaatatgggagatagggaactatagaaagactggagggaagggagaagtcttttctgaattaaccccccatgtgcttgtgagacccttggaccccaaagggctcctgactggctgggcccactccttctgtatatggagccatagggcttttctctggcttcccagttgaagcattgtcatatgctttgatgctcagcatttctggcaattttaccttgtaaaactcccgtttcgacattctgaacttaagtagaaaatgggaatataagcctcttgttttttgctcttaatccattccagaactataagtccctgggttcttggcaacaacaggtgttcctctagattttaaatcaagtgtgtgtgtgtgtgtgtgtgtgtgtgtgtgtgtgtggaatatctcactaaaacgacagtgttgtcatcaattttggcttcatggagggtcttgagctgggtggagatgggaggaagctatccttgtccatgcggtttggagacttgctctaaaaaagagaccgtctacttccctcgtagtttttgttttgaagacatttgtagtgtttaccaggagcatacagttatctggaaaatgcagactgatgacaatagtatttgaaatgctcgtagatctgcttctaggttgatggaaagaatgtatgttccaaggtacaagtgcttgttgacttttcggatgcagccagtctgtgctctttgtcaggtaactcggaatcactccacagtagctgtccttcctgtccatttgacaagcacttaaaagggcagtcttaagtactttacttcatagtttattaattttttcccttctttagtttgcaatctactctcagaaactgaaaaataatttcttttacctttttcccctttctcatttaagctgaaattgatttctttctgagcgtgtaaggaaaaaaataaaaagaagctgtggttaatacaattgcctttgctgaacaaaaataaaagactagtttaccttcaagctgtgctgtatcagagtaaggatctgttctgtcactcccaggagattttgataagagagagtacaaaatttcataatatctacatagcataattgtagcaaatataagaagtattataattgtttatttggggatgagggtggggatgggtttttttgtgtgtgtttggaaaattgggaagctagaagatactcttaaatccatcagcttcttcagaactcttctcaaatgcttattgtcatgacaaccatctcttcataaaccaaggctctgagtgtaatacagatggggctggtaacaaggtaggattgccatctgctttgttttctctaagggacattatttttttgtgatccccaagtctcatcctcaaatctgaaagtcttgacttatttttctttctggagtcagatagagctgtgagattaattcttttctcctgacaaactacttcctcacctgtcatcaaacaacttacttgttaaatggagggaaaaaagggtaattatgtgtgtgtgtgtgtgtgtgtgtgtgtgtgtgaaaacaaatatacatattttaatgatttatttatctgtctactttgcaaagtctcctatgaggtacctagtaattcaataatacagtcaagtgtgagatccagaaacaagtaggagtgaggcaatcattttcccatgaaacaagtaaaattattactgcttttgaacattttaacacctgtgcttcctgatggtggggctgcatgaagtttgtaagtagtgtcatcattctcttattaggtaacaaataggagcataattatttaggagaggaaaaccttttcttgttaccaaatttggtgagaaatttgccaaagaaatgaatgcttacaggagggatacagcttaatatcttcatactcccctttgtcaaagatacaagtgacattttctcatatcaacttcctacaaaaagtgaggccatacatttttaaaaagaatatacccagtattttaaaagtttgtatatatcgtgatcagaattaagatatatccagcttttgaaaaaatatacatatttagccattaaaaggtatgtggtaaactaaactgatgaataaaggttatgggaaatatttcataaaatttatatttaaagtaaagaacaatttctctctttgtattatagttattttacataagctacaatctataattctaaaccttaatttcttgaagggctttataagcctcatttttctttctaaatagagaagttcatacactgcttttatgtcctatatgttgatgtgtaaaaataatcaaatactagtgggtgagtagaatgtaactacaatatggtacacacacagacacacaggatgactaaactgtgcacatgagctgatgtccagagcagggaaaagctgctaaagaatgtttgaagtagagtcagaatgcagttttgtggttaagaccaataatttttcaaaggagcattataaggagtgtggttacttgggttggagttgaaaaccaagcaacagttgggcagacttgaaaccagttttgtctttaggtttcttttcttaatgattggtctacaatgagttagccactcatcgtatttaaggtcttggtaaaggagagactttctttgccatgtagtttatacctcatgtatgcattacagtagagagctccaggaaatactgcaggccttctactggtttttatcctctatttagataaaaatgaggaagaagtagaattatcataaacaatcctgttggcttttagttgagatatactacagaaacatcacctttgaatctgtatatgtaaaaaaaaaaaatctcagagtaagatataagttctgttgcattgttctgaaggaatgggtaatgtcagaaatgtcttcattacattacataaagtaatacacagtgctgttatcagattgcatagtcaaagttggcaagtttgcatatttacatagatatcatgaccttcatcattcatacttctttcaagaaatggtcatcttaatatggggtatttatttgaggcatacaccatttattttaagaagcagtacctttgtttattgtaaaacttctggactaaattcttcaattttttctcaaatgaattcagttttttgttttttttcttaccactggtttttactgcatagcgtttgcctgaagaacaccactttgtttcccaaggcaagtagtcactacaaggcgagttttgttctgtctatcccaaggcaaatagacagcagcaaacatagtgtggagggctgctgggttcagtagaaaaccatcaactatttctaattgggggtgtatgaagacagctgcattctggacatgctaaacatcttgaaatgcttggagtgtaataaaaccccaaacgtgctttcttttctcatttctttgcacatgtattttaggcaaaatataatccacatgctgttgttccctctccttttaaatcatgaaaaatgttgtttttatagggctatttgaggtccaggatgcctttttatcctttttattaggttatttttatatataaatgaaaagaaaaacataaacagaaaatacaacatgacatcatgaatgagggcaagcacaaactgcccaatttaagtttgaagttataaacctaaaatattttaaaacaaaacatttctcttggtgttttgcaaaagtaatgctttcttctattgggcagaaattgcagcctgttgggatgaaggagagaatagaactaatatttattgagtagctaccatggctttctgggacatctgctaaccccttaatttttacagtaaccctaagtggtaggccagtccatctgcattttacagacaggaaatgctttcagagatattaattagatggctgtagaccacagaagaattgaaagtcaggtcttcttgacttttcagctcatgacttcattacttctgccctcctcattctgttgggagatgggtgggagtgtgccctccagataatcagaaagtcccatgttgggatgtgatttggaaatagctagtcctcaggcctgtggacgaaggctattgtagcctcaagaaagaaagagtcagcttgatattggcaaggtggttatttagtgtggcagctgcataagagcattggataggtggtatgatatgggatgggggtcagggtgagagctgctcacacctccttattatggcagggcaaaaccagaatagtcacagtgatggtgatgtagggtaggttcaagagaaagctcatgttcttctagcattgagactcacaatatttcaaaacaagtgattatttattccattactatttattttggtgatggatgttgcatttattttattttctaacagtagtacatatttggtaagagttgtttgtgacattctttatttaaatggattaaacccattttcttaatttttttaagtgtttgtcctgttatcgaagatatcttaaagtgttacagaatgggaatcatagctttaatgagtctgcatataagagagtccaaacattttttttttggagagaaggaaggtcatttcaactgtactataggaacgatagggcccatgatccatggtatggcaataagttactattttttggagaataaatcgcaaaatatgggacgtgaagactgcttggtatttacacaggagtctagttcttctgatactatatattctataagctttctcccagaaatccaaagttttatttagcttgtagccatcattgtgccacaatcttccttccatgatttatttttttcccatactgtactcttgcactgagtctttggcattgaaagggaaagagatgaattattcaactcttcttctattgagtgaaaagccaatacagaaaaaaaaatgcaacaggcaatttcatgactgacattttggagaacattggggtcatcatatgactgagaaaatgattatctcttttgaaggtcaggaaaccaatatttttttttccagtgaatctactgagggaaccaaccacctagctttttgtaaatagcactcctcagggaaatgttgcttttttcttttgcagtgtttctgctttgaatcttcatagactgtatcccatggaagcaggaatataattactgtagtgcttactttttttcatgtctctactgaagagccaccctgatttatcttttactccaaggtagtgtggaactttacaaggatttagattatattagtcattttgcttggagatctctgcacacttttcaacatcatttctaagagccttgccacactcagtaaaagagggtgaggcttcaagaatctctgtgtaccaacgcaaattaggcaatttgtaagaagtccctagtagagcagagtggagagagggctcaggatctcgtgaggcctggggcagggaggtggacatgctatgagctcctttcacagtcaacacaaagttaacacatcttggaggaaaaaccgttactgtataatggaaggcagaaggcagggatgtctgtgcttggtgatgaagctataagtagtgagctgatttatgatggaggggtgaattgtgccatgaaagagactcccctcaattttattagccagattactgcccatatgcggggtgaggagtgagcattttaaggcatttctctctgttttcaatatatcatctctggattcactattactgggacttgtgaatatagactagggattaaaacctgagtttgcttttaaaaaactttattttattaaatgagatttctgagcaactgatttcacttagtcttatcaaactattcttactactttttcgatatgcattctgtccctcctgtgtgaaggccacaggcccatggcccccatgtcattgagatggaggtcctcaagtgatggcgggatgaatcacctctcacttgccacctgcgagaatgtctatcactgatacctctccttatgtcccgtagcagctgctaagctcccacgttattattggtgacactttctagtgttcaggagagaacaggatttactttttgaatgcctctaaattattatagcaggttcatttggaattgcatatcagaacattcttcctgtttatcaggaaaataaaccaaggtcttgaaacttgatgtctatccatagttggcccaatataattttgcatgtggtttattacattatttttctccattggctatgaactgaaggcatctcatggatatttattccacgctgaaaaggtagttagatgctgtgtggtaaagtcgagaccagtatattggaagaaaagagacactccatctaataatctttcatagtcttcacattcattgagaaggttacttgataacctttcctgcacactctctcccaaccctgacccaggaacttgcagagcacactgtgcaacagatctaataacccttgtctgcaggcaatatgcacagctgggaaagataattaaatagactctttttgagaggtctacaacatttcaacggaaggatcgagcaatccggctggcctccgactgaaaactattaccaacagacatacttcagacagattccatgatcaccatccttatgtggcacaagtgcttatggggagcttctttggcttccaaaaggccactaaaactgtcagacctaaataatactggaactgtaaagtgggaattaaaaattataattccatttaacatgtaacgttctcatcagaaaggggcctttggttttccatattgatcggttacatggtcagctgcaattgtaactcatgagacacacccagatatcatcctgcgtgattctttttgaggtttatttagggtgttagataatccgaggtaggtttcatcatgggtggcaatattacatatagcaaatgtcatgagaagagagacaaagcctcccttttgaatgttaacatttatgcaataagtttaatgtaccgtgtaagtttacattactattatgagacttaacctaaaaaattggggtgaaatcttaaagaattatgaaattaagaaatgaaaccagagggctttcgttttaagtactgatcacttaaaccaaaaccagtttggcttatcctatgagatcgtgggtattgagaaagaggaacatttgctgctgcatccggagatgtttccttgagagcaagactagttccttttgacccagtacatcctacaggtaaatcactgcatgctgtagagatacctgttccctcccttcccccaccccctggggatctgaaaccaaatcattgctttttcatcttttacttatccttataaacaaagccacccactttctcatttttcctgatgccaagtccttctttacatgagtggaatccactcttttcaatggaaagacacctgctctgagacaggctacactgtcatgagccttggcatggctgagtgatgtagtggaaggtgcaataaatttaaactttaaaaatgcaaaatttgatggaatcatgcatcttgtgcctacagtgagtatatggtctagcaggatttacagatacatgcatgattaatttgaatacagtatagcaaaatgatctgttaaaaaacacatgaaaagatgtgcaaccttattagtcattaggaaaatgcacataaaaccaccgttcctgtgtgatacctctgtacgtctattagaatgtataaaatttaaaaagactgaacatagcaagcactggtgaggttgtagaccaactggtgctttcatgttttgctggtgagaatgtaaacattacaactactttgaaaacagtttgacagtttcttaaaaagctaaaacatccacctggcatgctatatacagatatcctactcttacgtatttaaccaagagaaataaaagcatatatccattcaaaaacttgtaaataaattgctcctagcagctttatttgtaatagccaaaaactagaaacaacccaaatgtccaatgaaaggatacatcgtatttatttataggacatatccatgcaatggaataccacttaggaatagaaagaatcaactgttcatcatacatacaaccacatggctaagtcttaaaaataattatgcttagttaagaagtcagacaaaaaggtaagagactgttaggagctgaattagcgggttaacagtggcaatgaaaaaggagaaaattatagctacctcaagatagaattgatgagctttgattatgaaatgatggtaagagagagggccatagactctgaggtctgtctctagctttggtttccctatgtggatcattatctatttggatataagaattatctgaaagatgatacttaagcatgtaagaaatgataagctacctgtttaatgatgagcttttgttacctccaagaaccccaagaagaaacatcctgtaagcagaccattcatacttgattgttaaaggaaaacaaaattccattttgtctttttcatcaacacaagtatagctggctaataaaagtgtaatattcatgagagaaaaagaaaagaacacgcacacacatacactcaacaggattcctagagtcattcccatggataagagggaaggaaaggttgagggcaaagagagaagggaaggaaacagactatgatgggatatcttagggcaaaagaatagggggctgatttggaaggcagcaaacttcatggatacttgatttatttaatgtccctgtcactctggcgtatcttaatgtgtgtggtgtgtgatttatttagtcacttgatccaaattctctaattaagtggagctcagaagatatagtgtccattgtggtcaagggcacaggttttggcttcaacagacaaataagctctctgagcatctgtcttctcatctgtacaatgggaatatcagttcctacctcttcaggttgttgtattcataaataaattgtgtacaagaaacattaggattgcttcttacccgtagtaagggctcaacaaaccttctcctccttctcctcctccttctccttcttcttcttcctcctcctcctcttcttcttccttttgcttttctccttccttcttccttcctttcttcctacctccttctcattattgttattaggtaaccacaatattatcagtaatgattggagaaagctttaatctcctagttcaccattagaaaacaagaacacattttggtggttattacccgaagtaatcataatgtcacctttttttccatctgactcattatcccaagtgatttatttatatatggagttttctgagtctttcttttacatattacaaaaaaagagtgtgatttagggacgaagcaaagaaataaaaatttagtgactttcattctgcctgtgccccaattcctattgggcataaggcaagtaatttaaatttcttagcaccttagcatcttctactcaaacagaaatgaggaacagtcacaggttactattatagctgtctaagtagaaggcacacaagttttcacactgagtataacactttatagaaagctaagtgtgttgctcaagttggtacatttctgtagatgtgacactatggcactaagaaacttaatgccacattgaaattcattgagatagctagactttaaaaataattacttgacttcactataaagtatgttcgtattgcatttactccatctagtagaaaatagaccttgtcagttcaaatccctgttgcattaatttcaccagtaatgagtctttttcatttgagtcagcagggtttttcttgcttgttttcaggctttgtggatttgaccctccatgatcaggtccaccttctagaatgtgcctggctagagatcctgatgattggtctcgtctggcgctccatggagcacccagggaagctactgtttgctcctaacttgctcttggacaggtaagtgacctggctgtagcttaggagtagcatgttctttacgatcatagttcattcatgaaactattttattcatctctcggtgaagcttcagagaactttattaggtatgtttacttaacaaaagagtgcattgggggtgatgaagcctagtcaaattcacagaaagctaaggataactttctgctagacattacctcagaagaattctattatttctaatacacacacacacacacacacacacacacactcacactctctctctctctctctctgtcattatgaatggtaattttctaactccatcttcaacttgtatcatataaaaattataataacctctctttaattaaaatctgttgttgcttcttgtacatccataccacaatagcctattcattttcttctccaattttcccatccgtaaaatgaagaaatttgaccagagttctgaaggtcacattcaggtcgacaaattcattttcatgttcaaatatgttaccttctttaacataccattctggggttgccttggaatgtgggtcccattgttttttttttttcagtcattgcttagagtcatagaatttagatattactcaatagcagctgccactgatagagtctccaccctgcaccagctgtgatgctaaacactttacatatattatctcatttaatcatcaccggactcctaggaggcaggaatgtcatcatccatgttttaccagaaaggaaactaaatctcagagacatcctgctacttgcaaaaagaggaaagctcactaaatggtggagccagagttcaaattcaagatctttctggctccggtatgctctgttacctcctgtgctgggcacatggtcttcccactctcatgttcagtgatgcctcctttggtctgctgccatagcattctgttttccaggtaaatcttgtctttgtgggctacataacattttggatgagaaagaaccattttgttgtttcttgcaatcctattttgcctccgtgccaagcagtctaaggctgccaggctgcccacagtgcatctctgcatggtactttacttcagagcatttcactatctttctacacctgccaagtgcctggagcagagttgcagcaaaatttatttaagtactggagaattgtacaaggtgttggttattgttttgtcattttgtgttaacacagcttttgaaaaacagtggtgatacagtttaagaagcatattttgctgtctgtggaatatattttgatatcacagtttcacaaaattatcaagaatggccactagtcttgttccttagaactgtactcacaatgtattctgtcagccttatagaggatgtctttaccgtgttctttcctttccattccttgtttcctttccattttttcctatctcatttctcctctcttttcctttgctttcctctttctttctttctcctgtcctcttaatttttttgtcttatagcagtgtggttttgtaaccaagtgatatcaagtttgcaaatgaaaatcttagaccacacgtaacatttccctgcctactgcctggagtgtctaccatgtttaggtttttggacattcataactcatttgctccttgatttccatcctcatctgtatctatcttcttttttaaaatttaattcaatttttataatgttgacacaattatgcagattcatagggtacacagtgatgttttgatacatataatgtgtggtgatcttatctatcttcttgagtacacacttgcttgaggacagtcatcaaataattgcattttgaatatgtgaaggtttttgacattactgcacccaaaaaactcatattgccagtgaggtgatatggcctaggattttatcagctacagccttctctttcctttctgtacactccagtggtggctaattttctttcctctctcacagaagtatgaatgactaaaagttctcatctctattcattcctactttctaaaacttcagatcggaaatttgaattacctctagaccaggatttgtcagtctctttactattgacattttgggtcagatcattctttcattctttcttggttgaggggttgatattgtttggtgtgtcctcatccaaatctcaaattatagctcccatagttcccatgtgtcatgggagggacccggtgggaggtaactgaaacataagcaaaggtctttcccttgctcctctcatgacagtgaatttctcatgatatctaatggttttataaaggggagttcccctgcacacgctctctctcttccccgttgccatgaaagatgtgactttgctccttcttccccatgattgtgaggcttccccagtcacaaggaactgtgagtccattaaacctttttctttgtaaattacccagtctcaggtaggtctttattagcagcttgagaacagactaatacaaggggctgtcttgtgcattttaggatgtttaacagcatccctggactccaccagctagctgccagtagcaacctccacttcctccagttacgataactaacaatgtctccggacatttctacctatcttctgttgtgtcagggggtgggggaggtaaaattgcctctggttgagaatcaccactctatgctttcccaactcaatgttctataagctcctcagacacactgtactctaaactgcacactcactttatcttctgcaacaagtctattccttttcttttctgtcttggtgacatcaacactcacctagacactaaagctagaagctttaagttacccatggattctaccttctccctcaccaaattatccagttaactatcaagtcatctatgaactgcttctgaatccagacctcccctctatccccattcctctgcatggcacaggtcctggttgtctctgctgcagattccttgcttctgcaccactgactcactttcttggtcctgctactctttctttcagtccatctcccatactgctgctggagaaggctttctaaggcacagctgtgatcatgatgcttcctgactcacctttcagtggctctctcaattcttaacatggagaaaccccatctctactaaaaatacaaagttagccgggcatggtggctcatgcctgtaatcccagctacccgagaggctgaggcaggagaatcgcttgaacatgggaggcagacgttgcagtgagccaaaattgtgccattgcactccagcctggatgaaattccgtctcaaaaaaagaaaagaaaatactgtgtagacttcttatcttaataatcatgaatcactattacaagtgtctcaaacgcaaatttctgaagagtcacaggtgacccagtgagcccttgtccaggctaaaaacacctggtggctgaattctgaacttcacacattcgaatttctcttccattgcttctgagagaccctccaccattacggttccttatcagagtcatatctacctataaggctcatttcaggtgccgtctttttataaaatcttccttcaaactttctcctcttcagcctttatatatacaaaaggcttttctttcttttccttttctaagtttccattgtagttggtttgcttttctctttattattctcattccaccatattagttatgattggatgtcttcttaccttccctactagctcataaacttcttgtggtcaaaaaccagatcttgttcgtcttgtgtgatctccagtttacgaagtcttccatgatacccaacaaatatttgttaattgcactggccaaggtcacccaggtggcttgtggccaaatcagaagaatctacatcttctgatagtcatggcagctgttattcccatccaactcacttcctgtgcattgctactcatcaattccccacttattcttttaaaaacattgcataaatacatatctatgtgttttggaaagattttcttaatctataagcgcatttggcgtgtgcttatatgtctgcttatatgccaaatttgaaattccaaatttcgccatttggaatttcaaatggggaaaagcaaagactcttatctttcccataatcaaaccccttatgaagtaaaatccttaaggtctctctacaggttaaaaataattaggtgatatgatttggctgtgttcccacccaaatttcacctttaattgtaataatccccaggtgtcaagggtggggccaggtggagataattgaatcatgtgggttgttttccccatactgttagtgtggtagtgaataagtttcatgagatctgatgattttataaatgggagtccccctgcacaagctctcttgcctgctgccatgtaagatgtgactttgctcctccttgccttccgccatgattatgaggcctcctcagccatgtggaactctgagtcaattaaacctctttcttttatatattacccagtctcaagtatgtctttattagcagcatgagaacaaactaatacattaggtatatgttaatgatgaggaatgtgtataaatacatatacatttatatttaaatattaatatttacaaataacatgatattaaaaatatggtctataattttttaaattatctacctttctaatcattaacatgcactaaatattattaaatttcaatattatttaattaccatcttggtgtttgaggataactgcttttgatggcattgatagatgcattaacagtgaaacattgtgaccaacagcccatctgagattttatacttgtagaagatggcttctgagtgacagctgagaagactgtttatgtctttgccctacaggttcctaacccctttaataaaatagagctctctgatgcagatgacacatgggccttctttttgctcttgtcccatcacattgtacatagtaaacatttttagcaacaaatagtagatacttataactttaaaagctaagtggttgacagctgagaggcagatgatggtaatttcatcatttttctcatatctcaggcattgtgacactacctctgcaaggtcaactgtctccataggctgttttcatttgcgtagaaataggggggaaagatagcttgaagtcatcaggagccctgcaacccatggattaccaatgttgctaactggagcctggatttcatcttcaattgattgtaatgatgtcttgttctctcaaatctttgcaaatctaggcatttataagactatcctgtaggtcccttacaagaccatgaggatgctagaacttaccctagtcttttcttgtaattgcttaaatgttagtattggcaaatggggtttggtgattataagagtaaaaaaacctgctgccatcccacatttgtgagcagaggatacatttcctatcttgtgtccatttaaaaagaatgattgcttgattggcttcctgatgacaacccatgatatactgattctgttagtttataactttcctagtagtcatatgcttataggccaattttatccttgccatgcttagcctagtcacaatttccagttcttctctgtatcctgcagcagtgagttccaaacacttaatgctgtcatctcttcctgtgaaaaccaagaaataaaggttattataaggtataaataaagaacccagtgatttctcttggggtgtgtgtgtccatgtgtgtgtatattttacaagaggaaagttaaagatactaagaatgcctgtgaaagtaatcaggaaatggagaaaacttgttttccattctagcacctaatcctttggtgtgttttggcactagaaaacacaaaatatgtcctctgtgtctactaggaatgcctctcttcatttagtcctgcttgagtgctcatgatggaaaaatatagactgaaaacaggagcaaaagtgttcatcctactcattccttgtggggtctctttgctttcaaagagatgttagagatgttagtaaatggtgttagaagaaacaatgtaaattgcctgtttagtaagatgatccagtttctaaggaactgttttactggcgccttccatgctaacaaattctgagaaatatttgcggatttctcagtgaagccaaagcactctcttttgactcctatttactctcagagaaaaaaactattcatccatttgaaaagcagggaccaattcaacaagcagaatattccctctactaagcctttggccagagagttgttgctctagttctttccatcactcctgcatcaggaggctctgcctgcaggaaatagtattggacaacagaaagttcttttacttgtaggcataaaataggtataaattctcctccttaagagaaaaacaggttgctgcaccagaaagaacatttgcctcttctactaaatagcagactgttttccaatgataaactcattttaaatagaaaaaaaaaagtcattctctacaaacaagaacattttcattttaggttgtttattgtaaatatttcagaaaaaaaatatgcaaaaaaaaaaatgcccaaaaatcaatgcttccagtaacatatttattagctatctttttccccatgtaaaacttcaggtaaattctttgcagattttttacttggaagaatgataaaaaaaaaaaaaaaagttctgaaatgagttgaattcatccacgtgtgtttttgattcacatgaagagtaggacctccctttatattccctcttcaaattctcccctctgaaaaatgggtggtacactcaaaggaggaccgcacacattgtagtagctggggtgttgggggtggaaggagcaggttttgggggtgggaggagcggtgagtgatcctttcagcaaagtcagtcctgggcaggagacggcttcaggaatactgtcagctttactggattccaccatcgctttccaggactgtttaggccctgggcccttgaagggtttgcgtgcctcttgtctccattcatacctcaagaactttgttcatgttaattttttttcactctatcatatggaattgagtaaaaaaagaaaaaaaaaaggaagccaacacttacttaactgctttaatgtataggactctgttctgggttgtcttctaaagtacatttcattcaatgtgtgagaatacagataggaaagagttttgctagtttctattttgactgtggatccattgattctttctggttctcagcccttaattgctcagctgcagataccacttgactgactcttaaaagtctttggtttattgcccaattctgggaatatgaaactgtaagctgtttaggagaaaaactgagaccaaaagaaaggagaagtgagccaaactgccattatgattgccacttcttattgaagataaatcaaaatatccatttgactagaaatcaattgaattatgcactttaaatgggccaattcaatgatacctgaactatatctcaaaaaaggtgttaaacacaggcacacacgtacacacacatattggaattccaggaaaagtcactttgatcaacaagattatcagtcgtcaaagtgcttggaagggttctattggaataaagctaaaaaaaatcaaagtaaaatttctccttaaaaacaaaagcaattaagcagcactgtattataaaatatgctaagctgcaagtcaaaattcttaagagacttaagcctgtaagcagtaaggatccactcaatgtaaagttactccagagggaaaggcgtgagcccaaccaaagtattaatgctgtgtaggaggttaatttgactcttcttaacatttttacacaccacattatgttttattcaccatttattattaactgaatttattaaagtataatcaaaataatatttttgagagtttaaaaatattctcataatatttaagttacatatactatgcagggaccgtcaagtaattccactctgacatctgtatttattaccgccacctgtccatgatggtattagaaagatcctcatttgttttaatggatgcctctaaaatccagttttaagagggggttggccactttcaagtttgtgttagttaggattcagtttaacccagctcaacaggtatttactaagtaccagcatatccatagggcgctgtatgatactggaggacacacagaaaagagatgtgtaagaggcactcctgccctcaaggagtttactattgagtggtagtgtaatatgaataaacaaatgaatctgctcaaaagcagggcattctaagcaggccctcaccagcctctcatcattcgcatctagtgtatcttcctccatcactttctaaacgctatggattctgagtgaaaaccaatcgctggtttatgcatgtatgcatatatgaattcattcttgacattgccatgcagagtatttagtataactagacttattatccatttatcctgcctagaagcatactgtaatatatataactctaaaagcaagatcattccttatgttggttcttttgagctggcagggggaaagcagagtatgttggcaggggtctatcaaggtagatgagcccagtttgtacatctttgaaatattaccaatgccaatgtgttagtactgcttggatctcttctatgacaacagttcttattttttccttttttttgtttgtttgttttttgagacaggatcttgctctgtcacctagacaggagcgcagtggtgtgatctcggctcacttcaatttccgcctcctgggttcaagcaattcttgtgcctcagcctacagagtagttgggattacacgcatgcgccatcatgcctggctaatttttgcatttttagtagagatggggttttgccacgttggccaggctggtctggaactcctggcctcaagtgatctacccgccttggcctcccaaattgctgggattacaggtgtgagccaccacacctggcccatcagttcttaatttgatgaatggatagaggtttggcttcaaataaatggagttagtctgtcaatatctgtttcttttaaaatttgcaattgtttttatttttagtgtcatgctatgtgcccttaatgatctagaattaagttctaatctctaccctggtataattgatttctccctgtggactcctaatataagaagaaagatgaagtttttcttttaatgcttttagagatcttcattaagtcatttaatattcttatttgctatttcccagtgtgatggttaagactgagtgtcaacttgattggattgaaggatacaaagtattaatcctgggtgtgtctgtgagggtgttaccaaaggagattaatatttgagtcagtgggctgtggaaggcacacccacccttaatctggtgggcacgatctagtcagctgccagcgaatataaggcaggcagaaaaacgtgaaaggggaaactggcctagcctcccagcctacatctttctaccatgctgaatgcttcctgcccttgagcattggactccaaaatattcagttttgggactcggactggctctccttgctgctcagcttgcagacagtctattgtgggaccttccgatcatgtaagttaatgcttaataaactcccctttatatatatcctattagttctgtccctctagagaaccctgactaatacacccagtgtgttcttttatcaaatagaaaggagatatagaaaacacactgtccatgtctttgattgttacaccacagcagagaatcaatttcttaacctgcttacttatttaccattctccaatggtgccaattctgagatccatcatgactttatcattagaaaaggggatgtaacaaacatgatgagatgttacgggaatactgcttactgcatgatatggtttggctgtgtcctcacccaaatctcatcctgaattttagcctcccatacttcccacgtgttgtgggagggacccggtgggaggtaattgactcatgggggtggtttctcccatactcttctcctggtagtgaataagtctcacaagatctgatggttttataaggggttttccctttcacttggctctcattctctcttgcctgctgccatgcaagacatccctttgctcctccttcatcttctgccatgattgtgaggcctccccagccatgtggaactgtaagtcctttaaacctcttttctttataaattgcccagtctcatgtatgtctttatcagcagcatgaaaacggactaatacactgcatgctcagtatactcaggcactctggaaactaggtttctacttcggacccctgagagaaaagggttatgtatctccaagtgtggtcatgacaagcatcactactgtgtgcagttgggcctgtggattgagccaccttagcctctgaggaaagcagaacctgagagcatgcttaattcagagtgtgtgtgacttggagtgtggaaaggaagatttccagatttgcggtattggcgactgaaaggatggcaatgctattgatggagattaggaagacaggagagaggtagatgttgagcaactccgaggagtttttcctcatacatgttgggtttaaagtgcagggaagatatcctgaaggttattccaggcagaaatttggagctcagaagagacatcagagctgaagataaaaaatttgagagccatctccgtagctaaaagtgtggaacgaatgtgatgactagacaagataatagagaaaaggtatccaaaggcctgaggtcaagacctcttaaaaggctgacttttcagggttttaggtagaaaaagagtcccagcctggggttaaggcagttttttgagaagtagaggtacaaagttccttgttccaaaggaagggtgtctttcaaaagaaaaaggatgggccgggtgcagtggcttacgcatgtaatctcagcactttgggaggccgaggctggcagatcacctgaggttgggagttcgagaccaacctgaccaacatggagaaaccctgtctctactaaaaataaaaattaaaaattagccgggcttggtggtgcatgcctgtaatcccagctactcaggaggctgaggcaggagaatcacttgaacctgggaggcagaggttgcagtgagccaagttctcaccattgcactccagcctgggcaacaagagtgaaactctgtctcaaaaaaaaaaaaaagagagaaaaagaaaaaggatggtgaattgggccaaataccagagagatggacaggagtaagaagaaaaagactaaagatttgttcatcaggagctcattgtttctaaagggagcatttttagtaaagaggaaggaggaataaaatgcagtataatatgttaaatctaagtataatttataaaagagaaaatgggggtggaatattacaaaaaaggaactataaaccagctctttgaggtatttgtcagtgaggaaaacagtggggctctagttcttgatagcaacagcatcaaagggaactttttaaactaaaatattatgaatttttcaaactgaaataatgagagaataatacaaagaaatctcatctgcccataatccaaattcagtagttattaaaattttgccccattgcttaatccattaatctcatttcattttgtcctttttctttgtttatttttggctaaaacattttaaagacaaatcctaaatatcatacatcagtaggcatttacaaaaaaatttcttgataatcacaatgccattctcataccaaataaacttataataattctgtgatatcatctaaaaaacagttcttttctacatcaaaaatgtctgtttatagttgatttattcaatttagagtccaaacaagttccatatattagacattaggtcattaatgtctgtctatttgaatttataacaatttccacttaatttattttctccagcttctgtggagactaggtaagttgttttgtagctgtctggatatatctgattgcttccttgcagtgtcatttaacttgttcctctatcctcagtgtttctgcacatggaagttagcctcatagcaggggttggcatactttagcctgcttaccaaatacgactcccttacatttttgtaaataaaatgttactggaacactgtcacatatgttggggtcatagaaagagtttccgtgtccccaagttataattgcattcacttatattttcttctagtaatttttacccttacatgttaggggtcaatttggaatttctcttcatatacagtatgcaataaagaatggcttttatatttttccaaatgtatattcagttgtctcaacaccatttattaaaaagtcagtatttctcaagtgatttgagatctacccttaattacacagtaaatttctctgtagtcttgtctttttctaatatgttaattctgtaccaagatttgttcatctgttaatggatcaatatcacatagctttgtttatagaaggtgcacattgttttaattctggccatgctatcccaccaaatattgccctttgatttttcagagttttactgtggattcttccttgtttctttttctatgtgacctttagaattaacttgtttaattctataagtaaatttattgataattttattgaaattatgtaaaatataaattggcttaggaagtactgatatctccatgacattaagtcttcatatctaagaccaaggggtgccttttcatttgttaaatattctttgtgtctttcagaagtgttttcaagtatccttatgtgtattttgcacaaatttttatatttgtgcctattggatgccacgtgcagggttaaaaaatattatacctattttatatagatatatatgtatataaaaatatatattatatatagttataatacataaatatattttaatatattatataatatatacatgtattgtatttatatatgtatttatatgtgtatctataaatatacatatgtatgtatctataaatatacatatatgtgtacatatacacatatatacacattatatgtatgtttatacacatataaatacatatataaatatactagaaagtccatggggtttcctagtatacaatcatattgtctgcaaatagagattgttataccttttcctttctattccaaagtgtctaaaagattttttcttagctagtggcattggatgacacctataatgtcttctaaaaatagtagcagtcataggcaccatttccttattcttgaatattcattcatgttacaaagtttataggaatttctgaattattaagtacttttaataggaatgaaggttattgtcattattgcatcaaaattcataagaaagtttggtgtcaaatttgtgctttgtgtgtagtcttcatcacgcttttttttttttttttaaccagtgttaaacatccattacaaaaatagtttggaagtttttctttttttttttcctatgtcctggaaggtgtaagtagaattggaattatttgatctttaagatgtaaaatattggtagaattctacctttgaaagcacctggacatggtgacttcttaataggtgagctctctggaaactttatttcttctgtagtaattggtctccttagactttctattccttctggagtcagatattgtatatcaaatcttctgttaaagttatccatttcttcttggtattccaatttttcacattgagttggacaaatgatttctgatgatgtttaaaaaatttctctggtttgatggttactttgaccgtataatttattttgtgtatttatgcttcttttagaaacttaggtcagctagtggttcctctattttgttgatttttttcctcaaagaactagcttaagtttattttattagttctactagttttctgctttctaactcattaagttatgcctttagctttattaaatccttcattctgctatcagttaatgttttagttcattttctagctttttgagttgatacttaatttattttcatttcatgatataggtaactgatgctcagaattttctcaaagcatcagttttcgctgggccccatagattctaatatatcttactttcattattattatttcctaaaaattcagcaatttcatgttttattttgtctctcattcaagagttgtttaatagacagtttaatttccaggttggatgggcattttgttttgttttttgttttactgtttataaatacttttggccttgggatcagagaatactgcttgtattacaattattactgaatatttttgtggtctaatataatttcagctttcaaaaatgttccatgtatacttgaaatgaaggtttatttacccttataaagatagaaggtaaaatattagctatgcccatgtctatttatcttcttataatttttgcttaatgaaaactaatgatccattatctgctgcataagtatttatgtttgctttctctttattatgccttatggcttttgtgttaaaaagctattgatttatttaatattttggaaggattgaattttaccctatctaatatcaagatcatgactcttgattcctttttgttttcacttgtttaatatatctttcttctatctttattttaaacacaagaagagctttgctttcagtttctattttacatagtctacaatcggattttacttattagtcaatctgagaatacttttaatagatgagttacacaaatacatatttattgataccaacacagtttaattcaagatctgttctattattttatatttattatatattaaattgtcttctttatttactatttattgatttattgaatttattttgctttcaggtattttgtatttttgttctggttgttagatttatgcatacttagtcaactcttcctttataaagcattcattttctactatgagcagtagtaaaattagctagtaacttaattcctgagcactctccagtcctagtcactagtaataacgcagtgttaaactttgtaatattaattatacttaagcctctattacttggtttgtcaaaatcaaatgatatcaatttacttccagcacatacctatgaggtaattgtaagctaataataacttccatggctattcttttctctttttttttttttgaggtggagtttcgctcttgttgcccaggttggagtgcaatggcacgatctcggctcactgcaacttctgcctcccgggttccagcgattctcctgcctcagcctcccaagtacctgggattacaggcatcagccaccccacctggctaattttgtatttttagtagagacagggtttccccatgttggccaggctggtcacgaattcctgacctcaagtgatccaccccatcagtctcccacagtgctgggattacaggtgtaagccaccgcacccagcctctcatcttatttttattacttatatttttctacattattagaagttataatacttatatgcacatttctttagccccaaccccattttttgatattagctctattattaagatatttaatgtccaccttcagattatatgctgatgattctctaatcgtttttggttgtctggagcttttttactgtttattttcaggaagagctcatgataactcttttttctttggtttaaaaatgtttataattatttttctctggactttatgctgagaaatttggctgcatacaaaatctctggcttacattttttctcaagtatttttaagtgattgcttcattattttgtagtataaaatgttgctcacaagaagctggatgccagtctgattttctttccctcattagtgatttggactttttgtctgaatgttgaatttttaaatataaacataaattgcaatacttttattaagatatgtctctgcattgaacattttaggtccgttttcctaggtacatagaatgtcttttgaatatgtagactcaaatcagacttcttgattctcttgatttatagttttaaatatttgtttgggggcgggtgcggtggctcatgcctgtaatcccagcactttgggaggccgaggcgggtagatcataaggttagcagattgagaccatcctggataacatggtgaaaccccgtctctactaaaaatacaaaaaatatagccaggtgtggtggcaggcacctgtagtcccagctactcgggaggctgaggcaggagaatggcctgaatccgggaggcagagcttgcagtgagccaagatcacgccattgcactccagccttggcgacagagtgagactttgtctcaaaaaaaaataaatagggggcggttccaagatggcagaataggaacagctccagtctgcagctcccagcatgagtgatgcagaagacaaatgatttttgcatttccaaatgaggtaccaggttcatctcactggggactattggacagtgggtgcaggacagtgggtgcagcgcaccaagcatgagccgaagcagagcgaggcatcgcctcacctgggaagtgcaaggggtcagggaattccctttcctagccaaggaaaggggtgacagacggcacctggaaaatcgggtcactcccaccctaatactgggcttttccgatggtcttagcaaacggcacaccaggagattatatcctgtgcctggctcagagggtcctacacccacagagcctcgctcattgctagcacagcagtctgagatcaaactgcaaggtgcaagcgaggctaggggacgggcgcctgccattgctgaggcttgagtaggtaaacaaagtggctgggaagctggaactgggtggagcccaccgcagctcaaggaggcctgcctgtctctgtagactccacctctgggggcagggcatagccaaacagaaggcagcagaaacctctgcagactgaaatgtccctgtctgacagctttgaagacagtagtggttctcccagcatgcagcttgagatctgagaacgggcagactgcctcctcaagtgggtccctgacccccgagtagcctaactgggcacaccccagtaggggcagactgacacctcacatggccgggtactcctctgagacaaaacctccagaggaacgatcaggcagcaacatttgctgttcaccaatatccgctgttctgcagcctccgctgctgatacccaggcaaacagggtctggagtggacctccagcaaactctgacagacctgcagctgagggtcctgactgttagaaggaaaactaacaaacagaaaggacatccacaccaaaatcccatctgtacgtcaccatcatcaaagaccaaaggtagataaatccacaaagatggggaaaaaacagagtagaaaaacagaaagttctaaaaatcagagcacctctcctcctccaaaggaacgcagctcctcaccagcaacggaacaaagctggatggagaatgactttgactaattgagaaaagaaggcttcagacgatgaaaattttctgaactaaaggaggaagttcgaacccacggcaaagaagttaaaaaccttgaaaaaagattagacgaatggctacctagaataaccaatgcacagaagtccttaaaggacctgatggagctgacaaccaaggcacaagaactatgtgacgaatgcacaagcttcagtagctgattcaatcaactggaagaaaggttatcagtgatggaagatcaaatgaatgaaatgaagtgagaagagaagtttagagaaagaagaataaaaagaaatgaacaaagcctccaagaaatatgggactatgtgaaaagaccaaatctacgtctgattggtgtacctgaaagtgatggggagaatggaaccaagttggaaaacactctgcaggatattatccaggagaacttccccaatctagcaaggcaggccaacattcacattcaggaaatacagagaacaccacaaagatactccttgagaagagcaactccaagacacataattgacagattcaccaaagttgaaatgaaggaaaaaatgttaagggcagccagagagaaagattgggttacccacaaagggaagcccatcagactaacagcggatctcttggcagaaactctacaagccagaagagagtgggggccgatattacacattcttaaagaaaagaattttcaacccagaatctcatatccagccaaactaagcttcataagtgaaggagaaataaaatactttacagacaagcaaatgctgagagattttgtcaccaccaggcctgccctacaagagctcctgaaggaagcactaaacacggaaaggaacaactagtaccagccactgcaaaaacatgcaaattggaaagaccatcgaggctaggaagaaactgcaactaacaagcaaaataagcagctaacatcataatgacaggatcaaattcacacataacaatattaaccttaaatgtaaatgggctaaatgcttcaattaaaagacacagactggcaaattggataaagagtcaagacccatcagcgtgctgtattcaggaaacccatctcacgtgcagagacacacataggctcaaaataaagggatggaggaaaatctaccaagcaaatggaaaacaaaaaaaggcaggggttgcaatcctagtctctgttaaaacagactttaaaccaacaaagatcaaaagagacaaagaaggccattacataatagtaaagggatcaattcaacaagaagagctaactatcctaaatatatatgcacccaatacaggagcacccagattcataaagcaagtccttagtgacctacaaagagacttagactcccacacaataataatgggagactttaacatcccactgtcaacattagacagatcaacgagacagaaaattaacaaggatatccaggaactgaactcagctctgcaccaagcagacctaatagacatctacagaactctccaccccaaatcaacagattatacattcttctcagcaccacgccggacttaatccaaaattgaccacatagttggaagtaaaacactcctcagcaaatgtaaaagaacagaaattataacaaactatctctcagaccacagtgcaatcaaactagaactcaggattaagaaactcactcaaaaccgctcaactacatggaaactgaacaacctgctcctgaatgactactgggtacataacgaaatgaaggcagaaataaagatgttctttgaaaccaatgagaacaaagacacaacataccagaatctctgggacacattcaaagcagtgtgtagagggaactttatagcactaaatgcccgcaagagaaagcaggaaagatctaaaattgacaccctaacatcacaattaaaagaactagagaagcaagagcaaacacgttcaaaagctagcagaagacaagaaataactaagatcagagcagaactgaaggagatagagacacaaaaaacccttcaaaaaatcaatgaatctaggagctggttttttgaaaagatcaataaaactgatagactgctagcatgactaataaagaagaaaagagagaagaatcaaatagatgcaataaaaaatgataaaggggatatcaccactgatcccacagaaatacaaactaccatcagagaatactataaacacctctacacaaataaactagaacatctagaagaaatggataaattcctcgacacatacaccctcccaagactaaaccaggaagaagctgaatctctgaatagaccaataataggctctgaagttgaggcaataattaatagcttaccaatcaaaaaaaagtccaggaccagacggattcacagctgaattctaccagaggtacaaggaggagctggtaccattccttcggaaactattccaatcaatagaaaaagagggaatcctccctaactcattttatgaggccagcatcatcctgataccaaagcctggcagagacacaacaaaaaaagagaattttagaccaatatccctgatgaacatcgatgcaaaaatcctgagtaaaatactggcaaaccgaatccagcagcacatcaaaaagcttatccaccatggtcaagtgggcttcatccctgggatgcaaggctggttcaacatatgcaagtcaataaacataatccagcatataaacagaaccaatgacaaaaaccacatgattatctcaatagatgcagaaaaggcctttgacaaaattcaacaacccttcatgctaaaaactctcaataaattaggtattgatgggacctatctcaaaataataagagctatctatgacaaacccacagccaatatcatactgaatggacaaaacctgggagcattccctttgaaaactggcacaagacagggatgccctctctcaccactcctattcaacatagtgttggaagttctggttagggcaatcaggcaggagaaggaaataaagggtattcaattaggaaaagaggaagtcaaattgtccctgtttgcagatgacatgattgcatatctagaaaactccatcgtctcagcgcaaaatctccttaagctgataagcagcttcagcaaagtctcaggatacaaaatcaatgtgcaaaaatcacaggcattcttatacaccaataacagacaaacagagagccaaatcatgagtgaacttccattcacaattgcttcaaaggaataaaatacctaagaatccaacttacaagggatgtgaaggacctcttcaaggagaactacaaaccactgctcaacaaaataaaagaagatacaaacaaatgcaagaacattccatgcttatgggtaggaagaatcaatatcatgaaaatggccatactgcccaaggtaatttatagattcaatgctatccccatcaagctaccaatgactttcttcacagaattggaaaaaactactttaaagttcatatggaaccaaaaaagagcccgcattgccaagtcaatcataagccaaaagaacaaagctggaggcatcatgctacctgacttcaaactatactacaaggccacagtaacgaaaacagcatggtactggtaccaaaacagagatatagaccaatggaacagaacagagccctcagaaataatgccatgtatctacaactgtctgatctttgacaaacctgagaaaaacaagaaatggggaaaggattccctatttaataaatggtgctgggaaaactggctagccatatgtagaaagctgaaactggatcccttccttacaccttatacaaaaattaattcaagatggattaaagacttaaatgttagatcaaaaaccataaaaaccctagaagaaaacctaggcaataccattcaggacataggcatgggcaaggacttcatgtctaaaacaccaaaagcaatggcaacaaaagccaaaattgacaaatgggatctaattaaactaaagagcttctgcacagcaaaagagaaactaccatcagagtgaacaggcaacctacagaatgggagaaaatctttgcaatctactcatttgacaaagggctaatatccagaatctacaaagaactcaaacaaatttacaagaaaaaaacaaacaacaccatcaacaaatgggcgaaggatatgaacagacacttctcaaaagaagacatttatgcagccaacagacacatgaaaaaatgctcatcatcactggccatcagagaaatgcaaatcaaaaccacaatgagataccatctcacaccagttagaatggtgattattaagaagtcagggaaaaacaggtgctggagaggatgtggagaaataggaagacttttacactgttggtgggactgtaaactagttcaaccattgtggaattcagtgtggcgattcctcagggatctagaactagaaataccatttgacccagccatcccattactgggtatatacccaaaggattataaatcatgctgctataaagacacatgcacacgtatgtttattgcagcactattcacaatagcaaagacttggaaccaacccaaatgtccaacaatgatctggattaagaaaatgtggcacatatataccatggaatactatgcagccataaaaaaggatgagttcatgtcctttgtaaggacatggatgaaactggaaaccatcattgtgagcgaactatcacaaggacaaaaaaccaaacaccacatgttctcactcataggtgggagttgaacaatgagaacacgtggacacaggaaggggaacatcacacaccggggccttttgtgtggttggggaggggggagggatgcattaggagatacacctaatgtaaatgacgagttaatgggtgcagcacaccaacatggcacatgtatgcatatgtaacaaacctgcccattgtgcacatgtaccctaaaacttaaagtacaataaaaataaataaataaataaataaataaatactagtttggctgcattgctttgattttcttctttaggggcctgttcatacatacaaaggataccatttacctgtcttctatatgactttctcttaaatcctttcattcttttgttttaatttttatcttcacatcctttatttctgtcactgtgctgtccatagagtttgtctgctgtgtgtccttataattaagtctatgttctgaatgtttttccttttttagaaattcagttctaaagtgtttaatttctcgtatttttttttctgttgcctcaccatatcatttctgagttttctgtttctgaaatgtgcaactaactctttccaagcattgaccagattcttcagtctttttaattcattctgaaataactgggctacagtttcatctgctttgtggacagaagtgccacaaagagccgaattgtcagtgcagacccacatgaatcatagatcttaacgaggtttttactaacgactagcaaaggatacaagctaaaaatgggtacaagcaaacacagcatcattcatcactgtaaagactctgaactatcacatggaacttcaaaaggattcttcttctttgctgcaatgtgttttgatttttggagtgatagatgtttgctaactacgcacgtgacaaaaatttgcttagaggaagccatgttagttttgatgctactcaactttgtattttgttagtcatgagatagaaagcctgtgagattacatgcccttcttttagcagctgcatcccataaaattaaaaattccagttttttttaaaccatgaacaatttagtcagacttaatatatcctatttaaaacattcttagataagaacttcctttgttattttgctaatatacaatcccaccaaatgtctacaagaagggctagttaatcaatataccaaagatcaaatctatttatatgaaccacaaatacaaaacataattatacatatataattttatataatttaatttataattatatacattttatagcagcaatcaaaactatcatgtattttgggatgaatttaataaagaaatagcaggagctctgtagaaacaagtatgaacttctattggaagacagtaatatcataaaacattaaaaagaaagctataccatgtttatggttaggaagattcactattgaaaatacatcaattcttgacaaatcggtctataaattcagtgcgtgtccaatcaatatatttagctcatttaaaatgtatatggaaaaggctaagtaccaaagataatcaaatgctcttggaagatgaaaattaaagttgagagatggggtaaaaaactataattcagttctaaagtcataaagctataataattaatacagtatggcattggcatagaaataagcacattgactaaataaatagaaaatacagttcagaaaccaaactctacatttatgaaaaactgacctatatcagaacgagcattgagactacaggagaacagaggaactagtaagttaatggtcctgacacaattgatatctacatagaaaaaaaaacttaatccttctttactctatgtaaaaacaataattccagatgaatgtaggacttaatggagagccaaaactaaaacttttataggaaaacatagaaaaatacctatctcagggtcgggaattaggtcttcaacatggcaccaaaagtactaaccataaaagaaacattaataaagtcaacctttttaaaatcaacaactttaattaaaaaaccagggccaggcacggtggctcacgcctgtaatcccaggaggccaaggcaggcagatcacgaggtcaggagatcgagaccagcctggccaacatggcaaaacctcgtctctactaaaaatataaaaattagctgggcgtggtggcgggtgcctgtagtcccagctacttgggaggctgaggcaggagaatggtgtgaacctgggaggcggagcttgcagtgagctgaaatcgtgccactgcactccagcctgggcaacagtgcgagactccatctcaaaaaacaaaaacaaaaacaaaatcaaaacaaaaaacaaaacacaggttgctggaaaaatggttacttatttttattatgctttataaaatatacagttatattatcaaacttcttttgtaagaatcaaatattatgctgagaatatggcaagaaaaaagaaaacaacctccaccactcataatggcattccctttacccaccatcaactaagtcaggaacctgcgtcactttcaagttttctgaaatgtggactgaaaatacttgtggagggccctcacagatgcatttagaaatggtatgttctggaaaatggaactgaaatgttttagcgagagtcatgggcccaaaccttgaaatagaggttggtggagttttctatagtatgtaattacaacacaataagactcaaaagcatttcaataaagtgtggatggaagacaagatactgcttcaagggcttcattagctttctgcagactggagctctccaaggcatggggaggaaccctgtttacctttggttcagaatcagttttaaccatcattgagggctaggtcagagtgcagcattaagtccttgggaatacactgggtggggaaagaaaagagagcaatgtttttctaaaagcccagaaatgggcttactgtgtttaccagttgtttccaggataatgtattgcagtgccaattgctgatgagatggtaggattatacttcagtccctgctttacatttatttcttaaagaagcttctggtaaattagagcaatagcatcggcttagtttagtgttgttctgttggactaaggatatcagttctatccgtatggtcgggcctaaagcctgggaaatatttaatgaagggagagagggggagagagtgagcatgcaaaagagagagagaaaaacaaataacaaaacaaaaaccaagacatttccctttatagtaagaatgatgaggaaaacatgtttagccatacaagatatcaagataatctcttatttctttcttgaaaatgcaagtacaaatgcctgcaaagataaaatatcctctggatggagtggaaaggttcaccaggcctctgaaatcacgtgaatgatgttgcgctttgctgttaatgaagctcggtgcatttttcatttcagtttctactaagcatttatgagctattacccttcccttccctaaactgcgttgttttttaaaaagccttagaggcattccttctagaaaataaaggtaagtgttaagtggtgataatttggtaataggtgtcatgcttgtggttcataatgtgttaccctacacattttctcaattatccctagaacagctttttgaggatatgaagttagaccttacaaagcacatctttctgctggaaaaatgaggtttatagtggttaagtttagttgcttatgatcacaaggctagagagtggccggaatcaagactcttcaccctgatttcagtgctttttcactccaccattaatatttattgttgataaataatatcaacactttcctaggtgtattagggtcatctagagggacaagactaataggatagatgtatatatgaaaaggagtttattaaggagtattgactcacaccatcacaaggtgacgtcccacaataggccatctgcaagctgaggggcaaggaagccagtctgagtcccaaaacctcaaaaatagggaagctgacagtgcagccttcagtctgtggccaaaggcccaagagcccctggcaaaccactagtgtaggtccaagagtccaaaagctaaaggattggagtccaatgtttgagggcaggaagcatccgtcatgggagaaagatggaagccagaagacagccagtctagtccttccacgttcctctgcctgcttttatcctagccacgctggcatgatgatgacatggtgcccgcccagattgaggatgggtctccatcttccagttcactgacacaaatgttaatcttctttggtaataccctcacagacacacccaaggacagcactttgcatccttcaatacaatcaagttggcactcaataataaccatcacaagtccacaccttgtcaacttgatcccacatacatctccttaaatcatacatgatctccaaatacagacaataatgtcataattacactgaacataatacaactatcgttcatacaaccagaaatgcaccaattcccaaaccaaatgttattacataaagttaacaacacttaaatgctgatatgaagtcaataaatactttttttttttaaaagatggagtcttgctttgttgcccaggctggaatgcagtggtgcgatattggctcactgcaacctccgcctcctgggttcaagcaattctctgcctcagcctcccgagtagctgggattacaggcacccaccgccacacctggctaatttttgtatttttagtaaagacggggttttgccatcctggccaggctggtcttgaactcctgacctcgtgatccacccacctcggcctcccaaagtggtggatcccaaagtgatccacccaccttgacctcccaggttgtaagccactaaatcttatgtcacatgatacaggaaaaagaaaggaagtaaaatgaagatattgtcttagtacaagtgtatacaggcagaaagatgttcttaacaaaataaggaggaaatactcatgacaattacagtaacctggttgctgcaactcatcacatggtcgtagctgttattgatgactaccttcttctacaacccattctgttttccctttgcctctagcaagtacctcagcaggtcatggttctttacctggtggagtgtcccaaaccttcattcctgaagggtctgggccatttgtagtcctgcctggatcgagttgttgtcattttttattgaccttaatcacagggcttggtaatactaagagacaccctaaggaatttcctgtattccacacatgttcttccttaccttcattatggagtagtagactgagttcatcttgataggctaagtcagtcaccccagccaacactaactcctttcttagcctgttgacttagaggtaggaggagcccgagattgcaatcttaatttccagtttaatgaaatcattattgtgtctcctggtggcaacattcatcgccctggaactgagacttctaggccagcagaacgtaatgctgtggaacaagaagcacaaattttactagcgggtctctaggggtgatggtgagtggtgccacttccatttccaccccttgattcctggacccatgaatcctggctatgggagaaacagtaccaaatactggacactgatccggagcatacacagccttctggaaaactttgccccagccctgcaaagtattgtcgcctagttggcattgtaattgtgacttcaaaaggccattccaccgttctatcaatccggctgcttcaggatgatggggaacagggtaagaccagtgaattccatgagcatgagcccactgctgcacttctttagccataaagtgagtgccttgttcagaggcaatgctgtgtgtaatgccatgacagtgggtaaagcattccataagtccatggatggtagtcttggcagaagcattgcctgcaggataggcaaacccatatctagagtaagtgcccattccagtgaagacaaaccactgttctttctgtgatggaagaggttcagtataatcaacctgccaccaagtagctggctgatcaccccgaggaatggtgccatatcgcggcctcagtgtttgtctctgctgctggcaaattgggcactcagttgtggctgtagccaggtcagccttggtacgtggaagtccatgttgctgagcccgtgcgtaacctccatccctgccaccacagccactttgttcatgggcctattgggcgatgacaatggtggctggggaaagaggctgagtggtatccacagaacgagtcatccaatccacttgattattaaaatcctcctctgctgaggtcatcttttggtgagcactcacatgagatacaaatatcttcacagtttttgaccactcagagaggtccatccatccacatacctcttccccaaatttcattgtcatcagttttccaatcatgcttctttcaagtccctgaccatccagccaaaccattggctacctcccatgaatcagtatataatcacacatcttgccatttctccttctaagcaaagtgcacagccaggggcactgctcaaagttctgtgcattgggaagatttcccttcactgctgcccttcagggatgtcctagaaaggggctgtagtgctgcagctgtccacttttgggtggtgactgcatcatgcagagccatctgtaaaccaggcccttgtcttctcttcctctgtcagcttatcatagggaactccccatgaggccatcagtgcaggctgggggagagaaggcagggtggcaggagtggggaccatgggcatttgagctacttcctcatgtaaagtacttgttccctcaggacttgcttgagcctgatcatgtatataccacttccatttgatgatggaatgctgctgtgcacaacccactttatggctagatgggtcaaaaagcatctagttcatgataggcaatttaggtagcttggtgatttgatgacctgtagtcaaacatacagtttctaccaaagcacagtaacaggccaagagttgtctctcacaaggagagtagttatctgcaggagatgcagggccttgctccaaattcctagaggcctcccctgtgattgacctataggggcctgccaagggctccaaacaacatccctatctgccattgacacttcaagcaccattggatctgccaggtcatatggcccaagtggcagagcagcttgccaagcattctggacctgttgcagagccttctcttctggatcccactcaaaactggaagcctttcaggtcacttgataaatgggctggagtaacacacccaaatgaggaatgtgttgcctccaaaatccaaataggcctgctaggcattgtgcctctttcttagttgtaggaggggccaaaggcagcaacttatccttcatcttagaaggaatatcttgacagaccccacaccactggacccctagaagttttactgaggtagaagttccctaaattttagtcaattttatttcccaacctctggcacacaagtgtctcaccaataaatccagtatgtttgctacttcttgctcattggatccaatcagcataatgtcatgaatataatggaccagtgtgatatcttgtggaagagaaaaactatcaagatctctccaacaagattatgacacaaagctggagaatcgatatacccctgaggtaggacagtaaaggtatattgctggccttgccagctgaaggcaaattgcttctgctgggccttatggacaggaatggagaaaaagccatttgccaaatcaatggttgcataccaggtaccaggagatgtgttaatttgctcaagtaatgaacccatatctggtccagcagctgcaattggagtcaccgcttagttaagcttacaataatccactgtcattttacaagatccatctgtcttctgcacaggccaaacaggaaagttgaacggggatgtggtgggaatcgccacccctgcatctttgaagtccttgatggtggcactaacttcctccagggatgcaatgttgtgtttgatttactatttttctaggtagaggcagctgtaatggcttccatttggcctttcccaccataatagccctcaccctaccagtcagggagccaatgtgggggttctgccagctgctaaatatgtctatgccaattatgcattctggcactagggaaatgaccacaggatgagtctgggacccaccagccccactgtaagttggacctgagctaaaattccattaattaactgatctccaaaagcccctactttaactggaggaccacgtgatgttttgggtcccctggaatcaacgtcagctcagagccagtgtccagttgtccctgaaatgtctgattatttccctttccccagtgcacagttaccctggtaaaaggccagaggtctcctgggggaaggatgggagaaagagtcactgcataaattgtcagcaatgtagtggggtccttcctcaaggggacccagcctccccttcattcaaggggttctgggtctataaactggttcaagtctgcaaattgattgaggggccgtgattctttgtataatttttaatttaaattagtcttttgtctacttgacctggaagttttctgcttatataaattaagcaagaatgcagtaggcctcttgtcaatttcactttttaggaacactgagattaagtagccaatgccagagctctacacaagtcagactattctgattgttgccttgcctccgctgacctttacggtaattgcacccaccttgccttgatgcttgagtgccaccacttggcccatgccacctcaggatccaattattaccattgtatttaaattttgtagttgaatgactgtggttcccactataatatctagcatgcagagaagagcaatcacagagctcttcaaggatgcaggtgctgccctcacaaatctatttcacaatgtgctggtcaaaggtatatcttctgtactctcccagctgggatgagtaggactaaagtgacgaattcactccaccatcccagtctccataagcctttggatcccttcctctacattaaaccaagggagatcaggcatttccagctcactcacagtgggccaccttttgatctatatttcagctaaccaagcaaataaactattagaaccttttttaactctccaagctgcaacattaaatgcagaattcctgcttagtgggcccaaatcaatcaattcagcctgatccaactttatgttccttctaccattagcccacacccttaatatccattcccatgcctgttctccagatttctgcttatataaattagaaaactcaagcagttcttttggagtgtagcgcacctccttgtgggtcacattctgaacctcatctctaaggggcctgccgggactttagtctagttctataactataagcaaacaggggtattgagggtgggtccttaggagaatcaacatcgtctttcctggcaactgcctcaagggaggtcatcgctgatgccttaggcagtgcagggttaaactcctcagacaaaggtgaaaagcctgatggcagcgcaggtggaggaggggatgttgcctcccctctgttgcctgtttcctctggtaaaaaagattcatcagaatttagaagctcagtgcccccagccttatcagggtcctcccgcatggccccattccaagttgcagggtaccattctttttcaatcaatgctgtcactttaacagtaaacacctggcaaggctgtgcacgtacctttcgttgcaggtcagccactcacatgataagagcttgtgtctgattttccgcaatttcagctctttctctacaggagataagactcgaacccagggcaatcttagaagatttgaggctcagaatgtggttctggagctgggagatagaatccctgagttcatcatttttttgttttcatcactttgattagtgaacttaggagcaaccaactagcttcattatattccttggttctccacatatggttaaaggtattaggtataaagtcactaaactccttgcctctcatgagcagtgaatcaggagtatcagatgcatttattttgcataactctctacacagttcacagcaaggactatcagtgttctccacactattagaagtagagtccctagcattttggcgtctaatcatatttagcagccaactccagaaaccccaaaaccatctaaagaaatccatccgtaaaattctgttcctctagaaccactcctggtaccaaaatctgtattagtcaggattcgctagagggacaggactcacaggtgagatgtatatataaaaggcagtttattaaggagaattgactcacacaatcacaaggtgaagtcccacaatagtctgcaagctgaggagcaaggaagccagtccaagtcccaaaacctcaaaactagggaagccgacagtgcggccttcagtctctggctgacagcctgagggcccctggcaaaccactggtgtaggtctaagagttcaaaagctgaagaacttgtagtccaatgttcgaggccaggaagcatccagcacaggagaaagatggaagccggaagacttagccagtctagtccttccatgttcctctgcctgtttttgtcctagccatgctggcagctgattagatggtgcccacccagattgaggatgggtctctatcttccagtccactgactcaaatgttaatctcctttgacaacaccctcacagactcactcaggaacaatactttgcatccttcaattcaatcacattgacactcagtattaaccatcaccctaggctttagggatatagggaaaaacatgactcattgctgttatctgagagtacataatctattggaagaaaggaaaacagttacatgtaaggcttatatcagtataaattagataactgccaagtgagagaggtagaggtagcaagtgctgtctgtgggtgcatgttaactcagtcttaatcttggaagaagtggcagtgtggaatggtacatggagaagcagaagggacaattactgtgagcagatgaatggctcactacaggggcatgggaagaagatgcaagagcagtggaatctttaattgacgaacctggtaccaagctgccaagtcttagtctcaggactggcaccgtgcacttggccattgaaaagactttggaacctgtggagtgaagagttgtaggaattttcaaagcctcagtgtaggaacaatgtgagaatcaaccagggcatggtactccacactgtgttcagatccactggtgttaggggagtcccagagaggcctcttagagggttgaggaggatgggatcaggcatagcagagactagggacacgaggctctgcggccagactgcaggtctgctaccaactacagtgtgattttgcctagtcacataatctctgtgggcctcagttttttgttttttgtttttttctattacaacagtacctttctcgtggagttgtagtgattagatatctagtaccaagactatgcttgttagcactgagtaagaactcaatgaaggttagctttcattggtactatattactattgttagtgttggtggtggagctgaatcgcctaacctatgaggttggtgctgaaatgaagaaaagaataacagtgttttaagaagtttggtcactagaagtggagcttagtaattaaagaaaaagagtgaacacttttaccttcttgtggaagttagtaagtctacaaaatgttaattctgcatttgaatgatcatttgggagactcttattgtcctatttgcactgaaaaagtcactgaatcattattttagaactggaataacacctgagatctaggccagcactttgcaagttgtgctctatgggacttttcatggaagtggctgaggagttgccttgaaggaaggcagagggagtgggtcttgggacacccttccagttataaaacaggacgttgattctgttttgtagttgcagcatcacatataatttcatttattaagaggatcccacttctaaaaataagttgaaaaccagtgatttaatccagcctctctgtttgcatatgaggaaatggaattccggaaaggttaggtgatttgtccaaggttgcagactagactatttattaatagagtagggtcaggaacaaaagcctgctccttgctggtccagcgcctgttactagttacataatgaatgctacctattgctgcacagtgccaaatcattgcacttttcagattttactctaatcaaaagaaaaaaattaaagtgcacttccaaatcagtacttatatgcaagagcttcaagaaacaaactagtatttaacttggtggttacatattgactgtattttcattgagtgaggttagaagagattgagaagcgtgaaatgaagttacaaagtagaaactatatggtgaactcaaggcaaagattgtccatagtaaaagaagacaaaataagaatgaaagagacaaaagaattgccaatgagttgtaatcttaaaagaaagatatatttaataaaataggattgattgttttgaatgtgggctgagaagtcctgccatcttccattgactctgctcacaggccttgtttgtgaactggcttccatggatagcattactctcctgacagctgcagctccaaattcagcatgaaaaggctggcaattctaagaggaagggagggtttaggccacttttaattctactttgtcattgcaagtttcttgcattgtttggcatttgttggtcttccttactggatttcaaaactagaacactttttcctagccttggaaccaaccacgaaaataaccacctcttaccctcatgaagaacactttaagtttttctcttttaaaaatgaagtctggaatatctccaaacatcttgtcactcacgccttcatttaaatgtcaccctctcaaccacaaggaaactaaaattgccccctacttgctactccctgttttattttctagtagaacatactacagttggagatcatctcatccatgtcttttattcttgtctcttatttgcctctgcttgactgtaagctcatggaaagtaagggaccttcaccaactagcaaagtgcttaggacctagtaagaacctggtcaattctaactgagtgaatgactgaattcctgtgagaagtcaacatgaaaattcctaggtcattagtttagttattggaactccaacactgtattggaaaagtgtcatggaaaggatagttagtggattaaggttttttataagaggaagagggaaagaaaggcattgtctttacttcaggcatttcaatggccatgatggagcctaaatattgtatgttgcattttgttttggttttgtgtgcacaagagctttgccagtggaatgaacgggttaagaaatgtgagttttcttttcgtcctgctatagagacttaaggaattgccctgtgtgagttccttgagggcagagaaaaatacaaatccatgaatacctgaaagccctgatttggcccctaagcaatctatgcatgtaacaacattgcatttgtaccctgtacatttataaacattttaagaaaagtataagagaaacacaaattttggttactcctctctgaaaactgctctgatgtcgcttggccgggggaattaagacctcttcctgtggcctgtggccactatgtgctcccggctctccaggctcatccagggccactgtgcctggggtcacagggttccaggatgtcaaccttctttcagcttctctgactcctcaagcatttttttttttttttttttttttttttggtttccaagacccaaacgcatgtctctcttcttggcacactcttcccaacttttttcctggctaattgctagttatttagtttgagattaaatgtcactccctcggcctggcgcggtggctcacgcctgtaatcccagcactttcggacgccgaggcaggtggaccacgaggtcaagagatcgagaccatcctggccaacatggtgaaaccccgtctctattaaaaatacaaaaaaatttagccaggcgtggtggcatgtgcctgaagtcccagttactcgggaggctgaggcaggggaattgcttgaggaggatattgcagtgagctggatcgcacctctgcactccagcctggagacaaagtgtgacaccatctcaaaaaaaaaaaaaaaaaaaaaagtcactcactcacagagggcttctcagatactcccctgccccctcatttaagttgcaccgcttgttgtattctcttataagccccattctttttcttcttggcattgatcaaattaacagctttattttattttagcattgtgagtgtatatgtgtgtttaatgtctgcttcctgactaaactgtaccctgcaggaaggcaaaactgtgtctgtgttgctcattgttaaaccttcagcactgaactcagtgcctcgaacataggagattccaactcaatatttactgcgtgaaggaatgaatgaatctttatgtccctcgtgcctaacataaagtctgccatatacaatggactaaaaaatagtattcagctaaaactgagttacggagaagatgaagtattaattgtattttttacagagaaacaatggtcagtgtatcaaaaatagagaccctgctctcagatatataacatagaactctcttcattcatttgccttcattttagttaacagaatctgttcatctaagtagagcagagaaaaacttcataattgctgtgtctgtgttattccaaaacatttaaacaaaaggtgaataactgaggtattcttccctgtggtacatacttgatgtgggcattttaaaagattgatcaaatctcctttcagctggatatttgagtaggcacaaccattacagaattttcctctagggacttactttagctcttaaactatgtaaactgaacaagcaaactcaagtggatcattatctatagaagtatagaattccatctccctttggcaaaccattcaaacccagaagtgtgttttacacattctgacagaagcatctgtaataaccaactctaacctcctttcttaccactttagcacccaaaagtataagaaaagaggacatgttaaggcttgttctatttattagaaaatatataaagagttcttggcttaagaactcttctggctatcagctccctgatgtgaaaaaagtaaatagcaaggggtagcatggagtcttactcccgtgtgacagacagcttaagaaagacaattggacatcatgtgactacatgattcaagctaaagtccagacacatcttttccataggccaattgaacattttctctgtaatttccacaataaccatttgcaccagcatgaatggagagggtctgagttcttctgggtgagtaaagggtgtgtcagttatctgtcctctgtcaccaggatttagaggcaggctcatagtgactcttgtaaagttgaggttcgcctgtggaggctgcaaaaaagagggaggaagagagagtgaggttcctcttggctctgtgccagggatgtgattagaggaccctgggagggccttcatcctacgaggggttggaagtggaaatggatgtgtgtggatgacctggtaacgtgtcacagccccttccaccccatagtagtcagggatttaatgcctcaacaaggcaggtctctgaaggagactgacttttctctctctctctgaatgatactgcctgagagaaatactcacttcctgctttgttttcaacaagtatggacttccttacacaaaaagaatcttttttgcttttgtccccccatttcactggaaatctatccactgggcaccactgttggtcggcttcctttcatagattcctttatgtttcaaattttaaaaagacaataatagcaacaaagtgaaatatggatttaagctaggaaaagcagagaactgaaacttttttcctgcaatcatacttcccagcttcttcagcaagatctgcttggctgggaacatgccttctgagaactttacatttctaaactgctgctaaattgctccatgcatttattcctcaaactctcagggaagatagcactggctttcagtctcataccaaccattcttaaggtaatggaccaaaagtcatttccacttgaaatataatccttctaaatgaatcatgcaacatggtttttacctccattttttcagaataactctaagatgatgatcctctggccatattttaaaaatttctcttatttgttttaattttcttccattttttctttaattttgtatccagacactgccttctctaagaggaatgacatttttgcgggttatcatgatatatttttagcccatttgccttgacccatagcttacaacagttaaagaacactaacatataaacatagagttttgcccaaggccattagctttgcatgttaacaagtgggcagaaggaaggctgggcatgagagagacatgctgtccttctggtgagatcatcaatcccattcttgtaaccttcagatcattgcatttgttcagaatcgtctgcagaggaaaacatctcaaagttgggaggttgttgaagagtcagaaacaaatgggtcctgggtaatttttcaaaacaggctttttgtcaattgtcagtcagctgtttaatattgtcaattgtcactcagctgtttaatattgtgctttgttataaggataggcactgtggtctcagcgttcttaagcttttacatttccgttatttggttttacttctgcattagtagagtagatgtagagtagatgccacctctaggaactctaccagcagcagcatcttagactagaagctccctctctaggactataatctctgtagcctccacttctcccaccccttatttcttctgagcctacttggtctttgatacttactataatactttctattctcttatcataaacagttttctgatttcattttcctctggtttcatactaagccccataatcagtcactgcaagattaccttctagaatttctgtgccctcaagttttctccatatctagagagtcagtcatctgccttgagccacatccactgtctactttcttcacttccatccccaggctgcccagcactgatgaaaacaaaacaaaaacaaacaagcaaacaaaaacaaaaacaaaaaactgactaggatctcaatctggtttgcaaatccatgcttctctttcttgacttctttagacatcaacagaattgacgcctcctcttgacctgggacacccctcctccttggtgctttcttctacctccccctgcttctctgtatgtcttttgcaggctcatctttctcttaggctgtcttcccccatctctcctctccctctgtgcactcaggcagtctcccttgtcaacccccaaaactgcttctccagaccccagtgtttctctgagctccaggtccacaattctctaaggtccatgtggatgtccctcaccatttcaaacttctcttccacagggagtccctggttttcactaccatagcatattaaaataccatgcatttcccttcataccactaattacaactttgtttcttttttttaaattttatgttttaaaaattatcttctgcccacatatattgtaagctccttttgagaaagacacattgctgtcctggtcattactatattcttatcaactagcaggggccgtggtcggggcttgtgttagtccattttgtgctgctataaaggaatacctgatgccaggtaatttataaagaaaataggttttatttggctgagggttttgcaaactgtacaagaagcatggcgccagcatctacttctggtgaggcctgaagcagcttttactcatggtggaaggcaaagagagagcaggcgtttcacacagcaaaggagagagcaagagagatgccaggctccttaaacgacctgctctcccataaactgaaagagcaagaactcactcattacggtaggatggcaccaaaacagttgtgagggatccaccccgaacacctcccaccatgccccacctccaacactgggatcaaatttcaatgtgagatttggagggaacacacatctaccctttatcagtgctcaataagctcgctgaatgtagaacagtgatataaggcaggggtgggccaagtataggcgacaggacaaatttggcctgctactgcttttgtaaataaagttttatttatttacaaaataaataaataaatacagtcataatcattggttacattttgttcaaagctgcttttgcattaaaacatcagagttgagtagctgccacagagactgtaatggccccacaaagcctaaaatagttaccccgtggtcttttacagaaaaagtctgatataaaggacagtctcacagcacagaggaaaagcatataacccaaaggaggaaagagtagagcatagtggactgcagagaagtttccctggacccaatctgtgccttgaaagatgagaaggacacctccaaaggcaggatgggggtgaggtggggtgggcggtggtgatccaagcagacagcacgggcaagtgttctggaaaacttgcctttgttcacaagtatcctgcagtataaattgcaaggtgggcagtaagaaatggacttgaagagattgaacaagggccagaaacccaaggacattgctaaggagtttgtgccttagcccatccagggccctagcagccttgactgatgcttctgaggtcctggaggctccaggctcaggctaccttcccatattctctgaaaataatatcacatttgtttagtaaaaaaattgtttactaaaaaattaacctctcagaatttctgcattcacatttatttcttctcatatcctcgagttagaaaaaggtatttctctcctgtgatgatattgattctgcactcatctattttcattactgtctccttccctccctccctcagtccctcccttagtctatctcactctaccatcttttcctttggaccttctgacattcaacatcttggtcgctcctatccagccacacctctgaccattcaaccatttgtcctcttgattggttccaccttgtcctatcacgctggatatgctgttgctcccaaattgcctccctctgccagtttcttgccccttcctccttctgtctctccctctctctttcaatctttctctccctcagtctctctttctctttccctctctctcaatctttctcttcctcagtctctctcccattctctttctctttctctcaatctctctcttcctctctctcttatcctctctttctctcaatccctctctccctctttctctctctcaatctgtttctctgttaatctctctttctctctccttctttctgtctctgtctctgtctgcctctctctctctctctctcttccctagtgagctcacttgctttcccagtttcagtgatttctattcagatatctccaaaatttgtatttccggatattttatgctttcatggaggccgtgtaaattggtataacctttttggaaggcgttttggcaatacttagtaaaaggttaaatgtttacaccccttgattctgctattgtatgtgtaaggattttttcctgcatacttttgtaagtgtagaaaatatatgtataggaataattgcacattgtttgttgtagctgaaaattacaaataatataaatgcctataatcagagcagttaaatacatatgaacacagtgaaataccaggcactcatttaaaagatgaggtatattttaacaagcctatttgaaaagatacccaatcttagcaagtgaaaaaaaaactcatcaaattgatatatttagcgctgtccctctctcaagtttcagacccacattttcaattttctgttgtcatcttgacttagtctcgtgaaattttaaactaatcatttttaagggaagatattttcttcccttaaagatgaaattcagacaaacctctgaatttccctctttctgctaatttcttgctttcttttatcatctttccatccatcctaacatacagttccagaggagtctttaaatccttccttattgcctaacaccgaggatcatttgctgaaatcttgttgatttttccctcagatcaacaaaggattttcagaatagccctgatttctctttatcaccacggtgctgtctcacgtcttctctccccctcctcctacctgccgtctcttccagactcgtctctctcccccaatctcctttgcgcactgatgccaaattagtcacaacaaaagtgtttacgtgatcgtgtcacccactggtaagctcttaatggtcttcccttacacattatctcccatttcatctcaggagccttttattaaccactcttcacagtctgactcagccctctttgccagtttctctctttacaaatagcaaagtctatgagctagattactcaatattccctgaataggtcttctcttctcccacctcttggcctttccagtaccattcaatcttcctggaaattcctgaaatttctttcctcctcactcagtcaatgttttgagacccacatcaaatgctacatactatttgttcctttactgatcatgccagttggaaatgattcgaatctgtaaagcagggtgctatggaatgacatttcttatagccctttggtcatttgggagcaatgtctaacataatatcctccaggtccatccatgttgttgcaagcgacaggatttccttcttgttaaagggtgagtagcattccattgtacatatattccatattttatttttttttgctcatctgttgatggtcacccaggttgattccatatcttggctattgtgaataatgctacagtgaacttgggagtgcagatatctcttcaacatattgatttcatctcctttggatatatacccagtagtgggattattggatcaaatggtaattatatttctaattttgggcagggtcctctgtagtagctccataatggttgtagtaatttacatatccaaccaagcacgccagggttcacttttcctcatgtcctcgccgacaagcctgatattcttatttgcctcttagcgcttcagcctttccctcgtgacttaacggtgactcccttgagactacttgaaataataagtttggatggcaaggaaatacccttctgctgtcaccctttgccataagactgagttactttgtaaacaaagaagatttacttggtcttccatgcccaagacccttttacttttcatcgcttaatatttcatggccaaaaactgttggttcttccaatcttgtaaggccatttatctttgatcttgctccattcccttccatttttgcttgagaatcattcagttcttgcctgatctcacgtctttctcgcaacaccttactaatgactgccagagtgtccactacacactatcggtctcatttgaccacttccccaggcatgtagcctacctgccaagttctttcacagcagatattttgcagtgttgtaagagggctccctagcttgctatgttttcttactacctggcattaggaagtaaacatttttcatggcagtattcacttctagtaccaatttcttttttaatctacatgggctaactgctgtatcaaacagccatcaaatcccaatgttaattgcaacttacagtacgttgtgatgtttctgtctgtatactcattcagacacacagagtttaagtaatttgttcaagatcacaaagttagtaaatggtggcatcaagatttgaacccaggcagctggactcaagagtctaaacaactttcattttaaaattttcatctttcaagatatatcacttacaatttccatcttcatcactttcttccatacttcagagctgcatatgtaaacctcttgatcatataaatatttatggagtctcagcattttgagcacatgctgagagattttgtagcattgaagataaagaagctctttcaatacatctaatcttgccactattgattttgaacagcatcttaatttgttacctttttaaaaccatattgtacttttcttcaggacagctaaattaaatgtcttatgttgatcccctgggttttctcagtttccatttatcagtttccttttatataaagagatgctaatgggtaaccttacattttgtaaggcatatttttgaagatatacaagtgaaggtttgatctgtacaccctcgttagagctctttattttacatgtggaggaagagatgccttagggcttctgaaaaacaggcttttatttttatagaggagaaacttccagaacatgccatgagcaaatgctctgtcaagtctggttctttcccccattacttaagtgcaaatggaccacatttgaagaagataagtagcagaacaaggctccccacaaggggattttgaggtgctctgaaaaccagactgacataatccatggaggagttcatcactgcaggctgtcacgtctcacagtgggagtcataggctggtctgcttgataaactactcctgatctatgactggagctgtgtgcttcctttaaaaataaatcttgtttttccatgccccacgtgcttctcagcctcaggagcacctcatggatttagactaatcttgtcttttacagtcatgttacttccagattcctaggaatcaccccatttctgatgccgttgatccatccatacccactgaaattgagctaaatgctctagtctgatttgccccttgaagttagtatagaatattttggctagactttgaaaatgaagccctgaccacaatgcataatgacctcagtagacactgaagagttcctttgcatcaggcaagtgacaagtatctatctcatcttcaaagtccacatagatatgtgccagattgtaggttgacaaatcactttttcactagctcacgtggcaagccttttcaccttgtaataatgctagtgagagacccataggtaccacataattggccagtcattgagattcaagcaacatttggataatttagcaaaactatcatcagaatcatcaaagtacataatttttatttattatgcaaatagtggattcactaaagtgcttagaccaattaagttgctcccattgaaaaacagggtgattttattcttaagaatatatcaggataaacttcttgtagcctctaaacagtactgcttagaaaagtgcagcttcactgcattgcatatccccccagaagccctcaaagttcagatgtacaccacaaaaacatgaagcacatgtgtttgcatggaaggtaggctctggggtcttcctatcataatatgtctatgattaatagtcaacatatgccccctccacattccctaaaggaacatacacatgcatacacacacacacacacacacacacacacacacacgtgagaactgaattgagagtttggggtatacaccatccctgagggcacagtctacagctcctaatgtgtgctgtagagcaagcattatattgttgacaggtaataattcctggttaggaataacaggataccagtgagaagaggctgagaaaaatacactcttgctggaagagtcaagaacgagctcttcgtgggaaactatgggagacgattgaaaaaggacaagaagaaaggagaattgttaagtctccactttctcagtttctgaactgaacttttgctcacagcaggtgaacacaggctccctcccacactgagacttcctcccctgccggtgtgcagtctgttaagtaaacagagacgttatactgagaaaggaacatttctctattttggctatgagttgttatttttaaagattcatcctccaaacattttcccctatccttgtttattttttcactagaaagaagatgaactgcttaaaaaaaaaattcaactccatgacaagaaaaacccacttctagtatgttctccccactcaatgccaatggagcctttggtgtgagcactctgccagccacacattgggcagcatacctcttgaaattcctgctgtgcttctatagagcttccatgcatccagattttcaaagtcaaagcaaaatgcgcatggcagtttttttctgactccttcaagttaaaaggccaacctctcccttgacatatccatgtatttatcagtatatctataggtaatacttaaaggtgatccttactgtgtgccacactccattctaagcactttacataaaaaacctcatttcactttatttgggaataggatgacaatttcttcaatattgagatgggttgaatctggggagactgatgttagatgcaaaaagctttctttctagaacttttcctgtgtcttgcacctggctttctccctgtcttgttcccattctagatctctttactgacagtgcccatggctttcttcttggtactctagtgttgagcataagaactagtatgtaataggctctcagtaaatcttgttgaatgaattaaagtagagcaagaattaaagacagagaagaagatcttcttgttggagttctttcttttggttgataagcaatgtgatttgacgtaacaatcttgagtcctaatcccctcatttggaaaatggaaataataatgctaatcttagaggtcacttctaagaagtatatgaatatatcagaagtaatcaacatttttaagtcaaagaaaagaacgtctgtgaaaaagaaaactaatgagctagttgaaaaaatattaattggatctttttctcataccttacgccaaaataaatccccaaaaatggttgcacaacattgtgaatatactgaaatccattgaatcacactctttaaatgggcaaatgttatgttatgtgattatctttcaataaaattgttataaaaatgaataaaccccagaagatcaaaaatttaaacataaaagcatggaagtatactagaaggaaatataagataattttatatgttatgttgtatatatttatatgtaaaatttttttatttatttttctttgcttttaaaagcaagacataatgttctagaggaataaatgaaacgttcgataaatttaattctctattaaatgttttgcagaagaaaataccaggaacaacatcagaagaaaaaacaaaaaaccataagaaaaacaattaaaaatacatatgacaaatggcttattactataatatttaaagaactcttaaaaatctatgtgaaaatgacaaataacagaaaaatacgtgcaaaggacaagagcaggcagtttagagaaagcctataccaatatattctcttatcaacagtctattgtacaagctatttgaaagcaatgtttgaactatcaaaatttaaaatacataagcccttttatctagcaattctacttccaagaatttattctatagctatctgtacacatatccaaagacataaacataaggatagtcattgaatgaaatgtttttaacaacaaaatacttgaaagcaaagaaatgtcacccaagaaagaagtgattgaagaaagtttagcattcagttcagtgaagacatattgagctgtcaaaaaaagttgatggagaactccaaggtctattaactaaaaacaaaacaaggtgctaaacaatatgtttagtgttcatattttaattgtttatatatttcacaaatatatatttgtgcatgcagaaaatattttaacttctttctaaccccgtcaaacacatcatgccaaaattcaacttgtgataaagaaaaaaataaatgaattcctttcgtgtgtagtatctcatttaaattcttcaacaacccaataaaaaagatacagacacatgtttttaagtactacatgttatgcacctttggtttgcttatttaatagttgcaactactaatgttaaattcttgatgccaaggttaaaagcctgaacttctaaaaatatcaagatctgacatttttctgttttcacagattaatctgccatctcctgacctccaacacactgctctatgttctttcatgctgctcctcctcccatgtttaggaccctcttccaaggaggcattcacatctctgcttgatgaaaccctacattatcttaaagcgtccactcgaatgccacctacttatataccatctcctgcaataccaatgggctcagcccaaccatagcccatagttattgtagttgttcttgtacttcaaaagcactacaaaacacaaccatcaggacttgttacattatttgaaggctatgagcatcttcagccgaggccctgtttttattcccagaactaccacattgtttagaatatagtagcagatcaatatacgtgtattagataaatcgtttacccagatcttgatcattttcaattacccataggttgaagaactccatatttaacatggcagacttgaggactgaactacctacctcttctaagaagttgaaatgagaatgttttattgatgggaaattatttttttgttttgccttctagaattcaaatgaatgttcatattccatgaagacaatggctgatagttttttgttaaagatttagaaccagtggatttttatgaatgtgaaccctttcatgtcttgtggaagattttctgttttttaatctttttatttatttatttatttttgctatgttttcataggaaccagggaaaatgtgtagagggcatggtggagatcttcgacatgctgctggctacatcatctcggttccgcatgatgaatctgcagggagaggagtttgtgtgcctcaaatctattattttgcttaattctggtgagttgataacacaagataactcaatgctggatgaaatgtttatttgtagttttcaaccagatacgatctacccactccaaaggcataatgtcataaatagaaagaaactactgacacacgttttaaaataacctaccaacattgcagattccttataaaggtagaaccatgctagccaaatagacacatgaaaaattgtaatttggcattgaatcaaatggcctttgagctaaaatttttgtatgctttcacagataggatgtttttattcaaatggtacatgtatatagacatatgttagttgatagttatattatgtctgaaaataagtagaccaagtaattctgttaagaaattgtgaccaattccaggctccagatagtaaagaaagagggttatttgagacagaccatgtttctggtcaaaactgactagctaaaaatatagttggcttagagatagaaaaacctgtttctaaaacagaagaatgtggaatgcaataaattgtccagctgaaagaacattttccatttgctctatgaagtctgattctactgccccttcgtatttatttgtttgagaaagcttagctaagagcaacatctgttttttgtttttgtttttgtttttgttttgtttttgttttcaatgtagtgaggctggctgttgtataaagagttactctatgtcaccaagatggaaactatggttcagcctgaattagtgccctgactcctcctagcctcttggtatttggatctaagctctagctctacagcctctgggacctaaagctcacattgggtatcagcggtacagcagctccccttaagctccacctttccccctggctctaaccccactttgtgcctcatccctcattctcacatagaaatcatagctatttcgaatctctgggcctgacttagtttcttatgccattagacatagtctcaaattcctcaatagctgaattgcagcttgtattgatcacatacatagagaatccgcacttccttctctttccacgtgtttttgtctctttctgatgagtgctgatcccttcccgcttcccctaacaattttttacttttcttccccaccactgctaggaacctgtgttgcttaatggaatcagcccttccttctcttttcttgtgtttttgtctctttctgatgaatgccaatcccttcccacctcccatgacaattttccacttctcttccccaccaccactagtaacctgtgttgcctaatggttatcacagtaatcattctcacttatcaactaataaatggaaatctgtactattacactaggcaaggactgaattacagaatgaagtcccatgataaatgatgtgatccacactaaggaagtgattaccactccattcaggtttctgtttccactcacgtgcattgtgcttttcatttcagtcgtttgtcctgtacattgtagggtccagatcccacaatggctctttattggatgagagttctgggagcagtgccactcagctacatggtgccaggtcctgaacctgtgccttcttcggtggagggctggcacgtgctgacagctttcatgtgggcaatctgggaacttcagagaaggcaggcctattaagtgttaagactccccaccccgaacttttactgagaaaaagtaccccagacaggaagtaaaattagccagagttgtatgatccacacagtggatgctctgatctcagtaataaaaaatatttcctccagatccatatagacttttcctgcattattgttttgtttctgttcctatggcagagtgagtttttaaaactattatgcaagaaatatcaggatttttgccaccaaaaagttggattcatagacccagggtttttacaacccagggggaaaaaactttcagccctcacaagaaagtattttattaaagactggcacccaaacctcaagactatatttctcactgcaggatttggcccctgtctgcctccttctcagactagttaaactttcatactccatccttgttccttcgcttctttcttaggatcttctgtgcacttctttcttaggatcttcttggcacttctttgctcttgaagggcagaaacgctattgtagaaattcaaggcactgtatgctgacatagtttatagttgtctttttcatgagataacacagcggctggcagctcctcttttctatcaaggacagtactgctggctcaggagagcagtaagcaaacagaagctgtctctcaaccctgtacaaagcgaaaccactcttttccctacaaaagtgagctgtgccccagaaaagcggatctgctgctgagcagggctcctcatgtctcctattgttcctgaaaaacagcatcaaagagggcaagactgacacacaatgttgtagcattaggagcctttttcagaataaaaaagcaatcagtcatatgaagcatgtggtcataccacaatcaacaatttttctccacaactggagatatggatttttctaaaagtccaactgattcatggccctgatacggggcagctctacctctcatgagaccaatgaccaagtgactctgactccaggaatctctgacagaagccaagctggaaacggctcaggaaacctgagctagaagatgcctttctgacacctgggaaacaaaacctcagtgtatggaggaacccaaggttttgtgttgagccccagtcgccaaggtctgaggaggctgggtttatattggccccgattttgtggccgaggcacaaggggaattgaaaggttgttgtgaggacctgctcggaatgtctttctggaagtttggagagggtctctcaaatgccaaaatttccatgggaagccattttcacagctgcttgggagtgggagatattcgtcatcatttctgccccatttagaataatttattactttgcttgcaaaaacccggaatcatccataggacacacgttgtttgtttttccactccacaccaaaggacagaggcattcctggttaatccaagatgccaacttcaagacattctgagaactagctggacagagctgaagtgtagggcaaatcagaacagaagtgatgggcccagggcaaaagcccgtgaagcagcagctgcggaggaaggatgggagctggagctgcaaagggggccatggtggagactctgggcctgcccacagtatcccctctgcccaatgggaattcagttttcctgcatatcattcaattatttctcatttttctaggcctagctcaccttattcgggaagttttgcgactaccccagtccataatgaatttcctctctagactatttcaattcttacagtcactaccacacagtctaggatttggttagttggttgatttcattcatttattcaataattcaatgtttattgagttcctactatggactttgtgccaggtatacaaaactgaatgttactgttgtgtggggtttttttctttgcatttttaaagttaacctggatttttctctcactctttatgcatgtatttcctaatctccctataaactatttgtatttttttggattcctcacagtctatttagcctgtggactgtcaacagatgttacatcattcagtcatttaatagatgcttatttgctagacatggccaggaggacctggaaagtcctcatttttcccatctgttcttaccattctgaaatgtatcatgctcttcagtgctccacagatctagatgatatcaaattattaaatggaaaaagcaagacccatctccttgggttattgtggataagggggaaattgtgtaggaagtgtctattgcagttcatggaccattgtaggcccttagtcaacaacagctatcatcatcactgatgagaatctctggcttaaacggagtaggttcatgccttttttcagtgtaatgaagtgttttagtttaaaggcctttagcaaggcttacaataaaaaaaagtggggactccagggtaacaccaacaatagagacgaccatggctgttatcctgagactattaattatgccacgtaattagatttagagaaaacttagagcaacatttatcttgtggataataattgattgtgattgggacttaccgttgagagaccctgatgaaaactctatggtgaattgctgtggagtcacatgggttcatgcatcattcatcagtgggccctggttttagcctcataacccctgagaatccacctctatcagaaaaccaccctttcagtgaaagttcctcaaggctcagaaaaattcatatgtcaaagctttatatgtaaaaacctatactctctaataaatgtttggtttggccttcacattcaatttaataattatttctacctcttttctaaaccgctcttaaccacagttctatatatttaattctgaagagcctcatgcacctccctccctggtaactttatctacttctctccctggagaaccagccagaccatgattccagccctctgtcctctcttcaaccgtaggtcctctttttctctcctgactcctcaaaccttctccagttacccggatgccttctgtgtgctgctggcttctgcatcactgttcacagggccacttgttcccctggaaatgtcttatgctcttgaaatgctatagccttgttcacctgttcatccaaaactgatcctcagacaacatttctctttcaattggtggaataataaagaggaatatgggtcaaaattcatagacatttaaatacccaaggtgcaaagttaagagaaaacaagataggagaaagaattatggcaacatctctaaaatgtgtactttagtgtttagcaaaaaagggactatggaatgtcaaacaggaacatatatgttaaacataaacatgaagcagtgccagctgctgtctgccatatagtcatgctttgttaatgtggcacatcttgccatgatggtgccctcatgaaacgaaggatgctgaagcagatgccattctgtttgctttgatcagaagccactctctggtcaaggaagtatttgtttcatgcttaattataacaagttctcatcacaaagattattttctttctccagatctggattgtttgcactcactttatattattagacaattactcagattgtttatgtttttatgtttagaaataagacagtgattcagagagtccctgtatttagatgaaagaacattttttaaacatattgactgtaaacactgaaaatcattaattctcaacagttgaaggtttctgacaggaacggcccttggaaattgtaaatgggcacattgtcaggtatgcccattgttgtcacattggagaagattggattctgttctctctggagtcctttgtaatgccaagcttcctctccagggagctggcttattgtcattattttgagataaatggcacttcatagtattaagtggttcattgtctcttctctggctgactgtattctgtaggcagccactcaactttggagcctttctctgctaccaacttttggtgttgccttaagcaagcctcatggctcctgttagtttcctgacaacatggtatagcatggactttgtcatcagagaggactgaattcaggagaagcaactctgccacttccttggcggaagggggatgactgatgtcctttgataagctgcttccctgagcctaataaaaaaatagaaataatgatacttaaactcatggaattgttgtgacaattgaaagaatgaccagttagtaaagcatcttgcaccatgactagcacacagtagatgcacatgagtgttactaagctttagttatgccctccaaatacaaagccttagttttgtcactatgaaagaaattactctgttcttcttagcaggtaataaataaatccctgcctggaaattctgcaggagaatttcacccctgcgctttcaggttaaaagattgcattcccatgatgaagatgttctgtgaatagaatcatctcaagtcccaaatggtgtggatttgctcctgcatcttgttgcagagattctcttggagcttctgagaactggggaaaccaacaaaatgcctgtagcctggggaaaatgtagggagttttagcatgaatttgtgggaggtgagaaaaggcagcaggtgtcgaaagacgggaaatccttgcactgttctagatgttaggagccattttccattcacattcagcacctcaggaggagaagctgcctatcactttgtgtcctcaagagaaccaacaaaggagaatgccatctcccagatactcaatttgattaccattttatcctaccttctctagggtccagacattatgtaaattggcattaagtttgaataatgtatggacccagtttaaaaggaaaaaatgctttgggaggctgaggcaggtgcattgcctgaggtcaggagttccagaacagcctggccaacacagtgaaaccccatctctactaaaaatacaaaaagttagctgggcacggtggcgggcacctgtaatcccagctactcaggaggctaatgcaggagaatcacttgaacctgggaggcagaggttgcagtgagccaagatcgtgccattgcactccagcctgggtgatgagagcaaaactccatctcaaaaaatttttaaaacaaaaggaaaaaatgctcccctgattcccgcagtgctgacttaaaatgttcttagtatgccaatgttgcttaaatatgaatgactgtagcttctacttaaattggcaaccgcaccaaatataaactgcaaatgtttatagctcttatggaagtataaaagcaaaacaaattcttaaattaaatacaaactaagagggaaactgataataacaatgttttgtgtaattaagttgctgtttgcaacatgcctggggcagactcctggcctctttgcatctgacatgagaggctaccaaggatgtggccacaactgggctctcccagcacttgcttgcgctcagaactgtgccaaaacctttgctcgcccagtgttctgcaacgtccatcatttggatttggcaggaacacatcatttatgcattttgtttgcttctgtatttagttgagatacttaaaatataactgctagattctaaggcattcataagcatctgtgaaaaatgtgttacttaggaaacagttcagggttccatggattatctagattggtgagtcttaagactgaccacacaattcatctgggggtgtggaggaggggagctttgagaaaaatctctgtatgagcacccacacccagagattcctatgcaattatcccagagtggtgccttaatatcaacatttgtttaaaaccatcccccaagactaaaatttgtgaccgggatttagaaccattgacttaattgatgcagaacactctgatattaatgtcttcatgtcttttggaatgtcattaagacaaaagcataaaattttaaaattcttaaagacaatttaaagacctatgagaatccatctttaattctcaggagcgtgtggaacctagtttgaaaactactggcatcgacaattgaatttccactaaaataaaatagctctctagtatattacaaaactacccattctgcaaactgcaggggagctactgataatgcttggaactgtgccaggcactgcctgcataaaaatgagtaaggtccacttcctccatggactgggttgggtaggaggcaaagataattaaccaattattttaatattatgagttcagggttgtaatgaaagtaagtattgagtgctgcagagacccagcaaaggagtccttagctgagaactgatcctttgctgagtgacgaggacattattgaaccgccaggcttgatggcatgttgcatggtaacagaccagtacaccaaacagcaggagctgtagcagagaaagagtttaataattgtgtggcagccaaataaggagacagaaggaaaccttaaatccacctcttgaaaaagtctgggactaggctttttaagagaattctggcaagaaggaggctgaggagctgggggtaactgattggttaggacataggagaggagacaataaggatatagaaactccattcttgcactgggtcagttcctcggagggggtcctcactctggctggcagcagtgaacccattggaatgcaggatctgaaaaatatctcaaaatggcaaacttgaggggttttttttagcatcaaagatgttatctatagaagttaggacattgtgacaggggctacgtgactttgaagtgttaagtggctgtcagaaagtgagctattgcagcggtccccaacctttttggtaccagggaccagcttcatgaaagaaaatttttccacagatggggtggtgtagggtggggtgggggatagcttcgggatgaaactgatccacctcagatcatcaggcattagttagattctcatgaggggcacacagcccagatccctcacatgggcagttcacagtagggttcacagccctatgaggttctaatgccactgctgatctgacaggtggcagagctcaggcagtaatgcttccttgcccaccactcacctcttgctgtgtggctcagttcctgacagattacggactggtaccagtttgcaacccaggcagctggggacctctgggctgtagggtaggctggttaatgcttagctgtgtttctattcaaagcttatgcttttgtttaaaacctagtaatttggttttgttaattttatgaagatgatttcaaggatgagtaggattagctggagtcagaaggacacaagggaatttctggcaacaacattaaactaccagtgaatgtctcatcaagtcactggacctctaattctaagcagtactgtcaaccttataggttatgatgtataagatagtgttgtgatgtataagatagtgcaaaaacctaagttaatatcaccatttctgtagtactctctatttcaaaggaaaaaatcttcagcagtctaatatcctaccaaaattttgaagtcaagacctccctacactaatcaaagaactagaggtgcaatcaggccatatatttggaaaggccataggtgccctccctctgtccttgttcccagcatactaaactatagtctaaaagactttaaaacttgaactgaagaaaaccttaaggaaacaacaacaaaattccatttatttcctgagcccaaaagtagaataattgttttttaatgaatttttctttccctgtaagacatactatgaattgttcaatgggtattcaaagcatatgtaatagaatatgtatgctttcctgaaaaattgaaatggaacatcaaactgttctgtaaagttttgagtctagaccaaactcacagcacttagccagaatatgtacatctgcatctgccactcatggccccatcgctccctccaactgcttctcttgggctgaaggatggaaaagcaaactccctgcgttgagttccatcttgccctgcatggcctcccccatggggtcattgagctccccaccagcttccacctcttccagcactttccgccagagggcccgtggctgcttttgcagactgaaattgattgtggttgattcagctccatttcctctgagtttttactttagatgtgctttttaggttgcatttttcttctggcatataagaaagacagcaatattccatggaacagagtgatgtctgtggctttgtcaaccgttgtttgttagcttgatcaccatttattgagtgtttactactttccaggcattgtgctaagtggtagaaatacaaagttgcctttacttgattcatctgtaaaatagagagagtagtagttcctacagtgtggtttatgtgaggcatagggactgctccctgaaacctagggacaactccctagcagcgacccagtgctcatatcaatagtggtaacaattaggaaggcagctcctacctcaaagacctcatgatctgatggtaaagacagatataacaagaaaattattctttttaaatataaaattgtgatataaaaatagttttcactaaattggatttttatgaaatttacagaaacaaaataataaattaaggaaatataaaaccacctgacattagccaaccatgttgatgttctgattatgttactgcaaaggagtgtagggttaagaacgagggactttgattccaagatcaaatccctttctgctatttcttagctgtctgactttgggcaagttgcttaaaccctttgtgcttctgtgtgcttgcccatgagatggagattataatagcgtctacctcatagggctatttttaatacaagttagctcttagaacaatacctacttaaactaacaatttgtattaggtactgttttattctccatccgtactgtgtatgcatatattcatatttatatttacatattctaattaggattatatacatgtactttctttaaaaataagattttaaataactatataatcagtcatgatgatacaacatgatttattcaacaaatctgatgctgttaaacacgtaagtgatttctctttcaggattataaataacatttgttttgcatttttttcatacatcttttgctcacttttcattgttactttgtatatgttgctagaaattgatttgctggtgaaaagggtataaacatttttaaggctcttgctacatattgcctgctcttcatacatattgcctactgccccaggaaactatcaattacacttttaccaagagtgtatgaaagcattcacatgaatgtaaccttatcatgcaatgcgggccatgagacagaggagagttatggagggtagacggcttccccgctgctcagccactgccctctcctctaaaagagccacagtcaggaatttctacatagatcctattataaacatctccttttgaaacaaattatcttctctcagagcattttcttcattttagtatctatataggtccttgttatgttcattttaagttttgtgacctgcactgacacaatgctgatgaactgcttattccaggcagtctgagttctcaaagcatttgcattttatgtcttatcctctgttttgggctctttgccactgatttttttttaagtgatattttaagaaagaggagggttgatttgttgtgagtgtatctctctttaaaaacttttctagggaaggacaacctcttccaaagactttgtttgctttgtgtgattggcatgtccttccctggtcagtaatgagcttgaaggaagctactggacacgggactgaacatggggttttgctatcactctagctgtgcctttctctagtgatcaaggagatgttggttcagaaagtgccctgtctaatcccactaaggcccgaaggaccaagtgctgtctactctccagattctctgcaacagaaagcagatgcttaggcttcatggtccaggttggaacaggtagttagctctgaggacagttatgcatgaatgtcaggaggggactggcaggggaacatttatttattctttaaaattggaatcaacagttatggatgaaacccagaatgcaaaattcactttcatcttgacacgcaacacatctgtcctttctccttgaatagcaagtattaatcattgagttagacaatgtaaccttcacattcagtgaaagccaaacactcagcaccttctagaaaaatcttagtgccatgctttcttagcatattgcaagtcctctgaggatgatgtgttgttcagctttgaagctgatcttttgtacttgcttatgtagctcaggctagtgataacatgccagaggcatttccaactaagaaaattattcacagaagctttaattacaaatttgttagccccacttcctgctaaaatggccagccttgaagagtttcggaaatgtgagcagccttaaggatgatagaaatattatgaaatagtaataagtaatacccatacagcaaactcctttttctttggagtatccgttcatcagccagatttccctctcaacagtccaggtgttgacgaagatcatgaaataataggaatgaaagggacttcgattttgtatttttaatagcattttcctccaccaaattacgaaagtaatgcatgaccaaggcagagaacttggaaaatacagaaaagccaccaagcaaaataatctcccaagaaatcataatttcaccacacaaaagtaaccattgtcaacattttggtgaaaatccttaattaaagagactcttaaaagatgccagttcgctgccacttcttcctttctccacaccatcctgcaccctgttttgaaatgcagaattgtttctaaatcattccaggaaaattattagttttctcaaatatctctggaggcgagtctgttggcagtcatttctagtttctcacaaaccttgaagttaggaaattatttgtgtagcttttgagcactgggccagaagtgagggatgtttgggttctagttccagctctaccacttatttaccaactggccgacctcaggcaagtccaggctagcggccccgcctcaagcccctccagatgcagcatctgcaaccccacttccttcacttcctttcgcctcagtttcccaacaatttcctagtaattgtactgaatcattgtttttcattttttacattaaaatttatttattttattttatttttaaagactttattttttagaacagatttagatttgcagcaaaactgagtggcaagtacagaaagttcccatatattccctgcccccgcacatacacagtctccctcactatcagcatccccaaccagagaggtacattggttacagctgatgaatctacatcgacacgtcattatcacccaaagtgcatagtttgcattggcgttgggcattttagggcttttgacacatgtatgcactattgtaatatacagaataatttcactgccctaaaaatcctctgtgctcctcctattcattcctccctccaccccattcctggcaactactgatctttttactgtctccacagttttgccttctccagaatgtcatatacttggaatcataaggtttgtagccttttcagattggcttctttcactcagtgatatgcatttaaggttcttccatgtctttttatggcttgatagctcatttctttttatcattgaatactactactccattgtctggattatacacactttatttatacattcacctactgaaggatgtgttggttgcatccaagttttggcaattatgaatgaagttgctgtaaacacccatgtgcaggtaattgtgttgatatgcatttttaatgcttttggataaacactcaggagcacaattgctggattgtatagtatgtttagttttgaaggaaactgccaaaccatcttcccaagtggctgtaccattttccatttccaccaacaatgagtgagagttcctgttgctccacatcctcatcagcatttggtgttgtcaatgttctggattttggccattctaacaggtgtattgtggtatctcattttgttttaattcgcattttttggataatatcacatggagcatcttttaataagcttatttgccatctgtgtgtcttctgtggtgaggtgtctgttaaactctttggtccatttttttaatcaggttgtttgcgttcttatcgttgagttttaaaagctctttgtatattttggataacagtcatttagcaggtatgtcttttgcaaatattttctcccagtctgtggcttgtcttctcattctctggagatcagtggtttttacactttcttctgctgaagccaagtattgtggaagaagccatggggtcaccagaggataggtgcacagggtagtgagggaaagttggagtgaatgggcttggtgcccccaccccgcaacatttctcttgctttaatcagagaagtttgacttcatccagattgcatatgaggactttacatgaggttcacctttttatttgttttaaaaggggtttttactctcaaaatgttttaaagaccactgaactagatgatcatcttaaggcccttttaagatttatgattctggaattctgtggctctatttttccaatgtaatataaccttcctctcgttaaggaaaaatgagataaacctgttataaacactcatgtgacattttttcaatacagttttcatccacgtagtgctcacttagaatgatagaaacaatacacctaaagaatatgcaccattcacttgcccactcatctctcaaagagaggaactttcaacactagttatctttttaaaaataatcagagagaattctcagccacttcaattctagcccaggaaaaagagggaaaatacatttgaaggtttctggctcatgtgatacatttgcaaaccagaaccaagatgatatgactctcatttattagtgactattgcaaagctttgcaggctattaagcaggctgttcaatttagggtgatatggacatgtgcatattggccaattatatatgctcagatcgtatgtcattttttttctgcattttagtctaagtgtcagataggtgtttcatgatgttttataaacacttctgttcacattaacattgttcattgatttggcaaggaatgaaggggtgtcattttcatgattttatttgtatattatgtattccacagaggatttgaaatgccttaccaaattttatgtaatgtaaaggggtaaaccaatatttgacacatcattataaattgaggataatctgaagaaaataataaatccaggagacagattatcacactgatatggtttggctctgtgtccccacccaaatttcatcttgaattttactcacataatttccatgtgttgtgggagggacccagtgggagatcatttgaatcataggagtggtttcctccatgctgttctcattgtagtgaataagtctcatgagatctgatggttttatcaggggtttccacttttatatcttcctcattttttctcttgccaccaccatgtaagaagtgcctttcgcctcccaccatgattctgaattctgaaccctccccagccatgtggaactgtaaatccaattaaacctctttttcttcccagtcttgggtatgtctttatcagcagcacgaaaatggactaatacagtaaattggtacaaggagtggggtgttgctgaaaagatagccgaaaatgtggaagcaatcttggaactggatatcagggagaggttggagcagtttggagggctcaaaaaagacaggaaaatgtgggaaagtttgaaacttcctagagacttgttgaatggccttgacaagaatactgatagtgatatgaacaataaagtccaggctgaggtggtctcagatggggatgaagaaactgttgggaagtggagcaaaggtgactcttgttatatcttagcaaagagactggtggcattttacccctgctgtagagatttgtggaatttgaacttgagagaaatgatttggggtacctggtaaaagaaatttctaagcagcaaaacattcaaaaggtgacttgggtgttgttaaaaccattctgttttaaaagagaaacagcataaaagttcagaaaatttgcagcctgatgatgcagtaggaaagaaaaacccatttttttgaggagaaattcaagctggctgcagaaatttgcataagtaacaaggagccaaatgttaatccccaagacaatggggaaaatgtctccagagcatgtcataggtcttcatggcagcccctcccatcacagacccggaagcctaggaggaaaaaaacagttttgtgggccagtcccagggtccccatgctgtgtgcagcctaggaacttggtgccctgcatctcagctgctccagctattgctaaaaggggctgaggtaccacggtttcagaggttgcaagccccaaaccttggcagctttcatgtggtgttgagcctgtgtgtacacagaagttaagaattgaggtttgggaacctccacttatatttcagaagatacgtggaaatgcctggatacccaggcaaacatttgctgcagaggtggggccctcatggagggcctctgctagggcaatgaggaagggaaatgtggggttggaacccccacacagagtccccactggggcactgcctagtggagctgtaagaggaggaccactgtcctccagaccgcagaatagtagatccactgacagcttgcaccatgtgcctggaaaagccacagacactcaacgccagcctgtgaaagcagtcagggttggaggtggtggtggctataccctataaagccacaggggcagagctgcccaagactatgggaacctacctcttgcatcagcatgacctggatgtgagacattcagtcaaaggagatattttgaagctttagaatttgactgccctggtggattttagacttgtgtgggccctgtaacccctttgttttggccaatttctcccatttggagctgctgtatttacccaatgcctaaacccgcattgtatctaagaagtaactagcttgattttgattttacaggctcataggcaaaagggacttgccttgtctcagatgagactttggactgtggacttttggttaattctgaaatgagttaagactttgggggactgttgggaaggcatgattgcttttgaaatgtgaggacatgagatttggagaggccaggggtggaatgttatggtttggctctgtgtccccacccaaatctcatcttgaattatactcccataattccaaagtgttgtgagagggacctggtgggaaacaatttgaatcatgaggccagtttcccctatactgttcttgtggtagtgagtaagtctcacgagatctcatggttttatcagggtttccgcttttgcatcttcctcattttctcttgctgctgccatgtaagaagtgcctttcacaagcataaggaagaagtatacttgctggagcagcaggctgtccttcttctgacgatatggactgcaagtgcctatgtttactttgaccaagacggcctttcttttcacagaaagccagggttcaccccttgaatttcataagacctctctttgcagtgggttagatactcttttatatttttatactctgtcaggaaaacatttttttttccatttaaaaatcagtcttttggttgcttccaagatggctgaataggaacagctctggtctgtagctcccagcgagatcgaagcagaaggcaggtgatttctgcatttccaactgaggcacctggttcatctcactgggactggttggacagtgggtgcagcccacggagggtgagccgaactggggcagggtgtcacctcacccacaaagtgcaaggggtcaagggatttccctttcctagccaagggaagctgtgacagactgtagctggagaaacagtacactcctgacaaaatactgcacttttccccacagtcttgacaactggaagaccaggagataccctcccttgcctggctcagtgggtcacacgcccatggagacttgctcactgctagcgcagcagtctgagatcaacctgcaatgctgctgcttgatgcggggaggggcgtctgccattgatgaagcttgagtagctcacagcgtaaacaaagcagcagggaagcttgaactgggcagagcccacctcagctcagcaaggcctactgcctctctagattccacctctgggggcagcacatagcagaacaaaaggcagcagacagcttccgcagacttaaacatccctgtctgacacctctgaagagggcagtggttctctcagcacagtgttcaagctccaagaaccaagagaccgccacctcaagcaggtccctgacccccatgtagcctgactgggagacacctcccagtaggggccgacagacacttcaaacaggcaggtgccctctgggacgaagcttccagaggaaggatcaggcagcaatatttgttgttctgcagcctccgctggtgatacccaggcaaacagggtctggggtggacctccagcaaactccaacaggcctgcagctgaggggcctgacggttagaaggaaaactaacaaacagaaaggaatagcatcaacatcaacaaaaaggacatccacaccaaaaccccatctgtaggtcatcaacatcaaagaccaaaggtagataaaaccacaaagatggggagaaaccagagcagaaaagtggaaaattccaaaaaccagagcgcctcttctgctccaaaggattgcagctcctcaccagcaagggaacaaaacaggatggagaatgagtttgatgagttgacagaagtaggcttcagaaggtcggtaataacaaacttctccaagctaaaggaacatgttctaacccatcgcaaggaggctaaaaaccttgaaaaaaggttagatgaatggctaactagaataaacagtgtagagaagaacttaaatgacctgatggagctgaaaaccacagcatgagaacttcgtgatgtatgcacaagcttcgatagctgatttgatcaagtggaagaaaggatatcagtgattgaagatcaaattaatgaaataaagtgagaagacaagatgagagaaaagaagagtgaaaagaaacgaacaaagcctccaagaaatatgggactatgtgaaacaaacaaatatatgtttcattggtgtactgggaagtgatggggagaatggaaccaagttagaaaacactcttcaggatattatccaggacaacttccccaacctagcaaggcaggccaacattcaaattcaggaaatacagagaacactacaaagatactcctcaagaagagcaaccccaagacacataattttcagatttaaaaagtatgaaatgaaggaaaaaatgttaagggcagccagagagaaaagtcgggttacccacaaagggaagcccatcagactaatgggatctctcagcagaaaccctacaagccagaagagagtgggggccaatattcaacattcttaaaaattcttaaaaaaagaagaattttcaacccagaatctcatatccagccaaactaagcttcataagtgaaggagaaataaaatcctttacagacaagcaaatgctgagagattttgtcaccaccaggcctgccttagaagagctcctgaaggaagcactaaacatggaaaggaacaaacagtaccagccactgcaaaaacattccaaattgtaaagaccatcgacgctatgaagaaactgcatcaattaacgggcaaaataaccagctaacatcagaatgacaggatcaaattcaaacataacaatattaaccttaaatgtaaatgggctaaatgctccaattaaaagacacagactggcaaattggataaagagtcaagactgtgctgtattcaggagacccatctcacgtgcaaaaatgcacataggctcaaaataaagggtcgaaggaaaatctactgagcaaatggaaagaaaaaaaaaagcaggggttgcaatcccagtctctgataaaacagaatttaaaccaacaaagatcaaaagagacaaagacagccacaacaagaagagctaactaccctaaatatatatgcacccagtacaggagcacccagattcataaagcaagtccttagagacctacgaagagacatagactcccacacaataataatgggagactgggagacaccccacagtcaatattaaacagatcaacaagacagaaggttaacaaggatatccaggacttgaactcagctctagaccaagtggacccaatagacatctacagaactctccacccctaatcaacagaatatacattcttctcagcaccacattgcacttattttaaaattgaccacataattggaagtaaaacactcctcagcaaatgtaaaagaacagaagtcacaacaaactgtctctcagaccacagtgcaatcaaattagaactcaggattaagaaactgactcaaatccacacagctacgtggaaactgaacaacctgctcctgaatgactactgggtaaataacaaaatgaaggcagaaataaagatgttctttgaaaccaatgagaacaaggacacaatgtaccagaatttctgggacacatttaaagcagtgtgtagagggaaatttatagcactaaatgcccacgagagaaagcaggaaagatctaaaatcgacaccctaacatcacaattgaaagaactagagaagcaagagcaaacaaattcaaaagcaagcagaaggcaagaaataactgagatcagagcagaactgaaggagacagagacacaaaaagccgttcaaaaaatcaatgaatccaggagctggttttttgaaaagatcaacaaaattgatagaccactagcaagactaataaagaagaaaagagagaagaatcgaatagatgcaataaaaaatgataaagggatatcaccaccgatcccacagaaatacaaactaccatcagagaatactataaactcctctacgcaaataaactggaaaatctagaagaaatggataaattcctggacacatacaccctcccaagactcaaccaggaagaagttgaatctctgaagacaccaataacaggtactgaaccaaggtttggaccaaccaaaaatgtccaggaccagatggattcacagttgaattctaccagaggtacaaagaggagctggtaccattccttcagaaactattccaatcaatagaaaaagagggactcctccctaactcattttatgaggccagcatcatcctggtaccaaaacctggcagagacacaacaaaaaaagagaattttaggctaatatccctgatgaacatcgatgtgaaaatcctcaataaaatactggcaaaccaaatccagcagcacatcaaaaagcctatccaccaaaaccaagtcagcttcattcctgggatgcaagactggttcaacatacgcaaataaataaatgtaatccatcacataaacagaaccaatgacaaaaaccacatgattatctcaatagatgcagaaaaggcctttgacaaaattcaacagcctttcttgctaaaaactctaaataaattaggtattgatggaacgtacctcaaaataataagagctatttatgacaaacccacagccaatatcatactgaatgggcaaaacctggaagcattccttttgaaaaccagcacaagataagaatgccctctctcaccactcctattcaacatagtgttggaagttctggctagggcaatcaggcaagagaaagaaataaagggtattcagttaggaaaagaggaagtcaaattgtctctgtttgcagatgacacgattgtatatttagaaaaccccattgtctcagcccaaaatctccttaagctgataagcaacttcagcaaagtctcaggatacaaaatcaatgtgcaaaaatcacaggcattcctgtacaccaataatagacaaacagagagccaaatcatgagtgaactcccattcagaattactacaaagagaataaaatacctaggaatccaacttacaagggatgtgaaggacctcttcaaggagaactacaaaccactgctcaacgaaataaaagaggacacaaacaaatggaagaacattccatgctcgtggataggaataatcaatatcacgaaaatggtcatactgcccaaggtaatttatagattcagtgctatccccatcaagctaccattgactttcttcacagacttggaaaaaaactactttaaagctcacatggaaccaaaaaagagcctgcatagccaagacaatcctaagcaaaaagaacaaagctggaggcatcacgctacctgacttcaaaccatactacaaggctacagtaactaaaacagcatggtactggtaccaaaatagatatatagactaatggaacagaacagaggcctcagaaatgacaccacacatctgcaaccatctgatctttgacaaacctgacaaaaacaagaaatggggaaaggattccctatttaataaatggtgctaggaaaactggctagccatatgtagaaagctgaaactggatcccttccttacactgtatacaaaaattcgctcaagaaggattaaagacttaaatgtaagacctaacacctagaagaaaacctacacaataccattcaggacataggcatgggcaaagactttatgactaaaacaccaaaagcaatggcatcaaaagccaaaatagacaaattggatctaattaaactaaagagcttctgcacagcaaaggaaactatcatcagagtgaacaggcaacctacagagtgggagaaaatttttgcaatccacccatctgacaaagggctaatatctttgtagaatctgcaaagaacttaaacaaatttacaagaaaaaaacaaacccatcaaaaaagttggcaaaggatatgaacacctttacactgttggtgggagtgtaaattagttcaaccattgtggaagacagtgtggtgattcctcaaggatctagaactagaaataccatttgacccagtgatctcattagtgggtatatacccaaaggattataaatcatgctactataaagacacatgcacacatatgtttattgcagcactattcacaataccaaagacttggaaccaacccaaatgtccatcaacgatagactggataaagaaaatgtggcacatatacaccatggaatactatgcagccataaaaaaggatgagttcctgtcctttgcagggacgtggatgaagctggaaaccatcattctaagcaaactatcatgaggacataaaaccaaacactgcatattctcactcataggtgggagttgaacaatgagaacacatggacacagcacaaggaacatcacacaccggggcctgtcaggggttggggggctgggggagggatagcattaggagaaatacctaatgtaaatgatgagttgatgggtgcagcaaaccaacatggcacatgtatacctatgtaacaaacctgcacgttgtgcacctgtaccctagaacttaaagtataataaaaaaaaaagaaagaaaagaaaagaaaaaaagaagtggcttttacctcccaccacgattctaaggcctctcagccatgtggaactgtaagtccagttaaacctctttttcttcccagtctcgagtatgtctttatcagcagcgtgaaaatgaactcatacacacacacagaagtatatctttacagttcccaggatggactgcaagtttggctctaaatttcctttgcctggccaaaacaaaaagaacacattttattataaggttcacaggcttttaattgaaaaaaaaaaaaaaacagttattcaacatgaaaactctttttcagacaaagttctacaaagtctatttctcacaagtaggactttggcaaggattgtgcaactgacaattcaaagtcatttattccgtggtgagaaaccggagtacagatctttaccagttaggggatgatccatgtgttttgataatcaccttggtggtgatcagactaaaggtctcctatgaaaaccattaacttcacactatagctgataggatgcactgaagcccttaccgctgactgcatggatgagagctaggccagagcattggcaggcagagtttataacatggggcaggaagcattgaaattctattccacctcttggtgtatcaggtcaggagtcaaatctcctatcttcctcttctggatcgctttcctcctgaagtgtctggcatagatgtctaggtatagcttggcatggtggtgtgtgcctatagacccagctacttgagaggctgaggtgggaggattgcttgagcgcaggagtttgaggctgcagtgagccatgaccacaccactgtactccaccctgggtgacagagcaagaccctgtctctctttctaaaaaaacaaaacaaaacaaaacaaaacaaaaagaaaaaaagatgtccaagtgtgttctggttactgctgctgactgctaaatttaacagctagaaaaaaaaagtttggactctatggatcataaatttggataagacacagagggaatggcttgtctcatgttcacaatgtctaaagtcttaaatggggagagtcgtcaaccaggggtaatttgagagttatgggctggaaccaccttgaggcatctttattcataggctagcaattgatgttggctgttggctgggacgtcagctggagctgtcaactgggagacctgtctgatatcccagcataggacactagacctctcacaaagtggcgagggctccagaagcaagtgtcccagctgacaaaaaaaaaaagtttcatagccttctacgacatgcacttagaagtggtaatgtgtcacttccagccctttctgttgattacaagtgagtcatgagtccactcagtttcaagggagagggcccagatcccatatctccatgggaagagtgccaagaacaccttgtagaaaagcttgtggataggagatattggtgtggtcatccttggaaaacacaatcttccacagtgtgcaagagaggcttcctgctctgcattgtggcttaattattgttgaaataccaggcaagaaaaggtaggatccagggaaacaaatgtatccaggatgttttggtctgccttttctaggaaaaaaggaaaccctcaaagcctctgagactaggtcaccccagtgggactagaagttccacgtggaaccttctgcaaactcctctgcaatagatggactgtgcattttccaaatgtgtctttacatcatctcctttctgaccttgggaagatagtaactgactgtggagtaggaaatccagaaccctgattgttttaaaatatattacagatacaagtaatgaaaagaaggctcctaaggaaaatatagatccgtaatggctttgtcccaaaagctattaggctataatattttattacaaaggctatatttattacaaaatgtatttaaataaatacttaatatttatttaaataaataatatttattttattattaataattaatatttaaataaatataatatttcttacaaaaattaaaaactgggactttatatttaaaggtgagacattaaattctttttaaaaagtgcacatgtaagatttttttctcaagtcaactgaaccagttcatcagtttcactatgaatgtgttgatgcaccaagtatttactttctagaacatctagtagacgtcactaaaaaattataccaagttgaaaaatgtcactgaatcagaggcatgtggttggttatgtagtcatctgtgccagctgggaatatagcaataccaaatgaacagcactgccatgttcccttgctgggttccagcaaaaacactcatcttcactgtcgtctctctttgagacctcttagagaggactttgatgagtgtatggtcttatacacactagtctaacaatacacatttgctcaaggattgggttacgagcaggctcttcacctttcaggtgaacaattacacatcaggagaaggatggaagtttcctatctatgaacaaatatttcccaaagcaataatctctttattcaactcacgtaatgagaagtcagttcctctgtgccataaagcaatctctttgaagtcttcagtaatgttgagggttttaatgttgagggctttatcaaactaaacattcaagtgtcttaggctttccccttagttctttgagcaaattggttgctatgaagtatgttagtgttccgaatgaatggatagcatgatgggggatgtctaggtggttccagaatttagattgtggtttccataacaaagtgtggcagtattggcattagggaagagaaatgacttctaaataacttctcaaagtttactaaagccttaagaatgtaggaaatggccacaaaaggaggtatacaaaacattcctctgtctttattcctctttatacaaacgtattcactttcccatcagcaagtcataaaatacttctcatcttgcattacagtaggacacctactataacaataggctacaaaatgtataatggctcaaacacctagcagtttatttcttatttacttaacaatgctgggtccacgaacatgttgatatcataaccctcttttatgtggtcactcagggactcaggctgttgagggctctgtcatcatgaatgggtggcttccaaggtcatcctaagagttatctccattctaaccagtccagccagaaaggcagaacaggcttggaggagcaaagatatccatggatagatcatcctcttaaagtcattgtagcattgtgatagggctcttttcacaaaacaaggatacaatccatgttttcaagattttacattttagtagccacaatacaaagaagctaagatagtcttgtgatttgtcatcatggctgttaatggagagtgttttaaaagcactgatatcagggctccaccctcagcttccaatcatcaggtttggatggaacctgacagctgcatcttgtaaaactccacgggagattctgagagacagccaagtttgagattcacaaaccttaagagaaaaaatctccttgaattgccataacaaactaccagtctaataaaacatacatattcctttgactactcttaaatgaaacaaataggtaataatttacctgtacatataattaaaaatgtaatgtattttataaccatcatataaaggagaaattatattaaactatattaaaataaaatgatatatatttcaatgcagatgcccaagcttgactaaagtaggaaacataataaagtaataaaatgcttatatgtacttataatgaatgtttggctatgagaagatgatcagaggctttttgtccaagaagtacaggcatatgaaaaagtagagcaagagatggacaccagactgagaaaactaaaaactaaccaacctatttgtgtatgataaaaggagagaaaaaaaattgattagtaaaaatatgctaccgtgtggtctgaatgtttgtcccttccaaaactcatgttgaaatttaatcccccatgtggcagtattgagaagtgggacctttaagaggtgattggttcaagaagattctgcgctcatgagtggttgaaaccattcacggaataacgggttaatggatcaatgagttatcccaggagtgggactggtagctttataagagaaggaagagagacctgcgctagcacactcagcctccttgccatgtgagccatctcgggaccccgcagagtccccagcagaaagaaggccctcacccaatgtgtccccttgacctaggatttctcaacctccataactgtaggaaatgaattccttttctttgtaaattatccagtttcaggtattctgttataaacaacagaaaatgggctaaaacacacgccaagaaaattgaaagactgtggaagtgaaagaagaataaaaaatgaagtaattttgcaaatgagtctggctttattataagtgtattgtcaaagtgattccctttgttataagatgaaaaagagccaaaatggatagaaacatttatcctatcttaatatcccactacattcaaggcaaatattcagtcagtctctagaacttaacaaggccttggagatgatatcctttggatgagcaatggagaagacaagatggattggaaaaagaaacaaacaaacaaaaaaacatgtaagaggctatcaggaaaactctggagacgatgtcaatgtaaaacagatacaaacctcaaaataattttaatatgtacattgcacaaaaatggacttcttatcatgttacaaatttattttaaaacatataggaggcaacaacaatatttcattgatgtgtactattttataattgttggctctaattttatttagtagagtcttacatctcctctctacatatttacatccggttgtattgtttttgtagtacctgttcaactgcactgaaatcatttcaattaaatgcatggttgaaaagctgacataatacattcaattgccaaattgcataaagtgaataagctatttaataatttggagaaaatatattccatttaaacttccttctctataaccagttcatacactggaccttttgggcacagccatgtgaaagcttttcaaaacttttcactacttttttttgagacggagtctcactctgtcacccaggctagagtgcagtggtgcgatctcggctcactgcaacctccacctcccaggtccaagtgattctcctgcctcagcctcccgagtagctgggattacaggcacctgacaccacacctgactaatttttgtaattttagtagagacaggatttcaccatgttggccaggatggtcttgaactcctcatctcaagtgttccgcctgccttggcctcccaaagtggtgggttacaggcatgagtcattgcacccagccactatagatttttatttgtagtagatttagttcagtagggcatttgagaggcataggaactggaacaaatccttcattgtttgggactgccttgcacattgtaggacatccagtacagtcccttccacgaagtggcagcagcagcctcgggctgtccaaacaccctgttagggacagtgccactcccggaaccacagaagaaccacaatcataggagaagcatacaacaaacatggtgttcatcggacggtcagcttcagtggcacttttgagcaagcacactatttggttgtagaatgagtccctggcagggcacccgaaaggctgatgtgtgactcatccctagtacctctgagaatgctgaaatctcctccttctccaagctgagtggcagctttctggtccagactacttaaattgtgacttgaggaattttcttcactttttcctttgagtcattctgtttttgatcaagatgcgatagaatgaaggattctttcagggtctgctgtcacatccttcctttgggaacagatagaaaatctggcctggcttgaagagtttgtcctcaaacatctggtaagccataggcatcagcatctcttacttctccagagacatttagatgtgtcccccatctccatggaggcccatggcaaggggacccctagttgtactagagcttccttctgggcacagccacactgcacaggcactgcagtgtacagcacagctcctccagcttccggaagactccattatctagaaaagtgctgagaaatggagaagggggttcctctggggtggagatggaaagaggaaaaggaagggagaatgtccaagcaaaatgagagaaggtgagggacagcagaattgagagtgagggagagggagagagcaagagactgggttgagatgtcttgtacatgggggtgtcctcgtggcttcactcccctttcgtttgtaccactccgtttccccagactcttcctaaatatccccttattggctacaagatccagattgcctagaagctcagatttgctgacagcacctcaagactggttaatgacatatagattttttcttgtttccatctttgggatttggagcaatctccagagttgcaaaagcctaccttgttgcaataatatttttgagggtccacactgtctgctcacttgctctatgctgctgggcacagtggtgtggccatccagggtgggagagaccacctgggatatggaaattaccagcagccctgttttgctcttgcagtggttgccaaagcacacgaagcattctgggaatctgaatttctgttgtttctcccaaatggcccctgaagggcaacaggcagcacacatggaccacattttgccagaaaagtccagaaagaagatggctgctgttgtcaactattggcatttcaacaggacctatttttatttctcagtatatttgcacaactaggaagcatctgctgacaaggatgagatgataagctgtattgatgccaaaacaggactagaaaaacaggcttgttctgcaaatggcctgcatcctgagagtccgtgttaagaacattctctctctgcaggcaatatctgctcttaaaacattaacacttgaaaggaagtaaggcttggaatccttagcatagttatgcacacactaggttctcagtaagtgcatgctggatgaaggaacccacacgcaccttaaggaggatggaacattggcagcccaagaaaaaagtcactccccagaggccgagtgccatgtctttgacctccattggcaaggtcatggtgaaatgctgtctcttggggtcgctgaggtcccagcacggtgtgccttcacaccgtgcaaacaataagaagagaagagaggcacacatgtcacctttccaggcagggacatccaagctgtttgagtctccattctgtgatttccttgaacaagaaagggattattggtgaatgaggccagcatcctgggtgggaacaaaaaaggaaattggtagaaagtacatcatccaagttggtctaactcagccctgtggcttgggcgggttcctaggatatccagccggaggcagaggctctaggggatgtgaggctgcactgggtatgatgacacagaggactgtgagaggcagagaaaaggggagaagagctgaggccctcctagccctaacctcccagctcacaccctctgctatctgccacctccaggccaacaccacggccatgggaggtttgcccatttttctgatagacacattctttctctaccaacagcccacatggaacataagtgaagtaattcagcagtttccttcttaatctgctgtctctctgctgcttactcattctgtgctgtttgctttagatcaaggtgccccagggtaccaaaagctacattatcctattcaactgcatggaaaccaagatcccgaccattctggtttgttgttattgttacgttagttttaaaccttagccattttccattctgggcagttacaactctgtttactatgagttcctctgtgtagatgtttctcccatacccgacctacaaactgctacgtgagcagtaacttgtcaccatgttatccgcataaagtcagggattgataaacttgggagcctttagctcttgaaggtgctgatgaggaaggggagaggggaggagaaggaatttgtttgtgagatggtcagtgggctgaatagtggtatgaccaaaaagtgttactataaatacacataatctggaaaacaaaaatactgtcatctcctccctgcaaggcgctgtgattaggcaggatggagcttattgctgcagtgttagaaaatgcatccttgtagtcccaaaaaagcctgttccatcctgcccatctcttgttctcaatacttcttacttcacggtcactcattttcttcctgtccttgggttgaggtctcaacttcttgtaatagaattttttcagttataagcgacagaaatccaactcacactatcttaagcaaaaaggggattcactgatacgtgtgttaggaaggatatatgaaagctcaaaggtcaagtgaaaaaaatgtaagaacgaagaccttggtggggtgtggtggctcacgcctatcatcccagcattttgggaggccgaggcaggtgggtcacaaggtcaggagttcaagaccagcctgcccaagatggcgaaaccccatctctactaaaaacacaaaaattagccgggtgtggtggcaggctcgtgtattcccagctacttgggaggctgaggcagagaactgtttaaacctgggaggcagaggttgcagtgagttgagatgtgccactgcactccagcctgggcggcagagtgagactctgtctcaaaaaaaaaacaaaaaaaaaaaactaagaccttgtgactactacctactacctggatctctctctctctctctctctctttctctctctctctctcacccccccacactatcacctctccttgttgtcagtagtttcctttctgcagacaggagttccatgtgctgggaagagggccattcagagcctcgtattctcatcctctcagtttagcaatgcgagaagaaaaaacttttgtctcacaacctcatatacaatctcagaaataggctcttattttctggcttgaggctgtccagaggcaccaggcactgtgatggtctcactttctgtcctggatctatctgtggggcccagacagcaggatctatttcagaaaataggcgagagaggaaaaacttatgtggccgtaaaaaacagcggttttcaagtggcctatccccatccacatcataaacttttctgctgaggcttaaaagtgcttagacttcaaacttgatctaaaacagaaatcctacctggaaaaatcaccttccagtcacttagccaatcgctcatcactctctgtctctgaatctcaccacgagtttcacacgtctcccgagagagcgtctcctttatcattaccacatgtggagtcagcagattgggtttcatctaagctctgcctacctactatccgagttccctagcaggttagtgactttctctgcctttttgtttcttgatgtgaaaaggaacaacagtggctgctcacagggttgtcagttacatggtcaattacattacatggacaattcatgatggtgtcagacctgtctgagaaaaggtggcccatgttaccatgacaggccccaaggctaacagcccctttctgggtcatggctcatacaaaggagccctccttggtttgctggtgctctgagacttgcaaatgcattaggaattttacactgtaacccggttttaaatgggtccagagcatccccattgctagactactgtgctgaggaagggcactggctcattgttacatcccatgaacactctgggtctcctagacctcatcctctttgagcttctctctctcactctctctctgcgcattcaggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgaggtgaggcatctgtgggcttcctacaggagagacataaagaaaacatgcccccaaacctatgtgacagctggccgggaaggactggtgcctgcatatggagagtgcacttgtgacagttcctggcatagaataagcataaatgctataggaggacagaagagagaggttttaaatctgcgagggtcacagggcaagtgtcagagaaggcatagaggaagcgatacttacgcttggtttaaagcatgtgctttggggcagtggtttagagattgggtgcagtgtgcaaataggaggagggggccaatcaagagaagtcagtgaatgtacacatcccctgaagacactgggtacatctgttatggtccctgtgtacactcggatattctaaatgagagaccctggggccagatgcctcacggagagtcaatgttgactccttcatacctgttgaaccccttcttgtcacccccattgccattaccctagtctaagccaccatcattttctgttggacttctgtgccagtcactggatgtgtatccctgcctgtaatctagatcctttctagatcattctccatgatgtgtccagagtgatcattctagaaccaaatctgatgtcatcctcctgcttaaaacccttaagttctcattgcttttagataaaatataaaccctttcactttgtttttattactccttatgattcagcccttacttagcttacaaggacttcctcatgtctgagctctagccataatagctgactgttggatgagtcatcggggggaggttcttcttcaggctgcagattggcggtcagccctcctgacctcagctgggctcactcacacatctgggaggcagctgcctgaggccagggtgactctgctctatgcctcatgtctcctatcctccttgtacccacatgttagctgggacatgttctcatcgcaatggcagaggcatgagaaagtgatcctgtttgggcaagtgcattttactcttttctttcactcatgtccactgacatttcattggccaagcaagcatgtggggtgtccagagtcaaagagggacatgttgccggggtcatgactatttctgagcaataatctaacctatcatagctcagatctcactccttgcaggaagcatctcctaatccttaagacctagttggaacttacactctgttcttccctatggttactgcccattttactatttttctgcccatttagactgacaactctgtgagccatcattgcaatatggcttgcttacccaatatcacctagcatgatgtagagcacatagtaaatactcaacaaatgttttctgtaagagctaaggaaggacatcagcagacaaaatagagtctttctatgccaaaaggtgaaagtggtctcatttccattccattgtcctgtaatttggctagccaaaagcttagtggataccattcccataagtagacctcatctgcagggaagagatggggagactgctgggttatttattgcagactccagcaggaccacaacaatgattcaaagtgatgacactagaaggacaggtgccaatgggtgatgaagccacaatgacgatgtcaagtggtatcactaaaaggacaggtactcatgggtgatgaagcctacatgaccttgggcttacaccaggaccctaggaatgaaagagattccttctcttagtggctatgagggaaggcctatgatgtcttaaggaggttggattcgatcatttttaagggaccttcaaatccttagaccgtgattcaaatggtaagaagtttgctccattaaactcccaggcaccaccctttccggagacaacctttaaaaattattagctcctgggaaaatagatgtatctcattttctttgtatccctcacagtgccatatgtgaagacttacatatggtatatgcccccaacattactactgagtgagtgaacaaagaaatgagggaatgaatggatgaacaaatggctcatggcaggcttttccctgccctgtgaattcggcttctacttggtaagaaggtgattgcaatctctgttctcagggttccccaaggattcctgtgtaacagatgaaggaatcacgtctgtcaggtagagtaggtgactgcattcagagtatacatttccagtctcccctgtctttttcttttgatgagtattatagagagggaagccatgagtggattagatgccaaaatccctggctgagagaataaccttaccctggaggaaaacatattagctttgactctgagctgggaatttcggtgatgttgtagattcaatgcattgcagttgggtgtttttatttgttgaaaggaattgctgaattttcaaatccattaactgcttgtcagcattagcaagcctaattagttaatactaagtaaatttgcactaaatatacaaccctggctgattttactggccacgtctggcagagggcagcagcagggagaaagctctgtagagtttctgttggaatcgtgtgaaaagctggagaggttgcttctctttctctctctctctctttctctctctctctctgacacacacacacacatacacacacacacaattgtaataataataatattttggttcctcactaagccaatctaaatcgcagaagtctattttgttaagtaagcttggccccagccatatgttgctactcagagaatttaatatcagatttcatctgactgtaaacgtgaatcatcaggttgcacaaggaacacagtggcagatccagggggcatttaacttttatagcattttaaatgaaaaaaaaaaaaagtaacacttaaacaagtaatttagatcatgctgtaggccctgaatagctttgatgttgtgttttcatggcaagtctccaacttgagctgaattttcccctactaaaaatgcaaatttttctaagaccttctcttggtcctgctactcatgatacatatttcttttaaaagaaatttcaaactagataatagttgttctcctccccacccccgccaccagtagtgtggtggggcagcagagttgtggctagtggaggagagcagaggaggagagtagggaaaggagaatgccatttgcctacatttccctctgcccatttcccgctgcccatttccccccttgttttcctgaacgtgaactgagctctgggcactgttttaggcctagcaggggacaggataaagcctgcttctctaggaattcgcactgagggtgtgagtgtgtgcacgtgtgtgtttggaggcgggagaataaacacaaataaataaaaaggagaatttcaggcagtgataagagtgctgagaaaaacagaacggtgtgaaagaggaaggctgagcctgcagaggcttgaggctgctgccactgggtagcggtaggcctttccgaggaggcggcatttgaagaccggaggaaggttcatcccagcaagtaggaacagcaagtgtaggtcccctaagtcttgggggagcttagttcctttaagggcagcacaaaaatcagtgtggctccggagagcacattaggggagagaggcaggaagagcttggagacatggatggaagctggaccagttgggccttgttgaacatggaaaggcatttagatcgtattctgagttaaatgggaagtgacgtgagagatttaacaatggagcgtcttgaactgctttactcatttaaaatacccactcctgcttggctgaatatctcatgttgtctttttagaagctttggcgatcctatttgaatgcatttaggtcctattggaggggaataggatctcatttgaggccacggaggtccatggaagtcacctgcatagcaaataccctgaaagtggctgcagggagagtgtgagggtgggaccgccctggtaggaggtggaaaatgaaaaacacacggccatgagttccagattagggcttctgaaagccctcagctttcccagctcccatcctaaagtgggtctttaaacaggaagaaagaaagattgctaagtgtctttggagttcctcttccttccccttctagggatttcagcactcctggggctcgggttggctctaaagtagtcctttctgtgtcttcccacctacagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctgagagctccctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcattccccctataggaatacaaggggcacacagggaaggcagatcccctagttggcaagactattttaacttgatacactgcagattcagatgtgctgaaagctctgcctctggctttccggtcatgggttccagttaattcatgcctcccatggacctatggagagcagcaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggccctggtgttgcatttagccctggggcatggagctgaacagtacttgtgcaggattgttgtggctactagagaacaagagggaaagtagggcagaaactggatacagttctgaggcacagccagacttgctcagggtggccctgccacaggctgcagctacctaggaacattccttgcagaccccgcattgccctttgggggtgccctgggatccctggggtagtccagctcttcttcatttcccagcgtggccctggttggaagaagcagctgtcacagctgctgtagacagctgtgttcctacaattggcccagcaccctggggcacgggagaagggtggggaccgttgctgtcactactcaggctgactggggcctggtcagattacgtatgcccttggtggtttagagataatccaaaatcagggtttggtttggggaagaaaatcctcccccttcctcccccgccccgttccctaccgcctccactcctgccagctcatttccttcaatttcctttgacctataggctaaaaaagaaaggctcattccagccacagggcagccttccctgggcctttgcttctctagcacaattatgggttacttcctttttcttaacaaaaaagaatgtttgatttcctctgggtgaccttattgtctgtaattgaaaccctattgagaggtgatgtctgtgttagccaatgacccaggtgagctgctcgggcttctcttggtatgtcttgtttggaaaagtggatttcattcatttctgattgtccagttaagtgatcaccaaaggactgagaatctgggagggcaaaaaaaaaaaaaaagtttttatgtgcacttaaatttggggacaattttatgtatctgtgttaaggatatgtttaagaacataattcttttgttgctgtttgtttaagaagcaccttagtttgtttaagaagcaccttatatagtataatatatatttttttgaaattacattgcttgtttatcagacaattgaatgtagtaattctgttctggatttaatttgactgggttaacatgcaaaaaccaaggaaaaatatttagtttttttttttttttttgtatacttttcaagctaccttgtcatgtatacagtcatttatgcctaaagcctggtgattattcatttaaatgaagatcacatttcatatcaacttttgtatccacagtagacaaaatagcactaatccagatgcctattgttggatactgaatgacagacaatcttatgtagcaaagattatgcctgaaaaggaaaattattcagggcagctaattttgcttttaccaaaatatcagtagtaatatttttggacagtagctaatgggtcagtgggttctttttaatgtttatacttagattttcttttaaaaaaattaaaataaaacaaaaaaaaatttctaggactagacgatgtaataccagctaaagccaaacaattatacagtggaaggttttacattattcatccaatgtgtttctattcatgttaagatactactacatttgaagtgggcagagaacatcagatgattgaaatgttcgcccaggggtctccagcaactttggaaatctctttgtatttttacttgaagtgccactaatggacagcagatattttctggctgatgttggtattgggtgtaggaacatgatttaaaaaaaaactcttgcctctgctttcccccactctgaggcaagttaaaatgtaaaagatgtgatttatctggggggctcaggtatggtggggaagtggattcaggaatctggggaatggcaaatatattaagaagagtattgaaagtatttggaggaaaatggttaattctgggtgtgcaccagggttcagtagagtccacttctgccctggagaccacaaatcaactagctccatttacagccatttctaaaatggcagcttcagttctagagaagaaagaacaacatcagcagtaaagtccatggaatagctagtggtctgtgtttcttttcgccattgcctagcttgccgtaatgattctataatgccatcatgcagcaattatgagaggctaggtcatccaaagagaagaccctatcaatgtaggttgcaaaatctaacccctaaggaagtgcagtctttgatttgatttccctagtaaccttgcagatatgtttaaccaagccatagcccatgccttttgagggctgaacaaataagggacttactgataatttacttttgatcacattaaggtgttctcaccttgaaatcttatacactgaaatggccattgatttaggccactggcttagagtactccttcccctgcatgacactgattacaaatactttcctattcatactttccaattatgagatggactgtgggtactgggagtgatcactaacaccatagtaatgtctaatattcacaggcagatctgcttggggaagctagttatgtgaaaggcaaatagagtcatacagtagctcaaaaggcaaccataattctctttggtgcaggtcttgggagcgtgatctagattacactgcaccattcccaagttaatcccctgaaaacttactctcaactggagcaaatgaactttggtcccaaatatccatcttttcagtagcgttaattatgctctgtttccaactgcatttcctttccaattgaattaaagtgtggcctcgtttttagtcatttaaaattgttttctaagtaattgctgcctctattatggcacttcaattttgcactgtcttttgagattcaagaaaaatttctattcttttttttgcatccaattgtgcctgaacttttaaaatatgtaaatgctgccatgttccaaacccatcgtcagtgtgtgtgtttagagctgtgcaccctagaaacaacatattgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaattttctcaacctatttgatgttcaaataaagaattaaactaaa

SEQ ID NO: 2 - protein, 595 aa, Homo sapiens [sequence omitted]

SEQ ID NO: 3 - protein, 595 aa, Homo sapiens
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYGLLLEMLDAHRLHAPTSRGGASVEETDQSHLATAGSTSSHSLQKYYITGEAEGFPATV

SEQ ID NO: 4 - protein, 595 aa, Homo sapiens
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLSDLLLEMLDAHRLHAPTSRGGASVEETDQSHLATAGSTSSHSLQKYYITGEAEGFPATV

SEQ ID NO: 5 - protein, 595 aa, Homo sapiens
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLQCAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 6 - protein, 595 aa, Homo sapiens
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLCDLLLEMLDAHRLHAPTSRGGASVEETDQSHLATAGSTSSHSLQKYYITGEAEGFPATV

SEQ ID NO: 7 - protein, 595 aa, Homo sapiens
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLNDLLLEMLDAHRLHAPTSRGGASVEETDQSHLATAGSTSSHSLQKYYITGEAEGFPATV

SEQ ID NO: 8 - protein, 595 aa, Homo sapiens
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLHDLLLEMLDAHRLHAPTSRGGASVEETDQSHLATAGSTSSHSLQKYYITGEAEGFPATV

SEQ ID NO: 9 - protein, 595 aa, Homo sapiens
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLDDLLLEMLDAHRLHAPTSRGGASVEETDQSHLATAGSTSSHSLQKYYITGEAEGFPATV

SEQ ID NO: 10 - protein, 595 aa, Homo sapiens
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPHYDLLLEMLDAHRLHAPTSRGGASVEETDQSHLATAGSTSSHSLQKYYITGEAEGFPATV

SEQ ID NO: 11 - protein, 595 aa, Homo sapiens
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPPYDLLLEMLDAHRLHAPTSRGGASVEETDQSHLATAGSTSSHSLQKYYITGEAEGFPATV

SEQ ID NO: 12 - protein, 595 aa, Homo sapiens
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPRYDLLLEMLDAHRLHAPTSRGGASVEETDQSHLATAGSTSSHSLQKYYITGEAEGFPATV

SEQ ID NO: 13 - DNA, 15 bp, synthetic construct
  source 1..15: mol_type = other DNA
ctctatgacctgctg

SEQ ID NO: 14 - DNA, 15 bp, synthetic construct
  source 1..15: mol_type = other DNA
ctctatggcctgctg

SEQ ID NO: 15 - DNA, 15 bp, synthetic construct
  source 1..15: mol_type = other DNA
ctctctgacctgctg

SEQ ID NO: 16 - DNA, 15 bp, synthetic construct
  source 1..15: mol_type = other DNA
ctctgtgacctgctg

SEQ ID NO: 17 - DNA, 15 bp, synthetic construct
  source 1..15: mol_type = other DNA
ctcaatgacctgctg

SEQ ID NO: 18 - DNA, 15 bp, synthetic construct
  source 1..15: mol_type = other DNA
ctccatgacctgctg

SEQ ID NO: 19 - DNA, 15 bp, synthetic construct
  source 1..15: mol_type = other DNA
ctcgatgacctgctg

SEQ ID NO: 20 - DNA, 15 bp, synthetic construct
  source 1..15: mol_type = other DNA
cactatgacctgctg

SEQ ID NO: 21 - DNA, 15 bp, synthetic construct
  source 1..15: mol_type = other DNA
ccctatgacctgctg

SEQ ID NO: 22 - DNA, 15 bp, synthetic construct
  source 1..15: mol_type = other DNA
cgctatgacctgctg

SEQ ID NO: 23 - DNA, 15 bp, synthetic construct
  source 1..15: mol_type = other DNA
cagtatgacctgctg

SEQ ID NO: 24 - DNA, 15 bp, synthetic construct
  source 1..15: mol_type = other DNA
ctcagtgacctgctg

SEQ ID NO: 25 - DNA, 15 bp, synthetic construct
  source 1..15: mol_type = other DNA
ctctcagacctgctg

SEQ ID NO: 26 - DNA, 16 bp, synthetic construct
  source 1..16: mol_type = other DNA
ctagaatgtgcctggc

SEQ ID NO: 27 - DNA, 16 bp, synthetic construct
  source 1..16: mol_type = other DNA
ctacaatgtgcctggc

SEQ ID NO: 28 - DNA, 72 bp, Homo sapiens
  source 1..72: mol_type = genomic DNA
aagtgcaagaacgtggtgcccctctatgacctgctgctggagatgctggacgcccaccgcctacatgcgccc

SEQ ID NO: 29 - DNA, 72 bp, Homo sapiens
  source 1..72: mol_type = genomic DNA
gggcgcatgtaggcggtgggcgtccagcatctccagcagcaggtcatagaggggcaccacgttcttgcactt

SEQ ID NO: 30 - DNA, 71 bp, Homo sapiens
  source 1..71: mol_type = genomic DNA
ctccatgatcaggtccaccttctagaatgtgcctggctagagatcctgatgattggtctcgtctggcgctc

SEQ ID NO: 31 - DNA, 71 bp, Homo sapiens
  source 1..71: mol_type = genomic DNA
gagcgccagacgagaccaatcatcaggatctctagccaggcacattctagaaggtggacctgatcatggag

SEQ ID NO: 32 - DNA, 13 bp, synthetic construct
  source 1..13: mol_type = other DNA
caagaacgtggtg

SEQ ID NO: 33 - DNA, 30 bp, synthetic construct
  source 1..30: mol_type = other DNA
cgcatgtaggcggtgggcgtccagcatctc

SEQ ID NO: 34 - DNA, 13 bp, synthetic construct
  source 1..13: mol_type = other DNA
catgatcaggtcc

SEQ ID NO: 35 - DNA, 31 bp, synthetic construct
  source 1..31: mol_type = other DNA
ccagacgagaccaatcatcaggatctctagc

(19) United States
(12) Patent Application Publication
(10) Pub. No.: US 2024/0254539 A1
(43) Pub. Date: Aug. 1, 2024

(54) DETECTION OF VARIANT ESR1 SEQUENCES

(71) Applicant: Saga Diagnostics AB, Lund (SE)

(72) Inventors: Miguel Alcaide, Lund (SE); Anthony Miles George, Lund (SE); Lao Hayamizu Saal, Lund (SE)

(21) Appl. No.: 18/240,453

(22) Filed: Aug. 31, 2023

Related U.S. Application Data
(60) Provisional application No. 63/402,515, filed on Aug. 31, 2022.

(51) Int. Cl. C12Q 1/6809

(52) U.S. Cl. CPC C12Q 1/6809

(57) ABSTRACT

The present invention provides a method for detecting variant nucleic acid sequences in ESR1, which are often found in low-abundance, using dPCR and patterns in multi-dimensional plots indicative of a particular variant sequence.

### TECHNICAL FIELD

The present invention relates to the field of methods for detection of variant ESR1 nucleic acid sequences.

### SEQUENCE LISTING

This application contains a sequence listing which has been submitted in eXtensible Markup Language (XML) format via the Patent Center and is hereby incorporated by reference in its entirety. The XML-formatted sequence listing, created on Oct. 12, 2023, is named SAGA-009-01US-Seqs.xml and is 325 kilobytes in size.

### BACKGROUND

The ESR1 gene encodes an estrogen receptor that regulates transcription of many estrogen-inducible genes that play roles in growth and development. The receptor encoded by this gene plays a key role in breast cancer, endometrial cancer, osteoporosis, and other diseases. This gene is reported to have dozens of transcript variants due to the use of alternate promoters and alternative splicing, however, the full-length nature of many of these variants remain uncertain. In recent years, evidence has emerged that tumor tissue DNA and cell-free DNA (cfDNA), and in particular, circulating tumor DNA (ctDNA), holds critical information in the ongoing, study, treatment, and monitoring of patients afflicted with breast cancer. Broadly, high levels of ctDNA are associated with poor overall survival in breast cancer patients. At a more granular level, within this tumor DNA and ctDNA, different variant estrogen receptor 1 (ESR1) sequences have been correlated to clinical outcomes, disease progression/regression, and responses to differing treatment regimes. For example, the presence of some ESR1 variant sequences in tumor DNA and ctDNA have been correlated with poor clinical effects when using endocrine treatments in patients with metastatic breast cancer.

### SUMMARY

The present invention provides methods and systems for quickly identifying more than one ESR1 variant sequences in DNA from a patient sample, despite the low abundance of the variants in the sample. The invention uses the insight that certain variant sequences detected by digital polymerase chain reaction (dPCR) assays have identifiable fingerprints in the dPCR data, allowing for simultaneous detection of many variants using only a few or even a single, detectable label to identify multiple variants.

Digital PCR is a method that partitions a PCR reaction into many smaller individual reactions so that each reaction partition contains zero to only a very few target sequence molecules. The partitioning of all molecules is random and generally follows a Poisson distribution. Partitioning is intended to make accessible a rare variant sequence among an abundance of wild-type sequence. The result is a potential increase in sensitivity. However, traditional dPCR often fails to detect the presence of low-abundance target nucleic acids in a biological sample due to various issues, such as tumor clonality and variants that are very close to each other, e.g., on the same template in a dPCR partition. The difficulty in detecting low abundance sequences is more pronounced when using traditional dPCR to simultaneously detect multiple variant sequences in a single assay, especially when those variants are in close proximity to one another, such as on the same dPCR amplicon.

The present invention recognizes that using multi-dimensional plots reveals patterns of variant ESR1 sequences, allowing for their identification using dPCR with fewer detectable labels than the number of variant sequences interrogated—even when the variants are at extremely low abundance in a sample. In particular, even when variants are proximal to one another, e.g., on the same dPCR amplicon, methods of the invention are useful to detect and quantify those variants through the use of probe sets.

In certain aspects, the present application provides methods for detecting variant nucleic acids. An exemplary method includes partitioning a biological sample comprising target ESR1 nucleic acid into reaction partitions and performing digital PCR in the reaction partitions. The dPCR reactions are performed using a wild-type probe set that includes a first optical label and a first variant probe set specific for ESR1 variant sequences that contains a second optical label. The first and second optical labels may be the same or different. Preferably, the first variant probe set includes a probe specific for a first ESR1 variant sequence, a probe specific for a second ESR1 variant sequence, and a probe specific for a third ESR1 variant sequence. Using a dPCR assay, optical signals are detected from the probes in each partition. The signals from the wildtype and variant sequence probes are used to generate a multi-color plot. For convenience, the invention is exemplified using two-color plots. Subsequently, the presence of one or more ESR1 variant sequences is identified based on a deviation in the plotted signal of the ESR1 variant from an expected wild-type cluster plot. That deviation may be a recognized pattern, characteristic of a particular variant sequence, whether in a subject or historically associated with a particular disease or condition.

In certain methods, a relatively small amount of a discrimination probe comprising a third optical label, where the probe is specific for a fourth ESR1 variant sequence. In such cases, the third optical label discriminates between the first ESR1 variant sequence and the second and third ESR1 variant sequences, which were all represented in the same probe set and thus had identical labels. In such methods of the invention may include generating a two-color plot using the dedicated channel for the second optical label versus the dedicated channel for the third optical label, wherein the presence of the first ESR1 sequence is based on a shift in the deviation from a two-color plot of the first and second optical channels and the two-color plot of the second and third optical channels. Thus, the invention, through use of the third label, makes use of multi-dimensional label detection to increase resolution between first, second, and third probes.

In certain methods, a first set of probes includes a probe having the first optical label and specificity for a first variant sequence and a probe having a third optical label and specificity for the first variant sequence. Such methods may further include generating a two-color plot from the first optical label and third optical label to identify the presence of the first variant sequence based on a shift in the deviation. In such cases, the third optical label may help discriminate the first ESR1 variant sequence from the second and third ESR1 variant sequences. Further, in certain aspects, the dPCR step includes an unlabeled, wildtype-specific probe that binds to the same genomic location as the first ESR1 variant sequence.

In certain aspects, the dPCR step further includes the use of a second set of probes. Each probe in the second set includes a fourth optical label, wherein each different probe of the set specifically binds to amplicons that comprise a different variant sequence, wherein said different variant sequences comprise a fifth ESR1 variant sequence, a sixth ESR1 variant sequence, and a seventh ESR1 variant sequence.

In certain aspects, the dPCR step further includes the use of a third set of different probes. Each probe in the second set includes a fifth optical label, wherein each different probe of the set specifically binds to amplicons that comprise a different, variant sequence, wherein said different variant sequences comprise an eighth ESR1 variant sequence, a ninth ESR1 variant sequence, and a tenth ESR1 variant sequence.

In certain methods, prior to the dPCR step, the method includes an amplifying step that specifically amplifies two different ESR1 sequences, thereby producing a first amplicon and a second amplicon. In preferred aspects, the first and second amplicons are less than about 65 bp in length. In some methods, the first amplicon includes the genomic locations of the second, third, fourth, fifth, sixth, seventh, eighth, ninth, and tenth ESR1 variant sequences and the second amplicon includes a genomic location of the first variant ESR1 sequence.

In some methods, the first amplicon includes an ESR1 sequence encoding amino acids 526 through 538 of SEQ ID NO: 2 (which is the amino acid sequence of ESR1). In some aspects, the second amplicon includes an ESR1 sequence encoding amino acid 380 of SEQ ID NO: 2.

In some preferred methods of the invention, a frequency for each ESR1 variant sequence is known, e.g., a relative abundance, or allele frequency, or mutant allele fraction (MAF). The frequencies may be determined or measured from a single sample or individual. Those frequencies for the variant sequences may be obtained at a first time for a subject, and the sample comprising the target nucleic acids for variant sequence detection are taken at a second and/or later time point(s). In some embodiments, frequencies of the variants are determined at one point, and those frequencies are used to design probes for dPCR assays for those variants at a subsequent time. For example, a tumor biopsy may be analyzed by deep sequencing or a multiplex mass spectrometry-based technology, such as the platform sold under the name MASSARRAY by Sequenom, Inc. Such an assay can be used to determine the MAF, or “frequency”, for each variant in a tumor. Those determined frequencies can be used in designing optical probes for use in multiplex dPCR. For example, variants with similar frequencies may be given a discrimination probe or a dominant (or very rare) variant may be given a dedicated probe color. With such a designed probe set, dPCR according to the disclosure may be used for identifying the presence or absence of one or more of the variant sequences, which may indicate a progression or regression of the diseased state—for example, by detecting minimal residual disease (MRD) or a change in those frequencies in response to a treatment.

In certain preferred embodiments, the fourth ESR1 variant sequence has the highest frequency. In some methods, the first, second, and third ESR1 variant sequences have a higher historical prevalence relative to the fifth, sixth, seventh, eighth, ninth, and tenth ESR1 variant sequences. In certain methods, one of more of the optical labels are selected from FAM, HEX, CY5, ROX, and ATTO550 (optionally, labels may use any one or any combination of FAM, HEX, SUN, VIC, TAMRA, ATTO550, Cy5, ROX, ATTO700, Cy5.5, Yakima Yellow, ABY, and JUN).

Any suitable biological sample comprising nucleic acid may be used including, for example, a tissue biopsy sample, a liquid biopsy such as a blood or plasma sample, a cellular sample such as a circulating tumor cell (CTC), one or isolated extracellular vesicles, a swab or clipping or other sample, or any other suitable sample. In certain methods, the sample comprises higher molecular weight target nucleic acids, for example DNA from fresh frozen tumor tissue, and the sample may comprise highly fragmented target nucleic acids, for example, cell-free DNA (cfDNA), circulating tumor DNA (ctDNA), and/or a sample from a formalin-fixed, paraffin-embedded (FFPE) tissue sample. In some methods of the invention, the first variant sequence comprises the sequence of SEQ ID NO: 3; the second variant sequence comprises the sequence of SEQ ID NO: 4; the third variant sequence comprises the sequence of SEQ ID NO: 5; the fourth variant sequence comprises the sequence of SEQ ID NO: 6; the fifth variant sequence comprises the sequence of SEQ ID NO: 7; the sixth variant sequence comprises the sequence of SEQ ID NO: 8; the seventh variant sequence comprises the sequence of SEQ ID NO: 9; the eighth variant sequence comprises the sequence of SEQ ID NO: 10; the ninth variant sequence comprises the sequence of SEQ ID NO: 11; and/or the tenth variant sequence comprises the sequence of SEQ ID NO: 12.

In certain aspects, the presence of one or more ESR1 variant sequences detected by methods and systems of the invention indicates the presence of a diseased state, e.g., breast cancer or a type of breast cancer. Alternatively or additionally, the presence of one or more ESR1 variant sequences indicates the presence of minimal residual disease (MRD) in the subject.

In certain circumstances, each set of probes may be specific for a tranche of different ESR1 variant sequences having similar relative frequencies during the diseased state. The present Inventors discovered that, while not necessary, dividing variant sequences into tranches of similarly-frequent variants helps provide more robust detection of the varied sequences in each tranche.

A sample assayed using the methods of the invention may include a small number and up to about 80,000 or even more total genome copies in a single reaction. Methods of the invention may be used with probes that bind to amplicons of less than about 100 bp in length or even less than about 65 bp in length during the digital PCR step. It is noted that this size is well under the typical size for cfDNA, allowing the methods to be used for these highly fragmented, yet critically important nucleic acids.

### BRIEF DESCRIPTION OF THE DRAWINGS

FIG. 1 provides a schematic showing target ESR1 nucleic acids, variant ESR1 sequences that occur at the genomic locations of the target ESR1 sequences, probe sequences for each ESR1 variant, and primers used in an exemplary method of the invention.

FIG. 2 summarizes the probes, ESR1 variants, and labels of the method of FIG. 1.

FIG. 3 provides two-color plots from dPCR reactions to identify variant sequences.

FIG. 4 provides two-color plots from dPCR reactions to identify variant ESR1 sequences.

FIG. 5 provides composite two-color plots from dPCR to identify ESR1 variants.

FIG. 6 provides schematic fingerprints or patterns for dPCR results used to identify certain ESR1 variant sequences.

FIG. 7 is composite two-color plots from dPCR reactions to identify variant sequences.

FIG. 8 provides two-color plots from dPCR reactions to identify ESR1 variant sequences.

FIG. 9 is composite two-color plots from dPCR reactions to identify ESR1 variants.

FIG. 10 provides two-color plots from dPCR reactions to identify variant sequences.

FIG. 11 shows probe reaction concentrations for example assay designs.

### DETAILED DESCRIPTION

The present invention provides methods for detection of nucleic acids, including structural variants and mutants, found in the gene encoding the estrogen receptor 1 (ESR1) nucleic acid (SEQ ID NO: 1). In one aspect, the invention provides an optimized method for the detection of several variant sequences in ESR1 simultaneously in a single dPCR reaction using multi-dimensional signals. As a result, methods of the present invention enhance the sensitivity for sample detection, especially in detecting the presence of low-abundance targets in the biological sample.

Methods of the invention are especially useful as they enable fast, accurate detection of more than one ESR1 variant sequences simultaneously that may even be low-abundance targets in a sample. Especially when the sample used include highly-fragmented nucleic acids, such as cell-free DNA (cfDNA), circulating tumor DNA (ctDNA), and/or nucleic acids from a formalin-fixed, paraffin-embedded (FFPE) tissue sample. In certain methods, the target ESR1 variants are associated with early tumorigenesis (a truncal variant) associated with breast cancer or a type of breast cancer, or as resistance variants associated with innate or acquired resistance to certain treatments such as endocrine therapies in breast cancer or a type of breast cancer.

The present invention includes methods for detecting variant ESR1 nucleic acids. An exemplary method includes partitioning a sample from a subject comprising target ESR1 nucleic acids into reaction partitions and performing digital PCR in the reaction partitions. The dPCR reactions may be performed using sets of probes specific for ESR1 variant sequences. Preferably, the probes are nucleic acid probes with optically detectable labels (e.g., hydrolysis or TaqMan probes). Probes within each set have the same optical label, but the optical labels may differ between probes of different sets. Further, in each set of probes, the probes are specific for, and thus hybridize to, different target ESR1 variant sequences. Additionally, for a reference, a probe for a wildtype ESR1 sequence may be included. Using a dPCR assay optical signals may be detected from the probes in each partition. The signals from the wildtype and ESR1 variant sequence probes are used to generate a two-color plot. Subsequently, the presence of one or more ESR1 variant sequences may be identified based on a deviation in the plotted signal from an expected wild-type cluster plot. This deviation may be a recognized pattern, characteristic of a particular ESR1 variant sequence, whether in a subject or historically associated with a particular disease or condition.

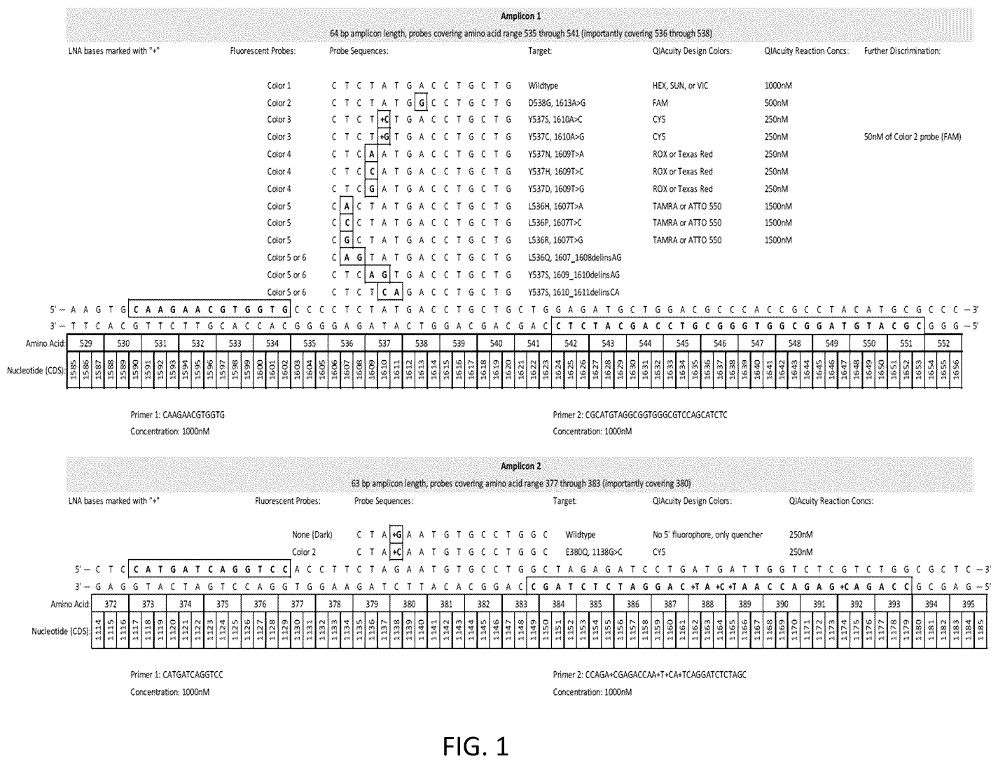

FIG. 1 provides an overview for exemplary sets of probes, including a wildtype probe, used to detect variant estrogen receptor 1 (ESR1) sequences in a sample obtained from a subject. The ESR1 protein regulates the transcription of many estrogen-inducible genes known to contribute to functions of growth, proliferation, metabolism, sexual development, gestation, and other reproductive functions. Critically, ESR1 expression and sequence variants are a hallmark in certain forms of breast cancer. Among these variants are recurrent variant ESR1 sequences, which are frequently found in patients newly diagnosed with metastatic and loco-regional recurrence of endocrine-treated breast cancer.

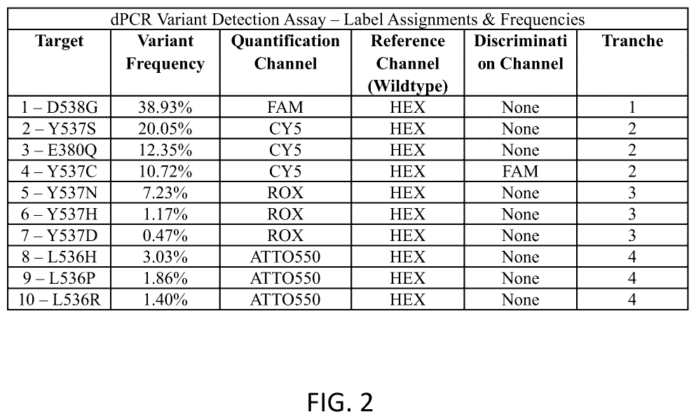

FIG. 2 summarizes probe sets from FIG. 1. As shown in FIG. 2, ten unique probes were created for variant ESR1 sequences in addition to a labeled, wildtype probe. The column labeled “Target” identifies the particular ESR1 variant sequence targeted by each probe, which is indicated using the resulting amino acid mutation relative to the wildtype ESR1 sequence. In this case, the relative frequencies of the variant sequences are known, however, methods of the invention may be used or include steps to elucidate the variant sequence frequencies. The “Quantification Channel” refers to the optical label, and thus, optical channel used to detect the presence of a labeled probe in a dPCR petition. The “Reference Channel” indicates the label, and corresponding optical channel, used to detect the wildtype sequence probe in the reaction partitions. The “Discrimination Channel” refers to a probe for one of the variant sequences in which an additional optical label is used, as will be described in greater detail, for further discriminating a variant sequence. Finally, as shown, the variants are separated into four different tranches, each using the same optical label. Tranche 1 includes the most prevalent mutant sequence. The remaining tranches contain variant sequences with fairly similar frequencies and/or corresponding genomic locations of the mutation giving rise to the variant.

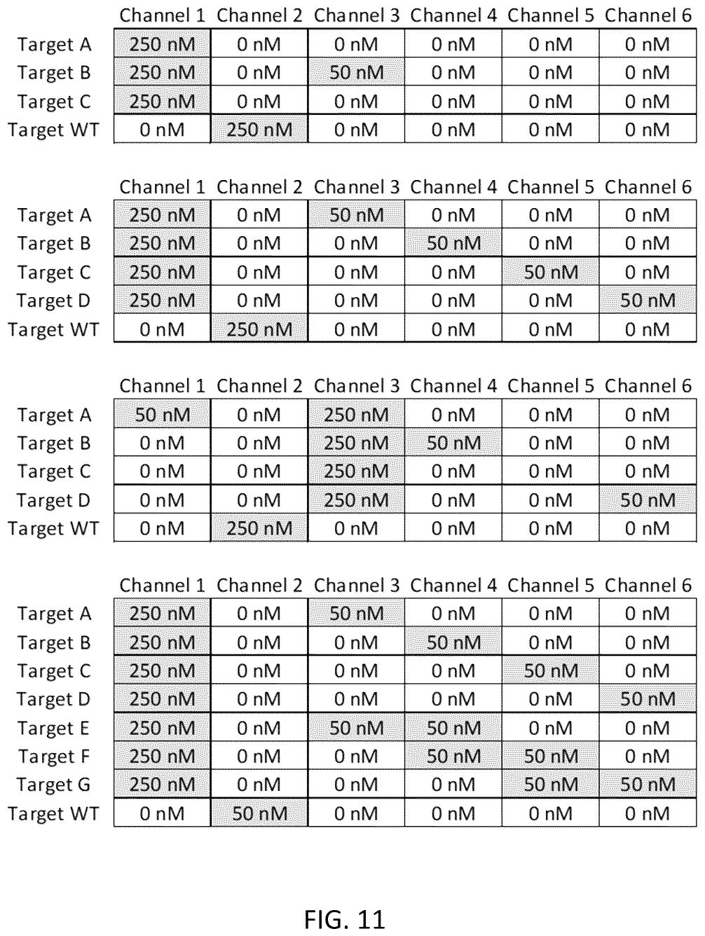

FIG. 11 shows probe reaction concentrations for example assay designs for certain embodiments. Targets may be detected in channels with target specific probes at higher concentrations (e.g., 250 nM) and discrimination may be facilitated with target specific probes at lower concentrations (e.g., 50 nM). In practice, methods of the invention may include the steps of: (i) providing a sample comprising one or more target nucleic acids, in this case ESR1 sequences; (ii) optionally, partitioning the sample into partitions such as droplets; (iii) a pre-amplification such as an asymmetrical incremental polymerase reaction (AIPR) in the droplets; and (iv) PCR within the droplets (optionally with primers that were also used in iii). In such embodiments, methods include both incremental copying (e.g., AIPR) and then exponential signal generation (e.g., PCR) all within the dPCR compartment (e.g., droplets), with all the ingredients added at the start. Such embodiments may use amplification and/or reaction step as shown in U.S. Pat. No. 11,066,707, incorporated by reference. Other approaches and embodiments may include (i) providing a sample comprising one or more target nucleic acids, in this case ESR1 sequences; (ii) optionally, performing a pre-amplification to selectively increase copy number of the target nucleic acid in the sample, in this case using the primers for each amplicon described in FIG. 1; (iii) aliquoting the sample into a plurality of subsamples, each in its own partition with the requisite set or sets of probes; (iv) conducting dPCR reaction on the subsamples; and (v) detecting the resulting optical signals from the probes, fluorescent probes such as hydrolysis probes that show the presence of a wildtype or variant sequence being produced in each partition. The optical signals form each partition are then detected and plotted to identify the presence or absence of the interrogated variant sequences.

In the exemplary assay outlined in FIGS. 1-2, the most common mutation, p.D538G is detected based on its optical label using a dedicated channel (FAM/Green). The next three most common mutations (p.Y537S, p.E380Q, and p.Y537N) are detected based on the fluorophore for that tranche of probes/variants using CY5/Crimson channel. In certain methods of the invention, and as exemplified for variant p.Y537C, a second probe is used with a different optical label, in this case, FAM detected by the Green channel. Although a discriminating probe is not necessarily required, even when used, the variants may still be identified simultaneously using less optical labels/channels than variants detected. Continuing, in the ROX/Red channel, a tranche of three (3) less frequent mutations (p.Y537N, p.Y537H, and p.Y537D) are detected and in the fourth tranche three (3) less frequent mutations are detected in the ATTO550/Orange channel (p.L536H, p.L536P, and p.L536R).

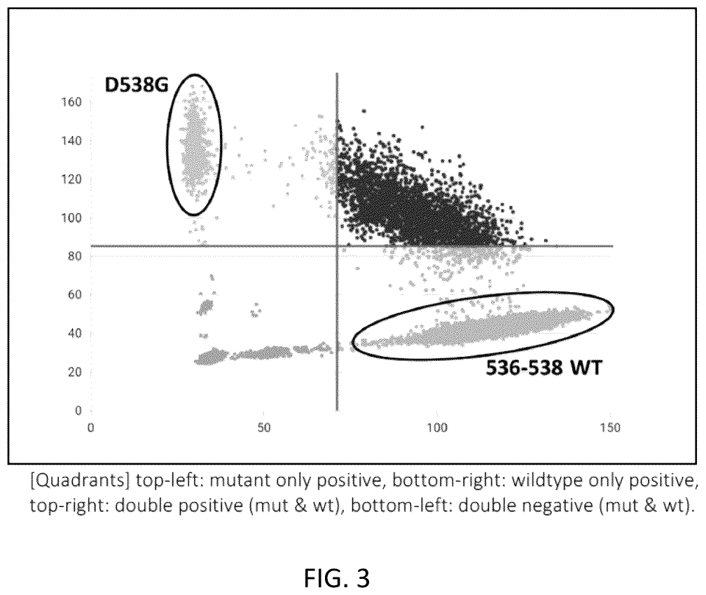

FIG. 3 provides a resulting two-color plot for the p.D538G mutation (using the D538G FAM/Green channel) against the wildtype probe signals (Hex/Yellow channel) for the dPCR reaction. In the plot, the Y-axis is the FAM/Green channel (variant) probe fluorescence intensity, and the X-axis is the HEX/Yellow channel (wildtype) fluorescence intensity. The two-color plot is divided into quadrants, in which: top-left is variant probe signal positive only positive; bottom-right is wildtype only positive; top-right is double positive (variant & wildtype); and bottom-left is double negative (variant & wildtype). In certain aspects, as was done here, it is advantageous to dedicate a channel to the most frequently occurring variant to maximize sensitivity and provide an internal validation of the assay. As shown, the signals from the FAM/Green channel from the D538G probes produce a clearly identifiable pattern when plotted against the HEX/Yellow channel, which indicates the presence of the variant in the sample.

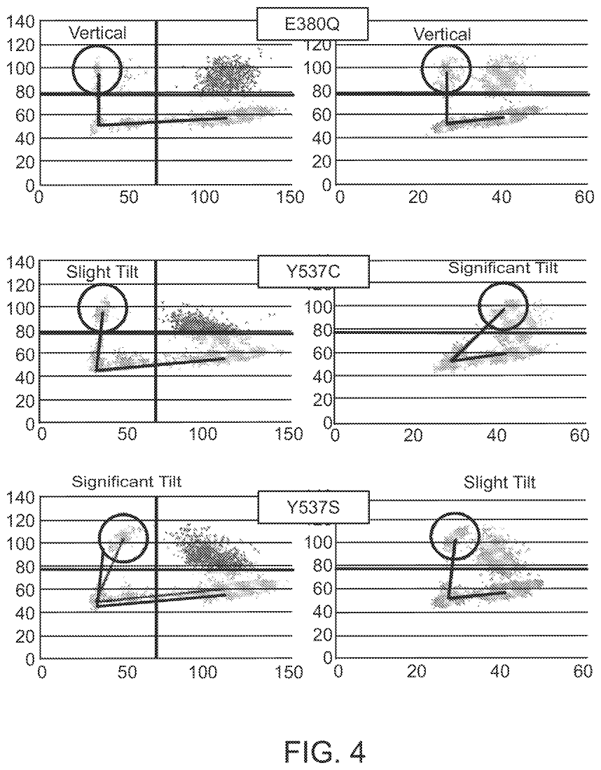

FIG. 4 provides similar plots for the tranche of variants containing variants p.Y537S, p.E380Q, and p.Y537N that are detected based on their CY5 probes using the Crimson channel. In the left plot for each variant, the X-axis represents the wildtype probe signal (HEX/Yellow) and the Y-axis represents the variant probe signals (CY5/Crimson). As shown, when plotted, each variant produces a signature pattern in its deviation relative to the wildtype plot—vertical for p.E380Q, slightly tilted for Y537C. and significantly tilted for Y537S. As described in FIGS. 1-2, included in this assay was a small amount of the Y537H probe, which had a FAM label detectable using the Green channel. The righthand plot for each variant shows the results of plotting the Green channel signals versus the Crimson channel signals. As shown, the deviation from the wildtype plot changes when these channels are interrogated, which helps discriminate the presence of the Y537C and Y537H probes.

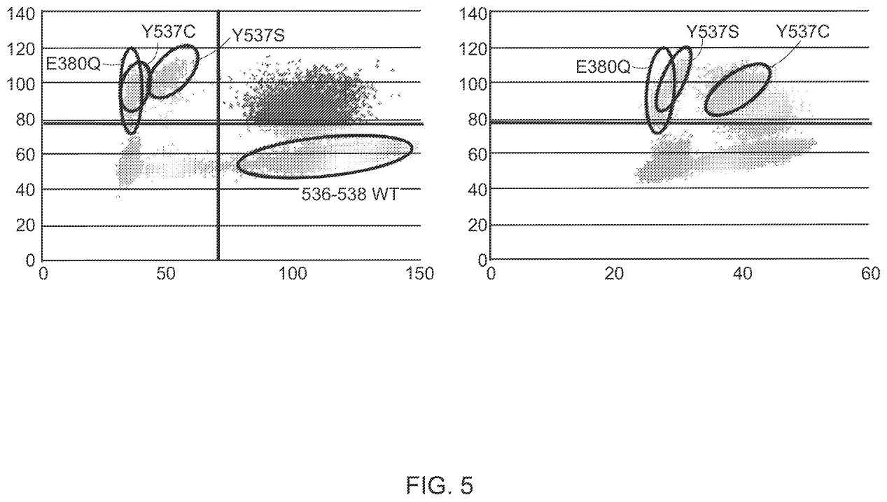

FIG. 5 provides a composite of both the right and left plots of FIG. 4. As shown, each

5 mutation provides a characteristic pattern or fingerprint relative to the wildtype signals, which may be used to identify the presence or absence of each variant sequence in the sample.

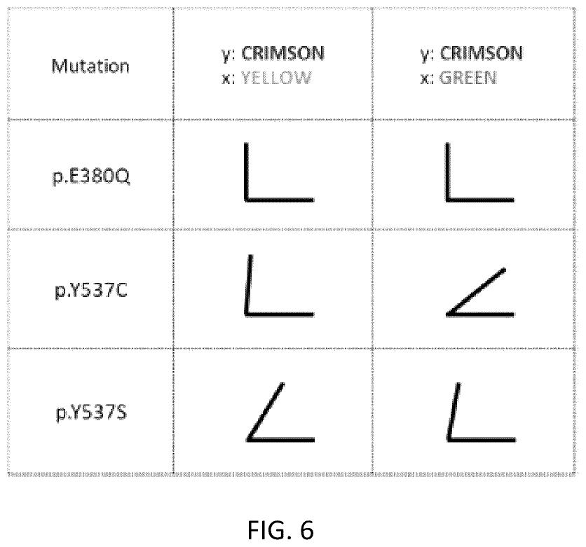

FIG. 6 provides schema for each variant's distinct fingerprints/patterns when interrogated using probes as described herein. Thus, methods of the invention may be used not only to detect variant sequences based on their characteristic dPCR pattern/fingerprint relative to the wildtype probe signal, but also used to discern the patterns/fingerprints, whether for an individual subject or for a population-wide assay.

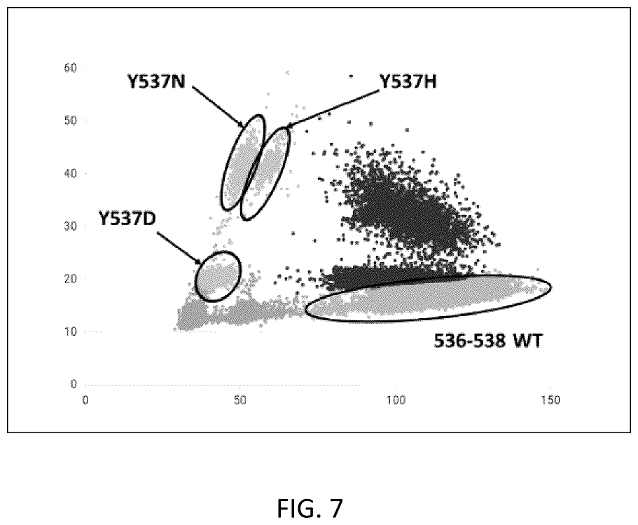

FIG. 7 provides a composite of the dPCR signals plotted for the variants in the tranche containing Y537D, Y537N, and Y537H using the ROX/Red channel corresponding to the variant probes and HEX/Yellow for the wildtype. The sum of the three variant frequencies is estimated to be 8.7%. Signal quality of the two more common variants (Y537N and Y537H, 8.2% combined) is readily detected. Further, the signal amplitude for Y537D, which is extremely rare (0.47%) is low, but also detectable.

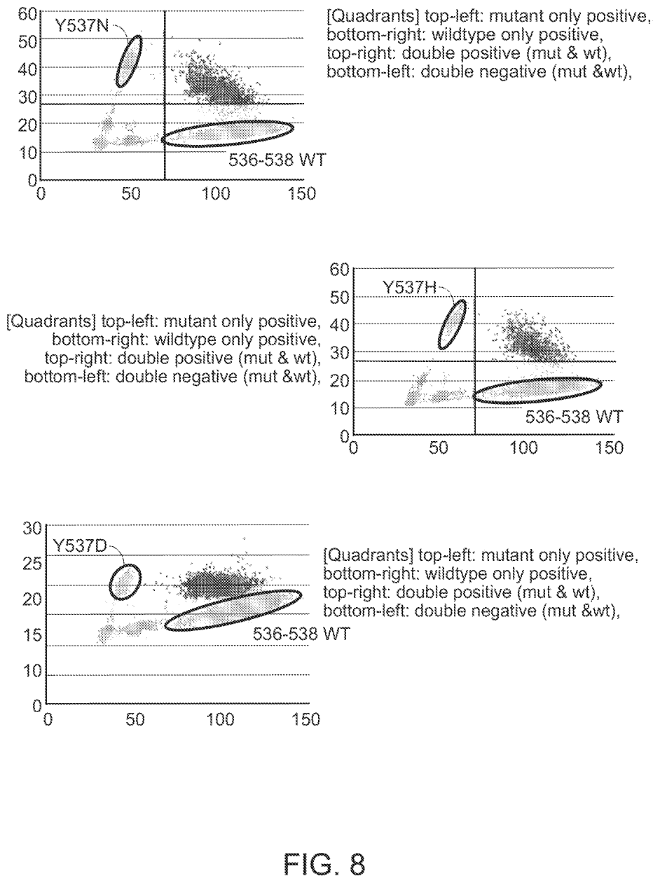

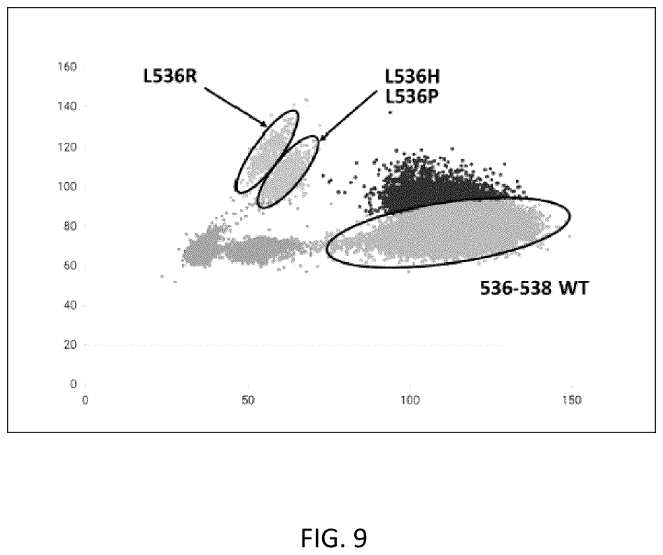

FIG. 8 provides the individual plots for the Y537D, Y537N, and Y537H variants, which were used to discern the patterns/fingerprints for each variant. FIG. 9 provides a composite of the dPCR signals plotted for the variants in the tranche containing L536R, L536H, and L536P using the ATTO550/Orange channel corresponding to the variant probes and HEX/Yellow for the wildtype. As shown, a pattern/fingerprint is clearly evident for each of these rare variant sequences when plotted against the wildtype channel.

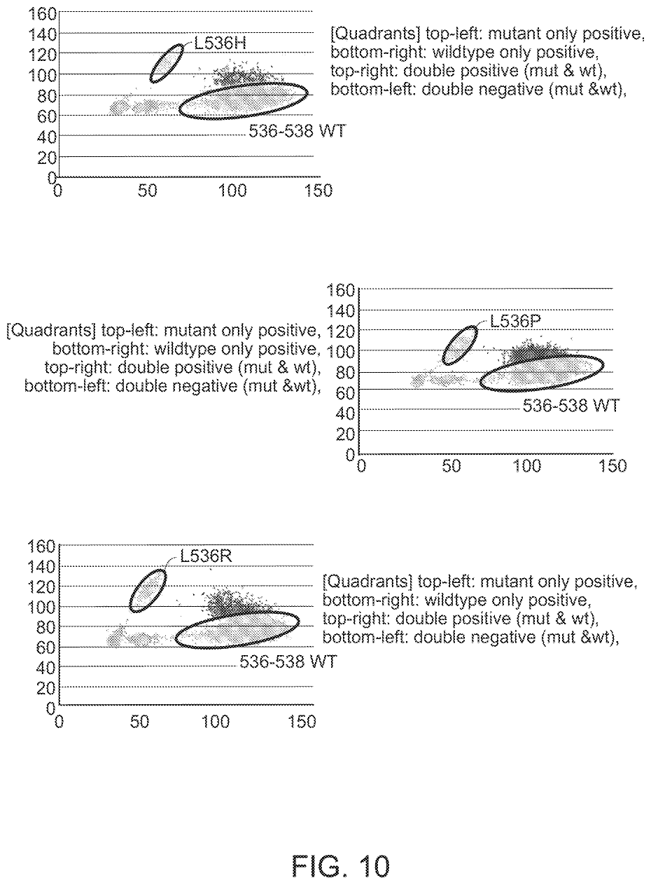

FIG. 10 provides the individual plots for the L536R, L536H, and L536P variants, which were used to discern the patterns/fingerprints for each variant.

Methods of the present invention are useful for detecting whether variant ESR1 sequences are present in a sample, which may comprise a mixture of target nucleic acids wherein only a portion of the target nucleic acids may comprise ESR1 variant sequences. In particular, the methods are useful for detecting the presence of a plurality of ESR1 variant sequences in a sample comprising target nucleic acids of which only a minor fraction may potentially comprise an ESR1 variant sequence.

In certain methods of the invention detecting the presence of variant sequences, the methods may include providing a sample comprising one or more target nucleic acids, partitioning the sample into partitions such as droplets; (iii) a pre-amplification such as an asymmetrical incremental polymerase reaction (AIPR) in the droplets; and (iv) PCR within the droplets (optionally with primers that were also used in iii). In such embodiments, methods include both incremental copying (e.g., AIPR) and then exponential signal generation (e.g., PCR) all within the dPCR compartment (e.g., droplets), with all the ingredients added at the start. Such embodiments may use amplification and/or reaction step as shown in U.S. Pat. No. 11,066,707, incorporated by reference. Methods may use primers that include Primer-H and Primer-L, discussed in detail below, allowing an AIPR and then PCR to happen sequentially within a single droplet. The PCR may preferably be digital PCR (e.g., with fluorescent probes, and methods preferably include detecting the presence of one or more ESR1 variant sequences.

Methods of the invention may comprise the use of an AIPR, i.e., to increase relative abundance of a target ESR1 sequence within a sample. Certain embodiments discussed herein perform the asymmetrical amplification using a primer-H and a primer-L. See also U.S. Pat. No. 11,066,707 and U.S. Pub. 2022/0056533 A1, both incorporated by reference.

The methods of the invention have a very low limit of detection. This enables a detection of target variant sequences that are often present at very low levels, and/or detection of the presence of variant sequences potentially present at very low levels in mixtures comprising other target nucleic acid sequences.

### Sample and Target Nucleic Acids

The sample may be any sample in which it is desirable to detect whether said variant sequence is present. For example, if the variant sequence is indicative of a clinical condition, the sample may be a sample from an individual at risk of acquiring said clinical condition. The variant sequences may differ from the wild-type sequence by substitution(s), deletion(s) and/or insertions(s).

The methods of the invention further provide that the one or more targeted nucleic acid molecules are associated with an ESR1 variant sequence, wherein the ESR1 variant sequence may be a variant of a wildtype nucleic acid sequence. In particular, methods are providing for detecting single nucleotide variants (SNVs) of ESR1.

In some embodiments, methods of the invention include one or more steps that diagnoses a subject based on the detection of one or more ESR1 variant sequences. Such a diagnosing step may, for example diagnose a subject with breast cancer or a type of breast cancer using a detected variant sequence, reporting a likelihood that the patient has or will develop such disease based on the identification of one or more variant sequence, and assessing the relative frequencies of identified variants to assess disease progression or disease response to a treatment.

Variant sequences identified using methods of the invention may be variants associated with a particular type or stage of breast cancer, or of a breast cancer having a particular characteristic (e.g., metastasized, drug resistant, and drug responsive). For example, certain ESR1 mutations are known to be associated with patient outcomes and/or specific types of breast cancer. In certain aspects, methods of the invention may provide information used in therapeutic decisions, guidance and monitoring, as well as development and clinical trials of therapies for a for breast cancer. For example, treatment efficacy can be monitored by comparing detected ESR1 variants and/or ESR1 variant frequencies from before, during, and after treatment. Longitudinal monitoring may be used to assess increases or decreases in variant sequences or frequencies, identify new or absent variant sequences after treatment, which may, for example, guide subsequent treatment decisions. In certain aspects, diagnosing a subject includes diagnosing the subject with a particular stage or type of breast cancer associated with one or more detected ESR1 sequence variant. The sample may be any biological sample, including a bodily fluid sample comprises bile, blood, plasma, serum, sweat, saliva, urine, feces, phlegm, mucus, sputum, tears, cerebrospinal fluid, synovial fluid, pericardial fluid, lymphatic fluid, semen, vaginal secretion, products of lactation or menstruation, amniotic fluid, pleural fluid, rheum, or vomit.

### Amplification and Pre-Amplification

The methods of the current invention may include the use of amplification reactions.

Amplification of a nucleic acid is the generation of copies of said nucleic acid.

The methods of the invention also include a pre-amplification step such as AIPR. In preferred embodiments, methods may include (i) providing a sample comprising one or more target nucleic acids, (ii) partitioning the sample into partitions such as droplets; (iii) a pre-amplification such as an asymmetrical incremental polymerase reaction (AIPR) in the droplets; and (iv) PCR within the droplets (optionally with primers that were also used in iii). In such embodiments, methods include both incremental copying (e.g., AIPR) and then exponential signal generation (e.g., PCR) all within the dPCR compartment (e.g., droplets), with all the ingredients added at the start. Such embodiments may use amplification and/or reaction step as shown in U.S. Pat. No. 11,066,707, incorporated by reference. Methods may use primers that include Primer-H and Primer-L, discussed in detail below, allowing an AIPR and then PCR to happen sequentially within a single droplet. The PCR may preferably be digital PCR (e.g., with fluorescent probes, and methods preferably include detecting the presence of one or more ESR1 variant sequences.

In other optional embodiments, the pre-amplification may be described as a variant enrichment sample preparation reaction step. Such a pre-amplification may amplify only select targets by a limited, controlled, or known amount. For example, ten cycle of linear pre-amplification with a primer specific for a variant would increase abundance of copies of that variant in the sample about tenfold. Because the abundance is increased by a known amount, if sequences in the reaction mixture are later quantified (e.g., by digital PCR), the quantities of those sequences in the original sample can be determined (using the know amount by which abundance of the select variants was increased). Additionally, the pre-amplification greatly increases the probability of detection of those variants, which aids in detection of very rare sequences in a sample. The pre-amplification may proceed using un-paired primers (e.g., single primers) that get extended through, and copy, a target of interest. In some embodiments, an incremental pre-amplification comprises the use of at least a couple of primers, wherein only one primer is active in the pre-amplification. The said active primer may be active due to a difference in the melting temperature (Tm) and annealing temperatures of the primers. The use of these primers for asymmetrical incremental amplification is disclosed in U.S. Pat. No. 11,066,707, which is hereby incorporated by reference in its entirety.

Methods of the invention may use an incremental pre-amplification to increase the abundance of a target of interest in a sample, even when that target is present in very low numbers. This may alleviate problems with molecular detection assays that rely on PCR to amplify target, in which due to the stochastic nature of PCR, targets present only in very low numbers may go undetected and also in which PCR reactions are plagued by “dead volumes” that go undetected. Those problems are addressed by selectively increasing the abundance of a target of interest in what is described here as a pre-amplification step. In one embodiment, the pre-amplification step is not-exponential, i.e., is not PCR. Instead, the pre-amplification step is preferably incremental which may be taken to mean that extension products from one round of pre-amplification are not substrates for copying by any reverse primer. Rare targets of interest are increased in abundance by this pre-amplification step. The increase in abundance is approximately linear (not exponential) over a cycle of pre-amplification.

It may be preferable to perform incremental pre-amplification (and not exponential) so that other material in the sample is also still present an accessible to subsequent amplification steps. Preferred embodiments use pre-amplification that is specific to a genetic sequence selected for clinical significance, such as a structural variant that is likely to persist, e.g., even after cancer treatment such as chemotherapy.

Preferably, in methods of the invention, a sequence containing a possible variant is selected and then pre-amplified. The pre-amplification (e.g., AIPR) specifically increases the abundance of copies of the selected variant of the sample, after which digital PCR (dPCR) provides a signal indicated the presence of the selected variant(s). In fact, embodiments of the invention may be multiplexed using, e.g., differently labeled fluorescent hydrolysis probes to interrogate the sample for multiple variants simultaneously, while always using fewer distinct fluorescent reporters than variants detected. Assays of the invention are useful for multiplexed detection of very rare targets in a sample, and may be particularly useful for detecting cancer-specific variant sequences such as in circulating tumor DNA (ctDNA) in a blood or plasma sample, such as in a liquid biopsy. Moreover, as discussed further herein, the incremental preamplification and the exponential amplification of dPCR can proceed in the presence of the same set of reagents (primers, dNTPs, polymerase, ions, probes, etc.) without requiring a clean-up step, or a reagent change, or the addition of reagents as the assay progresses. In fact, embodiments discussed herein use a primer pair that functions to incrementally pre-amplify a target under one thermocycle and exponentially amplify the target for dPCR under a different thermocycle.

In methods of the invention nucleic acid polymerase enzymes used may have different elongation temperatures. The elongation temperature is a temperature allowing enzymatic activity of the nucleic acid polymerase following primer annealing. Typically, a nucleic acid polymerase has activity over a temperature range, and thus the elongation temperature may be any temperature within that range. Most nucleic acid polymerases have a temperature optimum but retain activity at other temperatures than the temperature optimum. In such cases, the elongation temperature may be any temperature allowing the primer to anneal and the nucleic acid polymerase has activity even if the temperature is not the optimum temperature. At the elongation temperature of a nucleic acid polymerase, the enzyme is capable of catalyzing synthesis a new nucleic acid strand complementary to the template strand at the elongation temperature. In certain methods, the elongation temperature is near the melting temperature of the primer-H. Thus, a nucleic acid polymerase may be chosen, which has polymerase activity at a temperature near the melting temperature of primer-H and/or the primer-H may be designed to have a melting temperature near the elongation temperature.

In preferred embodiments, the product produced during AIPR in partitions (e.g., droplets) is subject to PCR within the partitions. In some embodiments, primers used in the invention are a part of a plurality of pairs of primers capable of amplification of different target nucleic acid sequences. Pre-amplification can be performed under a first temperature control then, after optionally adding any further reagents to the tube and optionally without any clean-up, PCR may be performed under a second temperature control. Methods of the invention may be performed without any cleanup steps, which could lead to loss of analyte material.

Methods of the invention may involve the use of PCR reagents for the sample preparation reactions and the dPCR. The sample preparation reaction and/or dPCR may include at least part of the sample, the set of primers, and sufficient PCR reagents to allow a polymerase reaction. Methods and reagents useful for performing a PCR reaction are well known to the skilled person. For example, the PCR reaction may comprise any of the nucleic acid polymerase and PCR reagents, described herein below in the section “PCR reagents”. Depending on the mode of detecting whether the sample preparation product comprises the variant sequence, the sample preparation reaction may also comprise detection reagents. PCR reagents are reagents which are added to a PCR in addition to nucleic acid polymerase, sample and set of primers. The PCR reagents at least comprise nucleotides. In additional the PCR reagents may comprise other compounds such as salt(s) and buffer(s).

For most purposes the PCR reagents comprise nucleotides. Thus, the PCR reagents may comprise deoxynucleoside triphosphates (dNTPs), in particular all of the four naturally-occurring deoxynucleoside triphosphates (dNTPs).

The PCR reagents frequently comprise deoxyribonucleoside triphosphate molecules, including dATP, dCTP, dGTP, dTTP. In some cases, dUTP is added.

The PCR reagents may also comprise compounds useful in assisting the activity of the nucleic acid polymerase. Thus, the PCR reagent may comprise a divalent cation, e.g., magnesium ions. Said magnesium ions may be added on the form of e.g., magnesium chloride or magnesium acetate (MgCl2) or magnesium sulfate is used.

The PCR reagents may also comprise one or more of the following:
non-specific blocking agents such as BSA or gelatin from bovine skin, betalactoglobulin, casein, dry milk, or other common blocking agents, o non-specific background/blocking nucleic acids (e.g., salmon sperm DNA),
biopreservatives (e.g., sodium azide),
PCR enhancers (e.g., Betaine, Trehalose, etc.),
inhibitors (e.g., RNAse inhibitors).

The PCR reagent a may also contain other additives, e.g., dimethyl sulfoxide (DMSO), glycerol, betaine (mono)hydrate (N,N,N-trimethylglycine=[caroxy-methyl]trimethylammonium), trehalose, 7-Deaza-2′-deoxyguanosine triphosphate (dC7GTP or 7-deaza-2′-dGTP), formamide (methanamide), tettrmethylammonium chloride (TMAC), other tetraalkylammonium derivaties (e.g., tetraethyammonium chloride (TEA-Cl) and tetrapropylammonium chloride (TPrA-Cl), non-ionic detergent (e.g., Triton X-100, Tween 20, Nonidet P-40 (NP-40)), or PREXCEL-Q.

The PCR reagents may comprise a buffering agent.

In some cases, a non-ionic Ethylene Oxide/Propylene Oxide block copolymer is added to the aqueous phase in a concentration of about 0.1%, 0.2%, 0.3%, 0.4%, 0.5%, 0.6%, 0.7%, 0.8%, 0.9%, or 1.0%. Common biosurfactants include non-ionic surfactants such as Pluronic F-68, Tetronics, Zonyl FSN. Pluronic F-68 can be present at a concentration of about 0.5% w/v.

A wide range of common, commercial PCR buffers from varied vendors can be substituted for the buffered solution.

The methods of the invention generally include the use of a nucleic acid polymerase. The nucleic acid polymerase may be any nucleic acid polymerase, such as a DNA polymerase. The nucleic acid polymerase should have activity at the elongation temperature.

The nucleic acid polymerase may be a DNA polymerase with 5′ to 3′ exonuclease activity. This may in particular be the case in the methods of the invention, wherein the methods or kits involves use of a detection probe, such as a TaqMan detection probe.

Any DNA polymerase, e.g., a DNA polymerase with 5′ to 3′ exonuclease activity that catalyzes primer extension can be used. For example, a thermostable DNA polymerase can be used. Preferably, the nucleic acid polymerase is a Taq polymerase, enabling the use of TaqMan probes to identify variant sequences as described herein.

The methods of the invention include a sample preparation reaction step, which could be either asymmetric incremental amplification or symmetrical exponential amplification or a combination of the two. Incremental amplification may proceed using unpaired, e.g., single primers (even if multiple primers are used, each may be “single” in the sense of not being paired with a reverse primer that anneals to the extension product of the single primer). In some embodiments, incremental, pre-amplification may include the use of at least two primers, wherein only one primer is active in the sample preparation reaction. For example, the said active primer is active due to a difference in the melting temperature (Tm) and annealing temperatures of the provided primers. More preferably, the step of asymmetrical incremental amplification includes: (i) providing a pair of primers capable of amplification of a target nucleic acid, wherein the pair of primers comprises a primer-H and a primer-L. According to the methods of the invention, the melting temperature of primer-H may be about 10° C. to about 22° C. higher than the melting temperature of primer-L, and primer-L comprises a sequence complementary to a fragment of the elongation product of primer-H, and the sample preparation reaction is performed. The sample preparation reactions also include nucleic acid polymerase having polymerase activity, primer(s), and PCR reagents.

In the embodiments with primer-H and primer-L, the incremental preamplification will proceed using an annealing temperature at which primer-H (but not primer-L) anneals. During that incremental amplification, primer-H will function as a “single” primer (despite the presence of primer-L). Primer-L will not anneal to anything to any meaningful extent because the reaction mixture is not brought down to the annealing temperature of primer-L. The incremental preamplification may be run for a predetermined number of cycles (e.g., one, or five, or sixty-four, etc.) or for a fixed amount of time, or until the sample exhibits a result (change in optical density, cleavage of a fluorescent probe, etc.). After the incremental preamplification, then the reaction mixture may be subject to exponential amplification. For the exponential amplification, at the annealing step, the temperature is brought down to the annealing temperature of primer-L, which promotes annealing of both primer-H and primer-L.

It is important to note that preceding paragraphs describes the function of primer-H and primer-L as those primers may function among many other primers. For example, tens, hundreds, or thousands, or more loci can be probed in parallel using a corresponding number of primer pairs. For an example, in a particular assay, 24 loci are being probed in parallel (but that number 24 is arbitrary, and could just as easily be 1, or 2, or 3, or 6, or 17, or 96, or 99, or 384, or 1,000, or 1,536, or an integer multiple of any of those numbers, etc.) An assay of the invention may use a primer pair for each loci, e.g., may use 24 primer pairs. Any one or any number of those primer pairs may fit the description of primer-H and primer-L. However, and this is important, it may be desirable to pre-amplify only certain loci, so any number of the primer pairs may include forward and reverse primer that each have an annealing temperature essentially the same as for a primer-L.

In other embodiments, the pre-amplification proceeds with unpaired “single” primers (e.g., that operate at the primer-H annealing temperature. After that, the reaction mixture (original sample, plus added reagents, plus preamplification product) may be subject to conditions for exponential amplification. The exponential amplification may use primer pairs, any one of which may “match” all or part of the single primer, or than anneal to targets within the length of the extension product of the unpaired single primers. Those reactions may proceed at different temperatures, or the paired primers may not be made available until after the incremental preamplification. For example, the paired primers may be added (e.g., by microfluidic handling) or released from confinement or attachment (e.g., by chemical, temperature, or photo lysis of a hydrogel bead).

In methods of the invention, an asymmetrical incremental amplification may be used, which includes: providing a pair of primers capable of amplification of a target nucleic acid, wherein the pair of primers comprises a primer-H and a primer-L, wherein the melting temperature of primer-H is about 10° C. to about 22° C. higher than the melting temperature of primer-L, and wherein primer-L comprises a sequence complementary to a fragment of the elongation product of primer-H; providing a nucleic acid polymerase having polymerase activity at an elongation temperature; and preparing sample preparation reactions, each comprising a part of the sample, the set of primers, the nucleic acid polymerase, and PCR reagents. The melting temperature of primer-H may be about 10° C. higher than the melting temperature of primer-L. Preferably, the melting temperature of primer-H is about 12° C. higher than the melting temperature of primer-L. More preferably, the melting temperature of primer-H is about 16° C. higher than the melting temperature of primer-L. In certain methods, an asymmetric incremental amplification may also include a set of primers, wherein at least one primer is specifically capable of amplification of only one strand of the target nucleic acid sequence, and performing the sample preparation reaction in a solution including a nucleic acid polymerase having polymerase activity, and the sample preparation reaction is performed. The sample preparation reactions also include nucleic acid polymerase having polymerase activity, primer(s), and PCR reagents.

The invention may further include a step of symmetric exponential amplification, wherein the symmetric exponential amplification include the steps of (i) providing a set of primers specifically capable of amplification of the target nucleic acid sequence; (ii) providing a nucleic acid polymerase having polymerase activity at an elongation temperature; (iii) preparing sample preparation reactions each comprising a part of the sample, the set of primers, the nucleic acid polymerase, and PCR reagents; and performing the sample preparation reaction.

The methods of the current invention may include an asymmetrical incremental amplification followed by symmetrical exponential amplification. The steps involved in asymmetrical incremental amplification and symmetrical exponential amplification are outlined above.

The methods of the current invention may include AIPR followed by PCR in partitions such as droplets. In certain methods, the AIPR and the PCR are performed using the same set of primers.

In certain methods of the invention an asymmetric incremental amplification may be activated at a higher temperature as compared to symmetric exponential amplification. For example, a thermocycler may be programmed to go down only to the higher annealing temperature for the incremental amplification but to go to a lower annealing temperature for exponential amplification.

Methods of the invention may include a step of conducting a plurality of PCR reactions on the sample, which has undergone the sample preparation reaction, wherein the step includes a pair of primers capable of specific amplification of a target nucleic acid, a nucleic acid polymerase having a polymerase activity at an elongation temperature, preparing PCR reactions, wherein each PCR reaction comprises a part of the sample, the set of primers, the nucleic acid polymerase, PCR reagents; and performing symmetrical exponential amplification. Preferably, these PCR reactions are conventional PCR reactions. Preferably, the PCR is digital PCR (dPCR), and uses variant sequence discriminating probes as described. The sample including the products following the sample preparation reaction step may be used as direct sample input for the PCR. Optionally, the pair of primers used in the invention are a part of a plurality of pairs of primers capable of amplification of different target nucleic acid sequences.

The methods of the invention may further include the use of a plurality of pairs of primers capable of amplification of different target nucleic acid sequences. Preferably, the methods of the invention include the use of multiplex PCR.

### Primer-H and Primer-L

Methods of the invention may include the use of several primers including primers named as primer-H and primer-L. Primer-H is a primer having a high melting temperature, whereas primer-L is a primer having a low melting temperature. The melting temperature of a primer is the temperature at which 50% of the primer forms a stable double helix with its complementary sequence and the other 50% is separated to single strand molecules. The melting temperature may also be referred to as Tm or Tm. Preferably, the Tm as used herein is calculated using a nearest-neighbor method based on the method described in Breslauer, 1986, Predicting DNA duplex stability from the base sequence, PNAS 83:3746-50 (incorporated by reference) using a salt concentration parameter of 50 mM and primer concentration of 900 nM. For example, the method is implemented by the software “Multiple Primer Analyzer” from Life Technologies/Thermo Fisher Scientific Inc. The methods of the invention may use of a set of primers comprising a primer-H and a primer-L, wherein the melting temperature of primer-H is about 10° C. to about 22° C., preferably at least 10° C., more preferably at least 15° C. higher than the melting temperature of primer-L, and wherein primer-L contains a sequence complementary to the elongation product of primer-H.

The primer-H is preferably designed as a primer for amplification of the target sequence or the sequence complementary to the target sequence. Thus, the primer-H is preferably capable of annealing to either the target nucleic acid sequence or to the sequence complementary to the target nucleic acid sequence. For example, primer-H may be capable of annealing to the complementary strand of the target nucleic acid sequence at the 5′-end or close to the 5′-end of the target nucleic acid sequence, or the primer-H may be capable of annealing to the target nucleic acid sequence at the 3′-end or close to the 3′-end of the target nucleic acid sequence. Thus, the primer-H may comprise a sequence identical to the 5′-end of the target nucleic acid sequence. The primer-H may even consist of a sequence identical to the 5′-end of the target nucleic acid sequence. The primer-H may also comprise a sequence identical to the target nucleic acid sequence. Thus, the primer-H may comprise a sequence complementary to the 3′-end of the target nucleic acid sequence. The primer-H may even consist of a sequence complementary to the 3′-end of the target nucleic acid sequence.

Similarly, primer-L is preferably designed as a primer for amplification of the target sequence or the sequence complementary to the target sequence. If the primer-H is designed for amplification of the target sequence, the primer-L is preferably designed for amplification of the sequence complementary to the target sequence and vice versa. Thus, the primer-L is preferably capable of annealing to either the target nucleic acid sequence or to the sequence complementary to the target nucleic acid sequence. If primer-H is capable of annealing to the target nucleic acid sequence, then primer-L is preferably capable of annealing to the sequence complementary to the target nucleic acid sequence and vice versa. For example, primer-L may be capable of annealing to the complementary strand of the target nucleic acid sequence at the 5′-end or close to the 5′-end of the target nucleic acid sequence, or the primer-L may be capable of annealing to the target nucleic acid sequence at the 3′-end or close to the 3′-end of the target nucleic acid sequence. Thus, the primer-L may comprise a sequence identical to the 5′-end of the target nucleic acid sequence. The primer-L may even consist of a sequence identical to the 5′-end of the target nucleic acid sequence. The primer-L may also comprise a sequence identical to the target nucleic acid sequence. Thus, the primer-L may comprise a sequence complementary to the 3′-end of the target nucleic acid sequence. The primer-L may even consist of a sequence complementary to the 3′-end of the target nucleic acid sequence.

Primer-H may have a nucleotide sequence identical to the sequence at the 5′-end of the target nucleic acid sequence and the primer-L comprises or consists of a sequence identical to the complementary sequence of the 3′-end of the target nucleic acid sequence.

Primer-L may have a nucleotide sequence identical to the sequence at the 5′-end of the target nucleic acid sequence and the primer-H comprises or consists of a sequence identical to the complementary sequence of the 3′-end of the target nucleic acid sequence.

Primer-H and primer-L are designed to have the melting temperatures as indicated herein. The skilled person will be capable of designing primer-H and primer-L to have the desired melting temperature by adjusting the sequence of the primers, the length of the primers and optionally by incorporating nucleotide analogues as described herein above in the section “Set of primers”.

Primer-H is designed so that it has an annealing temperature which is significantly higher than the annealing temperature of primer-L, for example at least 10° C. higher. Thus, the melting temperature of primer-H may be at least 12° C. higher, for example at least 15° C. higher, preferably at least 14° C. higher, even more preferably at least 16° C. higher, yet more preferably 18° C. higher, such as at least 20° C. higher, for example in the range of 15 to 50° C., such as in the range of 15 to 40° C., for example in the range of 15 to 25° C. higher than the melting temperature of primer-L.

In general, it may be preferred that the melting temperature of primer-H is as high as possible, but not higher than the highest functional elongation temperature of at least one nucleic acid polymerase. Said elongation temperature does not need to be the optimum temperature for said nucleic acid polymerase, but it is preferred that at least one nucleic acid polymerase has activity at the melting temperature primer-H. Thus, the melting temperature of the primer-H may approach or may even exceed 80° C.

Since it is also preferred that the melting temperature of primer-L is sufficiently high to ensure specific annealing of primer-L to the target nucleic acid sequence/the complementary sequence of the target nucleic acid sequence, and the melting temperature of primer-H should be significantly higher than the melting temperature of primer-H, then frequently, the melting temperature of primer-H is at least 60° C. The melting temperature of primer-H may also frequently be at least 70° C. The melting temperature of primer-H may for example be in the range of 60 to 90° C., for example in the range of 60 to 85° C., such as in the range of 70 to 85° C., for example in the range of 70 to 80° C.

The melting temperature of primer-L is preferably sufficiently high to ensure specific annealing of primer-L to the target nucleic acid sequence/the complementary sequence of the target nucleic acid sequence, but also significantly lower than the melting temperature of primer-H. Frequently, the melting temperature of the primer-L is in the range of 30 to 55° C., such as in the range of 35 to 55° C., preferably in the range of 40 to 50° C.

The methods of the invention may also include the use of a set primers or a plurality of primers. A set of primers or a plurality of primers contain two or more different primers. A set of primers contains at least a pair of primers specifically capable of amplification of a target nucleic acid. Furthermore, a set of primers according to the invention contains at least a primer-H and a primer-L. Thus, wherein the set of primers contains only two different primers, then set of primers contains a primer-H and a primer-L, wherein the primer-H and primer-L are capable of amplification of a target nucleic acid.

### Detection

The methods of the invention in general comprise a step of detecting, whether the sample comprises one or more ESR1 variant sequence. The detection reagent may include detection probes. Detection probes may include nucleotide oligomers or polymers, which optionally may comprise nucleotide analogues. Frequently, the detection probe may be a DNA oligomer. Typically, the detection probe is linked to an optically detectable label (e.g., a fluorescent label), for example by a covalent bond. The detectable label may be any type of detectable label, but preferably is an optically detectable label such as a fluorophore.

The detection probe is in general capable of specifically binding a target ESR1 nucleic acid sequence. For example, the detection probe may be capable of specifically binding the target ESR1 nucleic acid comprising a variant sequence. Thus, the detection probe may be capable of annealing to the target ESR1 nucleic acid sequence or to the sequence complementary to the target ESR1 nucleic acid sequence. Thus, the detection probe may comprise a sequence identical to a fragment of the target nucleic acid sequence or the sequence complementary to the target nucleic acid sequence. It is generally preferred that the detection probe comprises a sequence different to the sequence of any of the primers of the set of primers.

### Quantification

Methods of the invention may include steps of providing a sample comprising one or more target ESR1 nucleic acids; aliquoting the sample into a plurality of subsamples; conducting asymmetric incremental polymerase reaction (AIPR) and then polymerase chain reaction (PCR) on the subsamples; and detecting ESR1 variant nucleic acid sequences. Using targeted pre-amplification, an increased number of copies of the target is available may be available for subsequent PCR reactions used to identify the variant sequences. An advantage of the AIPR is that it reduces false positives. Any suitable sample comprising DNA from a subject may be assayed for variants of ESR1.

The invention also provides a reference assay for detection of patient's wild-type copy number. In particular, the invention provides a method for detecting wildtype DNA and any variant ESR1 sequences of interest included within that DNA. Accordingly, the invention provides an assay, comprising the use of a pair of primers and probe, to detect and/or quantify a wildtype (reference) sequence of DNA. The invention further provides an assay to calculate variant allele fraction (VAF) for the target ESR1 nucleic acids in the sample. In certain aspects, a target nucleic acid may be a variant of the wild-type nucleic acid sequence in the sample.

### Example

An exemplary method of the invention was used to simultaneously detect the ESR1 variants (D538G, Y537S, Y537C, Y537N, Y537H, Y537D, L536H, L536P, and L536R) described in FIGS. 1-10. Two ESR1 target sequences were amplified to produce a first and a second amplicon using a primer pair for each target sequence. The first amplicon included the nucleotides of the wildtype ESR1 nucleotide sequence encoding amino acids 529-552 of the ESR1 protein sequence. The second amplicon included the nucleotides of the wildtype ESR1 nucleotide sequence encoding amino acids 372-395 of the ESR1 protein sequence. Thus, between the two amplicons, the genomic location of each variant was included.

FIGS. 1-2 show the probe sequences, the ESR1 variants they interrogate, and the associated optical labels for each probe.

In the assay, the most common mutation, p.D538G, was detected based on the corresponding probe's optical label using a dedicated channel (FAM/Green). The next three most common mutations (p.Y537S, p.E380Q, and p.Y537N) were detected based on the fluorophore for that tranche of probes/variants using CY5/Crimson channel. For variant p.Y537C, a second probe was used with a different optical label, in this case, FAM detected by the Green channel. As shown in FIG. 1, the concentration of this second probe for p.Y537C was provided in a low concentration relative to the other probes. Continuing, in the ROX/Red channel, a tranche of three (3) less frequent mutations (p.Y537N, p.Y537H, and p.Y537D) were detected and in the fourth tranche three (3) less frequent mutations were detected in the ATTO550/Orange channel (p.L536H, p.L536P, and p.L536R).

As described above, all variant sequences were detected. For each variant, a characteristic dPCR signal was discerned relative to the expected wildtype sequence dPCR signal.

### INCORPORATION BY REFERENCE

References and citations to other documents, such as patents, patent applications, patent publications, journals, books, papers, web contents, publicly accessible databases, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for detecting a plurality of recurrent variant estrogen receptor 1 (ESR1) nucleic acid sequences, the method comprising:
obtaining a sample comprising a ESR1 nucleic acids from a subject;
partitioning the sample into reaction partitions;
performing a digital polymerase chain reaction (dPCR) in each partition using:
a probe that comprises a first optical label and specifically binds to amplicons containing a wildtype ESR1 sequence;
a first set of different probes, wherein each probe comprises a second optical label, wherein each different probe of the set specifically binds to amplicons that comprise a different, variant sequence, wherein said different variant sequences comprise at least a first ESR1 variant sequence, a second ESR1 variant sequence, and a third ESR1 variant sequence;
generating a two-color plot from the detected optical signals using a dedicated channel for each optical label; and
identifying the presence of one or more ESR1 variant sequences based on a deviation from an expected wild-type cluster on the two-color plot.

2. The method of claim 1, wherein the dPCR step further uses a probe that comprises a third optical label, wherein the probe is specific for a fourth ESR1 variant sequence.

3. The method of claim 2, wherein the dPCR step further uses a probe that comprises a third optical label, wherein the probe is specific for the first ESR1 variant sequence.

4. The method of claim 3, wherein the generating step comprises generating a two-color plot using the dedicated channel for the second optical label versus the dedicated channel for the third optical label, wherein the presence of the first ESR1 sequence is based on a shift in the deviation from a two-color plot of the first and second optical channels and the two-color plot of the second and third optical channels.

5. The method of claim 4, wherein the amplicons are further contacted with an unlabeled probe that specifically binds to a wildtype sequence at the genomic location of the first ESR1 variant sequence.

6. The method of claim 4, wherein the dPCR step further uses: a second set of different probes, wherein each probe comprises a fourth optical label, wherein each different probe of the set specifically binds to amplicons that comprise a different, variant sequence, wherein said different variant sequences comprise a fifth ESR1 variant sequence, a sixth ESR1 variant sequence, and a seventh ESR1 variant sequence.

7. The method of claim 6, wherein the dPCR step further uses: a third set of different probes, wherein each probe comprises a fifth optical label, wherein each different probe of the set specifically binds to amplicons that comprise a different, variant sequence, wherein said different variant sequences comprise an eighth ESR1 variant sequence, a ninth ESR1 variant sequence, and a tenth ESR1 variant sequence.

8. The method of claim 7, wherein prior to the dPCR step, the method includes an amplifying step that specifically amplifies two different ESR1 sequences, thereby producing a first amplicon and a second amplicon.

9. The method of claim 8, wherein the first and second amplicons are less than 65 bp in length.

10. The method of claim 9, wherein the first amplicon includes genomic locations of the second, third, fourth, fifth, sixth, seventh, eighth, ninth, and tenth ESR1 variant sequences and the second amplicon includes a genomic location of the first variant ESR1 sequence.

11. The method of claim 10, wherein the first amplicon includes an ESR1 sequence encoding amino acids 526 through 538 of SEQ ID NO: 2.

12. The method of claim 11, wherein the second amplicon includes an ESR1 sequence encoding amino acid 380 of SEQ ID NO: 2.

13. The method of claim 12, wherein a mutant allele frequency for each ESR1 variant sequence is known.

14. The method of claim 7, wherein the optical labels comprise FAM, HEX, CY5, ROX, and ATTO550.

15. The method of claim 1, wherein the sample comprises highly fragmented target nucleic acids.

16. The method of claim 15, wherein the sample comprises cell-free DNA (cfDNA).

17. The method of claim 15, wherein the sample is selected from the group consisting of: a tissue sample, a circulating tumor cell, a tissue biopsy sample, a formalin-fixed, paraffin-embedded (FFPE) tissue sample, and a liquid biopsy sample.

20. The method of claim 7, wherein: the first variant sequence comprises the sequence of SEQ ID NO: 3; the second variant sequence comprises the sequence of SEQ ID NO: 4; the third variant sequence comprises the sequence of SEQ ID NO: 5; the fourth variant sequence comprises the sequence of SEQ ID NO: 6; the fifth variant sequence comprises the sequence of SEQ ID NO: 7; the sixth variant sequence comprises the sequence of SEQ ID NO: 8; the seventh variant sequence comprises the sequence of SEQ ID NO: 9; the eighth variant sequence comprises the sequence of SEQ ID NO: 10; the ninth variant sequence comprises the sequence of SEQ ID NO: 11; and/or the tenth variant sequence comprises the sequence of SEQ ID NO: 12.
